SEQUENCE LISTING (171 sequences)

SEQ ID NO: 1 - protein, 184 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 2 - protein, 170 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - protein, 175 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 203 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 5 - protein, 178 aa, Homo sapiens
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 6 - protein, 164 aa, Homo sapiens
LDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 7 - protein, 169 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 8 - protein, 197 aa, Homo sapiens
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 9 - protein, 378 aa, Artificial Sequence
  dimeric hu 4-1BBL (71-254) connected by (G4S)2 linker
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 366 aa, Artificial Sequence
  dimeric hu 4-1BBL (71-248) connected by (G4S)2 linker
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 11 - protein, 360 aa, Artificial Sequence
  dimeric hu 4-1BBL (80-254) connected by (G4S)2 linker
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 12 - protein, 416 aa, Artificial Sequence
  dimeric hu 4-1BBL (52-254) connected by (G4S)2 linker
PWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 13 - protein, 7 aa, Mus Musculus
GFSFSSY

SEQ ID NO: 14 - protein, 3 aa, Mus Musculus
GGR

SEQ ID NO: 15 - protein, 9 aa, Mus Musculus
TGRVYFALD

SEQ ID NO: 16 - protein, 11 aa, Mus Musculus
SESVDTSDNSF

SEQ ID NO: 17 - protein, 3 aa, Mus Musculus
RSS

SEQ ID NO: 18 - protein, 6 aa, Mus Musculus
NYDVPW

SEQ ID NO: 19 - protein, 120 aa, Mus Musculus
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSS

SEQ ID NO: 20 - protein, 111 aa, Mus Musculus
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIK

SEQ ID NO: 21 - protein, 120 aa, Artificial Sequence
  humanized variant -heavy chain variable domain VH of PD1-0103-0312, -0313, -0314 and -0315
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 22 - protein, 111 aa, Artificial Sequence
  humanized variant -light chain variable domain VL of PD1-0103-0312
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 23 - protein, 111 aa, Artificial Sequence
  humanized variant -light chain variable domain VL of PD1-0103-0313
DVVMTQSPLSLPVTLGQPASISCRASESVDTSDNSFIHWYQQRPGQSPRLLIYRSSTLESGVPDRFSGSGSGTDFTLKISRVEAEDVGVYYCQQNYDVPWTFGQGTKVEIK

SEQ ID NO: 24 - protein, 111 aa, Artificial Sequence
  humanized variant -light chain variable domain VL of PD1-0103-0314
EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIK

SEQ ID NO: 25 - protein, 111 aa, Artificial Sequence; identical to SEQ ID NO: 24
  humanized variant -light chain variable domain VL of PD1-0103-0315

SEQ ID NO: 26 - protein, 8 aa, Artificial Sequence
  heavy chain HVR-H1, PD1-0098
GYSITSDY

SEQ ID NO: 27 - protein, 3 aa, Artificial Sequence
  heavy chain HVR-H2, PD1-0098
YSG

SEQ ID NO: 28 - protein, 9 aa, Artificial Sequence
  heavy chain HVR-H3, PD1-0098
HGSAPWYFD

SEQ ID NO: 29 - protein, 12 aa, Artificial Sequence
  light chain HVR-L1, PD1-0098
SQNIVHSDGNTY

SEQ ID NO: 30 - protein, 3 aa, Artificial Sequence
  light chain HVR-L2, PD1-0098
KVS

SEQ ID NO: 31 - protein, 6 aa, Artificial Sequence
  light chain HVR-L3, PD1-0098
GSHFPL

SEQ ID NO: 32 - protein, 120 aa, Artificial Sequence
  heavy chain variable domain VH, PD1-0098
DVQLQESGPGLVKPSQSLSLTCTVTGYSITSDYAWNWIRQFPGDKLEWLGYITYSGFTNYNPSLKSRISISRDTSKNQFFLQLNSVATEDTATYYCARWHGSAPWYFDYWGRGTTLTVSS

SEQ ID NO: 33 - protein, 112 aa, Artificial Sequence
  light chain variable domain VL, PD1-0098
DVLMTQTPLSLPVSLGDQASISCRSSQNIVHSDGNTYLEWYLQKPGQSPNLLIYKVSRRFSGVPDRFSGSGSGTDFTLKISRVEAEDLGVYYCFQGSHFPLTFGAGTKLELK

SEQ ID NO: 34 - protein, 8 aa, Artificial Sequence
  heavy chain HVR-H1, PD1-0050
GYSITSDY

SEQ ID NO: 35 - protein, 3 aa, Artificial Sequence
  heavy chain HVR-H2, PD1-0050
YTG

SEQ ID NO: 36 - protein, 9 aa, Artificial Sequence
  heavy chain HVR-H3, PD1-0050
MDYYGSTLD

SEQ ID NO: 37 - protein, 11 aa, Artificial Sequence
  light chain HVR-L1, PD1-0050
SESVDRYGNSF

SEQ ID NO: 38 - protein, 3 aa, Artificial Sequence
  light chain HVR-L2, PD1-0050
RAS

SEQ ID NO: 39 - protein, 6 aa, Artificial Sequence
  light chain HVR-L3, PD1-0050
NNEDPY

SEQ ID NO: 40 - protein, 120 aa, Artificial Sequence
  heavy chain variable domain VH, PD1-0050
DVQLQESGPGLVKPSQSLSLTCTVTGYSITSDYAWNWIRQFPGNKLEWMGYITYTGRTSYNPSLKSRISITRDTSKNQFFLQLNSVTTEDTATYYCAREMDYYGSTLDYWGQGTTLTVSS

SEQ ID NO: 41 - protein, 111 aa, Artificial Sequence
  light chain variable domain VL, PD1-0050
KIVLTQSPASLAVSLRQRATISCRASESVDRYGNSFIHWYQQKPGQPPKVLIYRASNLESGFPARFSGSGSRTDFTLTIDPVEADDAATYYCQQNNEDPYTFGSGTKLEIK

SEQ ID NO: 42 - protein, 7 aa, Artificial Sequence
  heavy chain HVR-H1, PD1-0069
GYTFTDY

SEQ ID NO: 43 - protein, 3 aa, Artificial Sequence
  heavy chain HVR-H2, PD1-0069
YSG

SEQ ID NO: 44 - protein, 7 aa, Artificial Sequence
  heavy chain HVR-H3, PD1-0069
GITTGFA

SEQ ID NO: 45 - protein, 11 aa, Artificial Sequence
  light chain HVR-L1, PD1-0069
SKGVSTSSYSF

SEQ ID NO: 46 - protein, 3 aa, Artificial Sequence
  light chain HVR-L2, PD1-0069
YAS

SEQ ID NO: 47 - protein, 6 aa, Artificial Sequence
  light chain HVR-L3, PD1-0069
SREFPW

SEQ ID NO: 48 - protein, 118 aa, Artificial Sequence
  heavy chain variable domain VH, PD1-0069
QVQLQQSGPELVRPGVSVKISCKGSGYTFTDYAMHWVKQSHARTLEWIGVISTYSGDTNYNQKFKDKATMTVDKSSSTAYLELARMTSEDSAIYYCARLGITTGFAYWGQGTLVTVSA

SEQ ID NO: 49 - protein, 111 aa, Artificial Sequence
  light chain variable domain VL, PD1-0069
DIVLTQSPASLAVSLGQRATISCRASKGVSTSSYSFMHWYQQKPRQPPKLLIKYASYLESGVPARFSGSGSGTDFTLNIHPVEEEDAATYYCHHSREFPWTFGGGTKLEIK

SEQ ID NO: 50 - protein, 7 aa, Artificial Sequence
  heavy chain HVR-H1, PD1-0073
GFTFSNY

SEQ ID NO: 51 - protein, 3 aa, Artificial Sequence
  heavy chain HVR-H2, PD1-0073
GGR

SEQ ID NO: 52 - protein, 5 aa, Artificial Sequence
  heavy chain HVR-H3, PD1-0073
YYGID

SEQ ID NO: 53 - protein, 7 aa, Artificial Sequence
  light chain HVR-L1, PD1-0073
SQDVTTA

SEQ ID NO: 54 - protein, 3 aa, Artificial Sequence
  light chain HVR-L2, PD1-0073
WAS

SEQ ID NO: 55 - protein, 6 aa, Artificial Sequence
  light chain HVR-L3, PD1-0073
HYSIPW

SEQ ID NO: 56 - protein, 116 aa, Artificial Sequence
  heavy chain variable domain VH, PD1-0073
EVKLVESGGGLVKPGGSLKLSCAASGFTFSNYGMSWIRQTPEKGLEWVATISGGGRDTYYPDSVKGRFTISRDNVKNNLYLQMSSLRSEDTAFYYCASYYYGIDYWGQGTSVTVSS

SEQ ID NO: 57 - protein, 107 aa, Artificial Sequence
  light chain variable domain VL, PD1-0073
DIVMTQPHKFMSTSVGDRVRITCKASQDVTTAVAWYQQKPGQSPKLLIYWASTRHTGVPDRFTGSGSGTEFTLTISSVQAEDLALYYCQQHYSIPWTFGGGTKLEIK

SEQ ID NO: 58 - protein, 7 aa, Artificial Sequence
  heavy chain HVR-H1, PD1-0078
GYTFTST

SEQ ID NO: 59 - protein, 3 aa, Artificial Sequence
  heavy chain HVR-H2, PD1-0078
SDS

SEQ ID NO: 60 - protein, 3 aa, Artificial Sequence
  heavy chain HVR-H3, PD1-0078
PFD

SEQ ID NO: 61 - protein, 7 aa, Artificial Sequence
  light chain HVR-L1, PD1-0078
SQDVSTA

SEQ ID NO: 62 - protein, 3 aa, Artificial Sequence
  light chain HVR-L2, PD1-0078
SAS

SEQ ID NO: 63 - protein, 6 aa, Artificial Sequence
  light chain HVR-L3, PD1-0078
HYSHPF

SEQ ID NO: 64 - protein, 114 aa, Artificial Sequence
  heavy chain variable domain VH, PD1-0078
QVQLQQPGAELVKPGASVKMSCKASGYTFTSTWMHWVKQRPGQGLEWIGAIDPSDSYTTYNQKFKGKATLTVDTSSTTAYMQLSSLTSEDSAVYYCTRSPFDYWGQGTTLTVSS

SEQ ID NO: 65 - protein, 107 aa, Artificial Sequence
  light chain variable domain VL, PD1-0078
DIVMTQSHKFMSTSVGDRVSITCKASQDVSTAVAWYQQKPGQSPKLLIYSASYRYTGVPDRFTGSGSGTDFTFAISSVQAEDLAVYYCQQHYSHPFTFGSGTKLEIK

SEQ ID NO: 66 - protein, 8 aa, Artificial Sequence
  heavy chain HVR-H1, PD1-0102
GYSITSGY

SEQ ID NO: 67 - protein, 3 aa, Artificial Sequence
  heavy chain HVR-H2, PD1-0102
SSG

SEQ ID NO: 68 - protein, 6 aa, Artificial Sequence
  heavy chain HVR-H3, PD1-0102
RNWYFD

SEQ ID NO: 69 - protein, 13 aa, Artificial Sequence
  light chain HVR-L1, PD1-0102
SQSLLNSGTQKNY

SEQ ID NO: 70 - protein, 3 aa, Artificial Sequence
  light chain HVR-L2, PD1-0102
WAS

SEQ ID NO: 71 - protein, 6 aa, Artificial Sequence
  light chain HVR-L3, PD1-0102
DYTFPL

SEQ ID NO: 72 - protein, 117 aa, Artificial Sequence
  heavy chain variable domain VH, PD1-0102
DVQLQESGPDLVKPSQSLSLTCTVTGYSITSGYSWHWIRQFPGNKLEWMGFIHSSGDTNYNPSLKSRISFTRDTSKNQFFLQLSSLTDEDTATYYCATYRNWYFDVWGAGTTVTVSS

SEQ ID NO: 73 - protein, 113 aa, Artificial Sequence
  light chain variable domain VL, PD1-0102
DIVMTQSPSSLTVTAGEKVTMRCKSSQSLLNSGTQKNYLTWYQQKPGQPPKLLIYWASTRESGVPNRFTGSGSGTDFTLTISSVQAEDLSVYYCQSDYTFPLTFGGGTKLELK

SEQ ID NO: 74 - protein, 722 aa, Artificial Sequence
  Dimeric hu 4-1BBL (71-254) - CL* Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 297 aa, Artificial Sequence
  Monomeric hu 4-1BBL (71-254) - CH1*
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 76 - protein, 722 aa, Artificial Sequence
  Dimeric hu 4-1BBL (71-254) - CL Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 297 aa, Artificial Sequence
  Monomeric hu 4-1BBL (71-254) - CH1
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 78 - protein, 710 aa, Artificial Sequence
  Dimeric hu 4-1BBL (71-248) - CL* Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 291 aa, Artificial Sequence
  Monomeric hu 4-1BBL (71-248) - CH1*
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSC

SEQ ID NO: 80 - protein, 710 aa, Artificial Sequence
  Dimeric hu 4-1BBL (71-248) - CL Fc knob chain
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 291 aa, Artificial Sequence
  Monomeric hu 4-1BBL (71-248) - CH1
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 82 - protein, 450 aa, Artificial Sequence
  anti-PD1 (0314) Fc hole chain
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 218 aa, Artificial Sequence
  anti-PD1 (0314) light chain
EIVLTQSPATLSLSPGERATLSCRASESVDTSDNSFIHWYQQKPGQSPRLLIYRSSTLESGIPARFSGSGSGTDFTLTISSLEPEDFAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - protein, 837 aa, Artificial Sequence
  anti-PD1(0314) Fc hole chain fused to dimeric hu 4-1BBL (71-254)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 85 - protein, 643 aa, Artificial Sequence
  anti-PD1(0314) Fc knob chain fused to monomeric hu 4-1BBL (71-254)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 86 - protein, 825 aa, Artificial Sequence
  anti-PD1(0314) Fc hole chain fused to dimeric hu 4-1BBL (71-248)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 87 - protein, 637 aa, Artificial Sequence
  anti-PD1(0314) Fc knob chain fused to monomeric hu 4-1BBL (71-248)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 88 - protein, 602 aa, Artificial Sequence
  Fc hole dimeric ligand (71-248) chain
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 89 - protein, 404 aa, Artificial Sequence
  Fc hole monomeric ligand (71-248) chain
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 90 - protein, 815 aa, Artificial Sequence
  anti-PD1(0314) Fc knob chain fused to dimeric hu 4-1BBL (71-248)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 91 - protein, 414 aa, Artificial Sequence
  Fc knob chain fused to monomeric hu 4-1BBL (71-248)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 92 - protein, 637 aa, Artificial Sequence
  anti-PD1(0314) Fc hole monomeric ligand (71-248) chain
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 93 - protein, 602 aa, Artificial Sequence
  Fc knob chain fused to dimeric hu 4-1BBL (71-248)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGL

SEQ ID NO: 94 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 95 - protein, 205 aa, Homo sapiens
MTPPERLFLPRVCGTTLHLLLLGLLLVLLPGAQGLPGVGLTPSAAQTARQHPKMHLAHSTLKPAAHLIGDPSKQNSLLWRANTDRAFLQDGFSLSNNSLLVPTSGIYFVYSQVVFSGKAYSPKATSSPLYLAHEVQLFSSQYPFHVPLLSSQKMVYPGLQEPWLHSMYHGAAFQLTQGDQLSTHTDGIPHLVLSPSTVFFGAFAL

SEQ ID NO: 96 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 97 - protein, 244 aa, Homo sapiens
MGALGLEGRGGRLQGRGSLLLAVAGATSLVTLLLAVPITVLAVLALVPQDQGGLVTETADPGAQAQQGLGFQKLPEEEPETDLSPGLPAAHLIGAPLKGQGLGWETTKEQAFLTSGTQFSDAEGLALPQDGLYYLYCLVGYRGRAPPGGGDPQGRSVTLRSSLYRAGGAYGPGTPELLLEGAETVTPVLDPARRQGYGPLWYTSVGFGGLVQLRRGERVYVNISHPDMVDFARGKTFFGAVMVG

SEQ ID NO: 98 - protein, 183 aa, Homo sapiens
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 99 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 100 - protein, 281 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 101 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 102 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 103 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 104 - protein, 281 aa, Homo sapiens [sequence omitted]

SEQ ID NO: 105 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 106 - protein, 249 aa, Homo sapiens
MAARRSQRRRGRRGEPGTALLVPLALGLGLALACLGLLLAVVSLGSRASLSAQEPAQEELVAEEDQDPSELNPQTEESQDPAPFLNRLVRPRRSAPKGRKTRARRAIAAHYEVHPRPGQDGAQAGVDGTVSGWEEARINSSSPLRYNRQIGEFIVTRAGLYYLYCQVHFDEGKAVYLKLDLLVDGVLALRCLEEFSATAASSLGPQLRLCQVSGLLALRPGSSLRIRTLPWAHLKAAPFLTYFGLFQVH

SEQ ID NO: 107 - protein, 250 aa, Homo sapiens [sequence omitted]

SEQ ID NO: 108 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 109 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 110 - protein, 251 aa, Homo sapiens
MAEDLGLSFGETASVEMLPEHGSCRPKARSSSARWALTCCLVLLPFLAGLTTYLLVSQLRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 111 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 112 - protein, 391 aa, Homo sapiens
MGYPEVERRELLPAAAPRERGSQGCGCGGAPARAGEGNSCLLFLGFFGLSLALHLLTLCCYLELRSELRRERGAESRLGGSGTPGTSGTLSSLGGLDPDSPITSHLGQPSPKQQPLEPGEAALHSDSQDGHQMALLNFFFPDEKPYSEEESRRVRRNKRSKSNEGADGPVKNKKKGKKAGPPGPNGPPGPPGPPGPQGPPGIPGIPGIPGTTVMGPPGPPGPPGPQGPPGLQGPSGAADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 113 - protein, 205 aa, Homo sapiens
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 114 - protein, 133 aa, Homo sapiens
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 115 - protein, 132 aa, Homo sapiens
VSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 116 - protein, 5 aa, Artificial Sequence
  Peptide linker G4S
GGGGS

SEQ ID NO: 117 - protein, 10 aa, Artificial Sequence
  Peptide linker (G4S)2
GGGGSGGGGS

SEQ ID NO: 118 - protein, 10 aa, Artificial Sequence
  Peptide linker (SG4)2
SGGGGSGGGG

SEQ ID NO: 119 - protein, 15 aa, Artificial Sequence
  Peptide linker (G4S)3
GGGGSGGGGSGGGGS

SEQ ID NO: 120 - protein, 14 aa, Artificial Sequence
  Peptide linker G4(SG4)2
GGGGSGGGGSGGGG

SEQ ID NO: 121 - protein, 20 aa, Artificial Sequence
  Peptide linker (G4S)4
GGGGSGGGGSGGGGSGGGGS

SEQ ID NO: 122 - protein, 10 aa, Artificial Sequence
  Peptide linker GSPGSSSSGS
GSPGSSSSGS

SEQ ID NO: 123 - protein, 8 aa, Artificial Sequence
  Peptide linker GSGSGSGS
GSGSGSGS

SEQ ID NO: 124 - protein, 8 aa, Artificial Sequence
  Peptide linker GSGSGNGS
GSGSGNGS

SEQ ID NO: 125 - protein, 8 aa, Artificial Sequence
  Peptide linker GGSGSGSG
GGSGSGSG

SEQ ID NO: 126 - protein, 6 aa, Artificial Sequence
  Peptide linker GGSGSG
GGSGSG

SEQ ID NO: 127 - protein, 4 aa, Artificial Sequence
  Peptide linker GGSG
GGSG

SEQ ID NO: 128 - protein, 8 aa, Artificial Sequence
  GGSGNGSG
GGSGNGSG

SEQ ID NO: 129 - protein, 8 aa, Artificial Sequence
  Peptide inker GGNGSGSG
GGNGSGSG

SEQ ID NO: 130 - protein, 6 aa, Artificial Sequence
  Peptide linker GGNGSG
GGNGSG

SEQ ID NO: 131 - DNA, 2166 bp, Artificial Sequence
  nucleotide sequence of dimeric hu 4-1BBL (71-254) - CL* Fc knob chain
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggcactgacagtggatctgcctcctgccagctccgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggcctgccttctccaagaagcgaaggcggaggcggatctggcggcggaggatctagagagggacccgaactgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgctgctggcgcagctgcactggctctgactgtggacctgccaccagcctctagcgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccctgccggactgccaagccctagatcagaagggggcggaggttccggagggggaggatctcgtacggtggctgcaccatctgtctttatcttcccacccagcgaccggaagctgaagtctggcacagccagcgtcgtgtgcctgctgaataacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgcgacaagacccacacctgtcctccatgccctgcccctgaagctgctggcggccctagcgtgttcctgttccccccaaagcccaaggacaccctgatgatcagccggacccctgaagtgacctgcgtggtggtggatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 132 - DNA, 891 bp, Artificial Sequence
  nucleotide sequence of monomeric hu 4-1BBL (71-254) - CH1*
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggctctgacagtggatctgcctcctgccagctccgaggcccggaatagcgcatttgggtttcaaggccggctgctgcacctgtctgccggccagagactgggagtgcatctgcacacagaggccagagccaggcacgcctggcagctgacacagggcgctacagtgctgggcctgttcagagtgacccccgagattcctgccggcctgcctagccctagatctgaaggcggcggaggttccggaggcggaggatctgctagcacaaagggccccagcgtgttccctctggcccctagcagcaagagcacatctggcggaacagccgccctgggctgcctggtggaagattacttccccgagcccgtgaccgtgtcctggaattctggcgccctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacgagaaggtggaacccaagtcctgc

SEQ ID NO: 133 - DNA, 1350 bp, Artificial Sequence
  nucleotide sequence of anti-PD1(0314) Fc hole chain
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 134 - DNA, 654 bp, Artificial Sequence
  nucleotide sequence of anti-PD1 (0314) light chain
gagatcgtgctgacccagagccccgccacactctccctgtctcctggcgaaagggctaccctttcatgcagagcaagcgagtccgtggacacttctgataacagtttcattcactggtatcagcagaagccagggcagagcccccgcctgctcatctatcggtcctctacactggagtcagggattcctgccaggtttagcggctccggttctgggaccgacttcactttgaccatcagtagcctggaaccagaggattttgctgtgtactattgtcaacagaattacgacgtcccctggacattcggccagggaaccaaagtggagataaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 135 - DNA, 2166 bp, Artificial Sequence
  nucleotide sequence of dimeric hu 4-1BBL (71-254) - CL Fc knob chain
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggcactgacagtggatctgcctcctgccagctccgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggcctgccttctccaagaagcgaaggcggaggcggatctggcggcggaggatctagagagggacccgaactgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgctgctggcgcagctgcactggctctgactgtggacctgccaccagcctctagcgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccctgccggactgccaagccctagatcagaagggggcggaggttccggagggggaggatctcgtacggtggccgctccctccgtgtttatctttcccccatccgatgaacagctgaaaagcggcaccgcctccgtcgtgtgtctgctgaacaatttttaccctagggaagctaaagtgcagtggaaagtggataacgcactgcagtccggcaactcccaggaatctgtgacagaacaggactccaaggacagcacctactccctgtcctccaccctgacactgtctaaggctgattatgagaaacacaaagtctacgcctgcgaagtcacccatcagggcctgagctcgcccgtcacaaagagcttcaacaggggagagtgtgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 136 - DNA, 891 bp, Artificial Sequence
  nucleotide sequence of monomeric hu 4-1BBL (71-254) - CH1
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggctctgacagtggatctgcctcctgccagctccgaggcccggaatagcgcatttgggtttcaaggccggctgctgcacctgtctgccggccagagactgggagtgcatctgcacacagaggccagagccaggcacgcctggcagctgacacagggcgctacagtgctgggcctgttcagagtgacccccgagattcctgccggcctgcctagccctagatctgaaggcggcggaggttccggaggcggaggatctgctagcaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgt

SEQ ID NO: 137 - DNA, 2511 bp, Artificial Sequence
  nucleotide sequence of anti-PD1(0314) Fc hole chain fused to dimeric hu 4-1BBL (71-254)
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggaggcggcggaagcggaggaggaggatccagagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggcactgacagtggatctgcctcctgccagctccgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggcctgccttctccaagaagcgaaggcggaggcggatctggcggcggaggatctagagagggacccgaactgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgctgctggcgcagctgcactggctctgactgtggacctgccaccagcctctagcgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccctgccggactgccaagccctagatcagaa

SEQ ID NO: 138 - DNA, 1929 bp, Artificial Sequence
  nucleotide sequence of anti-PD1(0314) Fc knob chain fused to monomeric hu 4-1BBL (71-254)
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccctgcagagatgagctgaccaagaaccaggtgtccctgtggtgtctggtcaagggcttctaccccagcgatatcgccgtggagtgggagagcaacggccagcctgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccccggcggaggcggcggaagcggaggaggaggatccagagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggcactgacagtggatctgcctcctgccagctccgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggcctgccttctccaagaagcgaa

SEQ ID NO: 139 - DNA, 2130 bp, Artificial Sequence
  nucleotide sequence of dimeric hu 4-1BBL (71-248) - CL* Fc knob chain
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggactgggaggcggcggatctggcggcggaggatctagagaaggacccgagctgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgcagcaggggctgcagcactggccctgactgtggacctgcccccagcttcttccgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccccgctggactgggcggaggcggttccggagggggaggatctcgtacggtggctgcaccatctgtctttatcttcccacccagcgaccggaagctgaagtctggcacagccagcgtcgtgtgcctgctgaataacttctacccccgcgaggccaaggtgcagtggaaggtggacaatgccctgcagagcggcaacagccaggaaagcgtgaccgagcaggacagcaaggactccacctacagcctgagcagcaccctgaccctgagcaaggccgactacgagaagcacaaggtgtacgcctgcgaagtgacccaccagggcctgtctagccccgtgaccaagagcttcaaccggggcgagtgcgacaagacccacacctgtcctccatgccctgcccctgaagctgctggcggccctagcgtgttcctgttccccccaaagcccaaggacaccctgatgatcagccggacccctgaagtgacctgcgtggtggtggatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 140 - DNA, 873 bp, Artificial Sequence
  nucleotide sequence of monomeric hu 4-1BBL (71-248) - CH1*
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggactgggaggcggaggttccggaggcggaggatctgctagcacaaagggccccagcgtgttccctctggcccctagcagcaagagcacatctggcggaacagccgccctgggctgcctggtggaagattacttccccgagcccgtgaccgtgtcctggaattctggcgccctgacaagcggcgtgcacacctttccagccgtgctgcagagcagcggcctgtactctctgagcagcgtcgtgacagtgcccagcagctctctgggcacccagacctacatctgcaacgtgaaccacaagcccagcaacaccaaggtggacgagaaggtggaacccaagtcctgc

SEQ ID NO: 141 - DNA, 2130 bp, Artificial Sequence
  nucleotide sequence of dimeric hu 4-1BBL (71-248) - CL Fc knob chain
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggactgggaggcggcggatctggcggcggaggatctagagaaggacccgagctgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgcagcaggggctgcagcactggccctgactgtggacctgcccccagcttcttccgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccccgctggactgggcggaggcggttccggagggggaggatctcgtacggtggccgctccctccgtgtttatctttcccccatccgatgaacagctgaaaagcggcaccgcctccgtcgtgtgtctgctgaacaatttttaccctagggaagctaaagtgcagtggaaagtggataacgcactgcagtccggcaactcccaggaatctgtgacagaacaggactccaaggacagcacctactccctgtcctccaccctgacactgtctaaggctgattatgagaaacacaaagtctacgcctgcgaagtcacccatcagggcctgagctcgcccgtcacaaagagcttcaacaggggagagtgtgacaagacccacacctgtcccccttgtcctgcccctgaagctgctggcggcccttctgtgttcctgttccccccaaagcccaaggacaccctgatgatcagccggacccccgaagtgacctgcgtggtggtggatgtgtcccacgaggaccctgaagtgaagttcaattggtacgtggacggcgtggaagtgcacaatgccaagaccaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 142 - DNA, 873 bp, Artificial Sequence
  nucleotide sequence of monomeric hu 4-1BBL (71-248) - CH1
agagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggactgggaggcggaggttccggaggcggaggatctgctagcaccaaaggcccttccgtgtttcctctggctcctagctccaagtccacctctggaggcaccgctgctctcggatgcctcgtgaaggattattttcctgagcctgtgacagtgtcctggaatagcggagcactgacctctggagtgcatactttccccgctgtgctgcagtcctctggactgtacagcctgagcagcgtggtgacagtgcccagcagcagcctgggcacccagacctacatctgcaacgtgaaccacaagcccagcaacaccaaggtggacaagaaggtggaacccaagtcttgt

SEQ ID NO: 143 - DNA, 2475 bp, Artificial Sequence
  nucleotide sequence of anti-PD1(0314) Fc hole chain fused to dimeric hu 4-1BBL (71-248)
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggaggcggcggaagcggaggaggaggatccagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagcaggcctgggaggcggcggatctggcggcggaggatctagagaaggacccgagctgtcccccgacgatcccgctgggctgctggatctgagacagggcatgttcgctcagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgcagcaggggctgcagcactggccctgactgtggacctgcccccagcttcttccgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatcccagccgggctc

SEQ ID NO: 144 - DNA, 1911 bp, Artificial Sequence
  nucleotide sequence anti-PD1(0314) Fc knob chain fused to monomeric hu 4-1BBL (71-248)
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccctgcagagatgagctgaccaagaaccaggtgtccctgtggtgtctggtcaagggcttctaccccagcgatatcgccgtggagtgggagagcaacggccagcctgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccccggcggaggcggcggaagcggaggaggaggatccagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattcctgccgggctc

SEQ ID NO: 145 - DNA, 1806 bp, Artificial Sequence
  nucleotide sequence of Fc hole dimeric ligand (71-248) chain
gacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggaggcggcggaagcggaggaggaggatccagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagcaggcctgggaggcggcggatctggcggcggaggatctagagaaggacccgagctgtcccccgacgatcccgctgggctgctggatctgagacagggcatgttcgctcagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgcagcaggggctgcagcactggccctgactgtggacctgcccccagcttcttccgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatcccagccgggctc

SEQ ID NO: 146 - DNA, 1212 bp, Artificial Sequence
  nucleotide sequence of Fc hole monomeric ligand (71-248) chain
gacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattcctgccgggctc

SEQ ID NO: 147 - DNA, 2445 bp, Artificial Sequence
  nucleotide sequence of anti-PD1(0314) Fc knob chain fused to dimeric hu 4-1BBL (71-248)
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccctgcagagatgagctgaccaagaaccaggtgtccctgtggtgtctggtcaagggcttctaccccagcgatatcgccgtggagtgggagagcaacggccagcctgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccccggcagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagcaggcctgggaggcggcggatctggcggcggaggatctagagaaggacccgagctgtcccccgacgatcccgctgggctgctggatctgagacagggcatgttcgctcagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgcagcaggggctgcagcactggccctgactgtggacctgcccccagcttcttccgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatcccagccgggctc

SEQ ID NO: 148 - DNA, 1242 bp, Artificial Sequence
  nucleotide sequence of Fc knob chain fused to monomeric hu 4-1BBL (71-248)
gacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccctgcagagatgagctgaccaagaaccaggtgtccctgtggtgtctggtcaagggcttctaccccagcgatatcgccgtggagtgggagagcaacggccagcctgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccccggcggaggcggcggaagcggaggaggaggatccagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattcctgccgggctc

SEQ ID NO: 149 - DNA, 1911 bp, Artificial Sequence
  nucleotide sequence of anti-PD1(0314) Fc hole monomeric ligand (71-248) chain
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggaggcggcggaagcggaggaggaggatccagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattcctgccgggctc

SEQ ID NO: 150 - DNA, 1806 bp, Artificial Sequence
  nucleotide sequence of Fc knob chain fused to dimeric hu 4-1BBL (71-248)
gacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccctgcagagatgagctgaccaagaaccaggtgtccctgtggtgtctggtcaagggcttctaccccagcgatatcgccgtggagtgggagagcaacggccagcctgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccccggcggaggcggcggaagcggaggaggaggatccagagagggccctgagctgagccctgatgatcctgccggactgctggacctgcggcagggaatgtttgcccagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagatctgctgctggcgccgctgctctggcactgacagtggatctgcctcctgccagcagcgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagcaggcctgggaggcggcggatctggcggcggaggatctagagaaggacccgagctgtcccccgacgatcccgctgggctgctggatctgagacagggcatgttcgctcagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgcagcaggggctgcagcactggccctgactgtggacctgcccccagcttcttccgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatcccagccgggctc

SEQ ID NO: 151 - DNA, 1335 bp, Artificial Sequence
  nucleotide sequence of DP47 Fc hole chain
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 152 - DNA, 645 bp, Artificial Sequence
  nucleotide sequence of DP47 light chain
gaaatcgtgttaacgcagtctccaggcaccctgtctttgtctccaggggaaagagccaccctctcttgcagggccagtcagagtgttagcagcagctacttagcctggtaccagcagaaacctggccaggctcccaggctcctcatctatggagcatccagcagggccactggcatcccagacaggttcagtggcagtggatccgggacagacttcactctcaccatcagcagactggagcctgaagattttgcagtgtattactgtcagcagtatggtagctcaccgctgacgttcggccaggggaccaaagtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 153 - protein, 445 aa, Artificial Sequence
  DP47 Fc hole chain
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 154 - protein, 215 aa, Artificial Sequence
  DP47 light chain
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 155 - DNA, 2496 bp, Artificial Sequence
  nucleotide sequence of DP47 Fc hole chain fused to dimeric hu 4-1BBL (71-254)
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccctccgtgttccccctggcccccagcagcaagagcaccagcggcggcacagccgctctgggctgcctggtcaaggactacttccccgagcccgtgaccgtgtcctggaacagcggagccctgacctccggcgtgcacaccttccccgccgtgctgcagagttctggcctgtatagcctgagcagcgtggtcaccgtgccttctagcagcctgggcacccagacctacatctgcaacgtgaaccacaagcccagcaacaccaaggtggacaagaaggtggagcccaagagctgcgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtggaggcggcggaagcggaggaggaggatccagagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggcactgacagtggatctgcctcctgccagctccgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggcctgccttctccaagaagcgaaggcggaggcggatctggcggcggaggatctagagagggacccgaactgtcccctgacgatccagccgggctgctggatctgagacagggaatgttcgcccagctggtggctcagaatgtgctgctgattgacggacctctgagctggtactccgacccagggctggcaggggtgtccctgactgggggactgtcctacaaagaagatacaaaagaactggtggtggctaaagctggggtgtactatgtgttttttcagctggaactgaggcgggtggtggctggggagggctcaggatctgtgtccctggctctgcatctgcagccactgcgctctgctgctggcgcagctgcactggctctgactgtggacctgccaccagcctctagcgaggccagaaacagcgccttcgggttccaaggacgcctgctgcatctgagcgccggacagcgcctgggagtgcatctgcatactgaagccagagcccggcatgcttggcagctgactcagggggcaactgtgctgggactgtttcgcgtgacacctgagatccctgccggactgccaagccctagatcagaa

SEQ ID NO: 156 - DNA, 1914 bp, Artificial Sequence
  nucleotide sequence of DP47 Fc knob chain fused to monomeric hu 4-1BBL (71-254)
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccctgcagagatgagctgaccaagaaccaggtgtccctgtggtgtctggtcaagggcttctaccccagcgatatcgccgtggagtgggagagcaacggccagcctgagaacaactacaagaccaccccccctgtgctggacagcgacggcagcttcttcctgtactccaaactgaccgtggacaagagccggtggcagcagggcaacgtgttcagctgcagcgtgatgcacgaggccctgcacaaccactacacccagaagtccctgagcctgagccccggcggaggcggcggaagcggaggaggaggatccagagagggccctgagctgagccccgatgatcctgctggactgctggacctgcggcagggcatgtttgctcagctggtggcccagaacgtgctgctgatcgatggccccctgtcctggtacagcgatcctggactggctggcgtgtcactgacaggcggcctgagctacaaagaggacaccaaagaactggtggtggccaaggccggcgtgtactacgtgttctttcagctggaactgcggagagtggtggccggcgaaggatctggctctgtgtctctggccctgcatctgcagcctctgagaagcgctgctggcgctgcagctctggcactgacagtggatctgcctcctgccagctccgaggcccggaatagcgcatttgggtttcaaggcaggctgctgcacctgtctgccggccagaggctgggagtgcatctgcacacagaggccagggctagacacgcctggcagctgacacagggcgctacagtgctgggcctgttcagagtgacccccgagattccagccggcctgccttctccaagaagcgaa

SEQ ID NO: 157 - protein, 832 aa, Artificial Sequence
  DP47 Fc hole chain fused to dimeric hu 4-1BBL (71-254)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 158 - protein, 638 aa, Artificial Sequence
  DP47 Fc knob chain fused to monomeric hu 4-1BBL (71-254)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 159 - DNA, 1335 bp, Artificial Sequence
  nucleotide sequence of DP47 heavy chain (hu IgG1 PGLALA)
gaggtgcaattgttggagtctgggggaggcttggtacagcctggggggtccctgagactctcctgtgcagcctccggattcacctttagcagttatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcagatgaacagcctgagagccgaggacacggccgtatattactgtgcgaaaggcagcggatttgactactggggccaaggaaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 160 - protein, 445 aa, Artificial Sequence
  DP47 heavy chain (hu IgG1 PGLALA)
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - DNA, 1350 bp, Artificial Sequence
  nucleotide sequence of PD1(0314) heavy chain (huIgG1 PGLALA)
gaagtgcagctgctcgaaagcggcgggggactggtccagcccggcggttccctgaggctgtcttgcgccgcttcagggttcagcttttcctcttacaccatgagttgggtcagacaggcacctggcaagggactggagtgggtcgccacaatcagcggtggcgggcgcgacatttattacccagattccgtgaaaggacggttcaccatctctagggacaactcaaagaatactctgtatttgcagatgaacagcctgagagctgaggatacagcagtttactactgtgtgctcctgaccggccgcgtctattttgcccttgactcctggggacaaggcactctggtgaccgtatctagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagctgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggcgcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 162 - protein, 450 aa, Artificial Sequence
  PD1(0314) heavy chain (huIgG1 PGLALA)
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 163 aa, Homo sapiens
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 164 - DNA, 681 bp, Artificial Sequence
  Nucleotide sequence of Fc hole chain
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtgcaccctgcccccatcccgggatgagctgaccaagaaccaggtcagcctctcgtgcgcagtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 165 - DNA, 1266 bp, Artificial Sequence
  Nucleotide sequence of human 4-1BB antigen Fc knob chain
ctgcaggacccctgcagcaactgccctgccggcaccttctgcgacaacaaccggaaccagatctgcagcccctgcccccccaacagcttcagctctgccggcggacagcggacctgcgacatctgcagacagtgcaagggcgtgttcagaacccggaaagagtgcagcagcaccagcaacgccgagtgcgactgcacccccggcttccattgtctgggagccggctgcagcatgtgcgagcaggactgcaagcagggccaggaactgaccaagaagggctgcaaggactgctgcttcggcaccttcaacgaccagaagcggggcatctgccggccctggaccaactgtagcctggacggcaagagcgtgctggtcaacggcaccaaagaacgggacgtcgtgtgcggccccagccctgctgatctgtctcctggggccagcagcgtgacccctcctgcccctgccagagagcctggccactctcctcaggtcgacgaacagttatattttcagggcggctcacccaaatctgcagacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccggaggcctgaacgacatcttcgaggcccagaagattgaatggcacgag

SEQ ID NO: 166 - protein, 227 aa, Artificial Sequence
  Fc hole chain
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 167 - protein, 422 aa, Artificial Sequence
  human 4-1BB antigen Fc knob chain
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQVDEQLYFQGGSPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKSGGLNDIFEAQKIEWHE

SEQ ID NO: 168 - protein, 15 aa, Artificial Sequence
  avi tag
GLNDIFEAQKIEWHE

SEQ ID NO: 169 - DNA, 594 bp, Artificial Sequence
  nucleotide sequence of human 4-1BB His
ctgcaggacccctgcagcaactgccctgccggcaccttctgcgacaacaaccggaaccagatctgcagcccctgcccccccaacagcttcagctctgccggcggacagcggacctgcgacatctgcagacagtgcaagggcgtgttcagaacccggaaagagtgcagcagcaccagcaacgccgagtgcgactgcacccccggcttccattgtctgggagccggctgcagcatgtgcgagcaggactgcaagcagggccaggaactgaccaagaagggctgcaaggactgctgcttcggcaccttcaacgaccagaagcggggcatctgccggccctggaccaactgtagcctggacggcaagagcgtgctggtcaacggcaccaaagaacgggacgtcgtgtgcggccccagccctgctgatctgtctcctggggccagcagcgtgacccctcctgcccctgccagagagcctggccactctcctcaggtcgacgaacagttatattttcagggcggctcaggcctgaacgacatcttcgaggcccagaagatcgagtggcacgaggctcgagctcaccaccatcaccatcac

SEQ ID NO: 170 - protein, 198 aa, Artificial Sequence
  human 4-1BB His
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQVDEQLYFQGGSGLNDIFEAQKIEWHEARAHHHHHH

SEQ ID NO: 171 - protein, 218 aa, Artificial Sequence
  anti-PD1 (0312) light chain
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2019/0185566 A1
(43) Pub. Date: Jun. 20, 2019

(54) Antigen Binding Molecules comprising a TNF family ligand trimer and PD1 binding moiety

(71) Applicant: Hoffmann-La Roche Inc., Little Falls, NJ (US)

(72) Inventors: Claudia Ferrara Koller, Schlieren (CH); Christina Claus, Schlieren (CH); Christian Klein, Schlieren (CH); Stefan Seeber, Penzberg (DE); Maria Amann, Schlieren (CH); Sandra Grau-Richards, Schlieren (CH); Peter Bruenker, Schlieren (CH); Pablo Umana, Schlieren (CH); Viktor Levitski, Schlieren (CH); Ekkehard Moessner, Schlieren (CH)

(73) Assignee: Hoffmann-La Roche Inc., Little Falls, NJ (US)

(21) Appl. No.: 16/189,041

(22) Filed: Nov. 13, 2018

Related U.S. Application Data
(63) Continuation of application No. PCT/EP2017/061241, filed on May 11, 2017.

(30) Foreign Application Priority Data
May 13, 2016 (EP) 16169487.2

(51) Int. Cl. C07K 16/28; C07K 14/705; A61P 35/00

(52) U.S. Cl. CPC C07K 16/2818; C07K 14/70575; A61P 35/00; C07K 2317/76; C07K 2317/55; C07K 2317/92; C07K 2317/24; C07K 2319/74

(57) ABSTRACT

The invention relates to novel TNF family ligand trimer-containing antigen binding molecules comprising (a) at least one moiety capable of specific binding to PD1 and (b) a first and a second polypeptide that are linked to each other by a disulfide bond, characterized in that the first polypeptide comprises two ectodomains of a TNF ligand family member or fragments thereof that are connected to each other by a peptide linker and in that the second polypeptide comprises only one ectodomain of said TNF ligand family member or a fragment thereof.

### FIELD OF THE INVENTION

The invention relates to novel TNF family ligand trimer-containing antigen binding molecules comprising (a) at least one moiety capable of specific binding to PD1 and (b) a first and a second polypeptide that are linked to each other by a disulfide bond, wherein the antigen binding molecules are characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof. The invention further relates to methods of producing these molecules and to methods of using the same.

### BACKGROUND

Ligands interacting with molecules of the TNF (tumor necrosis factor) receptor superfamily have pivotal roles in the organization and function of the immune system. While regulating normal functions such as immune responses, hematopoiesis and morphogenesis, the TNF family ligands (also called cytokines) play a role in tumorgenesis, transplant rejection, septic shock, viral replication, bone resorption, rheumatoid arthritis and diabetes (Aggarwal, 2003). The TNF ligand family comprises 18 genes encoding 19 type II (i.e. intracellular N terminus and extracellular C-terminus) transmembrane proteins, characterized by the presence of a conserved C-terminal domain coined the ‘TNF homology domain’ (THD). This domain is responsible for receptor binding and is thus critical for the biological activity of the TNF ligand family members. The sequence identity between family members is ˜20-30% (Bodmer, 2002). Members of the TNF ligand family exert their biological function as self-assembling, noncovalent trimers (Banner et al, Cell 1993, 73, 431-445). Thus, the TNF family ligands form a trimer that is able to bind to and to activate the corresponding receptors of TNFR superfamily.

4-1BB (CD137), a member of the TNF receptor superfamily, has been first identified as a molecule whose expression is induced by T-cell activation (Kwon and Weissman, 1989). Subsequent studies demonstrated expression of 4-1BB in T- and B-lymphocytes (Snell et al., 2011; Zhang et al., 2010), NK-cells (Lin et al., 2008), NKT-cells (Kim et al., 2008), monocytes (Kienzle and von Kempis, 2000; Schwarz et al., 1995), neutrophils (Heinisch et al., 2000), mast (Nishimoto et al., 2005) and dendritic cells as well as cells of non-hematopoietic origin such as endothelial and smooth muscle cells (Broll et al., 2001; Olofsson et al., 2008). Expression of 4-1BB in different cell types is mostly inducible and driven by various stimulatory signals, such as T-cell receptor (TCR) or B-cell receptor triggering, as well as signaling induced through co-stimulatory molecules or receptors of pro-inflammatory cytokines (Diehl et al., 2002; von Kempis et al., 1997; Zhang et al., 2010).

Expression of 4-1BB ligand (4-1BBL or CD137L) is more restricted and is observed on professional antigen presenting cells (APC) such as B-cells, dendritic cells (DCs) and macrophages. Inducible expression of 4-1BBL is characteristic for T-cells, including both αβ and γδ T-cell subsets, and endothelial cells (reviewed in Shao and Schwarz, 2011).

CD137 signaling is known to stimulate IFNγ secretion and proliferation of NK cells (Buechele et al., 2012; Lin et al., 2008; Melero et al., 1998) as well as to promote DC activation as indicated by their increased survival and capacity to secret cytokines and upregulate co-stimulatory molecules (Choi et al., 2009; Futagawa et al., 2002; Wilcox et al., 2002). However, CD137 is best characterized as a co-stimulatory molecule which modulates TCR-induced activation in both the CD4+ and CD8+ subsets of T-cells. In combination with TCR triggering, agonistic 4-1BB-specific antibodies enhance proliferation of T-cells, stimulate lymphokine secretion and decrease sensitivity of T-lymphocytes to activation-induced cells death (reviewed in (reviewed in Snell et al., 2011).

In line with these co-stimulatory effects of 4-1BB antibodies on T-cells in vitro, their administration to tumor bearing mice leads to potent anti-tumor effects in many experimental tumor models (Melero et al., 1997; Narazaki et al., 2010). However, 4-1BB usually exhibits its potency as an anti-tumor agent only when administered in combination with other immunomodulatory compounds (Curran et al., 2011; Guo et al., 2013; Morales-Kastresana et al., 2013; Teng et al., 2009; Wei et al., 2013), chemotherapeutic reagents (Ju et al., 2008; Kim et al., 2009), tumor-specific vaccination (Cuadros et al., 2005; Lee et al., 2011) or radiotherapy (Shi and Siemann, 2006). In vivo depletion experiments demonstrated that CD8+ T-cells play the most critical role in anti-tumoral effect of 4-1BB-specific antibodies. However, depending on the tumor model or combination therapy, which includes anti-4-1BB, contributions of other types of cells such as DCs, NK-cells or CD4+ T-cells have been reported (Melero et al., 1997; Murillo et al., 2009; Narazaki et al., 2010; Stagg et al., 2011).

In addition to their direct effects on different lymphocyte subsets, 4-1BB agonists can also induce infiltration and retention of activated T-cells in the tumor through 4-1BB-mediated upregulation of intercellular adhesion molecule 1 (ICAM1) and vascular cell adhesion molecule 1 (VCAM1) on tumor vascular endothelium (Palazon et al., 2011).

4-1BB triggering may also reverse the state of T-cell anergy induced by exposure to soluble antigen that may contribute to disruption of immunological tolerance in the tumor microenvironment or during chronic infections (Wilcox et al., 2004).

It appears that the immunomodulatory properties of 4-1BB agonistic antibodies in vivo require the presence of the wild type Fc-portion on the antibody molecule thereby implicating Fc-receptor binding as an important event required for the pharmacological activity of such reagents as has been described for agonistic antibodies specific to other apoptosis-inducing or immunomodulatory members of the TNFR-superfamily (Li and Ravetch, 2011; Teng et al., 2009). However, systemic administration of 4-1BB-specific agonistic antibodies with the functionally active Fc domain also induces expansion of CD8+ T-cells associated with liver toxicity (Dubrot et al., 2010) that is diminished or significantly ameliorated in the absence of functional Fc-receptors in mice. In human clinical trials (ClinicalTrials.gov, NCT00309023), Fc-competent 4-1BB agonistic antibodies (BMS-663513) administered once every three weeks for 12 weeks induced stabilization of the disease in patients with melanoma, ovarian or renal cell carcinoma. However, the same antibody given in another trial (NCT00612664) caused grade 4 hepatitis leading to termination of the trial (Simeone and Ascierto, 2012).

Collectively, the available pre-clinical and clinical data clearly demonstrate that there is a high clinical need for effective 4-1BB agonists. However, new generation drug candidates should not only effectively engage 4-1BB on the surface of hematopoietic and endothelial cells but also be capable of achieving that through mechanisms other than binding to Fc-receptors in order to avoid uncontrollable side effects. The latter may be accomplished through preferential binding to and oligomerization on tumor-specific or tumor-associated moieties.

Fusion proteins composed of one extracellular domain of a 4-1BB ligand and a single chain antibody fragment (Mueller et al., 2008; Hornig et al., 2012) or a single 4-1BB ligand fused to the C-terminus of a heavy chain (Zhang et al, 2007) have been made. WO 2010/010051 discloses the generation of fusion proteins that consist of three TNF ligand ectodomains linked to each other and fused to an antibody part.

Programmed cell death protein 1 (PD1 or CD279) is an inhibitory member of the CD28 family of receptors, that also includes CD28, CTLA-4, ICOS and BTLA. PD1 is a cell surface receptor and is expressed on activated B cells, T cells, and myeloid cells (Okazaki et al (2002) Curr. Opin. Immunol. 14: 391779-82; Bennett et al. (2003) J Immunol 170:711-8). The structure of PD1 is a monomeric type 1 transmembrane protein, consisting of one immunoglobulin variable-like extracellular domain and a cytoplasmic domain containing an immunoreceptor tyrosine-based inhibitory motif (ITIM) and an immunoreceptor tyrosine-based switch motif (ITSM). Activated T cells transiently express PD1, but sustained hyperexpression of PD1 and its ligand PDL1 promote immune exhaustion, leading to persistence of viral infections, tumor evasion, increased infections and mortality. PD1 expression is induced by antigen recognition via the T-cell receptor and its expression is maintained primarily through continuous T-cell receptor signaling. After prolonged antigen exposure, the PD1 locus fails to be remethylated, which promotes continuous hyperexpression. Blocking the PD1 pathway can restore the exhausted T-cell functionality in cancer and chronic viral infections (Sheridan, Nature Biotechnology 30 (2012), 729-730). Monoclonal antibodies to PD1 have been described, for example, in WO 2003/042402, WO 2004/004771, WO 2004/056875, WO 2004/072286, WO 2004/087196, WO 2006/121168, WO 2006/133396, WO 2007/005874, WO 2008/083174, WO 2008/156712, WO 2009/024531, WO 2009/014708, WO 2009/101611, WO 2009/114335, WO 2009/154335, WO 2010/027828, WO 2010/027423, WO 2010/029434, WO 2010/029435, WO 2010/036959, WO 2010/063011, WO 2010/089411, WO 2011/066342, WO 2011/110604, WO 2011/110621, WO 2012/145493, WO 2013/014668, WO 2014/179664, and WO 2015/112900. Two PD1 monoclonal antibodies, Opdivo (nivolumab) and Keytruda (pembrolizumab), are already on the market for the treatment of certain cancers. However, despite of the compelling anti-tumor activity, relaps of tumor growth after therapeutic PD1 blockade has increasingly been observed (Koyama et al., Nature Communications 2016, DOI: 10.1038/ncomms10501).

It has been shown that dual targeting of CD137/PD1 in the tumor microenvironment abolishes the local suppression and leads to functional rescue and expansion of effector T cells that subsequently kill cancer cells (Wei et al., 2014). It has been shown in a mouse model for subcutaneous tumor, that mice treated with a combination of an anti-4-1BB antibody and an anti-PD1 antibody had the best anti-tumor response compared to mice treated with one of the antibodies alone (Shindo et al., 2015). Administering the combination of an anti-PD1 antibody and a 4-1BB agonistic antibody in a tumor mouse model led to an effective long-lasting systemic antitumor response, however the known toxicity of the 4-1BB agonist as given alone was slightly increased (Chen et al., 2014). Thus, there is a need for new molecules that combine the positive effects of both the anti-PD1 antibody as well as the 4-1BB antibody with an improved safety profile.

The new antigen binding molecules of the present invention combine a PD1 moiety with a moiety that is capable of forming a costimulatory TNF ligand trimer and that is sufficiently stable to be pharmaceutically useful. Surprisingly, antigen binding molecules of the invention provide a trimeric and thus biologically active TNF ligand, although one of the trimerizing TNF ligand ectodomains is located on another polypeptide than the other two TNF ligand ectodomains of the molecule. Targeted by the anti-PD1 moiety the antigen binding molecules of the present invention have an increased activity in the tumor microenvironment and should thus impose less safety issues compared to conventional 4-1BB agonistic antibodies alone.

### SUMMARY OF THE INVENTION

In one aspect, the invention provides a TNF family ligand trimer-containing antigen binding molecule comprising

(a) at least one moiety capable of specific binding to PD1 and
(b) a first and a second polypeptide that are linked to each other by a disulfide bond,
wherein the antigen binding molecule is characterized in that the first polypeptide comprises two ectodomains of a TNF ligand family member or a fragment thereof that are connected to each other by a peptide linker and in that the second polypeptide comprises only one ectodomain of said TNF ligand family member or a fragment thereof.

In a particular aspect, the invention provides a TNF family ligand trimer-containing antigen binding molecule comprising

(a) at least one moiety capable of specific binding to PD1,
(b) a first and a second polypeptide that are linked to each other by a disulfide bond,
wherein the antigen binding molecule is characterized in that the first polypeptide comprises two ectodomains of a TNF ligand family member or a fragment thereof that are connected to each other by a peptide linker and in that the second polypeptide comprises only one ectodomain of said TNF ligand family member or a fragment thereof, and
(c) an Fc domain composed of a first and a second subunit capable of stable association.

In a further aspect, the invention provides a TNF family ligand trimer-containing antigen binding molecule, comprising

(a) at least one moiety capable of specific binding to PD1 and
(b) a first and a second polypeptide that are linked to each other by a disulfide bond,
wherein the antigen binding molecule is characterized in that
(i) the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of a TNF ligand family member or two fragments thereof that are connected to each other and to the CH1 or CL domain by a peptide linker and wherein the second polypeptide comprises one ectodomain of said TNF ligand family member or a fragment thereof connected via a peptide linker to the CL or CH1 domain of said polypeptide, or
(ii) the first polypeptide contains a CH3 domain and the second polypeptide contains a CH3 domain, respectively, and wherein the first polypeptide comprises two ectodomains of a TNF ligand family member or two fragments thereof that are connected to each other and to the C-terminus of the CH3 domain by a peptide linker and wherein the second polypeptide comprises only one ectodomain of said TNF ligand family member or a fragment thereof connected via a peptide linker to C-terminus of the CH3 domain of said polypeptide, or
(iii) the first polypeptide contains a VH-CL or a VL-CH1 domain and the second polypeptide contains a VL-CH1 domain or a VH-CL domain, respectively, wherein the second polypeptide is linked to the first polypeptide by a disulfide bond between the CH1 and CL domain, and wherein the first polypeptide comprises two ectodomains of a TNF ligand family member or fragments thereof that are connected to each other and to to VH or VL by a peptide linker and wherein the second polypeptide comprises one ectodomain of said TNF ligand family member or a fragment thereof connected via a peptide linker to VL or VH of said polypeptide.

In a particular aspect, the invention provides a TNF family ligand trimer-containing antigen binding molecule, comprising

(a) at least one moiety capable of specific binding to PD1 and
(b) a first and a second polypeptide that are linked to each other by a disulfide bond,
wherein the antigen binding molecule is characterized in that
(i) the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of a TNF ligand family member or two fragments thereof that are connected to each other and to the CH1 or CL domain by a peptide linker and wherein the second polypeptide comprises one ectodomain of said TNF ligand family member or a fragment thereof connected via a peptide linker to the CL or CH1 domain of said polypeptide, or
(ii) the first polypeptide contains a CH3 domain and the second polypeptide contains a CH3 domain, respectively, and wherein the first polypeptide comprises two ectodomains of a TNF ligand family member or two fragments thereof that are connected to each other and to the C-terminus of the CH3 domain by a peptide linker and wherein the second polypeptide comprises only one ectodomain of said TNF ligand family member or a fragment thereof connected via a peptide linker to C-terminus of the CH3 domain of said polypeptide.

In one aspect, the TNF ligand family member is one that costimulates human T-cell activation. Thus, the TNF family ligand trimer-containing antigen binding molecule comprises (a) at least one moiety capable of specific binding to a target cell antigen and

(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof, wherein the TNF ligand family member costimulates human T-cell activation. More particularly, the TNF ligand family member is selected from 4-1BBL and OX40L.

In a particular aspect, the TNF ligand family member is 4-1BBL.

In a further aspect, the ectodomain of a TNF ligand family member comprises the amino acid sequence selected from the group consisting of SEQ ID NO: 1, SEQ ID NO: 2, SEQ ID NO:3, SEQ ID NO:4, SEQ ID NO:5, SEQ ID NO: 6, SEQ ID NO:7 and SEQ ID NO:8, particularly the amino acid sequence of SEQ ID NO: 1 or SEQ ID NO:5. More particularly, the ectodomain of a TNF ligand family member comprises the amino acid sequence of SEQ ID NO:5.

In a further aspect, the TNF family ligand trimer-containing antigen binding molecule of the invention comprises

(a) at least one moiety capable of specific binding to PD1 and
(b) a first and a second polypeptide that are linked to each other by a disulfide bond,
wherein the antigen binding molecule is characterized in that the first polypeptide comprises the amino acid sequence selected from the group consisting of SEQ ID NO:9, SEQ ID NO: 11, SEQ ID NO: 12 and SEQ ID NO: 10 and in that the second polypeptide comprises the amino acid sequence selected from the group consisting of SEQ ID NO: 1, SEQ ID NO:3, SEQ ID NO:4 and SEQ ID NO:5.

In one aspect, the TNF family ligand trimer-containing antigen binding molecule of the invention comprises

(a) at least one moiety capable of specific binding to PD1 and
(b) a first and a second polypeptide that are linked to each other by a disulfide bond,
wherein the antigen binding molecule is characterized in that the first polypeptide comprises the amino acid sequence of SEQ ID NO: 10 and in that the second polypeptide comprises the amino acid sequence of SEQ ID NO:5.

In another aspect, the TNF family ligand trimer-containing antigen binding molecule of the invention comprises

(a) at least one moiety capable of specific binding to PD1,
(b) a first polypeptide containing a CH1 or CL domain and a second polypeptide containing a CL or CH1 domain, respectively, wherein the second polypeptide is linked to the first polypeptide by a disulfide bond between the CH1 and CL domain,
and wherein the antigen binding molecule is characterized in that the first polypeptide comprises two ectodomains of a TNF ligand family member or two fragments thereof that are connected to each other and to the CH1 or CL domain by a peptide linker and in that the second polypeptide comprises only one ectodomain of said TNF ligand family member or a fragment thereof connected by a peptide linker to the CL or CH1 domain of said polypeptide.

In one aspect, provided is a TNF family ligand trimer-containing antigen binding molecule comprising

(a) at least one moiety capable of specific binding to PD1,
(b) a first polypeptide containing a CH1 domain and a second polypeptide containing a CL domain, wherein the second polypeptide is linked to the first polypeptide by a disulfide bond between the CH1 and CL domain,
and wherein the antigen binding molecule is characterized in that the first polypeptide comprises two ectodomains of a TNF ligand family member or fragments thereof that are connected to each other and to the CH1 domain by a peptide linker and in that the second polypeptide comprises one ectodomain of said TNF ligand family member or a fragment thereof connected via a peptide linker to the CL domain of said polypeptide.

In another aspect, provided is a TNF family ligand trimer-containing antigen binding molecule comprising

(a) at least one moiety capable of specific binding to PD1,
(b) a first polypeptide containing a CL domain and a second polypeptide containing a CH1 domain, wherein the second polypeptide is linked to the first polypeptide by a disulfide bond between the CH1 and CL domain,
and wherein the antigen binding molecule is characterized in that the first polypeptide comprises two ectodomains of a TNF ligand family member or fragments thereof that are connected to each other and to the CL domain by a peptide linker and in that the second polypeptide comprises one ectodomain of said TNF ligand family member or a fragment thereof connected via a peptide linker to the CH1 domain of said polypeptide.

In a further aspect, the invention provides a TNF family ligand trimer-containing antigen binding molecule as defined herein before, wherein the moiety capable of specific binding PD1 is selected from the group consisting of an antibody, an antibody fragment and a scaffold antigen binding protein. In particular, the moiety capable of specific binding to PD1 is selected from the group consisting of an antibody fragment, a Fab molecule, a crossover Fab molecule, a single chain Fab molecule, a Fv molecule, a scFv molecule, a single domain antibody, an aVH and a scaffold antigen binding protein.

In one aspect, the invention provides a TNF family ligand trimer-containing antigen binding molecule as defined herein before, wherein the moiety capable of specific binding to PD1 is an antibody fragment. In particular, the moiety capable of specific binding to PD1 is selected from the group consisting of a Fab molecule, a crossover Fab molecule, a single chain Fab molecule, a Fv molecule, a scFv molecule, a single domain antibody and an aVH.

In one aspect, the invention provides a TNF family ligand trimer-containing antigen binding molecule as defined herein before, wherein the moiety capable of specific binding to PD1 is a scaffold antigen binding protein.

In a particular aspect, the invention is concerned with a TNF family ligand trimer-containing antigen binding molecule as defined above, wherein the moiety capable of specific binding to PD1 is a Fab molecule or a scFV molecule capable of specific binding to a target cell antigen. More particularly, the moiety capable of specific binding to PD1 is a Fab molecule.

The invention provides a TNF family ligand trimer-containing antigen binding molecule that comprises at least one moiety capable of specific binding to PD1. In a particular aspect, the TNF family ligand trimer-containing antigen binding molecule comprises one moiety capable of specific binding to PD1, meaning the TNF family ligand trimer-containing antigen binding molecule is monovalent. In another aspect, the invention provides a TNF family ligand trimer-containing antigen binding molecule comprising two moieties capable of specific binding to PD1, meaning the TNF family ligand trimer-containing antigen binding molecule is bivalent.

In one aspect, the invention provides a TNF family ligand trimer-containing antigen binding molecule, wherein the moiety capable of specific binding to PD1 comprises

(a) a VH domain comprising (i) HVR-H1 comprising the amino acid sequence of SEQ ID NO: 13,
(ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16 (v) HVR-L2 comprising the amino acid sequence of SEQ ID NO: 17, and (vi) HVR-L3 comprising the amino acid sequence of SEQ ID NO:18,
(b) a VH domain comprising (i) HVR-H1 comprising the amino acid sequence of SEQ ID NO:26,
(ii) HVR-H2 comprising the amino acid sequence of SEQ ID NO:27, and (iii) HVR-H3 comprising the amino acid sequence of SEQ ID NO:28, and a VL domain comprising (iv) HVR-L1 comprising the amino acid sequence of SEQ ID NO:29 (v) HVR-L2 comprising the amino acid sequence of SEQ ID NO:30, and (vi) HVR-L3 comprising the amino acid sequence of SEQ ID NO:31,
(c) a VH domain comprising (i) HVR-H1 comprising the amino acid sequence of SEQ ID NO:34,
(ii) HVR-H2 comprising the amino acid sequence of SEQ ID NO:35, and (iii) HVR-H3 comprising the amino acid sequence of SEQ ID NO:36, and a VL domain comprising (iv) HVR-L1 comprising the amino acid sequence of SEQ ID NO:37 (v) HVR-L2 comprising the amino acid sequence of SEQ ID NO:38, and (vi) HVR-L3 comprising the amino acid sequence of SEQ ID NO:39,
(d) a VH domain comprising (i) HVR-H1 comprising the amino acid sequence of SEQ ID NO:42,
(ii) HVR-H2 comprising the amino acid sequence of SEQ ID NO:43, and (iii) HVR-H3 comprising the amino acid sequence of SEQ ID NO:44, and a VL domain comprising (iv) HVR-L1 comprising the amino acid sequence of SEQ ID NO:45 (v) HVR-L2 comprising the amino acid sequence of SEQ ID NO:46, and (vi) HVR-L3 comprising the amino acid sequence of SEQ ID NO:47,
(e) a VH domain comprising (i) HVR-H1 comprising the amino acid sequence of SEQ ID NO:50,
(ii) HVR-H2 comprising the amino acid sequence of SEQ ID NO:51, and (iii) HVR-H3 comprising the amino acid sequence of SEQ ID NO:52, and a VL domain comprising (iv) HVR-L1 comprising the amino acid sequence of SEQ ID NO:53 (v) HVR-L2 comprising the amino acid sequence of SEQ ID NO:54, and (vi) HVR-L3 comprising the amino acid sequence of SEQ ID NO:55,
(f) a VH domain comprising (i) HVR-H1 comprising the amino acid sequence of SEQ ID NO:58,
(ii) HVR-H2 comprising the amino acid sequence of SEQ ID NO:59, and (iii) HVR-H3 comprising the amino acid sequence of SEQ ID NO:60, and a VL domain comprising (iv) HVR-L1 comprising the amino acid sequence of SEQ ID NO:61 (v) HVR-L2 comprising the amino acid sequence of SEQ ID NO:62, and (vi) HVR-L3 comprising the amino acid sequence of SEQ ID NO:63, or
(g) a VH domain comprising (i) HVR-H1 comprising the amino acid sequence of SEQ ID NO:66,
(ii) HVR-H2 comprising the amino acid sequence of SEQ ID NO:67, and (iii) HVR-H3 comprising the amino acid sequence of SEQ ID NO:68, and a VL domain comprising (iv) HVR-L1 comprising the amino acid sequence of SEQ ID NO:69 (v) HVR-L2 comprising the amino acid sequence of SEQ ID NO:70, and (vi) HVR-L3 comprising the amino acid sequence of SEQ ID NO:71.

In a further aspect, the invention provides a TNF family ligand trimer-containing antigen binding molecule, wherein the moiety capable of specific binding to PD1 comprises

(a) a VH domain comprising an amino acid sequence of SEQ ID NO: 19 and a VL domain comprising an amino acid sequence of SEQ ID NO:20,
(b) a VH domain comprising an amino acid sequence of SEQ ID NO:21 and a VL domain comprising an amino acid sequence of SEQ ID NO:22,
(c) a VH domain comprising an amino acid sequence of SEQ ID NO:21 and a VL domain comprising an amino acid sequence of SEQ ID NO:23,
(d) a VH domain comprising an amino acid sequence of SEQ ID NO:21 and a VL domain comprising an amino acid sequence of SEQ ID NO:24,
(e) a VH domain comprising an amino acid sequence of SEQ ID NO:21 and a VL domain comprising an amino acid sequence of SEQ ID NO:25,
(f) a VH domain comprising an amino acid sequence of SEQ ID NO:32 and a VL domain comprising an amino acid sequence of SEQ ID NO:33,
(g) a VH domain comprising an amino acid sequence of SEQ ID NO:40 and a VL domain comprising an amino acid sequence of SEQ ID NO:41,
(h) a VH domain comprising an amino acid sequence of SEQ ID NO:48 and a VL domain comprising an amino acid sequence of SEQ ID NO:49,
(i) a VH domain comprising an amino acid sequence of SEQ ID NO:56 and a VL domain comprising an amino acid sequence of SEQ ID NO:57,
(k) a VH domain comprising an amino acid sequence of SEQ ID NO:64 and a VL domain comprising an amino acid sequence of SEQ ID NO:65, or
(l) a VH domain comprising an amino acid sequence of SEQ ID NO:72 and a VL domain comprising an amino acid sequence of SEQ ID NO:73.

In one aspect, the invention provides a TNF family ligand trimer-containing antigen binding molecule, wherein the moiety capable of specific binding to PD1 comprises

(a) a VH domain comprising (i) HVR-H1 comprising the amino acid sequence of SEQ ID NO: 13,
(ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16, (v) HVR-L2 comprising the amino acid sequence of SEQ ID NO: 17, and (vi) HVR-L3 comprising the amino acid sequence of SEQ ID NO:18.

In a particular aspect, the invention provides a TNF family ligand trimer-containing antigen binding molecule, wherein the moiety capable of specific binding to PD1 comprises

(a) a VH domain comprising an amino acid sequence of SEQ ID NO: 19 and a VL domain comprising an amino acid sequence of SEQ ID NO:20,
(b) a VH domain comprising an amino acid sequence of SEQ ID NO:21 and a VL domain comprising an amino acid sequence of SEQ ID NO:22,
(c) a VH domain comprising an amino acid sequence of SEQ ID NO:21 and a VL domain comprising an amino acid sequence of SEQ ID NO:23,
(d) a VH domain comprising an amino acid sequence of SEQ ID NO:21 and a VL domain comprising an amino acid sequence of SEQ ID NO:24, or
(e) a VH domain comprising an amino acid sequence of SEQ ID NO:21 and a VL domain comprising an amino acid sequence of SEQ ID NO:25.

In one aspect, provided is a TNF family ligand trimer-containing antigen binding molecule as defined herein before, wherein the moiety capable of specific binding to PD1 comprises a variable heavy chain comprising an amino acid sequence of SEQ ID NO:21 and a variable light chain comprising an amino acid sequence of SEQ ID NO:22 or wherein the moiety capable of specific binding to PD1 comprises a variable heavy chain comprising an amino acid sequence of SEQ ID NO:21 and a variable light chain comprising an amino acid sequence of SEQ ID NO:24.

In a particular aspect, the invention relates to a TNF family ligand trimer-containing antigen binding molecule as defined above, wherein the peptide linker is (G4S)₂, i.e. a peptide linker of SEQ ID NO: 117. In one aspect, the peptide linker in all instances is (G4S)₂.

The invention is further concerned with a TNF family ligand trimer-containing antigen binding molecule as defined herein before, comprising an Fc domain composed of a first and a second subunit capable of stable association.

In one aspect, the TNF family ligand trimer-containing antigen binding molecule of the invention comprising (c) an Fc domain composed of a first and a second subunit capable of stable association further comprises (a) a Fab molecule capable of specific binding to a target cell antigen, wherein the Fab heavy chain is fused at the C-terminus to the N-terminus of a CH2 domain in the Fc domain.

In a further aspect, the Fc domain is an IgG, particularly an IgG1 Fc domain or an IgG4 Fc domain.

More particularly, the Fc domain is an IgG1 Fc domain. In a particular aspect, the Fc domain comprises a modification promoting the association of the first and second subunit of the Fc domain. In a specific aspect, the invention provides a TNF family ligand trimer-containing antigen binding molecule, wherein the first subunit of the Fc domain comprises the amino acid substitutions S354C and T366W (numbering according to Kabat EU index) and the second subunit of the Fc domain comprises the amino acid substitutions Y349C, T366S, L368A and Y407V (numbering according to Kabat EU index).

In another aspect, the invention is concerned with a TNF family ligand trimer-containing antigen binding molecule as defined herein before, comprising

(c) an Fc domain composed of a first and a second subunit capable of stable association, wherein the Fc domain comprises one or more amino acid substitution that reduces binding to an Fc receptor, in particular towards Fcγ receptor.

In particular, the Fc domain comprises amino acid substitutions at positions 234 and 235 (EU numbering) and/or 329 (EU numbering) of the IgG heavy chains. More particularly, provided is a trimeric TNF family ligand-containing antigen binding molecule according to the invention which comprises an IgG1 Fc domain comprising the amino acid substitutions at positions 234 and 235 (EU numbering) and/or 329 (EU numbering), in particular the amino acid substitutions L234A, L235A and P329G (EU numbering).

In a further aspect, the invention provides a TNF family ligand trimer-containing antigen binding molecule, wherein the antigen binding molecule comprises

a first heavy chain and a first light chain, both comprising a Fab molecule capable of specific binding to PD1,
a first peptide comprising two ectodomains of a TNF ligand family member or fragments thereof connected to each other by a first peptide linker fused at its C-terminus by a second peptide linker to a second heavy or light chain,
and a second peptide comprising one ectodomain of said TNF ligand family member fused at its C-terminus by a third peptide linker to a second light or heavy chain, respectively.

In another aspect, provided is a TNF family ligand trimer-containing antigen binding molecule, wherein the first peptide comprising two ectodomains of a TNF ligand family member or fragments thereof connected to each other by a first peptide linker is fused at its C-terminus by a second peptide linker to a CH1 domain that is part of a heavy chain,

and the second peptide comprising one ectodomain of said TNF ligand family member or a fragment thereof is fused at its C-terminus by a third peptide linker to a CL domain that is part of a light chain.

In yet another aspect, provided is a TNF family ligand trimer-containing antigen binding molecule, wherein the first peptide comprising two ectodomains of a TNF ligand family member or fragments thereof connected to each other by a first peptide linker is fused at its C-terminus by a second peptide linker to a CL domain that is part of a heavy chain,

and the second peptide comprising one ectodomain of said TNF ligand family member or a fragment thereof is fused at its C-terminus by a third peptide linker to a CH1 domain that is part of a light chain.

In a further aspect, the invention provides a TNF family ligand trimer-containing antigen binding molecule, wherein the first peptide comprising two ectodomains of a TNF ligand family member or fragments thereof connected to each other by a first peptide linker is fused at its C-terminus by a second peptide linker to a VH domain that is part of a heavy chain,

and the second peptide comprising one ectodomain of said TNF ligand family member or a fragment thereof is fused at its C-terminus by a third peptide linker to a VL domain that is part of a light chain.

In a particular aspect, the invention relates to a TNF family ligand trimer-containing antigen binding molecule as defined above, wherein the peptide linker is (G4S)₂, i.e. a peptide linker of SEQ ID NO: 117. In one aspect, the first, second and third peptide linker is (G4S)₂.

Provided is further a TNF family ligand trimer-containing antigen binding molecule, wherein in the CL domain adjacent to the TNF ligand family member the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E).

In a further aspect, provided is a TNF family ligand trimer-containing antigen binding molecule as described herein before, wherein the antigen binding molecule comprises

(a) a first heavy chain and a first light chain, both comprising a Fab molecule capable of specific binding to PD1,
(b) a second heavy chain comprising an amino acid sequence selected from the group consisting of SEQ ID NO:9, SEQ ID NO: 11, SEQ ID NO: 12 and SEQ ID NO: 10, and
a second light chain comprising an amino acid sequence selected from the group consisting of SEQ ID NO: 1, SEQ ID NO:3, SEQ ID NO:4 and SEQ ID NO:5.

In one aspect, the invention provides a TNF family ligand trimer-containing antigen binding molecule, wherein the antigen binding molecule comprises

(a) a Fab molecule capable of specific binding to PD1, and
(b) a second heavy chain comprising an amino acid sequence selected from the group consisting of SEQ ID NO:9, SEQ ID NO: 11, SEQ ID NO: 12 and SEQ ID NO: 10, and
a second light chain comprising an amino acid sequence selected from the group consisting of SEQ ID NO: 1, SEQ ID NO:3, SEQ ID NO:4 and SEQ ID NO:5.

In a particular aspect, provided is a TNF family ligand trimer-containing antigen binding molecule, wherein the antigen binding molecule comprises

(i) a first heavy chain comprising the VH domain comprising the amino acid sequence of SEQ ID NO: 19 and a first light chain comprising the VL domain comprising the amino acid sequence of SEQ ID NO:20 or
a first heavy chain comprising the VH domain comprising the amino acid sequence of SEQ ID NO:21 and a first light chain comprising the VL domain comprising the amino acid sequence selected from the group consisting of SEQ ID NO:22, SEQ ID NO:23, SEQ ID NO:24 and SEQ ID NO:25,
(ii) a second heavy chain comprising the amino acid sequence selected from the group consisting of SEQ ID NO:74, SEQ ID NO:76, SEQ ID NO:78 and SEQ ID NO:80, and
(iii) a second light chain comprising the amino acid sequence selected from the group consisting of SEQ ID NO:75, SEQ ID NO:77, SEQ ID NO:79 and SEQ ID NO:81.

In another aspect, the invention provides a TNF family ligand trimer-containing antigen binding molecule, wherein the antigen binding molecule comprises

(a) a first heavy chain comprising the amino acid sequence of SEQ ID NO:82, a first light chain comprising the amino acid sequence of SEQ ID NO:83 or SEQ ID NO:171, a second heavy chain comprising the amino acid sequence of SEQ ID NO:74 and a second light chain comprising the amino acid sequence of SEQ ID NO:75,
(b) a first heavy chain comprising the amino acid sequence of SEQ ID NO:82, a first light chain comprising the amino acid sequence of SEQ ID NO:83 or SEQ ID NO:171, a second heavy chain comprising the amino acid sequence of SEQ ID NO:76 and a second light chain comprising the amino acid sequence of SEQ ID NO:77,
(c) a first heavy chain comprising the amino acid sequence of SEQ ID NO:82, a first light chain comprising the amino acid sequence of SEQ ID NO:83 or SEQ ID NO:171, a second heavy chain comprising the amino acid sequence of SEQ ID NO:78 and a second light chain comprising the amino acid sequence of SEQ ID NO:79, or
(d) a first heavy chain comprising the amino acid sequence of SEQ ID NO:82, a first light chain comprising the amino acid sequence of SEQ ID NO:83 or SEQ ID NO:171, a second heavy chain comprising the amino acid sequence of SEQ ID NO:80 and a second light chain comprising the amino acid sequence of SEQ ID NO:81.

In yet another aspect, the invention provides a TNF family ligand trimer-containing antigen binding molecule, comprising

(a) at least one moiety capable of specific binding to PD1, and
(b) a first and a second polypeptide that are linked to each other by a disulfide bond,
wherein the antigen binding molecule is characterized in that the first polypeptide contains a CH3 domain and the second polypeptide contains a CH3 domain, respectively, and wherein the first polypeptide comprises two ectodomains of a TNF ligand family member or fragments thereof that are connected to each other and to the C-terminus of the CH3 domain by a peptide linker and wherein the second polypeptide comprises one ectodomain of said TNF ligand family member or a fragment thereof connected via a peptide linker to the C-terminus of the CH3 domain of said polypeptide.

In particular, such a TNF family ligand trimer-containing antigen binding molecule comprises two Fab domains capable of specific binding to PD1.

In particular, provided is a TNF family ligand trimer-containing antigen binding molecule as described herein before comprises

(a) a first heavy chain comprising the amino acid sequence of SEQ ID NO:84, a second heavy chain comprising the amino acid sequence of SEQ ID NO:85, and two light chains comprising the amino acid sequence of SEQ ID NO:83 or SEQ ID NO: 171, or
(b) a first heavy chain comprising the amino acid sequence of SEQ ID NO:86, a second heavy chain comprising the amino acid sequence of SEQ ID NO:87, and two light chains comprising the amino acid sequence of SEQ ID NO:83 or SEQ ID NO: 171.

In another particular aspect, the invention provides a TNF family ligand trimer-containing antigen binding molecule as described herein before, wherein the TNF family ligand trimer-containing antigen binding molecule comprises one Fab domain capable of specific binding to PD1.

In such aspect, provided is a TNF family ligand trimer-containing antigen binding molecule, wherein the antigen binding molecule comprises

(i) a fusion polypeptide comprising a first subunit of a Fc domain and two ectodomains of a TNF ligand family member or fragments thereof that are connected to each other and to the C-terminus of the CH3 domain by a peptide linker, (ii) a heavy chain comprising the VH domain of the Fab domain capable of specific binding to PD1, a second subunit of the Fc domain and one ectodomain of said TNF ligand family member or a fragment thereof connected via a peptide linker to C-terminus of the CH3 domain, and (iii) a light chain comprising the VL domain of the Fab domain capable of specific binding to PD1, or
(i) a fusion polypeptide comprising a first subunit of a Fc domain and one ectodomain of a TNF ligand family member or a fragment thereof that is connected to the C-terminus of the CH3 domain by a peptide linker, (ii) a heavy chain comprising the VH domain of the Fab domain capable of specific binding to PD1, a second subunit of the Fc domain and two ectodomains of said TNF ligand family member or fragments thereof that are connected to each other and to the C-terminus of the CH3 domain via a peptide linker, and (iii) a light chain comprising the VL domain of the Fab domain capable of specific binding to PD1.

In a particular aspect, provided is a TNF family ligand trimer-containing antigen binding molecule, wherein the antigen binding molecule comprises

(a) a fusion polypeptide comprising the amino acid sequence of SEQ ID NO: 88, a heavy chain comprising the amino acid sequence of SEQ ID NO:87 and a light chain comprising the amino acid sequence of SEQ ID NO:83 or SEQ ID NO:171,
(b) a fusion polypeptide comprising the amino acid sequence of SEQ ID NO:89, a heavy chain comprising the amino acid sequence of SEQ ID NO:90 and a light chain comprising the amino acid sequence of SEQ ID NO:83 or SEQ ID NO:171,
(c) a fusion polypeptide comprising the amino acid sequence of SEQ ID NO:91, a heavy chain comprising the amino acid sequence of SEQ ID NO:86 and a light chain comprising the amino acid sequence of SEQ ID NO:83 or SEQ ID NO:171,
(b) a fusion polypeptide comprising the amino acid sequence of SEQ ID NO:93, a heavy chain comprising the amino acid sequence of SEQ ID NO:92 and a light chain comprising the amino acid sequence of SEQ ID NO:83 or SEQ ID NO:171.

According to another aspect of the invention, there is provided an isolated polynucleotide encoding a TNF family ligand trimer-containing antigen binding molecule as defined herein before. The invention further provides a vector, particularly an expression vector, comprising the isolated polynucleotide of the invention and a host cell comprising the isolated polynucleotide or the vector of the invention. In some embodiments the host cell is an eukaryotic cell, particularly a mammalian cell.

In another aspect, provided is a method for producing the TNF family ligand trimer-containing antigen binding molecule of the invention, comprising culturing the host cell of the invention under conditions suitable for expression of the TNF family ligand trimer-containing antigen binding molecule, and isolating the antigen TNF family ligand trimer-containing binding molecule. The invention also encompasses a TNF family ligand trimer-containing antigen binding molecule produced by the method of the invention.

The invention further provides a pharmaceutical composition comprising the TNF family ligand trimer-containing antigen binding molecule of the invention and at least one pharmaceutically acceptable excipient.

Also encompassed by the invention is the TNF family ligand trimer-containing antigen binding molecule of the invention, or the pharmaceutical composition of the invention, for use as a medicament. In one aspect is provided the TNF family ligand trimer-containing antigen binding molecule of the invention, or the pharmaceutical composition of the invention, for use in the treatment of a disease in an individual in need thereof. In a specific embodiment, provided is the TNF family ligand trimer-containing antigen binding molecule of the invention, or the pharmaceutical composition of the invention, for use in the treatment of cancer. In another aspect, provided is the TNF family ligand trimer-containing antigen binding molecule of the invention, or the pharmaceutical composition of the invention, for use in up-regulating or prolonging cytotoxic T cell activity.

Also provided is the use of the TNF family ligand trimer-containing antigen binding molecule of the invention for the manufacture of a medicament for the treatment of a disease in an individual in need thereof, in particular for the manufacture of a medicament for the treatment of cancer, as well as a method of treating a disease in an individual, comprising administering to said individual a therapeutically effective amount of a composition comprising the TNF family ligand trimer-containing antigen binding molecule of the invention in a pharmaceutically acceptable form. In a specific embodiment, the disease is cancer. Also provided is a method of up-regulating or prolonging cytotoxic T cell activity in an individual having cancer, comprising administering to the individual an effective amount of the TNF family ligand trimer-containing antigen binding molecule of the invention, or the pharmaceutical composition of the invention.

In any of the above embodiments the individual is preferably a mammal, particularly a human.

### BRIEF DESCRIPTION OF THE DRAWINGS

FIG. 1 shows that blockade of PD1 with chimeric antibody clone PD1-0103 strongly enhances IFN-gamma secretion by allogenic stimulated primary human T cells. IFN-gamma secretion is higher compared to reference antibodies MDX5C4 or MK-3475.

FIG. 2 shows that blockade of PD1 humanized clones PD1-0103-0312 and PD1-103-314 more strongly increases interferon-gamma (IFN-g) secretion by allogenic stimulated primary human T cells as compared to chimeric clone PD1-0103.

FIG. 3 demonstrates that the blockade of PD1 with chimeric antibody clone PD1-0103 and humanized clones PD1-0103-0312 and PD1-103-314 strongly increases tumor necrosis factor alpha (TNF) secretion by allogenic stimulated primary human T cells. TNF-alpha secretion as compared to reference antibodies MDX5C4 or MK-3475.

FIG. 4A shows the frequency of CD4⁺ T cells producing Granzyme B in the presence of increasing concentrations of different anti-PD1 antibodies (chimeric antibody clone PD1-0103 and humanized clones PD1-0103-0312, PD1-0103-0313, PD1-0103-0314, PD1-0103-0315).

FIG. 4B shows the amount of IFN-γ detected by absorbance (Optical Density, O.D.) in the supernatant of the MLR in the presence of increasing concentrations of different anti-PD1 antibodies.

FIG. 5A shows the impact of PD1/PD-L1 blockade on reactivation of suppressed T cell receptor signaling in the presence of chimeric anti-PD1 antibody clone PD1-0103 in comparison to PDL1 alone.

FIG. 5B shows the impact of PD1/PD-L1 blockade on reactivation of suppressed T cell receptor signaling in the presence of different anti-PD1 antibodies (chimeric antibody clone PD1-0103 and humanized clones PD1-0103-0312, PD1-0103-0313, PD1-0103-0314, PD1-0103-0315).

FIG. 6A, FIG. 6B, FIG. 6C, FIG. 6D, FIG. 6E, and FIG. 6F show the components for the assembly of split trimeric human 4-1BB ligands. FIG. 6A shows the dimeric 4-1BB(71-254) ligand that is fused at the C-terminus to a human CL domain with mutations E123R and Q124K (charged variant) and FIG. 6B shows the monomeric 4-1BB(71-254) ligand fused at its C-terminus to human CH1 domain with mutations K147E and K213E (charged variant). FIG. 6C shows the dimeric 4-1BB(71-254) ligand that is fused at the C-terminus to a human CL domain without the mutations and FIG. 6D shows the monomeric 4-1BB(71-254) ligand fused at its C-terminus to human CH1 domain without the mutations. FIG. 6E shows the dimeric 4-1BB(71-254) ligand that is fused at the N-terminus to a human IgG1 Fc hole domain and FIG. 6F shows the monomeric 4-1BB(71-254) ligand fused at the N-terminus to a human IgG1 Fc knob domain.

FIG. 7A, FIG. 7B, and FIG. 7C, illustrate schematically the structures of the 4-1BBL-trimer-containing antigen binding molecules Constructs 7.1 to 7.6 of the invention. The preparation and production of these constructs is described in Example 2. The VH and VL domains are those of the anti-PD1 antibody (in particular 0103-0314), the thick black point stands for the knob-into-hole modification. * symbolizes amino acid modifications in the CH1 and CL domain (so-called charged variant). FIG. 7A shows the construct 7.1 with charged variants and hu 4-1BBL(71-254), a corresponding construct without charged variants is shown in FIG. 7B. Construct 7.4 as shown in FIG. 7D comprises three hu 4-1BBL(71-248) and charged variants, the corresponding construct without charged variants is illustrated in FIG. 7E. FIG. 7C shows a bivalent construct with hu 4-1BBL (71-254) at the C-termini of the Fc chains and a corresponding construct with hu 4-1BBL(71-248) is shown in FIG. 7F.

FIG. 8A, FIG. 8B, FIG. 8C, FIG. 8D, FIG. 8E, and FIG. 8F show the components for the assembly of split trimeric human 4-1BB ligands. FIG. 8A shows the dimeric 4-1BB(71-248) ligand that is fused at the C-terminus to a human CL domain with mutations E123R and Q124K (charged variant) and FIG. 8B shows the monomeric 4-1BB(71-248) ligand fused at its C-terminus to human CH1 domain with mutations K147E and K213E (charged variant). FIG. 8C shows the dimeric 4-1BB(71-248) ligand that is fused at the C-terminus to a human CL domain without the mutations and FIG. 8D shows the monomeric 4-1BB(71-248) ligand fused at its C-terminus to human CH1 domain without the mutations. FIG. 8E shows the dimeric 4-1BB(71-248) ligand that is fused at the N-terminus to a human IgG1 Fc hole domain and FIG. 8F shows the monomeric 4-1BB(71-248) ligand fused at the N-terminus to a human IgG1 Fc knob domain.

FIG. 9A, FIG. 9B, FIG. 9C, and FIG. 9D show the components for the assembly of split trimeric human 4-1BB ligand, wherein each of dimeric 4-1BB ligand and the monomeric 4-1BB ligand is fused at its N-terminus to another polypeptide chain, in particular one of the subunits of an Fc domain. FIG. 9A shows the dimeric ligand that is fused at the N-terminus and FIG. 9B shows the monomeric ligand fused at the N-terminus. Schematic drawings of the 4-1BBL-trimer-containing antigen binding molecules Constructs 7.11 and 7.12 are shown in FIG. 9C and FIG. 9D, respectively. In FIG. 9C is shown an antigen binding molecule, wherein the Fab domain capable of specific binding to PD1 is attached to the Fc knob chain, the dimeric 4-1BBL is fused to the C-terminus of the Fc hole chain and the monomeric 4-1BBL is fused to the C-terminus of the Fc knob chain. FIG. 9D shows an antigen binding molecule, wherein the Fab domain capable of specific binding to PD1 is attached to the Fc knob chain, the dimeric 4-1BBL is also fused to the C-terminus of the Fc knob chain and the monomeric 4-1BBL is fused to the C-terminus of the Fc hole chain.

FIG. 10A and FIG. 10B are schematic drawings of Constructs 7.13 and 7.14, respectively. In FIG. 10A is shown an antigen binding molecule, wherein the Fab domain capable of specific binding to PD1 is attached to the Fc hole chain, the dimeric 4-1BBL is fused to the C-terminus of the Fc hole chain and the monomeric 4-1BBL is fused to the C-terminus of the Fc knob chain. In FIG. 10B is shown an antigen binding molecule, wherein the Fab domain capable of specific binding to a target cell antigen is attached to the Fc hole chain, the dimeric 4-1BBL is also fused to the C-terminus of the Fc knob chain and the monomeric 4-1BBL is fused to the C-terminus of the Fc hole chain.

FIG. 11A, FIG. 11B, FIG. 11C, and FIG. 11D are schematic drawings of the control molecules. FIG. 11A and FIG. 11B show the “untargeted” variants of Constructs 7.1 and 7.3, respectively, wherein the anti-PD1 Fab domains are replaced by a DP47 (germline) Fab domain. The molecules are named Control B and Control C, respectively. FIG. 11C shows the untargeted variant of Construct 7.4 named Control D. The preparation of the control molecules is described in Example 2.9. FIG. 11D is a drawing of monospecific IgG1 molecules with the PGLALA mutations, wherein the Fab domains are DP47 Fab domains (Control F) or PD1 0103-0314 Fab domains (Control M). These molecules are described in Example 2.10.

FIG. 12A shows the setup of the SPR experiments for simultaneous binding of the PD1 targeted split trimeric C-terminal 4-1BB ligand-containing antigen binding molecules of the invention.

FIG. 12 B shows the simultaneous binding to human 4-1BB and human PD1 of the PD1(0314)-targeted split 4-1BBL (71-254) Fc kih antigen binding molecule Construct 7.1.

FIG. 12C shows the simultaneous binding to human 4-1BB and human PD1 of the bivalent PD1(0314) targeted split 4-1BBL (71-254) Fc kih antigen binding molecule (Construct 7.3).

FIG. 12D shows the simultaneous binding to human 4-1BB and human PD1 of the PD1(0314) targeted split 4-1BBL (71-248) Fc kih antigen binding molecule (construct 7.5).

FIG. 12 E shows the simultaneous binding to human 4-1BB and human PD1 of the bivalent PD1(0314) targeted split 4-1BBL (71-248) Fc kih antigen binding molecule (Construct 7.6).

FIG. 13A, FIG. 13B, FIG. 13C, FIG. 13D, FIG. 13E, FIG. 13F, FIG. 13G, and FIG. 13H show the binding of PD1-targeted 4-1BB split trimeric ligand Fc fusion antigen binding molecules to freshly isolated PBMCs as described in Example 5.1. Shown is the geo mean of fluorescence intensity of a PE-conjugated secondary detection antibody (y-axis) versus the concentration of tested constructs (x-axis). For a better overview the graphs were split in two, FIG. 13A, FIG. 13B, FIG. 13C and FIG. 13D and FIG. 13E, FIG. 13F, FIG. 13G and FIG. 13H, respectively, whereby the curves for Control F and Control M were shown in all graphs as references. Binding was monitored on fresh human CD45⁺ CD3⁺ CD4⁺ T cells (FIG. 13A and FIG. 13E), fresh human CD45⁺ CD3⁺ CD8⁺ T cells (FIG. 13B and FIG. 13F), fresh human CD45⁺ CD3⁺ CD4^{neg} CD8^{neg} γδ T cells (FIG. 13C and FIG. 13G) and CD45⁺ CD3^{neg} CD19⁺ B cells (FIG. 13D and FIG. 13H).

FIG. 14A, FIG. 14B, FIG. 14C, FIG. 14D, FIG. 14E, FIG. 14F, FIG. 14G, and FIG. 14H illustrate the binding of PD1-targeted split trimeric 4-1BB ligand Fc fusion antigen binding molecules to activated human PBMCs which was tested as described in Example 5.1. Shown is the geo mean of fluorescence intensity of PE-conjugated secondary detection antibody (y-axis) versus the concentration of tested constructs (x-axis). As negative control control F was used. For a better overview the graphs were split in two, i.e. in FIG. 14A, FIG. 14B, FIG. 14C, and FIG. 14D and in FIG. 14E, FIG. 14F, FIG. 14G, and FIG. 14H whereby the curves of Control F and Control M are shown in all graphs as references. After activation with agonistic anti-human CD28 and anti-human CD3 antibody 4-1BB expression is upregulated on the majority of CD45⁺ CD3⁺ CD8⁺ T cells (see FIG. 14A or FIG. 14E) or CD45⁺ CD3⁺ CD4⁺ T cells (see FIG. 14B and FIG. 14F) and to a lesser extend to the majority of human CD45⁺ CD3⁺ CD4^{neg} CD8^{neg} γδ T cells (FIG. 14D or FIG. 14H) or CD45⁺ CD3^{neg} CD19⁺ B cells (FIG. 14D or FIG. 14H).

FIG. 15A and FIG. 15B show the binding of PD1-targeted or untargeted split trimeric human 4-1BB ligand Fc (kih) fusion antigen binding molecules to human-PD1 expressing CHO-k1-huPD1 clone 5 cells. The method is described in Example 5.2. Binding was detected with R-Phycoerythrin-fluorochrome conjugated anti-human IgG Fcγ-specific goat IgG F(ab′)2 fragment and the geo mean measured by flow cytometry is shown on the y-axis. On the x-axis the used concentration of PD1 targeted 4-1BB split trimeric ligand Fc fusion antigen binding molecules and their controls are shown. For a better overview the graphs were split in two (FIG. 15A and FIG. 15B) whereby the curves for control F and control M are shown in both as references.

FIG. 16A and FIG. 16B show the binding of PD1-targeted or untargeted split trimeric human 4-1BB ligand Fc (kih) fusion antigen binding molecules to human-4-1BB expressing HeLa-hu4-1BBL-NFkB-luc clone 26 cells as tested as described in Example 5.2. Binding was detected with R-Phycoerythrin-fluorochrome conjugated anti-human IgG Fcγ-specific goat IgG F(ab′)2 fragment and the geo mean measured by flow cytometry is shown on the y-axis. On the x-axis the used concentration of PD1 targeted 4-1BB split trimeric ligand Fc fusion antigen binding molecules and their controls are shown. For a better overview the graphs were split in two, named FIG. 16A and FIG. 16B, whereby the curves of control F and control M are shown in both graphs as references.

FIG. 17 shows a scheme that illustrates the general principal of the NFkB activity assay described in Example 6 using a reporter cell line. Shown is the activation assay set up with human 4-1BB expressing HeLa reporter cell line. A crosslinking of 4-1BB expressed on the reporter cells induces NFKB activation and NFKB-mediated luciferase expression. After lysis of the cells luciferase can catalyze the oxidation of luciferin to oxyluciferin. This chemical reaction correlates positively with the strength of NFKB-mediated luciferase expression and can be measured by the strength of light emission (units of released light).

FIG. 18A, FIG. 18B, FIG. 18C, and FIG. 18D show the NFKB-activation-induced luciferase expression and activity as measured with the assay described in Example 6. Units of released light (URL) are measured for 0.5 s/well and plotted against the used concentration of PD1-targeted or untargeted split trimeric human 4-1BB ligand Fc (kih) constructs. Human 4-1BB-expressing HeLa-reporter cells were incubated for 6 h in the absence (FIG. 18A and FIG. 18B) or presence of crosslinking human-PD1 expressing CHO-k1-huPD1 clone 5 cells (FIG. 18C and FIG. 18D). The cell ratio of human 4-1BB-expressing HeLa reporter cell to CHO-k1-huPD1 clone 5 cells is indicated in each graph.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

As used herein, the term “antigen binding molecule” refers in its broadest sense to a molecule that specifically binds an antigenic determinant. Examples of antigen binding molecules are antibodies, antibody fragments and scaffold antigen binding proteins.

As used herein, the term “moiety capable of specific binding to PD1” or “antigen binding domain capable of specific binding to PD1” refers to a polypeptide molecule that specifically binds to PD1. In one aspect, the antigen binding moiety is able to activate signaling through PD1. In a particular aspect, the antigen binding moiety is able to direct the entity to which it is attached (e.g. the TNF family ligand trimer) to a target site, for example to a specific type of tumor cell bearing PD1 or on T cells bearing PD1. Moieties capable of specific binding to PD1 include antibodies and fragments thereof as further defined herein. In addition, moieties capable of specific binding to a target cell antigen include scaffold antigen binding proteins as further defined herein, e.g. binding domains which are based on designed repeat proteins or designed repeat domains (see e.g. WO 2002/020565).

In relation to an antibody or fragment thereof, the term “moiety capable of specific binding to a target cell antigen” refers to the part of the molecule that comprises the area which specifically binds to and is complementary to part or all of an antigen. A moiety capable of specific antigen binding may be provided, for example, by one or more antibody variable domains (also called antibody variable regions). Particularly, a moiety capable of specific antigen binding comprises an antibody light chain variable region (VL) and an antibody heavy chain variable region (VH).

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used within the current application denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds.

Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); and single domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments in which the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab (VLVH). On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab (CLCH1).

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen-binding site which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13: 695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), VNAR fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a kunitz type domain of human protease inhibitors, microbodies such as the proteins from the knottin family, peptide aptamers and fibronectin (adnectin).

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001).

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633.

An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 17, 455-462 (2004) and EP 1641818A1.

Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007).

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

A single-domain antibody is an antibody fragment consisting of a single monomeric variable antibody domain. The first single domain were derived from the variable domain of the antibody heavy chain from camelids (nanobodies or VHH fragments). Furthermore, the term single-domain antibody includes an autonomous human heavy chain variable domain (aVH) or VNAR fragments derived from sharks.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

The term “antigen binding domain” refers to the part of an antigen binding molecule that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH).

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

The term “capable of specific binding to PD1” refers to an antigen binding molecule that is capable of binding PD1 with sufficient affinity such that the antigen binding molecule is useful as a diagnostic and/or therapeutic agent in targeting PD1. The antigen binding molecule includes but is not limited to, antibodies, Fab molecules, crossover Fab molecules, single chain Fab molecules, Fv molecules, scFv molecules, single domain antibodies, and VH and scaffold antigen binding protein. In one aspect, the extent of binding of an anti-PD1 antigen binding molecule to an unrelated, non-PD1 protein is less than about 10% of the binding of the antigen binding molecule to PD1 as measured, e.g., by a radioimmunoassay (RIA). In particular, an antigen binding molecule that is capable of specific binding to PD1 has a dissociation constant (K_{d}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-PD1 antigen binding molecule binds to PD1 from different species. In particular, the anti-PD1 antigen binding molecule binds to human and cynomolgus PD1.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (Kd), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

A “target cell antigen” as used herein refers to an antigenic determinant presented on the surface of a target cell, for example a T-cell or B-cell, a cell in a tumor such as a cancer cell or a cell of the tumor stroma. In certain embodiments, the target cell antigen is an antigen on the surface of T cell. In one embodiment, the target cell antigen is PD1.

The term “PD1”, also known as Programmed cell death protein 1, is a type I membrane protein of 288 amino acids that was first described in 1992 (Ishida et al., EMBO J., 11 (1992), 3887-3895). PD1 is a member of the extended CD28/CTLA-4 family of T cell regulators and has two ligands, PD-L1 (B7-H1, CD274) and PD-L2 (B7-DC, CD273). The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD1 negatively regulates TCR signals. This is consistent with binding of SHP-1 and SHP-2 phosphatases to the cytoplasmic tail of PD1 upon ligand binding. While PD1 is not expressed on naïve T cells, it is upregulated following T cell receptor (TCR)-mediated activation and is observed on both activated and exhausted T cells (Agata et al., Int. Immunology 8 (1996), 765-772). These exhausted T-cells have a dysfunctional phenotype and are unable to respond appropriately. Although PD1 has a relatively wide expression pattern its most important role is likely as a coinhibitory receptor on T cells (Chinai et al, Trends in Pharmacological Sciences 36 (2015), 587-595). Current therapeutic approaches thus focus on blocking the interaction of PD1 with its ligands to enhance T cell response. The terms “Programmed Death 1,” “Programmed Cell Death 1,” “Protein PD-1,” “PD-1,” “PD1,” “PDCD1,” “hPD-1” and “hPD-I” can be used interchangeably, and include variants, isoforms, species homologs of human PD1, and analogs having at least one common epitope with PD1. The amino acid sequence of human PD1 is shown in UniProt (www.uniprot.org) accession no. Q15116 (SEQ ID NO:94).

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen binding specificity.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) Hypervariable regions (HVRs) are also referred to as complementarity determining regions (CDRs), and these terms are used herein interchangeably in reference to portions of the variable region that form the antigen binding regions. This particular region has been described by Kabat et al., U.S. Dept. of Health and Human Services, “Sequences of Proteins of Immunological Interest” (1983) and by Chothia et al., J. Mol. Biol. 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table A as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE A
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table A is according to the numbering conventions set forth by Kabat et al. (see below).
²“AbM” with a lowercase “b” as used in Table A refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

As used herein, the term “affinity matured” in the context of antigen binding molecules (e.g., antibodies) refers to an antigen binding molecule that is derived from a reference antigen binding molecule, e.g., by mutation, binds to the same antigen, preferably binds to the same epitope, as the reference antibody; and has a higher affinity for the antigen than that of the reference antigen binding molecule. Affinity maturation generally involves modification of one or more amino acid residues in one or more CDRs of the antigen binding molecule. Typically, the affinity matured antigen binding molecule binds to the same epitope as the initial reference antigen binding molecule.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody.

A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to C1q binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “Fc domain” or “Fe region” herein is used to define a C-terminal region of an antibody heavy chain that contains at least a portion of the constant region. The term includes native sequence Fc regions and variant Fc regions. An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU index of Kabat et al, Sequences of Proteins of Immunological Interest, 5th Ed. Public Health Service, National Institutes of Health, Bethesda, Md., 1991.

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “TNF ligand family member” or “TNF family ligand” refers to a proinflammatory cytokine. Cytokines in general, and in particular the members of the TNF ligand family, play a crucial role in the stimulation and coordination of the immune system. At present, nineteen cyctokines have been identified as members of the TNF (tumour necrosis factor) ligand superfamily on the basis of sequence, functional, and structural similarities. All these ligands are type II transmembrane proteins with a C-terminal extracellular domain (ectodomain), N-terminal intracellular domain and a single transmembrane domain. The C-terminal extracellular domain, known as TNF homology domain (THD), has 20-30% amino acid identity between the superfamily members and is responsible for binding to the receptor. The TNF ectodomain is also responsible for the TNF ligands to form trimeric complexes that are recognized by their specific receptors.

Members of the TNF ligand family are selected from the group consisting of Lymphotoxin a (also known as LTA or TNFSF1), TNF (also known as TNFSF2), LT(3 (also known as TNFSF3), OX40L (also known as TNFSF4), CD40L (also known as CD154 or TNFSF5), FasL (also known as CD95L, CD178 or TNFSF6), CD27L (also known as CD70 or TNFSF7), CD30L (also known as CD153 or TNFSF8), 4-1BBL (also known as TNFSF9), TRAIL (also known as APO2L, CD253 or TNFSF10), RANKL (also known as CD254 or TNFSF11), TWEAK (also known as TNFSF12), APRIL (also known as CD256 or TNFSF13), BAFF (also known as CD257 or TNFSF13B), LIGHT (also known as CD258 or TNFSF14), TL1A (also known as VEGI or TNFSF15), GITRL (also known as TNFSF18), EDA-A1 (also known as ectodysplasin A1) and EDA-A2 (also known as ectodysplasin A2). The term refers to any native TNF family ligand from any vertebrate source, including mammals such as primates (e.g. humans), non-human primates (e.g. cynomolgus monkeys) and rodents (e.g. mice and rats), unless otherwise indicated. In specific embodiments of the invention, the TNF ligand family member is selected from the group consisting of OX40L, FasL, CD27L, TRAIL, 4-1BBL, CD40L and GITRL. In a particular embodiment, the TNF ligand family member is selected from 4-1BBL and OX40L.

Further information, in particular sequences, of the TNF ligand family members may be obtained from publically accessible databases such as Uniprot (www.uniprot.org). For instance, the human TNF ligands have the following amino acid sequences: human Lymphotoxin at (UniProt accession no. P01374, SEQ ID NO:95), human TNF (UniProt accession no. P01375, SEQ ID NO:96), human Lymphotoxin 3 (UniProt accession no. Q06643, SEQ ID NO:97), human OX40L (UniProt accession no. P23510, SEQ ID NO:98), human CD40L (UniProt accession no. P29965, SEQ ID NO:99), human FasL (UniProt accession no. P48023, SEQ ID NO: 100), human CD27L (UniProt accession no. P32970, SEQ ID NO: 101), human CD30L (UniProt accession no. P32971, SEQ ID NO:102), 4-1BBL (UniProt accession no. P41273, SEQ ID NO:103), TRAIL (UniProt accession no. P50591, SEQ ID NO:104), RANKL (UniProt accession no. 014788, SEQ ID NO:105), TWEAK (UniProt accession no. 043508, SEQ ID NO:106), APRIL (UniProt accession no. 075888, SEQ ID NO: 107), BAFF (UniProt accession no. Q9Y275, SEQ ID NO:108), LIGHT (UniProt accession no. 043557, SEQ ID NO:109), TL1A (UniProt accession no. 095150, SEQ ID NO:110), GITRL (UniProt accession no. Q9UNG2, SEQ ID NO: 111) and ectodysplasin A (UniProt accession no. Q92838, SEQ ID NO:112).

An “ectodomain” is the domain of a membrane protein that extends into the extracellular space (i.e. the space outside the target cell). Ectodomains are usually the parts of proteins that initiate contact with surfaces, which leads to signal transduction. The ectodomain of TNF ligand family member as defined herein thus refers to the part of the TNF ligand protein that extends into the extracellular space (the extracellular domain), but also includes shorter parts or fragments thereof that are responsible for the trimerization and for the binding to the corresponding TNF receptor. The term “ectodomain of a TNF ligand family member or a fragment thereof” thus refers to the extracellular domain of the TNF ligand family member that forms the extracellular domain or to parts thereof that are still able to bind to the receptor (receptor binding domain).

The term “costimulatory TNF ligand family member” or “costimulatory TNF family ligand” refers to a subgroup of TNF ligand family members, which are able to costimulate proliferation and cytokine production of T-cells. These TNF family ligands can costimulate TCR signals upon interaction with their corresponding TNF receptors and the interaction with their receptors leads to recruitment of TNFR-associated factors (TRAF), which initiate signalling cascades that result in T-cell activation. Costimulatory TNF family ligands are selected from the group consisting of 4-1BBL, OX40L, GITRL, CD70, CD30L and LIGHT, more particularly the costimulatory TNF ligand family member is selected from 4-1BBL and OX40L.

As described herein before, 4-1BBL is a type II transmembrane protein and one member of the TNF ligand family. Complete or full length 4-1BBL having the amino acid sequence of SEQ ID NO: 103 has been described to form trimers on the surface of cells. The formation of trimers is enabled by specific motives of the ectodomain of 4-1BBL. Said motives are designated herein as “trimerization region”. The amino acids 50-254 of the human 4-1BBL sequence (SEQ ID NO: 113) form the extracellular domain of 4-1BBL, but even fragments thereof are able to form the trimers. In specific embodiments of the invention, the term “ectodomain of 4-1BBL or a fragment thereof” refers to a polypeptide having an amino acid sequence selected from SEQ ID NO:4 (amino acids 52-254 of human 4-1BBL), SEQ ID NO:1 (amino acids 71-254 of human 4-1BBL), SEQ ID NO:3 (amino acids 80-254 of human 4-1BBL) and SEQ ID NO:2 (amino acids 85-254 of human 4-1BBL) or a polypeptide having an amino acid sequence selected from SEQ ID NO:5 (amino acids 71-248 of human 4-1BBL), SEQ ID NO:8 (amino acids 52-248 of human 4-1BBL), SEQ ID NO:7 (amino acids 80-248 of human 4-1BBL) and SEQ ID NO:6 (amino acids 85-248 of human 4-1BBL), but also other fragments of the ectodomain capable of trimerization are included herein.

As described herein before, OX40L is another type II transmembrane protein and a further member of the TNF ligand family. Complete or full length human OX40L has the amino acid sequence of SEQ ID NO:98. The amino acids 51-183 of the human OX40L sequence (SEQ ID NO: 114) form the extracellular domain of OX40L, but even fragments thereof that are able to form the trimers. In specific embodiments of the invention, the term “ectodomain of OX40L or a fragment thereof” refers to a polypeptide having an amino acid sequence selected from SEQ ID NO: 114 (amino acids 51-183 of human OX40L) or SEQ ID NO: 115 (amino acids 52-183 of human OX40L), but also other fragments of the ectodomain capable of trimerization are included herein.

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 1 and 4, in particular 2, i.e. the peptides selected from the group consisting of GGGGS (SEQ ID NO: 116), GGGGSGGGGS (SEQ ID NO:117), SGGGGSGGGG (SEQ ID NO:118), (G₄S)₃ or GGGGSGGGGSGGGGS (SEQ ID NO:119), GGGGSGGGGSGGGG or G4(SG4)₂ (SEQ ID NO:120), and (G₄S)₄ or GGGGSGGGGSGGGGSGGGGS (SEQ ID NO: 121), but also include the sequences GSPGSSSSGS (SEQ ID NO:122), GSGSGSGS (SEQ ID NO:123), GSGSGNGS (SEQ ID NO:124), GGSGSGSG (SEQ ID NO: 125), GGSGSG (SEQ ID NO: 126), GGSG (SEQ ID NO: 127), GGSGNGSG (SEQ ID NO: 128), GGNGSGSG (SEQ ID NO: 129) and GGNGSG (SEQ ID NO:130). Peptide linkers of particular interest are (G4S)₁ or GGGGS (SEQ ID NO:86), (G₄S)₂ or GGGGSGGGGS (SEQ ID NO: 117) and GSPGSSSSGS (SEQ ID NO: 122), more particularly (G₄S)₂ or GGGGSGGGGS (SEQ ID NO:117).

The term “amino acid” as used within this application denotes the group of naturally occurring carboxy a-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “fusion polypeptide” or “single fusion polypeptide” as used herein refers to a single chain polypeptide composed of one or two ectodomains of said TNF ligand family member fused to a part of antigen binding moiety or Fc part. The fusion may occur by directly linking the N or C-terminal amino acid of the antigen binding moiety via a peptide linker to the C- or N-terminal amino acid of the ectodomain of said TNF ligand family member.

By “fused” or “connected” is meant that the components (e.g. a polypeptide and an ectodomain of said TNF ligand family member) are linked by peptide bonds, either directly or via one or more peptide linkers.

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain embodiments, amino acid sequence variants of the TNF ligand trimer-containing antigen binding molecules provided herein are contemplated. For example, it may be desirable to improve the binding affinity and/or other biological properties of the TNF ligand trimer-containing antigen binding molecules. Amino acid sequence variants of the TNF ligand trimer-containing antigen binding molecules may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table B under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

TABLE B

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include TNF family ligand trimer-containing antigen binding molecule with an N-terminal methionyl residue. Other insertional variants of the molecule include the fusion to the N- or C-terminus to a polypeptide which increases the serum half-life of the TNF ligand trimer-containing antigen binding molecules.

In certain embodiments, the TNF family ligand trimer-containing antigen binding molecules provided herein are altered to increase or decrease the extent to which the antibody is glycosylated. Glycosylation variants of the molecules may be conveniently obtained by altering the amino acid sequence such that one or more glycosylation sites is created or removed. Where the TNF ligand trimer-containing antigen binding molecule comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in TNF family ligand trimer-containing antigen binding molecule may be made in order to create variants with certain improved properties. In one aspect, variants of TNF family ligand trimer-containing antigen binding molecules are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the TNF family ligand trimer-containing antigen binding molecules of the invention include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain embodiments, it may be desirable to create cysteine engineered variants of the TNF family ligand trimer-containing antigen binding molecule of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the TNF family ligand trimer-containing antigen binding molecules provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc. In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

In another aspect, immunoconjugates of the TNF family ligand trimer-containing antigen binding molecules provided herein maybe obtained. An “immunoconjugate” is an antibody conjugated to one or more heterologous molecule(s), including but not limited to a cytotoxic agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein refers to proliferative diseases, such as lymphomas, carcinoma, lymphoma, blastoma, sarcoma, leukemia, lymphocytic leukemias, lung cancer, non-small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colorectal cancer (CRC), pancreatic cancer, breast cancer, triple-negative breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma and Ewings sarcoma, melanoma, multiple myeloma, B-cell cancer (lymphoma), chronic lymphocytic leukemia (CLL), acute lymphoblastic leukemia (ALL), hairy cell leukemia, chronic myeloblastic leukemia, including refractory versions of any of the above cancers, or a combination of one or more of the above cancers.

TNF Family Ligand Trimer-Containing Antigen Binding Molecules of the Invention

The invention provides novel TNF family ligand trimer-containing antigen binding molecules with particularly advantageous properties such as producibility, stability, binding affinity, biological activity, targeting efficiency and reduced toxicity.

In a first aspect, the invention provides a TNF family ligand trimer-containing antigen binding molecule comprising

(a) at least one moiety capable of specific binding to PD1 and
(b) a first and a second polypeptide that are linked to each other by a disulfide bond,
wherein the antigen binding molecule is characterized in that the first polypeptide comprises two ectodomains of a TNF ligand family member or a fragment thereof that are connected to each other by a peptide linker and in that the second polypeptide comprises only one ectodomain of said TNF ligand family member or a fragment thereof.

In a particular aspect, the invention provides a TNF family ligand trimer-containing antigen binding molecule comprising

(a) at least one moiety capable of specific binding to PD1,
(b) a first and a second polypeptide that are linked to each other by a disulfide bond,
wherein the antigen binding molecule is characterized in that the first polypeptide comprises two ectodomains of a TNF ligand family member or a fragment thereof that are connected to each other by a peptide linker and in that the second polypeptide comprises only one ectodomain of said TNF ligand family member or a fragment thereof, and
(c) an Fc domain composed of a first and a second subunit capable of stable association.

In a particular aspect, the TNF family ligand trimer-containing antigen binding molecule comprises (a) at least one moiety capable of specific binding to PD1 and

(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof, wherein the TNF ligand family member is costimulates human T-cell activation.

In another particular aspect, the TNF family ligand trimer-containing antigen binding molecule comprises (a) at least one moiety capable of specific binding to PD1 and

(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof, wherein the ectodomains of a TNF ligand family member are identical in all instances.

In a further aspect, provided is a TNF family ligand trimer-containing antigen binding molecule as defined herein before, comprising

(a) at least one moiety capable of specific binding to a PD1 and
(b) a first and a second polypeptide that are linked to each other by a disulfide bond,
wherein the antigen binding molecule is characterized in that
(i) the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of a TNF ligand family member or two fragments thereof that are connected to each other and to the CH1 or CL domain by a peptide linker and wherein the second polypeptide comprises one ectodomain of said TNF ligand family member or a fragment thereof connected via a peptide linker to the CL or CH1 domain of said polypeptide, or
(ii) the first polypeptide contains a CH3 domain and the second polypeptide contains a CH3 domain, respectively, and wherein the first polypeptide comprises two ectodomains of a TNF ligand family member or two fragments thereof that are connected to each other and to the C-terminus of the CH3 domain by a peptide linker and wherein the second polypeptide comprises only one ectodomain of said TNF ligand family member or a fragment thereof connected via a peptide linker to C-terminus of the CH3 domain of said polypeptide, or
(iii) the first polypeptide contains a VH-CL or a VL-CH1 domain and the second polypeptide contains a VL-CH1 domain or a VH-CL domain, respectively, wherein the second polypeptide is linked to the first polypeptide by a disulfide bond between the CH1 and CL domain, and wherein the first polypeptide comprises two ectodomains of a TNF ligand family member or fragments thereof that are connected to each other and to to VH or VL by a peptide linker and wherein the second polypeptide comprises one ectodomain of said TNF ligand family member or a fragment thereof connected via a peptide linker to VL or VH of said polypeptide.

In another aspect, provided is a TNF family ligand trimer-containing antigen binding molecule of as defined herein before, comprising

(a) at least one moiety capable of specific binding to a PD1 and
(b) a first and a second polypeptide that are linked to each other by a disulfide bond,
wherein the antigen binding molecule is characterized in that
(i) the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of a TNF ligand family member or two fragments thereof that are connected to each other and to the CH1 or CL domain by a peptide linker and wherein the second polypeptide comprises one ectodomain of said TNF ligand family member or a fragment thereof connected via a peptide linker to the CL or CH1 domain of said polypeptide, or
(ii) the first polypeptide contains a CH3 domain and the second polypeptide contains a CH3 domain, respectively, and wherein the first polypeptide comprises two ectodomains of a TNF ligand family member or two fragments thereof that are connected to each other and to the C-terminus of the CH3 domain by a peptide linker and wherein the second polypeptide comprises only one ectodomain of said TNF ligand family member or a fragment thereof connected via a peptide linker to C-terminus of the CH3 domain of said polypeptide.

In one aspect, the TNF family ligand trimer-containing antigen binding molecule comprises a TNF ligand family member that costimulates human T-cell activation which is selected from 4-1BBL and OX40L. More particularly, the TNF ligand family member is 4-1BBL.

In another aspect, wherein the ectodomain of a TNF ligand family member comprises the amino acid sequence selected from the group consisting of SEQ ID NO: 1, SEQ ID NO: 2, SEQ ID NO:3, SEQ ID NO:4, SEQ ID NO:5, SEQ ID NO: 6, SEQ ID NO:7 and SEQ ID NO:8, particularly the amino acid sequence of SEQ ID NO: 1 or SEQ ID NO:5. More particularly, the ectodomain of a TNF ligand family member comprises the amino acid sequence of SEQ ID NO:5.

In a further aspect, the TNF family ligand trimer-containing antigen binding molecule of the invention comprises

(a) at least one moiety capable of specific binding to PD1 and
(b) a first and a second polypeptide that are linked to each other by a disulfide bond,
wherein the antigen binding molecule is characterized in that the first polypeptide comprises the amino acid sequence selected from the group consisting of SEQ ID NO:9, SEQ ID NO: 11, SEQ ID NO: 12 and SEQ ID NO: 10 and in that the second polypeptide comprises the amino acid sequence selected from the group consisting of SEQ ID NO:1, SEQ ID NO:3, SEQ ID NO:4 and SEQ ID NO:5.

In one aspect, the TNF family ligand trimer-containing antigen binding molecule of the invention comprises

(a) at least one moiety capable of specific binding to PD1 and
(b) a first and a second polypeptide that are linked to each other by a disulfide bond,
wherein the antigen binding molecule is characterized in that the first polypeptide comprises the amino acid sequence of SEQ ID NO: 10 and in that the second polypeptide comprises the amino acid sequence of SEQ ID NO:5.

In a further aspect, the TNF family ligand trimer-containing antigen binding molecule of the invention comprises

(a) at least one moiety capable of specific binding to PD1 and
(b) a first and a second polypeptide that are linked to each other by a disulfide bond,
wherein the antigen binding molecule is characterized in that the first polypeptide comprises the amino acid sequence of SEQ ID NO:9 and in that the second polypeptide comprises the amino acid sequence of SEQ ID NO:1.

In another aspect, the TNF ligand family member is OX40L. In a particular aspect, provided is TNF family ligand trimer-containing antigen binding molecule, wherein the ectodomain of a TNF ligand family member comprises the amino acid sequence of SEQ ID NO: 114 or SEQ ID NO: 115, particularly the amino acid sequence of SEQ ID NO: 114.

In another aspect, the TNF family ligand trimer-containing antigen binding molecule of the invention comprises

(a) at least one moiety capable of specific binding to PD1,
(b) a first polypeptide containing a CH1 or CL domain and a second polypeptide containing a CL or CH1 domain, respectively, wherein the second polypeptide is linked to the first polypeptide by a disulfide bond between the CH1 and CL domain,
and wherein the antigen binding molecule is characterized in that the first polypeptide comprises two ectodomains of a TNF ligand family member or two fragments thereof that are connected to each other and to the CH1 or CL domain by a peptide linker and in that the second polypeptide comprises only one ectodomain of said TNF ligand family member or a fragment thereof connected by a peptide linker to the CL or CH1 domain of said polypeptide.

In one aspect, provided is a TNF family ligand trimer-containing antigen binding molecule comprising

(a) at least one moiety capable of specific binding to PD1,
(b) a first polypeptide containing a CH1 domain and a second polypeptide containing a CL domain, wherein the second polypeptide is linked to the first polypeptide by a disulfide bond between the CH1 and CL domain,
and wherein the antigen binding molecule is characterized in that the first polypeptide comprises two ectodomains of a TNF ligand family member or fragments thereof that are connected to each other and to the CH1 domain by a peptide linker and in that the second polypeptide comprises one ectodomain of said TNF ligand family member or a fragment thereof connected via a peptide linker to the CL domain of said polypeptide.

In another aspect, provided is a TNF family ligand trimer-containing antigen binding molecule comprising

(a) at least one moiety capable of specific binding to PD1,
(b) a first polypeptide containing a CL domain and a second polypeptide containing a CH1 domain, wherein the second polypeptide is linked to the first polypeptide by a disulfide bond between the CH1 and CL domain,
and wherein the antigen binding molecule is characterized in that the first polypeptide comprises two ectodomains of a TNF ligand family member or fragments thereof that are connected to each other and to the CL domain by a peptide linker and in that the second polypeptide comprises one ectodomain of said TNF ligand family member or a fragment thereof connected via a peptide linker to the CH1 domain of said polypeptide.

In another aspect, the invention provides a TNF family ligand trimer-containing antigen binding molecule comprising

(a) one moiety capable of specific binding to PD1 and
(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof.

In yet another aspect, the invention provides a TNF family ligand trimer-containing antigen binding molecule comprising

(a) more than one moiety capable of specific binding to PD1 and
(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof connected via a peptide linker to said polypeptide.

In one aspect, the invention provides a TNF family ligand trimer-containing antigen binding molecule comprising

(a) two moities capable of specific binding to PD1 and
(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof.

In a particular aspect, provided is a TNF family ligand trimer-containing antigen binding molecule, comprising

(a) at least one moiety capable of specific binding to PD1, and
(b) a first and a second polypeptide that are linked to each other by a disulfide bond,

wherein the antigen binding molecule is characterized in that the first polypeptide contains a CH3 domain and the second polypeptide contains a CH3 domain, respectively, and wherein the first polypeptide comprises two ectodomains of a TNF ligand family member or fragments thereof that are connected to each other and to the C-terminus of the CH3 domain by a peptide linker and wherein the second polypeptide comprises one ectodomain of said TNF ligand family member or a fragment thereof connected via a peptide linker to C-terminus of the CH3 domain of said polypeptide. Particularly, such TNF family ligand trimer-containing antigen binding molecule comprises two moieties capable of specific binding to a target cell antigen.

In one aspect, the invention provides a TNF family ligand trimer-containing antigen binding molecule comprising

(a) two moities capable of specific binding to PD1 and
(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises one ectodomain of said TNF ligand family member or a fragment thereof,
wherein the two moieties capable of specific binding to a target cell antigen bind to two different target cell antigens.

In a further aspect, the invention provides a TNF family ligand trimer-containing antigen binding molecule as defined herein before, wherein the moiety capable of specific binding to a target cell antigen is selected from the group consisting of an antibody, an antibody fragment and a scaffold antigen binding protein.

In one aspect, provided is a TNF family ligand trimer-containing antigen binding molecule as described herein before, wherein the moiety capable of specific binding to PD1 is selected from the group consisting of an antibody fragment, a Fab molecule, a crossover Fab molecule, a single chain Fab molecule, a Fv molecule, a scFv molecule, a single domain antibody, an aVH and a scaffold antigen binding protein. In one aspect, the moiety capable of specific binding to PD1 is an aVH or a scaffold antigen binding protein. In one aspect, the moiety capable of specific binding to a target cell antigen is a scaffold antigen binding protein capable of specific binding to a target cell antigen.

In particular, the TNF family ligand trimer-containing antigen binding molecule comprises one or two moieties capable of specific binding to PD1.

In a particular aspect, provided is a TNF family ligand trimer-containing antigen binding molecule, wherein the moiety capable of specific binding to PD1 is a Fab molecule or a crossover Fab molecule capable of specific binding to PD1. In particular, the moiety capable of specific binding to a target cell antigen is a Fab capable of specific binding to PD1.

In a further aspect, provided is a TNF family ligand trimer-containing antigen binding molecule according to the invention, wherein a peptide comprising two ectodomains of a TNF ligand family member or fragments thereof connected to each other by a first peptide linker is fused at its C-terminus to the CH1 domain of a heavy chain by a second peptide linker and wherein one ectodomain of said TNF ligand family member or a fragment thereof is fused at the its C-terminus to the CL domain on a light chain by a third peptide linker.

In another aspect, provided is a TNF family ligand trimer-containing antigen binding molecule according to the invention, wherein a peptide comprising two ectodomains of a TNF ligand family member or fragments thereof connected to each other by a first peptide linker is fused at its C-terminus to the CL domain of a heavy chain by a second peptide linker and wherein one ectodomain of said TNF ligand family member or a fragment thereof is fused at the its C-terminus to the CH1 domain on a light chain by a third peptide linker.

In a further aspect, the invention is concerned with a TNF family ligand trimer-containing antigen binding molecule according to the invention, wherein a peptide comprising two ectodomains of a TNF ligand family member or fragments thereof connected to each other by a first peptide linker is fused at its C-terminus to the CL domain of a light chain by a second peptide linker and wherein one ectodomain of said TNF ligand family member or a fragment thereof is fused at the its C-terminus to the CH1 domain of the heavy chain by a third peptide linker.

In a particular aspect, the invention relates to a TNF family ligand trimer-containing antigen binding molecule as defined above, wherein the peptide linker is (G4S)₂. In one aspect, the first peptide linker is (G4S)₂ (SEQ ID NO: 117), the second peptide linker is GSPGSSSSGS (SEQ ID NO:122) and the third peptide linker is (G4S)₂ (SEQ ID NO:117). In particular, the invention relates to a TNF ligand trimer-containing antigen binding molecule as defined above, wherein the first peptide linker is (G4S)₂ (SEQ ID NO: 117), the second peptide linker is (G4S)₂ (SEQ ID NO: 117), and the third peptide linker is (G4S)₂ (SEQ ID NO: 117).

In another aspect, the TNF family ligand trimer-containing antigen binding molecule as defined herein before comprises an Fc domain composed of a first and a second subunit capable of stable association.

In particular, the TNF family ligand trimer-containing antigen binding molecule of the invention comprises (a) a Fab molecule capable of specific binding to PD1, wherein the Fab heavy chain is fused at the C-terminus to the N-terminus of a CH2 domain in the Fc domain and (c) an Fc domain composed of a first and a second subunit capable of stable association.

In a further aspect, the Fc domain is an IgG, particularly an IgG1 Fc domain or an IgG4 Fc domain. More particularly, the Fc domain is an IgG1 Fc domain. In a particular aspect, the Fc domain comprises a modification promoting the association of the first and second subunit of the Fc domain.

Fc Domain Modifications Reducing Fc Receptor Binding and/or Effector Function

The Fc domain of the TNF family ligand trimer-containing antigen binding molecules of the invention consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other.

The Fc domain confers favorable pharmacokinetic properties to the antigen binding molecules of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular aspects, the Fc domain of the TNF family ligand trimer-containing antigen binding molecule of the invention exhibits reduced binding affinity to an Fc receptor and/or reduced effector function, as compared to a native IgG1 Fc domain. In one aspect, the Fc does not substantially bind to an Fc receptor and/or does not induce effector function. In a particular aspect the Fc receptor is an Fcγ receptor. In one aspect, the Fc receptor is a human Fc receptor. In a specific aspect, the Fc receptor is an activating human Fcγ receptor, more specifically human FcγRIIIa, FcγRI or FcγRIIa, most specifically human FcγRIIIa. In one aspect, the Fc domain does not induce effector function. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

In certain aspects, one or more amino acid modifications may be introduced into the Fc region of a TNF family ligand trimer-containing antigen binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In a particular aspect, the invention provides a TNF family ligand trimer-containing antigen binding molecule comprising

(a) at least one moiety capable of specific binding to PD1,
(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof, and
(c) an Fc domain composed of a first and a second subunit capable of stable association, wherein the Fc domain comprises one or more amino acid substitution that reduces binding to an Fc receptor, in particular towards Fcγ receptor.

In one aspect, the Fc domain of the TNF family ligand trimer-containing antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In particular, the Fc domain comprises an amino acid substitution at a position of E233, L234, L235, N297, P331 and P329 (EU numbering). In particular, the Fc domain comprises amino acid substitutions at positions 234 and 235 (EU numbering) and/or 329 (EU numbering) of the IgG heavy chains. More particularly, provided is a trimeric TNF family ligand-containing antigen binding molecule according to the invention which comprises an Fc domain with the amino acid substitutions L234A, L235A and P329G (“P329G LALA”, EU numbering) in the IgG heavy chains. The amino acid substitutions L234A and L235A refer to the so-called LALA mutation. The “P329G LALA” combination of amino acid substitutions almost completely abolishes Fcγ receptor binding of a human IgG1 Fc domain and is described in International Patent Appl. Publ. No. WO 2012/130831 A1 which also describes methods of preparing such mutant Fc domains and methods for determining its properties such as Fc receptor binding or effector functions. “EU numbering” refers to the numbering according to EU index of Kabat et al, Sequences of Proteins of Immunological Interest, 5th Ed. Public Health Service, National Institutes of Health, Bethesda, Md., 1991.

Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In another aspect, the Fc domain is an IgG4 Fc domain. IgG4 antibodies exhibit reduced binding affinity to Fc receptors and reduced effector functions as compared to IgG1 antibodies. In a more specific aspect, the Fc domain is an IgG4 Fc domain comprising an amino acid substitution at position S228 (Kabat numbering), particularly the amino acid substitution S228P. In a more specific aspect, the Fc domain is an IgG4 Fc domain comprising amino acid substitutions L235E and S228P and P329G (EU numbering). Such IgG4 Fc domain mutants and their Fcγ receptor binding properties are also described in WO 2012/130831.

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the bispecific antibodies of the invention is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

In a particular aspect, the Fc domain comprises a modification promoting the association of the first and second subunit of the Fc domain.

Fc Domain Modifications Promoting Heterodimerization

In one aspect, the TNF family ligand trimer-containing antigen binding molecules of the invention comprise (a) at least one moiety capable of specific binding to a PD1,

(b) a first and a second polypeptide that are linked to each other by a disulfide bond,
wherein the antigen binding molecule is characterized in that the first polypeptide comprises two ectodomains of a TNF ligand family member or two fragments thereof that are connected to each other by a peptide linker and in that the second polypeptide comprises only one ectodomain of said TNF ligand family member or a fragment thereof, and (c) an Fc domain composed of a first and a second subunit capable of stable association, wherein the Fc domain comprises one or more amino acid substitution that reduces binding to an Fc receptor, in particular towards Fcγ receptor. Thus, they comprise different moieties, fused to one or the other of the two subunits of the Fc domain that are typically comprised in two non-identical polypetide chains (“heavy chains”). Recombinant co-expression of these polypeptides and subsequent dimerization leads to several possible combinations of the two polypeptides. To improve the yield and purity of the TNF family ligand trimer-containing antigen binding molecules in recombinant production, it will thus be advantageous to introduce in the Fc domain of the TNF family ligand trimer-containing antigen binding molecules of the invention a modification promoting the association of the desired polypeptides.

Accordingly, the Fc domain of the TNF family ligand trimer-containing antigen binding molecules of the invention comprises a modification promoting the association of the first and the second subunit of the Fc domain. The site of most extensive protein-protein interaction between the two subunits of a human IgG Fc domain is in the CH3 domain of the Fc domain. Thus, said modification is particularly in the CH3 domain of the Fc domain.

In a specific aspect, said modification is a so-called “knob-into-hole” modification, comprising a “knob” modification in one of the two subunits of the Fc domain and a “hole” modification in the other one of the two subunits of the Fc domain. Thus, in a particular aspect, the invention relates to a TNF family ligand trimer-containing antigen binding molecule as described herein before which comprises an IgG molecule, wherein the Fc part of the first heavy chain comprises a first dimerization module and the Fc part of the second heavy chain comprises a second dimerization module allowing a heterodimerization of the two heavy chains of the IgG molecule and the first dimerization module comprises knobs and the second dimerization module comprises holes according to the knob into hole technology.

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular aspect, in the CH3 domain of the first subunit of the Fc domain of the TNF family ligand trimer-containing antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). More particularly, in the second subunit of the Fc domain additionally the threonine residue at position 366 is replaced with a serine residue (T366S) and the leucine residue at position 368 is replaced with an alanine residue (L368A). More particularly, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). The introduction of these two cysteine residues results in the formation of a disulfide bridge between the two subunits of the Fc domain. The disulfide bridge further stabilizes the dimer (Carter, J Immunol Methods 248, 7-15 (2001)).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Modifications in the CH1/CL Domains

To further improve correct pairing, the TNF family ligand trimer-containing antigen binding molecules can contain different charged amino acid substitutions (so-called “charged residues”). These modifications are introduced in the crossed or non-crossed CH1 and CL domains. In a particular aspect, the invention relates to a TNF family ligand trimer-containing antigen binding molecule, wherein in one of CL domains the amino acid at position 123 (EU numbering) has been replaced by arginine (R) and the amino acid at position 124 (EU numbering) has been substituted by lysine (K) and wherein in one of the CH1 domains the the amino acids at position 147 (EU numbering) and at position 213 (EU numbering) have been substituted by glutamic acid (E).

More particularly, the invention relates to a TNF family ligand trimer-containing antigen binding molecule, wherein in the CL domain adjacent to the TNF ligand family member the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E).

Thus, in a particular aspect, provided is a TNF family ligand trimer-containing antigen binding molecule comprising

(a) at least one moiety capable of specific binding to PD1,
(b) a first polypeptide containing a CL domain comprising the amino acid mutations E123R and Q124K and a second polypeptide containing a CH1 domain comprising the amino acid mutations K147E and K213E, wherein the second polypeptide is linked to the first polypeptide by a disulfide bond between the CH1 and CL domain,
and wherein the antigen binding molecule is characterized in that the first polypeptide comprises two ectodomains of a TNF ligand family member or fragments thereof that are connected to each other and to the CL domain by a peptide linker and in that the second polypeptide comprises one ectodomain of said TNF ligand family member or a fragment thereof connected via a peptide linker to the CH1 domain of said polypeptide.

Particular TNF Family Ligand Trimer-Containing Antigen Binding Molecules

In another aspect, the invention provides a TNF family ligand trimer-containing antigen binding molecule, wherein the antigen binding molecule comprises

a first heavy chain and a first light chain, both comprising a Fab molecule capable of specific binding to PD1,
a first peptide comprising two ectodomains of a TNF ligand family member or fragments thereof connected to each other by a first peptide linker fused at its C-terminus by a second peptide linker to a second heavy or light chain,
and a second peptide comprising one ectodomain of said TNF ligand family member fused at its C-terminus by a third peptide linker to a second light or heavy chain, respectively.

In a further aspect, provided is a TNF family ligand trimer-containing antigen binding molecule, wherein the first peptide comprising two ectodomains of a TNF ligand family member or fragments thereof connected to each other by a first peptide linker is fused at its C-terminus by a second peptide linker to a CH1 domain that is part of a heavy chain, and the second peptide comprising one ectodomain of said TNF ligand family member or a fragment thereof is fused at its C-terminus by a third peptide linker to a CL domain that is part of a light chain.

In yet another aspect, provided is a TNF family ligand trimer-containing antigen binding molecule, wherein the first peptide comprising two ectodomains of a TNF ligand family member or fragments thereof connected to each other by a first peptide linker is fused at its C-terminus by a second peptide linker to a CL domain that is part of a heavy chain, and the second peptide comprising one ectodomain of said TNF ligand family member or a fragment thereof that is fused at its C-terminus by a third peptide linker to a CH1 domain that is part of a light chain.

In one aspect, the invention provides a TNF family ligand trimer-containing antigen binding molecule, wherein in the CL domain adjacent to the TNF ligand family member the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E). These modifications lead to so-called charged residues with advantageous properties that avoid undesired effects such as for example mispairing.

In particular, the CL domain comprises the amino acid mutations E123R and Q124K and the CH1 domain comprises the amino acid mutations K147E and K213E.

In one aspect, the invention provides a TNF family ligand trimer-containing antigen binding molecule as defined herein before, wherein the moiety capable of specific binding to PD1 is a scaffold antigen binding protein.

In a particular aspect, the invention is concerned with a TNF family ligand trimer-containing antigen binding molecule as defined above, wherein the moiety capable of specific binding to PD1 is a Fab molecule capable of specific binding to a target cell antigen.

The invention provides a TNF family ligand trimer-containing antigen binding molecule that comprises at least one moiety capable of specific binding to PD1. In a particular aspect, the TNF family ligand trimer-containing antigen binding molecule comprises one moiety capable of specific binding to PD1, meaning the TNF family ligand trimer-containing antigen binding molecule is monovalent. In another aspect, the invention provides a TNF family ligand trimer-containing antigen binding molecule comprising two moieties capable of specific binding to PD1, meaning the TNF family ligand trimer-containing antigen binding molecule is bivalent.

In one aspect, the invention provides a TNF family ligand trimer-containing antigen binding molecule, wherein the moiety capable of specific binding to PD1 comprises

(a) a VH domain comprising (i) HVR-H1 comprising the amino acid sequence of SEQ ID NO: 13,
(ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16 (v) HVR-L2 comprising the amino acid sequence of SEQ ID NO: 17, and (vi) HVR-L3 comprising the amino acid sequence of SEQ ID NO:18,
(b) a VH domain comprising (i) HVR-H1 comprising the amino acid sequence of SEQ ID NO:26,
(ii) HVR-H2 comprising the amino acid sequence of SEQ ID NO:27, and (iii) HVR-H3 comprising the amino acid sequence of SEQ ID NO:28, and a VL domain comprising (iv) HVR-L1 comprising the amino acid sequence of SEQ ID NO:29 (v) HVR-L2 comprising the amino acid sequence of SEQ ID NO:30, and (vi) HVR-L3 comprising the amino acid sequence of SEQ ID NO:31,
(c) a VH domain comprising (i) HVR-H1 comprising the amino acid sequence of SEQ ID NO:34,
(ii) HVR-H2 comprising the amino acid sequence of SEQ ID NO:35, and (iii) HVR-H3 comprising the amino acid sequence of SEQ ID NO:36, and a VL domain comprising (iv) HVR-L1 comprising the amino acid sequence of SEQ ID NO:37 (v) HVR-L2 comprising the amino acid sequence of SEQ ID NO:38, and (vi) HVR-L3 comprising the amino acid sequence of SEQ ID NO:39,
(d) a VH domain comprising (i) HVR-H1 comprising the amino acid sequence of SEQ ID NO:42,
(ii) HVR-H2 comprising the amino acid sequence of SEQ ID NO:43, and (iii) HVR-H3 comprising the amino acid sequence of SEQ ID NO:44, and a VL domain comprising (iv) HVR-L1 comprising the amino acid sequence of SEQ ID NO:45 (v) HVR-L2 comprising the amino acid sequence of SEQ ID NO:46, and (vi) HVR-L3 comprising the amino acid sequence of SEQ ID NO:47,
(e) a VH domain comprising (i) HVR-H1 comprising the amino acid sequence of SEQ ID NO:50,
(ii) HVR-H2 comprising the amino acid sequence of SEQ ID NO:51, and (iii) HVR-H3 comprising the amino acid sequence of SEQ ID NO:52, and a VL domain comprising (iv) HVR-L1 comprising the amino acid sequence of SEQ ID NO:53 (v) HVR-L2 comprising the amino acid sequence of SEQ ID NO:54, and (vi) HVR-L3 comprising the amino acid sequence of SEQ ID NO:55,
(f) a VH domain comprising (i) HVR-H1 comprising the amino acid sequence of SEQ ID NO:58,
(ii) HVR-H2 comprising the amino acid sequence of SEQ ID NO:59, and (iii) HVR-H3 comprising the amino acid sequence of SEQ ID NO:60, and a VL domain comprising (iv) HVR-L1 comprising the amino acid sequence of SEQ ID NO:61 (v) HVR-L2 comprising the amino acid sequence of SEQ ID NO:62, and (vi) HVR-L3 comprising the amino acid sequence of SEQ ID NO:63, or
(g) a VH domain comprising (i) HVR-H1 comprising the amino acid sequence of SEQ ID NO:66,
(ii) HVR-H2 comprising the amino acid sequence of SEQ ID NO:67, and (iii) HVR-H3 comprising the amino acid sequence of SEQ ID NO:68, and a VL domain comprising (iv) HVR-L1 comprising the amino acid sequence of SEQ ID NO:69 (v) HVR-L2 comprising the amino acid sequence of SEQ ID NO:70, and (vi) HVR-L3 comprising the amino acid sequence of SEQ ID NO:71.

In a further aspect, the invention provides a TNF family ligand trimer-containing antigen binding molecule, wherein the moiety capable of specific binding to PD1 comprises

(a) a VH domain comprising an amino acid sequence of SEQ ID NO: 19 and a VL domain comprising an amino acid sequence of SEQ ID NO:20,
(b) a VH domain comprising an amino acid sequence of SEQ ID NO:21 and a VL domain comprising an amino acid sequence of SEQ ID NO:22,
(c) a VH domain comprising an amino acid sequence of SEQ ID NO:21 and a VL domain comprising an amino acid sequence of SEQ ID NO:23,
(d) a VH domain comprising an amino acid sequence of SEQ ID NO:21 and a VL domain comprising an amino acid sequence of SEQ ID NO:24,
(e) a VH domain comprising an amino acid sequence of SEQ ID NO:21 and a VL domain comprising an amino acid sequence of SEQ ID NO:25,
(f) a VH domain comprising an amino acid sequence of SEQ ID NO:32 and a VL domain comprising an amino acid sequence of SEQ ID NO:33,
(g) a VH domain comprising an amino acid sequence of SEQ ID NO:40 and a VL domain comprising an amino acid sequence of SEQ ID NO:41,
(h) a VH domain comprising an amino acid sequence of SEQ ID NO:48 and a VL domain comprising an amino acid sequence of SEQ ID NO:49,
(i) a VH domain comprising an amino acid sequence of SEQ ID NO:56 and a VL domain comprising an amino acid sequence of SEQ ID NO:57,
(k) a VH domain comprising an amino acid sequence of SEQ ID NO:64 and a VL domain comprising an amino acid sequence of SEQ ID NO:65, or
(l) a VH domain comprising an amino acid sequence of SEQ ID NO:72 and a VL domain comprising an amino acid sequence of SEQ ID NO:73.

In one aspect, the invention provides a TNF family ligand trimer-containing antigen binding molecule, wherein the moiety capable of specific binding to PD1 comprises

(a) a VH domain comprising (i) HVR-H1 comprising the amino acid sequence of SEQ ID NO: 13,
(ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16, (v) HVR-L2 comprising the amino acid sequence of SEQ ID NO: 17, and (vi) HVR-L3 comprising the amino acid sequence of SEQ ID NO:18.

In a particular aspect, the invention provides a TNF family ligand trimer-containing antigen binding molecule, wherein the moiety capable of specific binding to PD1 comprises

(a) a VH domain comprising an amino acid sequence of SEQ ID NO: 19 and a VL domain comprising an amino acid sequence of SEQ ID NO:20,
(b) a VH domain comprising an amino acid sequence of SEQ ID NO:21 and a VL domain comprising an amino acid sequence of SEQ ID NO:22,
(c) a VH domain comprising an amino acid sequence of SEQ ID NO:21 and a VL domain comprising an amino acid sequence of SEQ ID NO:23,
(d) a VH domain comprising an amino acid sequence of SEQ ID NO:21 and a VL domain comprising an amino acid sequence of SEQ ID NO:24, or
(e) a VH domain comprising an amino acid sequence of SEQ ID NO:21 and a VL domain comprising an amino acid sequence of SEQ ID NO:25.

In a further aspect, the moiety capable of specific binding to PD1 comprises a heavy chain variable region comprising an amino acid sequence that is at least about 95%, 96%, 97%, 98%, 99% or 100% identical to the amino acid sequence of SEQ ID NO:21 and a light chain variable region comprising an amino acid sequence that is at least about 95%, 96%, 97%, 98%, 99% or 100% identical to the amino acid sequence of SEQ ID NO:22.

In another aspect, the moiety capable of specific binding to PD1 comprises a heavy chain variable region comprising an amino acid sequence that is at least about 95%, 96%, 97%, 98%, 99% or 100% identical to the amino acid sequence of SEQ ID NO:21 and a light chain variable region comprising an amino acid sequence that is at least about 95%, 96%, 97%, 98%, 99% or 100% identical to the amino acid sequence of SEQ ID NO:24.

In one aspect, provided is a TNF family ligand trimer-containing antigen binding molecule as defined herein before, wherein the moiety capable of specific binding to PD1 comprises a variable heavy chain comprising an amino acid sequence of SEQ ID NO:21 and a variable light chain comprising an amino acid sequence of SEQ ID NO:22 or wherein the moiety capable of specific binding to FAP comprises a variable heavy chain comprising an amino acid sequence of SEQ ID NO:21 and a variable light chain comprising an amino acid sequence of SEQ ID NO:24.

In a particular aspect, the moiety capable of specific binding to PD1 comprises a heavy chain variable region comprising the amino acid sequence of SEQ ID NO:21 and a light chain variable region comprising the amino acid sequence of SEQ ID NO:22. In another particular aspect, the moiety capable of specific binding to PD1 comprises a heavy chain variable region comprising the amino acid sequence of SEQ ID NO:21 and a light chain variable region comprising the amino acid sequence of SEQ ID NO:24 In a specific aspect, the moiety capable of specific binding to PD1 comprises a VH domain consisting of amino acid sequence of SEQ ID NO:21 and a VL domain consisting of the amino acid sequence of SEQ ID NO:22 or a VH domain consisting of amino acid sequence of SEQ ID NO:21 and a VL domain consisting of the amino acid sequence of SEQ ID NO:24.

In a further aspect, the TNF family ligand trimer-containing antigen binding molecule of the invention comprises

(a) at least one moiety capable of specific binding to PD1 comprising a heavy chain variable region comprising the amino acid sequence of SEQ ID NO:21 and a light chain variable region comprising the amino acid sequence of SEQ ID NO:22 or a heavy chain variable region comprising the amino acid sequence of SEQ ID NO:21 and a light chain variable region comprising the amino acid sequence of SEQ ID NO:24, and
(b) a second heavy chain comprising an amino acid sequence selected from the group consisting of SEQ ID NO:9, SEQ ID NO:11, SEQ ID NO:12 and SEQ ID NO:10, and a second light chain comprising an amino acid sequence selected from the group consisting of SEQ ID NO: 1, SEQ ID NO:3, SEQ ID NO:4 and SEQ ID NO:5.

In a particular aspect, the TNF family ligand trimer-containing antigen binding molecule of the invention comprises

(a) at least one moiety capable of specific binding to PD1 comprising a heavy chain variable region comprising the amino acid sequence of SEQ ID NO:21 and a light chain variable region comprising the amino acid sequence of SEQ ID NO:22 or a heavy chain variable region comprising the amino acid sequence of SEQ ID NO:21 and a light chain variable region comprising the amino acid sequence of SEQ ID NO:24, and
(b) a first and a second polypeptide that are linked to each other by a disulfide bond,
wherein the antigen binding molecule is characterized in that the first polypeptide comprises the the amino acid sequence of SEQ ID NO: 10 and the second polypeptide comprises the amino acid sequence of SEQ ID NO:5.

In a further aspect, provided is a TNF family ligand trimer-containing antigen binding molecule as described herein before, wherein the antigen binding molecule comprises

(a) a first heavy chain and a first light chain, both comprising a Fab molecule capable of specific binding to PD1,
(b) a second heavy chain comprising an amino acid sequence selected from the group consisting of SEQ ID NO:9, SEQ ID NO: 11, SEQ ID NO: 12 and SEQ ID NO: 10, and
a second light chain comprising an amino acid sequence selected from the group consisting of SEQ ID NO: 1, SEQ ID NO:3, SEQ ID NO:4 and SEQ ID NO:5.

In one aspect, the invention provides a TNF family ligand trimer-containing antigen binding molecule, wherein the antigen binding molecule comprises

(a) a Fab molecule capable of specific binding to PD1 comprising a heavy chain variable region comprising the amino acid sequence of SEQ ID NO:21 and a light chain variable region comprising the amino acid sequence of SEQ ID NO:22 or a heavy chain variable region comprising the amino acid sequence of SEQ ID NO:21 and a light chain variable region comprising the amino acid sequence of SEQ ID NO:24, and
(b) a second heavy chain comprising an amino acid sequence selected from the group consisting of SEQ ID NO:9, SEQ ID NO: 11, SEQ ID NO: 12 and SEQ ID NO: 10, and
a second light chain comprising an amino acid sequence selected from the group consisting of SEQ ID NO: 1, SEQ ID NO:3, SEQ ID NO:4 and SEQ ID NO:5.

In one aspect, the invention provides a TNF family ligand trimer-containing antigen binding molecule, wherein the antigen binding molecule comprises

(a) (i) a first heavy chain comprising the VH domain comprising the amino acid sequence of SEQ ID NO: 19 and a first light chain comprising the VL domain comprising the amino acid sequence of SEQ ID NO:20 or
a first heavy chain comprising the VH domain comprising the amino acid sequence of SEQ ID NO:21 and a first light chain comprising the VL domain comprising the amino acid sequence selected from the group consisting of SEQ ID NO:22, SEQ ID NO:23, SEQ ID NO:24 and SEQ ID NO:25, and
(b) a second heavy chain comprising an amino acid sequence selected from the group consisting of SEQ ID NO:9, SEQ ID NO: 11, SEQ ID NO: 12 and SEQ ID NO: 10, and
a second light chain comprising an amino acid sequence selected from the group consisting of SEQ ID NO: 1, SEQ ID NO:3, SEQ ID NO:4 and SEQ ID NO:5.

In a particular aspect, provided is a TNF family ligand trimer-containing antigen binding molecule, wherein the antigen binding molecule comprises

(i) a first heavy chain comprising the VH domain comprising the amino acid sequence of SEQ ID NO: 19 and a first light chain comprising the VL domain comprising the amino acid sequence of SEQ ID NO:20 or
a first heavy chain comprising the VH domain comprising the amino acid sequence of SEQ ID NO:21 and a first light chain comprising the VL domain comprising the amino acid sequence selected from the group consisting of SEQ ID NO:22, SEQ ID NO:23, SEQ ID NO:24 and SEQ ID NO:25,
(ii) a second heavy chain comprising the amino acid sequence selected from the group consisting of SEQ ID NO:74, SEQ ID NO:76, SEQ ID NO:78 and SEQ ID NO:80, and
(iii) a second light chain comprising the amino acid sequence selected from the group consisting of SEQ ID NO:75, SEQ ID NO:77, SEQ ID NO:79 and SEQ ID NO:81.

In another aspect, the invention provides a TNF family ligand trimer-containing antigen binding molecule, wherein the antigen binding molecule comprises

(a) a first heavy chain comprising the amino acid sequence of SEQ ID NO:82, a first light chain comprising the amino acid sequence of SEQ ID NO:83, a second heavy chain comprising the amino acid sequence of SEQ ID NO:74 and a second light chain comprising the amino acid sequence of SEQ ID NO:75,
(b) a first heavy chain comprising the amino acid sequence of SEQ ID NO:82, a first light chain comprising the amino acid sequence of SEQ ID NO:83, a second heavy chain comprising the amino acid sequence of SEQ ID NO:76 and a second light chain comprising the amino acid sequence of SEQ ID NO:77,
(c) a first heavy chain comprising the amino acid sequence of SEQ ID NO:82, a first light chain comprising the amino acid sequence of SEQ ID NO:83, a second heavy chain comprising the amino acid sequence of SEQ ID NO:78 and a second light chain comprising the amino acid sequence of SEQ ID NO:79, or
(d) a first heavy chain comprising the amino acid sequence of SEQ ID NO:82, a first light chain comprising the amino acid sequence of SEQ ID NO:83, a second heavy chain comprising the amino acid sequence of SEQ ID NO:80 and a second light chain comprising the amino acid sequence of SEQ ID NO:81.

In a further aspect, the invention provides a TNF family ligand trimer-containing antigen binding molecule, wherein the antigen binding molecule comprises

(a) a first heavy chain comprising the amino acid sequence of SEQ ID NO:82, a first light chain comprising the amino acid sequence of SEQ ID NO: 171, a second heavy chain comprising the amino acid sequence of SEQ ID NO:74 and a second light chain comprising the amino acid sequence of SEQ ID NO:75,
(b) a first heavy chain comprising the amino acid sequence of SEQ ID NO:82, a first light chain comprising the amino acid sequence of SEQ ID NO: 171, a second heavy chain comprising the amino acid sequence of SEQ ID NO:76 and a second light chain comprising the amino acid sequence of SEQ ID NO:77,
(c) a first heavy chain comprising the amino acid sequence of SEQ ID NO:82, a first light chain comprising the amino acid sequence of SEQ ID NO: 171, a second heavy chain comprising the amino acid sequence of SEQ ID NO:78 and a second light chain comprising the amino acid sequence of SEQ ID NO:79, or
(d) a first heavy chain comprising the amino acid sequence of SEQ ID NO:82, a first light chain comprising the amino acid sequence of SEQ ID NO: 171, a second heavy chain comprising the amino acid sequence of SEQ ID NO:80 and a second light chain comprising the amino acid sequence of SEQ ID NO:81.

In particular, provided is a TNF family ligand trimer-containing antigen binding molecule, wherein the antigen binding molecule comprises a first heavy chain comprising the amino acid sequence of SEQ ID NO:82, a first light chain comprising the amino acid sequence of SEQ ID NO:83, a second heavy chain comprising the amino acid sequence of SEQ ID NO:78 and a second light chain comprising the amino acid sequence of SEQ ID NO:79.

In a further particular aspect, provided is a TNF family ligand trimer-containing antigen binding molecule, wherein the antigen binding molecule comprises a first heavy chain comprising the amino acid sequence of SEQ ID NO:82, a first light chain comprising the amino acid sequence of SEQ ID NO: 171, a second heavy chain comprising the amino acid sequence of SEQ ID NO:78 and a second light chain comprising the amino acid sequence of SEQ ID NO:79.

In yet another aspect, the invention provides a TNF family ligand trimer-containing antigen binding molecule, comprising

(a) at least one moiety capable of specific binding to PD1, and
(b) a first and a second polypeptide that are linked to each other by a disulfide bond,
wherein the antigen binding molecule is characterized in that the first polypeptide contains a CH3 domain and the second polypeptide contains a CH3 domain, respectively, and wherein the first polypeptide comprises two ectodomains of a TNF ligand family member or fragments thereof that are connected to each other and to the C-terminus of the CH3 domain by a peptide linker and wherein the second polypeptide comprises one ectodomain of said TNF ligand family member or a fragment thereof connected via a peptide linker to C-terminus of the CH3 domain of said polypeptide.

In particular, such a TNF family ligand trimer-containing antigen binding molecule comprises two Fab domains capable of specific binding to PD1.

In particular, provided is a TNF family ligand trimer-containing antigen binding molecule as described herein before comprises

(a) a first heavy chain comprising the amino acid sequence of SEQ ID NO:84, a second heavy chain comprising the amino acid sequence of SEQ ID NO:85, and two light chains comprising the amino acid sequence of SEQ ID NO:83, or
(b) a first heavy chain comprising the amino acid sequence of SEQ ID NO:86, a second heavy chain comprising the amino acid sequence of SEQ ID NO:87, and two light chains comprising the amino acid sequence of SEQ ID NO:83.

In a further aspect, provided is a TNF family ligand trimer-containing antigen binding molecule as described herein before comprises

(a) a first heavy chain comprising the amino acid sequence of SEQ ID NO:84, a second heavy chain comprising the amino acid sequence of SEQ ID NO:85, and two light chains comprising the amino acid sequence of SEQ ID NO:171, or
(b) a first heavy chain comprising the amino acid sequence of SEQ ID NO:86, a second heavy chain comprising the amino acid sequence of SEQ ID NO:87, and two light chains comprising the amino acid sequence of SEQ ID NO:171.

In another particular aspect, the invention provides a TNF family ligand trimer-containing antigen binding molecule as described herein before, wherein the TNF family ligand trimer-containing antigen binding molecule comprises one Fab domain capable of specific binding to PD1.

In such aspect, provided is a TNF family ligand trimer-containing antigen binding molecule, wherein the antigen binding molecule comprises

(i) a fusion polypeptide comprising a first subunit of a Fc domain and two ectodomains of a TNF ligand family member or fragments thereof that are connected to each other and to the C-terminus of the CH3 domain by a peptide linker, (ii) a heavy chain comprising the VH domain of the Fab domain capable of specific binding to PD1, a second subunit of the Fc domain and one ectodomain of said TNF ligand family member or a fragment thereof connected via a peptide linker to C-terminus of the CH3 domain, and (iii) a light chain comprising the VL domain of the Fab domain capable of specific binding to PD1, or
(i) a fusion polypeptide comprising a first subunit of a Fc domain and one ectodomain of a TNF ligand family member or a fragment thereof that is connected to the C-terminus of the CH3 domain by a peptide linker, (ii) a heavy chain comprising the VH domain of the Fab domain capable of specific binding to PD1, a second subunit of the Fc domain and two ectodomains of said TNF ligand family member or fragments thereof that are connected to each other and to the C-terminus of the CH3 domain via a peptide linker, and (iii) a light chain comprising the VL domain of the Fab domain capable of specific binding to PD1.

In a particular aspect, provided is a TNF family ligand trimer-containing antigen binding molecule, wherein the antigen binding molecule comprises

(a) a fusion polypeptide comprising the amino acid sequence of SEQ ID NO: 88, a heavy chain comprising the amino acid sequence of SEQ ID NO:87 and a light chain comprising the amino acid sequence of SEQ ID NO:83,
(b) a fusion polypeptide comprising the amino acid sequence of SEQ ID NO:89, a heavy chain comprising the amino acid sequence of SEQ ID NO:90 and a light chain comprising the amino acid sequence of SEQ ID NO:83,
(c) a fusion polypeptide comprising the amino acid sequence of SEQ ID NO:91, a heavy chain comprising the amino acid sequence of SEQ ID NO:86 and a light chain comprising the amino acid sequence of SEQ ID NO:83, or
(d) a fusion polypeptide comprising the amino acid sequence of SEQ ID NO:93, a heavy chain comprising the amino acid sequence of SEQ ID NO:92 and a light chain comprising the amino acid sequence of SEQ ID NO:83.

In another particular aspect, provided is a TNF family ligand trimer-containing antigen binding molecule, wherein the antigen binding molecule comprises

(a) a fusion polypeptide comprising the amino acid sequence of SEQ ID NO: 88, a heavy chain comprising the amino acid sequence of SEQ ID NO:87 and a light chain comprising the amino acid sequence of SEQ ID NO:171,
(b) a fusion polypeptide comprising the amino acid sequence of SEQ ID NO:89, a heavy chain comprising the amino acid sequence of SEQ ID NO:90 and a light chain comprising the amino acid sequence of SEQ ID NO:171,
(c) a fusion polypeptide comprising the amino acid sequence of SEQ ID NO:91, a heavy chain comprising the amino acid sequence of SEQ ID NO:86 and a light chain comprising the amino acid sequence of SEQ ID NO:171, or
(d) a fusion polypeptide comprising the amino acid sequence of SEQ ID NO:93, a heavy chain comprising the amino acid sequence of SEQ ID NO:92 and a light chain comprising the amino acid sequence of SEQ ID NO:171.

Polynucleotides

The invention further provides isolated polynucleotides encoding a TNF family ligand trimer-containing antigen binding molecule as described herein or a fragment thereof.

The isolated polynucleotides encoding TNF ligand trimer-containing antigen binding molecules of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes the entire TNF family ligand trimer-containing antigen binding molecule according to the invention as described herein. In particular, the isolated polynucleotide encodes a polypeptide comprised in the TNF family ligand trimer-containing antigen binding molecule according to the invention as described herein.

In one aspect, the present invention is directed to isolated polynucleotides encoding a TNF family ligand trimer-containing antigen binding molecule, wherein the polynucleotide comprises (a) a sequence that encodes a moiety capable of specific binding to a PD1, (b) a sequence that encodes a polypeptide comprising two ectodomains of a TNF ligand family member or two fragments thereof that are connected to each other by a peptide linker and (c) a sequence that encodes a polypeptide comprising one ectodomain of said TNF ligand family member or a fragment thereof.

In another aspect, provided is an isolated polynucleotide encoding a 4-1BB ligand trimer-containing antigen binding molecule, wherein the polynucleotide comprises (a) a sequence that encodes a moiety capable of specific binding to PD1, (b) a sequence that encodes a polypeptide comprising two ectodomains of 4-1BBL or two fragments thereof that are connected to each other by a peptide linker and (c) a sequence that encodes a polypeptide comprising one ectodomain of 4-1BBL or a fragment thereof.

In a further aspect, the invention is directed to an isolated polynucleotide comprising a sequence that encodes a polypeptide comprising two 4-1BBL fragments comprising an amino acid sequence that is at least about 90%, 95%, 98% or 100% identical to an amino acid sequence shown in SEQ ID NO:1, SEQ ID NO:2, SEQ ID NO:3, SEQ ID NO:4, SEQ ID NO:5, SEQ ID NO:6, SEQ ID NO:7 or SEQ ID NO:8, and to a polynucleotide comprising a sequence that encodes a polypeptide comprising one 4-1BBL fragment comprising an amino acid sequence that is at least about 90%, 95%, 98% or 100% identical to an amino acid sequence shown in SEQ ID NO: 1, SEQ ID NO:2, SEQ ID NO:3, SEQ ID NO:4, SEQ ID NO:5, SEQ ID NO:6, SEQ ID NO:7 or SEQ ID NO:8.

Furthermore, provided is an isolated polynucleotide encoding a OX40 ligand trimer-containing antigen binding molecule, wherein the polynucleotide comprises (a) a sequence that encodes a moiety capable of specific binding to PD1, (b) a sequence that encodes a polypeptide comprising two ectodomains of OX40L or two fragments thereof that are connected to each other by a peptide linker and (c) a sequence that encodes a polypeptide comprising one ectodomain of OX40L or a fragment thereof.

In further aspects, the invention relates to the polynucleotides comprising a sequence that is at least about 90%, 95%, 98% or 100% identical to the specific cDNA sequences disclosed herein. In a particular aspect, the invention relates to a polynucleotide comprising a sequence that is identical to one of the specific cDNA sequences disclosed herein.

In other aspects, the nucleic acid molecule comprises or consists of a nucleotide sequence that encodes an amino acid sequence as set forth in any one of SEQ ID NOs: 19, 20, 21, 22, 23, 24, 25, 32, 33, 40, 41, 48, 49, 56, 57, 64, 65, 72 or 73. In a further aspect, the nucleic acid molecule comprises or consists of a nucleotide sequence that encodes an amino acid sequence as set forth in any one of SEQ ID NOs:9, 10, 11 or 12.

In still other aspects, the nucleic acid molecule comprises or consists of a nucleotide sequence selected from the group consisting of SEQ ID NOs: 131, 132, 133, 134, 135, 136, 137, 138, 139, 140, 141, 142, 143, 144, 145, 146, 147, 148, 149 or 150.

In certain aspects, the polynucleotide or nucleic acid is DNA. In other embodiments, a polynucleotide of the present invention is RNA, for example, in the form of messenger RNA (mRNA). RNA of the present invention may be single stranded or double stranded.

Recombinant Methods

TNF family ligand trimer-containing antigen binding molecules of the invention may be obtained, for example, by solid-state peptide synthesis (e.g. Merrifield solid phase synthesis) or recombinant production. For recombinant production one or more polynucleotide encoding the TNF family ligand trimer-containing antigen binding molecule or polypeptide fragments thereof, e.g., as described above, is isolated and inserted into one or more vectors for further cloning and/or expression in a host cell. Such polynucleotide may be readily isolated and sequenced using conventional procedures. In one aspect of the invention, a vector, preferably an expression vector, comprising one or more of the polynucleotides of the invention is provided. Methods which are well known to those skilled in the art can be used to construct expression vectors containing the coding sequence of the TNF family ligand trimer-containing antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y. (1989). The expression vector can be part of a plasmid, virus, or may be a nucleic acid fragment. The expression vector includes an expression cassette into which the polynucleotide encoding the TNF family ligand trimer-containing antigen binding molecule or polypeptide fragments thereof (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the TNF family ligand trimer-containing antigen binding molecule of the invention or polypeptide fragments thereof, or variants or derivatives thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription.

Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit a-globin, as well as other sequences capable of controlling gene expression in eukaryotic cells. Additional suitable transcription control regions include tissue-specific promoters and enhancers as well as inducible promoters (e.g. promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the TNF family ligand trimer-containing antigen binding molecule or polypeptide fragments thereof is desired, DNA encoding a signal sequence may be placed upstream of the nucleic acid encoding a TNF family ligand trimer-containing antigen binding molecule of the invention or polypeptide fragments thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase.

DNA encoding a short protein sequence that could be used to facilitate later purification (e.g. a histidine tag) or assist in labeling the fusion protein may be included within or at the ends of the polynucleotide encoding a TNF family ligand trimer-containing antigen binding molecule of the invention or polypeptide fragments thereof.

In a further aspect of the invention, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one aspect, a host cell comprises (e.g. has been transformed or transfected with) a vector comprising a polynucleotide that encodes (part of) a TNF family ligand trimer-containing antigen binding molecule of the invention of the invention. As used herein, the term “host cell” refers to any kind of cellular system which can be engineered to generate the fusion proteins of the invention or fragments thereof. Host cells suitable for replicating and for supporting expression of antigen binding molecules are well known in the art. Such cells may be transfected or transduced as appropriate with the particular expression vector and large quantities of vector containing cells can be grown for seeding large scale fermenters to obtain sufficient quantities of the antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr-CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B.K.C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., YO, NS0, Sp20 cell). Standard technologies are known in the art to express foreign genes in these systems. Cells expressing a polypeptide comprising either the heavy or the light chain of an immunoglobulin, may be engineered so as to also express the other of the immunoglobulin chains such that the expressed product is an immunoglobulin that has both a heavy and a light chain.

In one aspect, a method of producing a TNF family ligand trimer-containing antigen binding molecule of the invention or polypeptide fragments thereof is provided, wherein the method comprises culturing a host cell comprising polynucleotides encoding the TNF family ligand trimer-containing antigen binding molecule of the invention or polypeptide fragments thereof, as provided herein, under conditions suitable for expression of the TNF family ligand trimer-containing antigen binding molecule of the invention or polypeptide fragments thereof, and recovering the TNF family ligand trimer-containing antigen binding molecule of the invention or polypeptide fragments thereof from the host cell (or host cell culture medium).

In the TNF family ligand trimer-containing antigen binding molecule of the invention, the components (at least one moiety capable of specific binding to a target cell antigen, one polypeptide comprising two ectodomains of a TNF ligand family member or fragments thereof and a polypeptide comprising one ectodomain of said TNF family ligand family member or a fragment thereof) are not genetically fused to each other. The polypeptides are designed such that its components (two ectodomains of a TNF ligand family member or fragments thereof and other components such as CH or CL) are fused to each other directly or through a linker sequence. The composition and length of the linker may be determined in accordance with methods well known in the art and may be tested for efficacy. Examples of linker sequences between different components of the antigen binding molecules of the invention are found in the sequences provided herein. Additional sequences may also be included to incorporate a cleavage site to separate the individual components of the fusion protein if desired, for example an endopeptidase recognition sequence.

In certain embodiments the moieties capable of specific binding to a target cell antigen (e.g. Fab fragments) forming part of the antigen binding molecule comprise at least an immunoglobulin variable region capable of binding to an antigen. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of immunoglobulin can be used in the invention. Non-limiting immunoglobulins useful in the present invention can be of murine, primate, or human origin. If the fusion protein is intended for human use, a chimeric form of immunoglobulin may be used wherein the constant regions of the immunoglobulin are from a human. A humanized or fully human form of the immunoglobulin can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osboum et al., Methods 36, 61-68 (2005) and Klimka et al., Br J Cancer 83, 252-260 (2000) (describing the “guided selection” approach to FR shuffling). Particular immunoglobulins according to the invention are human immunoglobulins. Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain aspects, the moieties capable of specific binding to a target cell antigen (e.g. Fab fragments) comprised in the antigen binding molecules of the present invention are engineered to have enhanced binding affinity according to, for example, the methods disclosed in PCT publication WO 2012/020006 (see Examples relating to affinity maturation) or U.S. Pat. Appl. Publ. No. 2004/0132066. The ability of the antigen binding molecules of the invention to bind to a specific antigenic determinant can be measured either through an enzyme-linked immunosorbent assay (ELISA) or other techniques familiar to one of skill in the art, e.g. surface plasmon resonance technique (Liljeblad, et al., Glyco J 17, 323-329 (2000)), and traditional binding assays (Heeley, Endocr Res 28, 217-229 (2002)). Competition assays may be used to identify an antigen binding molecule that competes with a reference antibody for binding to a particular antigen. In certain embodiments, such a competing antigen binding molecule binds to the same epitope (e.g. a linear or a conformational epitope) that is bound by the reference antigen binding molecule. Detailed exemplary methods for mapping an epitope to which an antigen binding molecule binds are provided in Morris (1996) “Epitope Mapping Protocols”, in Methods in Molecular Biology vol. 66 (Humana Press, Totowa, N.J.). In an exemplary competition assay, immobilized antigen is incubated in a solution comprising a first labeled antigen binding molecule that binds to the antigen and a second unlabeled antigen binding molecule that is being tested for its ability to compete with the first antigen binding molecule for binding to the antigen. The second antigen binding molecule may be present in a hybridoma supernatant. As a control, immobilized antigen is incubated in a solution comprising the first labeled antigen binding molecule but not the second unlabeled antigen binding molecule. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antigen binding molecule is competing with the first antigen binding molecule for binding to the antigen. See Harlow and Lane (1988) Antibodies: A Laboratory Manual ch. 14 (Cold Spring Harbor Laboratory, Cold Spring Harbor, N.Y.).

TNF ligand trimer-containing antigen binding molecules of the invention prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the TNF ligand trimer-containing antigen binding molecule binds. For example, for affinity chromatography purification of fusion proteins of the invention, a matrix with protein A or protein G may be used. Sequential Protein A or G affinity chromatography and size exclusion chromatography can be used to isolate an antigen binding molecule essentially as described in the Examples. The purity of the TNF ligand trimer-containing antigen binding molecule or fragments thereof can be determined by any of a variety of well-known analytical methods including gel electrophoresis, high pressure liquid chromatography, and the like. For example, the TNF ligand trimer-containing antigen binding molecules expressed as described in the Examples were shown to be intact and properly assembled as demonstrated by reducing and non-reducing SDS-PAGE.

Assays

The antigen binding molecules provided herein may be identified, screened for, or characterized for their physical/chemical properties and/or biological activities by various assays known in the art. Biological activity may include, e.g., the ability to enhance the activation and/or proliferation of different immune cells especially T-cells. E.g. they enhance secretion of immunomodulating cytokines (e.g. interferon-gamma (IFN-gamma) and/or tumor necrosis factor alpha (TNF alpha)). Other immunomodulating cytokines which are or can be enhanced are e.g IL12, Granzyme B etc. Biological activity may also include, cynomolgus binding crossreactivity, as well as binding to different cell types. Antigen binding molecules having such biological activity in vivo and/or in vitro are also provided.

1. Affinity assays

The affinity of the TNF family ligand trimer-containing antigen binding molecule provided herein for the corresponding TNF receptor can be determined in accordance with the methods set forth in the Examples by surface plasmon resonance (SPR), using standard instrumentation such as a BIAcore instrument (GE Healthcare), and receptors or target proteins such as may be obtained by recombinant expression. The affinity of the TNF family ligand trimer-containing antigen binding molecule for PD1 can also be determined by surface plasmon resonance (SPR), using standard instrumentation such as a BIAcore instrument (GE Healthcare), and receptors or target proteins such as may be obtained by recombinant expression. A specific illustrative and exemplary embodiment for measuring binding affinity is described in Example 4. According to one aspect, K_{D} is measured by surface plasmon resonance using a BIACORE® T100 machine (GE Healthcare) at 25° C.

2. Binding Assays and Other Assays

Binding of the TNF family ligand trimer-containing antigen binding molecule provided herein to the corresponding receptor expressing cells may be evaluated using cell lines expressing the particular receptor or target antigen, for example by flow cytometry (FACS). In one aspect, fresh peripheral blood mononuclear cells (PBMCs) expressing the TNF receptor are used in the binding assay. These cells are used directly after isolation (naïve PMBCs) or after stimulation (activated PMBCs). In another aspect, activated mouse splenocytes (expressing the TNF receptor molecule) were used to demonstrate the binding of the TNF family ligand trimer-containing antigen binding molecule of the invention to the corresponding TNF receptor expressing cells.

In a further aspect, cell lines expressing PD1 were used to demonstrate the binding of the antigen binding molecules to this target cell antigen.

In another aspect, competition assays may be used to identify an antigen binding molecule that competes with a specific antibody or antigen binding molecule for binding to the target or TNF receptor, respectively. In certain embodiments, such a competing antigen binding molecule binds to the same epitope (e.g., a linear or a conformational epitope) that is bound by a specific anti-target antibody or a specific anti-TNF receptor antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.).

3. Activity Assays

In one aspect, assays are provided for identifying TNF family ligand trimer-containing antigen binding molecules that bind to a specific target cell antigen and to a specific TNF receptor having biological activity. Biological activity may include, e.g., agonistic signalling through the TNF receptor on cells expressing the target cell antigen. TNF family ligand trimer-containing antigen binding molecules identified by the assays as having such biological activity in vitro are also provided.

In certain aspects, a TNF family ligand trimer-containing antigen binding molecule of the invention is tested for such biological activity. Assays for detecting the biological activity of the molecules of the invention are those described in Examples 5 and 6. Furthermore, assays for detecting cell lysis (e.g. by measurement of LDH release), induced apoptosis kinetics (e.g. by measurement of Caspase 3/7 activity) or apoptosis (e.g. using the TUNEL assay) are well known in the art. In addition the biological activity of such complexes can be assessed by evaluating their effects on survival, proliferation and lymphokine secretion of various lymphocyte subsets such as NK cells, NKT-cells or γδ T-cells or assessing their capacity to modulate phenotype and function of antigen presenting cells such as dendritic cells, monocytes/macrophages or B-cells.

Pharmaceutical Compositions, Formulations and Routes of Administration

In a further aspect, the invention provides pharmaceutical compositions comprising any of the TNF family ligand trimer-containing antigen binding molecules provided herein, e.g., for use in any of the below therapeutic methods. In one embodiment, a pharmaceutical composition comprises any of the TNF family ligand trimer-containing antigen binding molecules provided herein and at least one pharmaceutically acceptable excipient. In another embodiment, a pharmaceutical composition comprises any of the TNF family ligand trimer-containing antigen binding molecules provided herein and at least one additional therapeutic agent, e.g., as described below.

Pharmaceutical compositions of the present invention comprise a therapeutically effective amount of one or more TNF family ligand trimer-containing antigen binding molecules dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one TNF family ligand trimer-containing antigen binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the TNF family ligand trimer-containing antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the fusion proteins may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the fusion proteins of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The TNF family ligand trimer-containing antigen binding molecules may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

The formulations to be used for in vivo administration are generally sterile. Sterility may be readily accomplished, e.g., by filtration through sterile filtration membranes.

Therapeutic Methods and Compositions

Any of the TNF family ligand trimer-containing antigen binding molecules provided herein may be used in therapeutic methods.

For use in therapeutic methods, TNF family ligand trimer-containing antigen binding molecules of the invention can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, TNF family ligand trimer-containing antigen binding molecules of the invention for use as a medicament are provided. In further aspects, TNF family ligand trimer-containing antigen binding molecules of the invention for use in treating a disease, in particular for use in the treatment of cancer, are provided. In certain aspects, TNF family ligand trimer-containing antigen binding molecules of the invention for use in a method of treatment are provided. In one aspect, the invention provides a TNF family ligand trimer-containing antigen binding molecule as described herein for use in the treatment of a disease in an individual in need thereof. In certain aspects, the invention provides a TNF family ligand trimer-containing antigen binding molecule for use in a method of treating an individual having a disease comprising administering to the individual a therapeutically effective amount of the fusion protein. In certain aspects, the disease to be treated is cancer. Examples of cancers include solid tumors, bladder cancer, renal cell carcinoma, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer, melanoma, B-cell lymphoma, B-cell leukemia, non-Hodgkin lymphoma and acute lymphoblastic leukemia. Thus, a TNF family ligand trimer-containing antigen binding molecule as described herein for use in the treatment of cancer is provided. The subject, patient, or “individual” in need of treatment is typically a mammal, more specifically a human.

In another aspect, provided is a TNF family ligand trimer-containing antigen binding molecule as described herein for use in the treatment of infectious diseases, in particular for the treatment of viral infections. In a further aspect, provided is a TNF family ligand trimer-containing antigen binding molecule as described herein for use in the treatment of autoimmune diseases such as for example Lupus disease.

In a further aspect, the invention relates to the use of a TNF family ligand trimer-containing antigen binding molecule in the manufacture or preparation of a medicament for the treatment of a disease in an individual in need thereof. In one aspect, the medicament is for use in a method of treating a disease comprising administering to an individual having the disease a therapeutically effective amount of the medicament. In certain embodiments the disease to be treated is a proliferative disorder, particularly cancer. Thus, in one aspect, the invention relates to the use of a TNF family ligand trimer-containing antigen binding molecule of the invention in the manufacture or preparation of a medicament for the treatment of cancer. Examples of cancers include solid tumors, bladder cancer, renal cell carcinoma, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer, melanoma, B-cell lymphoma, B-cell leukemia, non-Hodgkin lymphoma and acute lymphoblastic leukemia. Other cell proliferation disorders that can be treated using a TNF family ligand trimer-containing antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan may recognize that in some cases the TNF family ligand trimer-containing antigen binding molecule may not provide a cure but may only provide partial benefit. In some aspects, a physiological change having some benefit is also considered therapeutically beneficial. Thus, in some aspects, an amount of TNF family ligand trimer-containing antigen binding molecule that provides a physiological change is considered an “effective amount” or a “therapeutically effective amount”.

In a further aspect, the invention relates to the use of a TNF family ligand trimer-containing antigen binding molecule as described herein in the manufacture or preparation of a medicament for the treatment of infectious diseases, in particular for the treatment of viral infections or for the treatment of autoimmune diseases, for example Lupus disease.

In a further aspect, the invention provides a method for treating a disease in an individual, comprising administering to said individual a therapeutically effective amount of a TNF family ligand trimer-containing antigen binding molecule of the invention. In one aspect a composition is administered to said individual, comprising a fusion protein of the invention in a pharmaceutically acceptable form. In certain aspects, the disease to be treated is a proliferative disorder. In a particular aspect, the disease is cancer. In another aspect, the disease is an infectious disease or an autoimmune disease. In certain aspects, the method further comprises administering to the individual a therapeutically effective amount of at least one additional therapeutic agent, e.g. an anti-cancer agent if the disease to be treated is cancer. An “individual” according to any of the above embodiments may be a mammal, preferably a human.

For the prevention or treatment of disease, the appropriate dosage of a TNF family ligand trimer-containing antigen binding molecule of the invention (when used alone or in combination with one or more other additional therapeutic agents) will depend on the type of disease to be treated, the route of administration, the body weight of the patient, the type of antigen binding molecule, the severity and course of the disease, whether the fusion protein is administered for preventive or therapeutic purposes, previous or concurrent therapeutic interventions, the patient's clinical history and response to the fusion protein, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The TNF family ligand trimer-containing antigen binding molecule is suitably administered to the patient at one time or over a series of treatments. Depending on the type and severity of the disease, about 1 μg/kg to 15 mg/kg (e.g. 0.1 mg/kg-10 mg/kg) of TNF family ligand trimer-containing antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein). An initial higher loading dose, followed by one or more lower doses may be administered. However, other dosage regimens may be useful. The progress of this therapy is easily monitored by conventional techniques and assays.

The TNF family ligand trimer-containing antigen binding molecules of the invention will generally be used in an amount effective to achieve the intended purpose. For use to treat or prevent a disease condition, the TNF family ligand trimer-containing antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the TNF family ligand trimer-containing antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the TNF family ligand trimer-containing antigen binding molecule may not be related to plasma concentration. One skilled in the art will be able to optimize therapeutically effective local dosages without undue experimentation.

A therapeutically effective dose of the TNF family ligand trimer-containing antigen binding molecules described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. TNF family ligand trimer-containing antigen binding molecules that exhibit large therapeutic indices are preferred. In one embodiment, the TNF family ligand trimer-containing antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with fusion proteins of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Other Agents and Treatments

The TNF family ligand trimer-containing antigen binding molecules of the invention may be administered in combination with one or more other agents in therapy. For instance, a fusion protein of the invention may be co-administered with at least one additional therapeutic agent. The term “therapeutic agent” encompasses any agent that can be administered for treating a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is another anti-cancer agent.

Such other agents are suitably present in combination in amounts that are effective for the purpose intended. The effective amount of such other agents depends on the amount of fusion protein used, the type of disorder or treatment, and other factors discussed above. The TNF family ligand trimer-containing antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the TNF family ligand trimer-containing antigen binding molecule of the invention can occur prior to, simultaneously, and/or following, administration of the additional therapeutic agent and/or adjuvant.

Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is a TNF ligand trimer-containing antigen binding molecule of the invention.

The label or package insert indicates that the composition is used for treating the condition of choice. Moreover, the article of manufacture may comprise (a) a first container with a composition contained therein, wherein the composition comprises a TNF ligand trimer-containing antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE C
(Sequences):

| SEQ | | |
|---|---|---|
| ID NO: | Description | Sequence |
| 1 | Human (hu) 4-1BBL (71-254) | REGPELSPDDPAGLLDLRQGMFAQLVAQN |
| | | VLLIDGPLSWYSDPGLAGVSLTGGLSYKE |
| | | DTKELVVAKAGVYYVFFQLELRRVVAGE |
| | | GSGSVSLALHLQPLRSAAGAAALALTVDL |
| | | PPASSEARNSAFGFQGRLLHLSAGQRLGV |
| | | HLHTEARARHAWQLTQGATVLGLFRVTP |
| | | EIPAGLPSPRSE |
| 2 | hu 4-1BBL (85-254) | LDLRQGMFAQLVAQNVLLIDGPLSWYSDP |
| | | GLAGVSLTGGLSYKEDTKELVVAKAGVY |
| | | YVFFQLELRRVVAGEGSGSVSLALHLQPL |
| | | RSAAGAAALALTVDLPPASSEARNSAFGF |
| | | QGRLLHLSAGQRLGVHLHTEARARHAWQ |
| | | LTQGATVLGLFRVTPEIPAGLPSPRSE |
| 3 | hu 4-1BBL (80-254) | DPAGLLDLRQGMFAQLVAQNVLLIDGPLS |
| | | WYSDPGLAGVSLTGGLSYKEDTKELVVA |
| | | KAGVYYVFFQLELRRVVAGEGSGSVSLAL |
| | | HLQPLRSAAGAAALALTVDLPPASSEARN |
| | | SAFGFQGRLLHLSAGQRLGVHLHTEARAR |
| | | HAWQLTQGATVLGLFRVTPEIPAGLPSPRSE |
| 4 | hu 4-1BBL (52-254) | PWAVSGARASPGSAASPRLREGPELSPDDP |
| | | AGLLDLRQGMFAQLVAQNVLLIDGPLSW |
| | | YSDPGLAGVSLTGGLSYKEDTKELVVAKA |
| | | GVYYVFFQLELRRVVAGEGSGSVSLALHL |
| | | QPLRSAAGAAALALTVDLPPASSEARNSA |
| | | FGFQGRLLHLSAGQRLGVHLHTEARARHA |
| | | WQLTQGATVLGLFRVTPEIPAGLPSPRSE |
| 5 | Human (hu) 4-1BBL (71-248) | REGPELSPDDPAGLLDLRQGMFAQLVAQN |
| | | VLLIDGPLSWYSDPGLAGVSLTGGLSYKE |
| | | DTKELVVAKAGVYYVFFQLELRRVVAGE |
| | | GSGSVSLALHLQPLRSAAGAAALALTVDL |
| | | PPASSEARNSAFGFQGRLLHLSAGQRLGV |
| | | HLHTEARARHAWQLTQGATVLGLFRVTP |
| | | EIPAGL |
| 6 | hu 4-1BBL (85-248) | LDLRQGMFAQLVAQNVLLIDGPLSWYSDP |
| | | GLAGVSLTGGLSYKEDTKELVVAKAGVY |
| | | YVFFQLELRRVVAGEGSGSVSLALHLQPL |
| | | RSAAGAAALALTVDLPPASSEARNSAFGF |
| | | QGRLLHLSAGQRLGVHLHTEARARHAWQ |
| | | LTQGATVLGLFRVTPEIPAGL |
| 7 | hu 4-1BBL (80-248) | DPAGLLDLRQGMFAQLVAQNVLLIDGPLS |
| | | WYSDPGLAGVSLTGGLSYKEDTKELVVA |
| | | KAGVYYVFFQLELRRVVAGEGSGSVSLAL |
| | | HLQPLRSAAGAAALALTVDLPPASSEARN |
| | | SAFGFQGRLLHLSAGQRLGVHLHTEARAR |
| | | HAWQLTQGATVLGLFRVTPEIPAGL |
| 8 | hu 4-1BBL (52-248) | PWAVSGARASPGSAASPRLREGPELSPDDP |
| | | AGLLDLRQGMFAQLVAQNVLLIDGPLSW |
| | | YSDPGLAGVSLTGGLSYKEDTKELVVAKA |
| | | GVYYVFFQLELRRVVAGEGSGSVSLALHL |
| | | QPLRSAAGAAALALTVDLPPASSEARNSA |
| | | FGFQGRLLHLSAGQRLGVHLHTEARARHA |
| | | WQLTQGATVLGLFRVTPEIPAGL |
| 9 | dimeric hu 4-1BBL (71-254) connected by | REGPELSPDDPAGLLDLRQGMFAQLVAQN |
| | (G4S)₂ linker | VLLIDGPLSWYSDPGLAGVSLTGGLSYKE |
| | | DTKELVVAKAGVYYVFFQLELRRVVAGE |
| | | GSGSVSLALHLQPLRSAAGAAALALTVDL |
| | | PPASSEARNSAFGFQGRLLHLSAGQRLGV |
| | | HLHTEARARHAWQLTQGATVLGLFRVTP |
| | | EIPAGLPSPRSEGGGGSGGGGSREGPELSP |
| | | DDPAGLLDLRQGMFAQLVAQNVLLIDGPL |
| | | SWYSDPGLAGVSLTGGLSYKEDTKELVVA |
| | | KAGVYYVFFQLELRRVVAGEGSGSVSLAL |
| | | HLQPLRSAAGAAALALTVDLPPASSEARN |
| | | SAFGFQGRLLHLSAGQRLGVHLHTEARAR |
| | | HAWQLTQGATVLGLFRVTPEIPAGLPSPRSE |
| 10 | dimeric hu 4-1BBL (71-248) connected by | REGPELSPDDPAGLLDLRQGMFAQLVAQN |
| | (G4S)₂ linker | VLLIDGPLSWYSDPGLAGVSLTGGLSYKE |
| | | DTKELVVAKAGVYYVFFQLELRRVVAGE |
| | | GSGSVSLALHLQPLRSAAGAAALALTVDL |
| | | PPASSEARNSAFGFQGRLLHLSAGQRLGV |
| | | HLHTEARARHAWQLTQGATVLGLFRVTP |
| | | EIPAGLGGGGSGGGGSREGPELSPDDPAGL |
| | | LDLRQGMFAQLVAQNVLLIDGPLSWYSDP |
| | | GLAGVSLTGGLSYKEDTKELVVAKAGVY |
| | | YVFFQLELRRVVAGEGSGSVSLALHLQPL |
| | | RSAAGAAALALTVDLPPASSEARNSAFGF |
| | | QGRLLHLSAGQRLGVHLHTEARARHAWQ |
| | | LTQGATVLGLFRVTPEIPAGL |
| 11 | dimeric hu 4-1BBL (80-254) connected by | DPAGLLDLRQGMFAQLVAQNVLLIDGPLS |
| | (G4S)₂ linker | WYSDPGLAGVSLTGGLSYKEDTKELVVA |
| | | KAGVYYVFFQLELRRVVAGEGSGSVSLAL |
| | | HLQPLRSAAGAAALALTVDLPPASSEARN |
| | | SAFGFQGRLLHLSAGQRLGVHLHTEARAR |
| | | HAWQLTQGATVLGLFRVTPEIPAGLPSPRS |
| | | EGGGGSGGGGSDPAGLLDLRQGNIFAQLV |
| | | AQNVLLIDGPLSWYSDPGLAGVSLTGGLS |
| | | YKEDTKELVVAKAGVYYVFFQLELRRVV |
| | | AGEGSGSVSLALHLQPLRSAAGAAALALT |
| | | VDLPPASSEARNSAFGFQGRLLHLSAGQR |
| | | LGVHLHTEARARHAWQLTQGATVLGLFR |
| | | VTPEIPAGLPSPRSE |
| 12 | dimeric hu 4-1BBL (52-254) connected by | PWAVSGARASPGSAASPRLREGPELSPDDP |
| | (G4S)₂ linker | AGLLDLRQGNIFAQLVAQNVLLIDGPLSW |
| | | YSDPGLAGVSLTGGLSYKEDTKELVVAKA |
| | | GVYYVFFQLELRRVVAGEGSGSVSLALHL |
| | | QPLRSAAGAAALALTVDLPPASSEARNSA |
| | | FGFQGRLLHLSAGQRLGVHLHTEARARHA |
| | | WQLTQGATVLGLFRVTPEIPAGLPSPRSEG |
| | | GGGSGGGGSPWAVSGARASPGSAASPRLR |
| | | EGPELSPDDPAGLLDLRQGMFAQLVAQNV |
| | | LLIDGPLSWYSDPGLAGVSLTGGLSYKED |
| | | TKELVVAKAGVYYVFFQLELRRVVAGEG |
| | | SGSVSLALHLQPLRSAAGAAALALTVDLP |
| | | PASSEARNSAFGFQGRLLHLSAGQRLGVH |
| | | LHTEARARHAWQLTQGATVLGLFRVTPEI |
| | | PAGLPSPRSE |
| 13 | heavy chain HVR-H1, PD1-0103 | GFSFSSY |
| 14 | heavy chain HVR-H2, PD1-0103 | GGR |
| 15 | heavy chain HVR-H3, PD1-0103 | TGRVYFALD |
| 16 | light chain HVR-L1, PD1-0103 | SESVDTSDNSF |
| 17 | light chain HVR-L2, PD1-0103 | RSS |
| 18 | light chain HVR-L3, PD1-0103 | NYDVPW |
| 19 | heavy chain variable domain VH, PD1- | EVILVESGGGLVKPGGSLKLSCAASGFSFS |
| | 0103 | SYTMSWVRQTPEKRLDWVATISGGGRDIY |
| | | YPDSVKGRFTISRDNAKNTLYLEMSSLMS |
| | | EDTALYYCVLLTGRVYFALDSWGQGTSV |
| | | TVSS |
| 20 | light chain variable domain VL, PD1-0103 | KIVLTQSPASLPVSLGQRATISCRASESVDT |
| | | SDNSFIHWYQQRPGQSPKLLIYRSSTLESG |
| | | VPARFSGSGSRTDFTLTIDPVEADDVATYY |
| | | CQQNYDVPWTFGGGTKLEIK |
| 21 | humanized variant-heavy chain variable | EVQLLESGGGLVQPGGSLRLSCAASGFSFS |
| | domain VH of PD1-0103-0312, -0313, -0314 | SYTMSWVRQAPGKGLEWVATISGGGRDI |
| | and -0315 | YYPDSVKGRFTISRDNSKNTLYLQMNSLR |
| | | AEDTAVYYCVLLTGRVYFALDSWGQGTL |
| | | VTVSS |
| 22 | humanized variant-light chain variable | DIVMTQSPDSLAVSLGERATINCKASESVD |
| | domain VL of PD1-0103-0312 | TSDNSFIHWYQQKPGQSPKLLIYRSSTLES |
| | | GVPDRFSGSGSGTDFTLTISSLQAEDVAVY |
| | | YCQQNYDVPWTFGQGTKVEIK |
| 23 | humanized variant-light chain variable | DVVMTQSPLSLPVTLGQPASISCRASESVD |
| | domain VL of PD1-0103-0313 | TSDNSFIHWYQQRPGQSPRLLIYRSSTLES |
| | | GVPDRFSGSGSGTDFTLKISRVEAEDVGVY |
| | | YCQQNYDVPWTFGQGTKVEIK |
| 24 | humanized variant-light chain variable | EIVLTQSPATLSLSPGERATLSCRASESVDT |
| | domain VL of PD1-0103-0314 | SDNSFIHWYQQKPGQSPRLLIYRSSTLESGI |
| | | PARFSGSGSGTDFTLTISSLEPEDFAVYYCQ |
| | | QNYDVPWTFGQGTKVEIK |
| 25 | humanized variant-light chain variable | EIVLTQSPATLSLSPGERATLSCRASESVDT |
| | domain VL of PD1-0103-0315 | SDNSFIHWYQQKPGQSPRLLIYRSSTLESGI |
| | | PARFSGSGSGTDFTLTISSLEPEDFAVYYCQ |
| | | QNYDVPWTFGQGTKVEIK |
| 26 | heavy chain HVR-H1, PD1-0098 | GYSITSDY |
| 27 | heavy chain HVR-H2, PD1-0098 | YSG |
| 28 | heavy chain HVR-H3, PD1-0098 | HGSAPWYFD |
| 29 | light chain HVR-L1, PD1-0098 | SQNIVHSDGNTY |
| 30 | light chain HVR-L2, PD1-0098 | KVS |
| 31 | light chain HVR-L3, PD1-0098 | GSHFPL |
| 32 | heavy chain variable domain VH, PD1- | DVQLQESGPGLVKPSQSLSLTCTVTGYSIT |
| | 0098 | SDYAWNWIRQFPGDKLEWLGYITYSGFTN |
| | | YNPSLKSRISISRDTSKNQFFLQLNSVATED |
| | | TATYYCARWHGSAPWYFDYWGRGTTLT |
| | | VSS |
| 33 | light chain variable domain VL, PD1-0098 | DVLMTQTPLSLPVSLGDQASISCRSSQNIV |
| | | HSDGNTYLEWYLQKPGQSPNLLIYKVSRR |
| | | FSGVPDRFSGSGSGTDFTLKISRVEAEDLG |
| | | VYYCFQGSHFPLTFGAGTKLELK |
| 34 | heavy chain HVR-H1, PD1-0050 | GYSITSDY |
| 35 | heavy chain HVR-H2, PD1-0050 | YTG |
| 36 | heavy chain HVR-H3, PD1-0050 | MDYYGSTLD |
| 37 | light chain HVR-L1, PD1-0050 | SESVDRYGNSF |
| 38 | light chain HVR-L2, PD1-0050 | RAS |
| 39 | light chain HVR-L3, PD1-0050 | NNEDPY |
| 40 | heavy chain variable domain VH, PD1- | DVQLQESGPGLVKPSQSLSLTCTVTGYSIT |
| | 0050 | SDYAWNWIRQFPGNKLEWMGYITYTGRT |
| | | SYNPSLKSRISITRDTSKNQFFLQLNSVTTE |
| | | DTATYYCAREMDYYGSTLDYWGQGTTL |
| | | TVSS |
| 41 | light chain variable domain VL, PD1-0050 | KIVLTQSPASLAVSLRQRATISCRASESVD |
| | | RYGNSFIHWYQQKPGQPPKVLIYRASNLE |
| | | SGFPARFSGSGSRTDFTLTIDPVEADDAAT |
| | | YYCQQNNEDPYTFGSGTKLEIK |
| 42 | heavy chain HVR-H1, PD1-0069 | GYTFTDY |
| 43 | heavy chain HVR-H2, PD1-0069 | YSG |
| 44 | heavy chain HVR-H3, PD1-0069 | GITTGFA |
| 45 | light chain HVR-L1, PD1-0069 | SKGVSTSSYSF |
| 46 | light chain HVR-L2, PD1-0069 | YAS |
| 47 | light chain HVR-L3, PD1-0069 | SREFPW |
| 48 | heavy chain variable domain VH, PD1- | QVQLQQSGPELVRPGVSVKISCKGSGYTF |
| | 0069 | TDYAMHWVKQSHARTLEWIGVISTYSGD |
| | | TNYNQKFKDKATMTVDKSSSTAYLELAR |
| | | MTSEDSAIYYCARLGITTGFAYWGQGTL |
| | | VTVSA |
| 49 | light chain variable domain VL, PD1-0069 | DIVLTQSPASLAVSLGQRATISCRASKGVS |
| | | TSSYSFMHWYQQKPRQPPKLLIKYASYLE |
| | | SGVPARFSGSGSGTDFTLNIHPVEEEDAAT |
| | | YYCHHSREFPWTFGGGTKLEIK |
| 50 | heavy chain HVR-H1, PD1-0073 | GFTFSNY |
| 51 | heavy chain HVR-H2, PD1-0073 | GGR |
| 52 | heavy chain HVR-H3, PD1-0073 | YYGID |
| 53 | light chain HVR-L1, PD1-0073 | SQDVTTA |
| 54 | light chain HVR-L2, PD1-0073 | WAS |
| 55 | light chain HVR-L3, PD1-0073 | HYSIPW |
| 56 | heavy chain variable domain VH, PD1- | EVKLVESGGGLVKPGGSLKLSCAASGFTF |
| | 73 | SNYGMSW1RQTPEKGLEWVATISGGGRD |
| | | TYYPDSVKGRFTISRDNVKNNLYLQMSSL |
| | | RSEDTAFYYCASYYYGIDYWGQGTSVTV |
| | | SS |
| 57 | light chain variable domain VL, PD1-0073 | DIVMTQPHKFMSTSVGDRVRITCKASQDV |
| | | TTAVAWYQQKPGQSPKLLIYWASTRHTG |
| | | VPDRFTGSGSGTEFTLTISSVQAEDLALYY |
| | | CQQHYSIPWTFGGGTKLEIK |
| 58 | heavy chain HVR-H1, PD1-0078 | GYTFTST |
| 59 | heavy chain HVR-H2, PD1-0078 | SDS |
| 60 | heavy chain HVR-H3, PD1-0078 | PFD |
| 61 | light chain HVR-L1, PD1-0078 | SQDVSTA |
| 62 | light chain HVR-L2, PD1-0078 | SAS |
| 63 | light chain HVR-L3, PD1-0078 | HYSHPF |
| 64 | heavy chain variable domain VH, PD1- | QVQLQQPGAELVKPGASVKMSCKASGYT |
| | 0078 | FTSTWMHWVKQRPGQGLEWIGAIDPSDS |
| | | YTTYNQKFKGKATLTVDTSSTTAYMQLSS |
| | | LTSEDSAVYYCTRSPFDYWGQGTTLTVSS |
| 65 | light chain variable domain VL, PD1-0078 | DIVMTQSHKFMSTSVGDRVSITCKASQDV |
| | | STAVAWYQQKPGQSPKLLIYSASYRYTGV |
| | | PDRFTGSGSGTDFTFAISSVQAEDLAVYYC |
| | | QQHYSHPFTFGSGTKLEIK |
| 66 | heavy chain HVR-H1, PD1-0102 | GYSITSGY |
| 67 | heavy chain HVR-H2, PD1-0102 | SSG |
| 68 | heavy chain HVR-H3, PD1-0102 | RNWYFD |
| 69 | light chain HVR-L1, PD1-0102 | SQSLLNSGTQKNY |
| 70 | light chain HVR-L2, PD1-0102 | WAS |
| 71 | light chain HVR-L3, PD1-0102 | DYTFPL |
| 72 | heavy chain variable domain VH, PD1- | DVQLQESGPDLVKPSQSLSLTCTVTGYSIT |
| | 0102 | SGYSWHWIRQFPGNKLEWMGFIHSSGDT |
| | | NYNPSLKSRISFTRDTSKNQFFLQLSSLTDE |
| | | DTATYYCATYRNWYFDVWGAGTTVTVSS |
| 73 | light chain variable domain VL, PD1-0102 | DIVMTQSPSSLTVTAGEKVTMRCKSSQSL |
| | | LNSGTQKNYLTWYQQKPGQPPKLLIYWA |
| | | STRESGVPNRFTGSGSGTDFTLTISSVQAE |
| | | DLSVYYCQSDYTFPLTFGGGTKLELK |
| 74 | Dimeric 4-1BB ligand (71-254) - CL* Fc | see Table 8 |
| | knob chain | |
| 75 | Monomeric 4-1BB ligand (71-254) - CH1* | see Table 8 |
| 76 | Dimeric 4-1BB ligand (71-254) - CL Fc | see Table 9 |
| | knob chain | |
| 77 | Monomeric 4-1BB ligand (71-254) - CH1 | see Table 9 |
| 78 | Dimeric 4-1BB ligand (71-248) - CL* Fc | see Table 11 |
| | knob chain | |
| 79 | Monomeric 4-1BB ligand (71-248) - CH1* | see Table 11 |
| 80 | Dimeric 4-1BB ligand (71-248) - CL Fc | see Table 12 |
| | knob chain | |
| 81 | Monomeric 4-1BB ligand (71-248) - CH1 | see Table 12 |
| 82 | anti-PD1(314) Fc hole chain | see Table 8 |
| 83 | anti-PD1(314) light chain | see Table 8 |
| 84 | anti-PD1(314) Fc hole dimeric 4-1BB | see Table 10 |
| | ligand (71-254) | |
| 85 | anti-PD1(314) Fc knob monomeric 4-1BB | see Table 10 |
| | ligand (71-254) | |
| 86 | anti-PD1(314) Fc hole dimeric 4-1BB | see Table 13 |
| | ligand (71-248) | |
| 87 | anti-PD1(314) Fc knob monomeric 4-1BB | see Table 13 |
| | ligand (71-248) | |
| 88 | Fc hole dimeric 4-1BB ligand (71-248) | see Table 14 |
| 89 | Fc hole monomeric 4-1BB ligand (71-248) | see Table 15 |
| 90 | anti-PD1(314) Fc knob dimeric 4-1BB | see Table 15 |
| | ligand (71-248) | |
| 91 | Fc knob chain fused to monomeric 4-1BB | see Table 16 |
| | ligand (71-248) chain | |
| 92 | anti-PD1(314) Fc hole monomeric 4-1BB | see Table 17 |
| | ligand (71-248) chain | |
| 93 | Fc knob dimeric 4-1BB ligand (71-248) | see Table 17 |
| 94 | Human PD1 | UniProt no. Q15116 |
| 95 | human Lymphotoxin α | UniProt no. P01374 |
| 96 | human TNF | UniProt no. P01375 |
| 97 | human Lymphotoxin β | UniProt no. Q06643 |
| 98 | human OX40L | UniProt no. P23510 |
| 99 | human CD40L | UniProt no. P29965 |
| 100 | human FasL | UniProt no. P48023 |
| 101 | human CD27L | UniProt no. P32970 |
| 102 | human CD30L | UniProt no. P32971 |
| 103 | human 4-1BBL | UniProt no. P41273 |
| 104 | human TRAIL | UniProt no. P50591 |
| 105 | human RANKL | UniProt no. 014788 |
| 106 | human TWEAK | UniProt no. 043508 |
| 107 | human APRIL | UniProt no. O75888 |
| 108 | human BAFF | UniProt no. Q9Y275 |
| 109 | human LIGHT | UniProt no. O43557 |
| 110 | human TL1A | UniProt no. O95150 |
| 111 | human GITRL | UniProt no. Q9UNG2 |
| 112 | human ectodysplasin A | UniProt no. Q92838 |
| 113 | human 4-1BBL(50-254) | ACPWAVSGARASPGSAASPRLREGPE |
| | | LSPDDPAGLLDLRQGMFAQLVAQNVL |
| | | LIDGPLSWYSDPGLAGVSLTGGLSYKE |
| | | DTKELVVAKAGVYYVFFQLELRRVVA |
| | | GEGSGSVSLALHLQPLRSAAGAAALA |
| | | LTVDLPPASSEARNSAFGFQGRLLHLS |
| | | AGQRLGVHLHTEARARHAWQLTQGA |
| | | TVLGLFRVTPEIPAGLPSPRSE |
| 114 | human OX40L (51-183) | QVSHRYPRIQSIKVQFTEYKKEKGFILT |
| | | SQKEDEIMKVQNNSVIINCDGFYLISLK |
| | | GYFSQEVNISLHYQKDEEPLFQLKKVR |
| | | SVNSLMVASLTYKDKVYLNVTTDNTS |
| | | LDDFHVNGGELILIHQNPGEFCVL |
| 115 | human OX40L (52-183) | VSHRYPRIQSIKVQFTEYKKEKGFILTS |
| | | QKEDEIMKVQNNSVIINCDGFYLISLK |
| | | GYFSQEVNISLHYQKDEEPLFQLKKVR |
| | | SVNSLMVASLTYKDKVYLNVTTDNTS |
| | | LDDFHVNGGELILIHQNPGEFCVL |
| 116 | Peptide linker G4S | GGGGS |
| 117 | Peptide linker (G4S)₂ | GGGGSGGGGS |
| 118 | Peptide linker (SG4)₂ | SGGGGSGGGG |
| 119 | Peptide linker (G₄S)₃ | GGGGSGGGGSGGGGS |
| 120 | Peptide linker G4(SG4)₂ | GGGGSGGGGSGGGG |
| 121 | Peptide linker (G₄S)₄ | GGGGSGGGGSGGGGSGGGGS |
| 122 | Peptide linker | GSPGSSSSGS |
| 123 | Peptide linker | GSGSGSGS |
| 124 | Peptide linker | GSGSGNGS |
| 125 | Peptide linker | GGSGSGSG |
| 126 | Peptide linker | GGSGSG |
| 127 | Peptide linker | GGSG |
| 128 | Peptide linker | GGSGNGSG |
| 129 | Peptide linker | GGNGSGSG |
| 130 | Peptide linker | GGNGSG |
| 131 | nucleotide sequence of dimeric hu 4-1BBL | see Table 8 |
| | (71-254) - CL* Fc knob chain | |
| 132 | nucleotide sequence of monomeric hu 4- | see Table 8 |
| | 1BBL (71-254) - CH1* | |
| 133 | nucleotide sequence of anti-PD1(0314) Fc | see Table 8 |
| | hole chain | |
| 134 | nucleotide sequence of anti-PD1(0314) | see Table 8 |
| | light chain | |
| 135 | nucleotide sequence of dimeric hu 4-1BBL | see Table 9 |
| | (71-254) - CL Fc knob chain | |
| 136 | nucleotide sequence of monomeric hu 4- | see Table 9 |
| | 1BBL (71-254) - CH1 | |
| 137 | nucleotide sequence of anti-PD1(0314) Fc | see Table 10 |
| | hole chain fused to dimeric hu 4-1BBL | |
| | (71-254) | |
| 138 | nucleotide sequence of anti-PD1(0314) Fc | see Table 10 |
| | knob chain fused to monomeric hu 4-1BBL | |
| | (71-254) | |
| 139 | nucleotide sequence of dimeric hu 4-1BBL | see Table 11 |
| | (71-248) - CL* Fc knob chain | |
| 140 | nucleotide sequence of monomeric hu 4- | see Table 11 |
| | 1BBL (71-248) - CH1* | |
| 141 | nucleotide sequence of dimeric hu 4-1BBL | see Table 12 |
| | (71-248) - CL Fc knob chain | |
| 142 | nucleotide sequence of monomeric hu 4- | see Table 12 |
| | 1BBL (71-248) - CH1 | |
| 143 | nucleotide sequence of anti-PD1(0314) Fc | see Table 13 |
| | hole chain fused to dimeric hu 4-1BBL | |
| | (71-248) | |
| 144 | nucleotide sequence of anti-PD1(0314) Fc | see Table 13 |
| | knob chain fused to monomeric hu 4-1BBL | |
| | (71-248) | |
| 145 | nucleotide sequence of Fc hole dimeric | see Table 14 |
| | ligand (71-248) chain | |
| 146 | nucleotide sequence of Fc hole monomeric | see Table 15 |
| | ligand (71-248) chain | |
| 147 | nucleotide sequence of anti-PD1(0314) Fc | see Table 15 |
| | knob chain fused to dimeric hu 4-1BBL | |
| | (71-248) | |
| 148 | nucleotide sequence of Fc knob chain fused | see Table 16 |
| | to monomeric hu 4-1BBL (71-248) | |
| 149 | nucleotide sequence of anti-PD1(0314) Fc | see Table 17 |
| | hole monomeric ligand (71-248) chain | |
| 150 | nucleotide sequence of Fc knob chain fused | see Table 17 |
| | to dimeric hu 4-1BBL (71-248) | |
| 151 | nucleotide sequence of DP47 Fc hole chain | see Table 18 |
| 152 | nucleotide sequence of DP47 light chain | see Table 18 |
| 153 | DP47 Fc hole chain | see Table 18 |
| 154 | DP47 light chain | see Table 18 |
| 155 | nucleotide sequence of DP47 Fc hole chain | see Table 19 |
| | fused to dimeric hu 4-1BBL (71-254) | |
| 156 | nucleotide sequence of DP47 Fc knob | see Table 19 |
| | chain fused to monomeric hu 4-1BBL (71- | |
| | 254) | |
| 157 | DP47 Fc hole chain fused to dimeric hu 4- | see Table 19 |
| | 1BBL (71-254) | |
| 158 | DP47 Fc knob chain fused to monomeric | see Table 19 |
| | hu 4-1BBL (71-254) | |
| 159 | nucleotide sequence of DP47 heavy chain | see Table 21 |
| | (hu IgG1 PGLALA) | |
| 160 | DP47 heavy chain (hu IgG1 PGLALA) | see Table 21 |
| 161 | nucleotide sequence of PD1(0314) heavy | see Table 22 |
| | chain (huIgG1 PGLALA) | |
| 162 | PD1(0314) heavy chain (huIgG1 | see Table 22 |
| | PGLALA) | |
| 163 | human 4-1BB ECD, aa 24-186 of Q07011 | |
| 164 | Nucleotide sequence of Fc hole chain | see Table 24 |
| 165 | Nucleotide sequence of human 4-1BB | see Table 24 |
| | antigen Fc knob chain | |
| 166 | Fc hole chain | see Table 24 |
| 167 | human 4-1BB antigen Fc knob chain | see Table 24 |
| 168 | avi tag | GLNDIFEAQKIEWHE |
| 169 | nucleotide sequence of human 4-1BB His | see Table 25 |
| 170 | human 4-1BB His | see Table 25 |
| 171 | anti-PD1 (0312) light chain | DIVMTQSPDSLAVSLGERATINCKASE |
| | | SVDTSDNSFIHWYQQKPGQSPKLLIYR |
| | | SSTLESGVPDRFSGSGSGTDFTLTISSL |
| | | QAEDVAVYYCQQNYDVPWTFGQGTK |
| | | VEIKRTVAAPSVFIFPPSDEQLKSGTAS |
| | | VVCLLNNFYPREAKVQWKVDNALQS |
| | | GNSQESVTEQDSKDSTYSLSSTLTLSK |
| | | ADYEKHKVYACEVTHQGLSSPVTKSF |
| | | NRGEC |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, Md. (1991). Amino acids of antibody chains are numbered and referred to according to the EU numbering systems according to Kabat (Kabat, E. A., et al., Sequences of Proteins of Immunological Interest, 5th ed., Public Health Service, National Institutes of Health, Bethesda, Md. (1991)) as defined above.

The following numbered paragraphs (paras) are aspects of the invention:
1. A TNF family ligand trimer-containing antigen binding molecule comprising
   (a) at least one moiety capable of specific binding to PD1 and
   (b) a first and a second polypeptide that are linked to each other by a disulfide bond,
   wherein the antigen binding molecule is characterized in that the first polypeptide comprises two ectodomains of a TNF ligand family member or a fragment thereof that are connected to each other by a peptide linker and in that the second polypeptide comprises only one ectodomain of said TNF ligand family member or a fragment thereof.
2. The TNF family ligand trimer-containing antigen binding molecule of para 1, further comprising
   (c) an Fc domain composed of a first and a second subunit capable of stable association.
3. The TNF family ligand trimer-containing antigen binding molecule of paras 1 or 2, comprising
   (a) at least one moiety capable of specific binding to PD1 and
   (b) a first and a second polypeptide that are linked to each other by a disulfide bond,
   wherein the antigen binding molecule is characterized in that
      a. the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of a TNF ligand family member or fragments thereof that are connected to each other and to the CH1 or CL domain by a peptide linker and wherein the second polypeptide comprises one ectodomain of said TNF ligand family member or a fragment thereof connected via a peptide linker to the CL or CH1 domain of said polypeptide, or
      b. the first polypeptide contains a CH3 domain and the second polypeptide contains a CH3 domain, respectively, and wherein the first polypeptide comprises two ectodomains of a TNF ligand family member or fragments thereof that are connected to each other and to the C-terminus of the CH3 domain by a peptide linker and wherein the second polypeptide comprises only one ectodomain of said TNF ligand family member or a fragment thereof connected via a peptide linker to the C-terminus of the CH3 domain of said polypeptide, or
      c. the first polypeptide contains a VH-CL or a VL-CH1 domain and the second polypeptide contains a VL-CH1 domain or a VH-CL domain, respectively, wherein the second polypeptide is linked to the first polypeptide by a disulfide bond between the CH1 and CL domain, and wherein the first polypeptide comprises two ectodomains of a TNF ligand family member or fragments thereof that are connected to each other and to to VH or VL by a peptide linker and wherein the second polypeptide comprises one ectodomain of said TNF ligand family member or a fragment thereof connected via a peptide linker to VL or VH of said polypeptide.
4. The TNF family ligand trimer-containing antigen binding molecule of any one of paras 1 to 3, wherein the TNF ligand family member costimulates human T-cell activation.
5. The TNF family ligand trimer-containing antigen binding molecule of any one of paras 1 to 4, wherein the TNF ligand family member is selected from 4-1BBL and OX40L.
6. The TNF family ligand trimer-containing antigen binding molecule of any one of paras 1 to 5, wherein the TNF ligand family member is 4-1BBL.
7. The TNF family ligand trimer-containing antigen binding molecule of any one of paras 1 to 6, wherein the ectodomain of a TNF ligand family member comprises the amino acid sequence selected from the group consisting of SEQ ID NO: 1, SEQ ID NO: 2, SEQ ID NO:3, SEQ ID NO:4, SEQ ID NO:5, SEQ ID NO: 6, SEQ ID NO:7 and SEQ ID NO:8, particularly the amino acid sequence of SEQ ID NO: 1 or SEQ ID NO:5.
8. The TNF family ligand trimer-containing antigen binding molecule of any one of paras 1 to 7, wherein the ectodomain of a TNF ligand family member comprises the amino acid sequence of SEQ ID NO:5.
9. The TNF family ligand trimer-containing antigen binding molecule of any one of paras 1 to 8, comprising
   (a) at least one moiety capable of specific binding to PD1 and
   (b) a first and a second polypeptide that are linked to each other by a disulfide bond,
   wherein the antigen binding molecule is characterized in that the first polypeptide comprises the amino acid sequence selected from the group consisting of SEQ ID NO:9, SEQ ID NO: 11, SEQ ID NO: 12 and SEQ ID NO: 10 and in that the second polypeptide comprises the amino acid sequence selected from the group consisting of SEQ ID NO: 1, SEQ ID NO: 3, SEQ ID NO:4 and SEQ ID NO:5.
10. The TNF family ligand trimer-containing antigen binding molecule of any one of paras 1 to 9, comprising
   (a) at least one moiety capable of specific binding to PD1, and
   (b) a first polypeptide containing a CH1 or CL domain and a second polypeptide containing a CL or CH1 domain, respectively, wherein the second polypeptide is linked to the first polypeptide by a disulfide bond between the CH1 and CL domain,
   and wherein the antigen binding molecule is characterized in that the first polypeptide comprises two ectodomains of a TNF ligand family member or fragments thereof that are connected to each other and to the CH1 or CL domain by a peptide linker and in that the second polypeptide comprises only one ectodomain of said TNF ligand family member or a fragment thereof connected via a peptide linker to the CL or CH1 domain of said polypeptide.
11. The TNF family ligand trimer-containing antigen binding molecule of any one of paras 1 to 10, wherein the moiety capable of specific binding to PD1 is selected from the group consisting of an antibody fragment, a Fab molecule, a crossover Fab molecule, a single chain Fab molecule, a Fv molecule, a scFv molecule, a single domain antibody, an aVH and a scaffold antigen binding protein.
12. The TNF family ligand trimer-containing antigen binding molecule of any one of paras 1 to 11, wherein the molecule comprises one moiety capable of specific binding to PD1.
13. The TNF family ligand trimer-containing antigen binding molecule of any one of paras 1 to 12, wherein the moiety capable of specific binding to PD1 is a Fab molecule capable of specific binding to PD1.
14. The TNF family ligand trimer-containing antigen binding molecule of any one of paras 1 to 13, wherein the moiety capable of specific binding to PD1 comprises
   (a) a VH domain comprising (i) HVR-H1 comprising the amino acid sequence of SEQ ID NO: 13, (ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16 (v) HVR-L2 comprising the amino acid sequence of SEQ ID NO: 17, and (vi) HVR-L3 comprising the amino acid sequence of SEQ ID NO: 18,
   (b) a VH domain comprising (i) HVR-H1 comprising the amino acid sequence of SEQ ID NO:26, (ii) HVR-H2 comprising the amino acid sequence of SEQ ID NO:27, and (iii) HVR-H3 comprising the amino acid sequence of SEQ ID NO:28, and a VL domain comprising (iv) HVR-L1 comprising the amino acid sequence of SEQ ID NO:29 (v) HVR-L2 comprising the amino acid sequence of SEQ ID NO:30, and (vi) HVR-L3 comprising the amino acid sequence of SEQ ID NO:31,
   (c) a VH domain comprising (i) HVR-H1 comprising the amino acid sequence of SEQ ID NO:34, (ii) HVR-H2 comprising the amino acid sequence of SEQ ID NO:35, and (iii) HVR-H3 comprising the amino acid sequence of SEQ ID NO:36, and a VL domain comprising (iv) HVR-L1 comprising the amino acid sequence of SEQ ID NO:37 (v) HVR-L2 comprising the amino acid sequence of SEQ ID NO:38, and (vi) HVR-L3 comprising the amino acid sequence of SEQ ID NO:39,
   (d) a VH domain comprising (i) HVR-H1 comprising the amino acid sequence of SEQ ID NO:42, (ii) HVR-H2 comprising the amino acid sequence of SEQ ID NO:43, and (iii) HVR-H3 comprising the amino acid sequence of SEQ ID NO:44, and a VL domain comprising (iv) HVR-L1 comprising the amino acid sequence of SEQ ID NO:45 (v) HVR-L2 comprising the amino acid sequence of SEQ ID NO:46, and (vi) HVR-L3 comprising the amino acid sequence of SEQ ID NO:47,
   (e) a VH domain comprising (i) HVR-H1 comprising the amino acid sequence of SEQ ID NO:50, (ii) HVR-H2 comprising the amino acid sequence of SEQ ID NO:51, and (iii) HVR-H3 comprising the amino acid sequence of SEQ ID NO: 52, and a VL domain comprising (iv) HVR-L1 comprising the amino acid sequence of SEQ ID NO:53 (v) HVR-L2 comprising the amino acid sequence of SEQ ID NO:54, and (vi) HVR-L3 comprising the amino acid sequence of SEQ ID NO:55,
   (f) a VH domain comprising (i) HVR-H1 comprising the amino acid sequence of SEQ ID NO:58, (ii) HVR-H2 comprising the amino acid sequence of SEQ ID NO:59, and (iii) HVR-H3 comprising the amino acid sequence of SEQ ID NO: 60, and a VL domain comprising (iv) HVR-L1 comprising the amino acid sequence of SEQ ID NO:61 (v) HVR-L2 comprising the amino acid sequence of SEQ ID NO:62, and (vi) HVR-L3 comprising the amino acid sequence of SEQ ID NO:63, or
   (g) a VH domain comprising (i) HVR-H1 comprising the amino acid sequence of SEQ ID NO:66, (ii) HVR-H2 comprising the amino acid sequence of SEQ ID NO:67, and (iii) HVR-H3 comprising the amino acid sequence of SEQ ID NO: 68, and a VL domain comprising (iv) HVR-L1 comprising the amino acid sequence of SEQ ID NO:69 (v) HVR-L2 comprising the amino acid sequence of SEQ ID NO:70, and (vi) HVR-L3 comprising the amino acid sequence of SEQ ID NO:71.
15. The TNF family ligand trimer-containing antigen binding molecule of any one of paras 1 to 14, wherein the moiety capable of specific binding to PD1 comprises
   (a) a VH domain comprising an amino acid sequence of SEQ ID NO: 19 and a VL domain comprising an amino acid sequence of SEQ ID NO:20,
   (b) a VH domain comprising an amino acid sequence of SEQ ID NO:21 and a VL domain comprising an amino acid sequence of SEQ ID NO:22,
   (c) a VH domain comprising an amino acid sequence of SEQ ID NO:21 and a VL domain comprising an amino acid sequence of SEQ ID NO:23,
   (d) a VH domain comprising an amino acid sequence of SEQ ID NO:21 and a VL domain comprising an amino acid sequence of SEQ ID NO:24,
   (e) a VH domain comprising an amino acid sequence of SEQ ID NO:21 and a VL domain comprising an amino acid sequence of SEQ ID NO:25,
   (f) a VH domain comprising an amino acid sequence of SEQ ID NO:32 and a VL domain comprising an amino acid sequence of SEQ ID NO:33,
   (g) a VH domain comprising an amino acid sequence of SEQ ID NO:40 and a VL domain comprising an amino acid sequence of SEQ ID NO:41,
   (h) a VH domain comprising an amino acid sequence of SEQ ID NO:48 and a VL domain comprising an amino acid sequence of SEQ ID NO:49,
   (i) a VH domain comprising an amino acid sequence of SEQ ID NO:56 and a VL domain comprising an amino acid sequence of SEQ ID NO:57,
   (k) a VH domain comprising an amino acid sequence of SEQ ID NO:64 and a VL domain comprising an amino acid sequence of SEQ ID NO:65, or
   (l) a VH domain comprising an amino acid sequence of SEQ ID NO:72 and a VL domain comprising an amino acid sequence of SEQ ID NO:73.
16. The TNF family ligand trimer-containing antigen binding molecule of any one of paras 1 to 14, wherein the moiety capable of specific binding to PD1 comprises
   (a) a VH domain comprising (i) HVR-H1 comprising the amino acid sequence of SEQ ID NO: 13, (ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16, (v) HVR-L2 comprising the amino acid sequence of SEQ ID NO: 17, and (vi) HVR-L3 comprising the amino acid sequence of SEQ ID NO: 18.
17. The TNF family ligand trimer-containing antigen binding molecule of any one of paras 1 to 16, wherein the moiety capable of specific binding to PD1 comprises
   (a) a VH domain comprising an amino acid sequence of SEQ ID NO: 19 and a VL domain comprising an amino acid sequence of SEQ ID NO:20,
   (b) a VH domain comprising an amino acid sequence of SEQ ID NO:21 and a VL domain comprising an amino acid sequence of SEQ ID NO:22,
   (c) a VH domain comprising an amino acid sequence of SEQ ID NO:21 and a VL domain comprising an amino acid sequence of SEQ ID NO:23,
   (d) a VH domain comprising an amino acid sequence of SEQ ID NO:21 and a VL domain comprising an amino acid sequence of SEQ ID NO:24, or
   (e) a VH domain comprising an amino acid sequence of SEQ ID NO:21 and a VL domain comprising an amino acid sequence of SEQ ID NO:25.
18. The TNF family ligand trimer-containing antigen binding molecule of any one of paras 2 to 17, wherein the Fc domain is an IgG, particularly an IgG1 Fc domain or an IgG4 Fc domain.
19. The TNF family ligand trimer-containing antigen binding molecule of any one of paras 2 to 18, wherein the Fc domain is an IgG1 Fc domain comprising the amino acid substitutions at positions 234 and 235 (EU numbering) and/or 329 (EU numbering).
20. The TNF family ligand trimer-containing antigen binding molecule of any one of paras 1 to 19, wherein the first subunit of the Fc domain comprises the amino acid substitutions S354C and T366W (numbering according to Kabat EU index) and the second subunit of the Fc domain comprises the amino acid substitutions Y349C, T366S, L368A and Y407V (numbering according to Kabat EU index).
21. The TNF family ligand trimer-containing antigen binding molecule of any one of paras 1 to 20, wherein the antigen binding molecule comprises
   a first heavy chain and a first light chain, both comprising a Fab molecule capable of specific binding to PD1,
   a first peptide comprising two ectodomains of a TNF ligand family member or fragments thereof connected to each other by a first peptide linker fused at its C-terminus by a second peptide linker to a second heavy or light chain,
   and a second peptide comprising one ectodomain of said TNF ligand family member fused at its C-terminus by a third peptide linker to a second light or heavy chain, respectively.
22. The TNF family ligand trimer-containing antigen binding molecule of any one of paras 1 to 20, wherein the first peptide comprising two ectodomains of a TNF ligand family member or fragments thereof connected to each other by a first peptide linker is fused at its C-terminus by a second peptide linker to a CH1 domain that is part of a heavy chain, and the second peptide comprising one ectodomain of said TNF ligand family member or a fragment thereof is fused at its C-terminus by a third peptide linker to a CL domain that is part of a light chain.
23. The TNF family ligand trimer-containing antigen binding molecule of any one of paras 1 to 20, wherein the first peptide comprising two ectodomains of a TNF ligand family member or fragments thereof connected to each other by a first peptide linker is fused at its C-terminus by a second peptide linker to a CL domain that is part of a heavy chain, and the second peptide comprising one ectodomain of said TNF ligand family member or a fragment thereof is fused at its C-terminus by a third peptide linker to a CH1 domain that is part of a light chain.
24. The TNF family ligand trimer-containing antigen binding molecule of any one of paras 1 to 20, wherein the first peptide comprising two ectodomains of a TNF ligand family member or fragments thereof connected to each other by a first peptide linker is fused at its C-terminus by a second peptide linker to a VH domain that is part of a heavy chain, and the second peptide comprising one ectodomain of said TNF ligand family member or a fragment thereof is fused at its C-terminus by a third peptide linker to a VL domain that is part of a light chain.
25. The TNF family ligand trimer-containing antigen binding molecule of paras 21 to 23, wherein in the CL domain adjacent to the TNF ligand family member the amino acid at position 123 (EU numbering) has been replaced by arginine (R) and the amino acid at position 124 (EU numbering) has been substituted by lysine (K), and wherein in the CH1 domain adjacent to the TNF ligand family member the amino acids at position 147 (EU numbering) and at position 213 (EU numbering) have been substituted by glutamic acid (E).
26. The TNF family ligand trimer-containing antigen binding molecule of any one of paras 1 to 20, wherein the antigen binding molecule comprises
   (a) a first heavy chain and a first light chain, both comprising a Fab molecule capable of specific binding to PD1,
   (b) a second heavy chain comprising an amino acid sequence selected from the group consisting of SEQ ID NO:9, SEQ ID NO:11, SEQ ID NO:12 and SEQ ID NO:10, and
   a second light chain comprising an amino acid sequence selected from the group consisting of SEQ ID NO: 1, SEQ ID NO:3, SEQ ID NO:4 and SEQ ID NO:5.
27. The TNF family ligand trimer-containing antigen binding molecule of any one of paras 1 to 20 or 26, wherein the antigen binding molecule comprises
   (i) a first heavy chain comprising the VH domain comprising the amino acid sequence of SEQ ID NO: 19 and a first light chain comprising the VL domain comprising the amino acid sequence of SEQ ID NO:20 or
   a first heavy chain comprising the VH domain comprising the amino acid sequence of SEQ ID NO:21 and a first light chain comprising the VL domain comprising the amino acid sequence selected from the group consisting of SEQ ID NO:22, SEQ ID NO:23, SEQ ID NO:24 and SEQ ID NO:25,
   (ii) a second heavy chain comprising the amino acid sequence selected from the group consisting of SEQ ID NO:74, SEQ ID NO:76, SEQ ID NO:78 and SEQ ID NO:80, and
   (iii) a second light chain comprising the amino acid sequence selected from the group consisting of SEQ ID NO:75, SEQ ID NO:77, SEQ ID NO:79 and SEQ ID NO:81.
28. The TNF family ligand trimer-containing antigen binding molecule of any one of paras 1 to 21, wherein the antigen binding molecule comprises
   (a) a first heavy chain comprising the amino acid sequence of SEQ ID NO:82, a first light chain comprising the amino acid sequence of SEQ ID NO:83, a second heavy chain comprising the amino acid sequence of SEQ ID NO:74 and a second light chain comprising the amino acid sequence of SEQ ID NO:75,
   (b) a first heavy chain comprising the amino acid sequence of SEQ ID NO:82, a first light chain comprising the amino acid sequence of SEQ ID NO:83, a second heavy chain comprising the amino acid sequence of SEQ ID NO:76 and a second light chain comprising the amino acid sequence of SEQ ID NO:77,
   (c) a first heavy chain comprising the amino acid sequence of SEQ ID NO:82, a first light chain comprising the amino acid sequence of SEQ ID NO:83, a second heavy chain comprising the amino acid sequence of SEQ ID NO:78 and a second light chain comprising the amino acid sequence of SEQ ID NO:79, or
   (d) a first heavy chain comprising the amino acid sequence of SEQ ID NO:82, a first light chain comprising the amino acid sequence of SEQ ID NO:83, a second heavy chain comprising the amino acid sequence of SEQ ID NO:80 and a second light chain comprising the amino acid sequence of SEQ ID NO:81.
29. The TNF family ligand trimer-containing antigen binding molecule of any one of paras 1 to 20, comprising
   (a) at least one Fab domain capable of specific binding to PD1, and
   (b) a first and a second polypeptide that are linked to each other by a disulfide bond,
   wherein the antigen binding molecule is characterized in that the first polypeptide contains a CH3 domain and the second polypeptide contains a CH3 domain, respectively,
   and wherein the first polypeptide comprises two ectodomains of a TNF ligand family member or fragments thereof that are connected to each other and to the C-terminus of the CH3 domain by a peptide linker and wherein the second polypeptide comprises one ectodomain of said TNF ligand family member or a fragment thereof connected via a peptide linker to C-terminus of the CH3 domain of said polypeptide.
30. The TNF family ligand trimer-containing antigen binding molecule of para 29, comprising two Fab domains capable of specific binding to PD1.
31. The TNF family ligand trimer-containing antigen binding molecule of para 29, wherein the antigen binding molecule comprises
   (a) a first heavy chain comprising the amino acid sequence of SEQ ID NO:84, a second heavy chain comprising the amino acid sequence of SEQ ID NO:85, and two light chains comprising the amino acid sequence of SEQ ID NO:83, or
   (b) a first heavy chain comprising the amino acid sequence of SEQ ID NO:86, a second heavy chain comprising the amino acid sequence of SEQ ID NO:87, and two light chains comprising the amino acid sequence of SEQ ID NO:83.
32. The TNF family ligand trimer-containing antigen binding molecule of para 29, comprising one Fab domain capable of specific binding to PD1.
33. The TNF family ligand trimer-containing antigen binding molecule of para 29 or 32,
   wherein the antigen binding molecule comprises
   (i) a fusion polypeptide comprising a first subunit of a Fc domain and two ectodomains of a TNF ligand family member or fragments thereof that are connected to each other and to the C-terminus of the CH3 domain by a peptide linker, (ii) a heavy chain comprising the VH domain of the Fab domain capable of specific binding to PD1, a second subunit of the Fc domain and one ectodomain of said TNF ligand family member or a fragment thereof connected via a peptide linker to C-terminus of the CH3 domain, and (iii) a light chain comprising the VL domain of the Fab domain capable of specific binding to PD1, or
   (i) a fusion polypeptide comprising a first subunit of a Fc domain and one ectodomain of a TNF ligand family member or a fragment thereof that is connected to the C-terminus of the CH3 domain by a peptide linker, (ii) a heavy chain comprising the VH domain of the Fab domain capable of specific binding to PD1, a second subunit of the Fc domain and two ectodomains of said TNF ligand family member or fragments thereof that are connected to each other and to the C-terminus of the CH3 domain via a peptide linker, and (iii) a light chain comprising the VL domain of the Fab domain capable of specific binding to PD1.
34. The TNF family ligand trimer-containing antigen binding molecule of para 31, wherein the antigen binding molecule comprises
   (a) a fusion polypeptide comprising the amino acid sequence of SEQ ID NO: 88, a heavy chain comprising the amino acid sequence of SEQ ID NO:87 and a light chain comprising the amino acid sequence of SEQ ID NO:83,
   (b) a fusion polypeptide comprising the amino acid sequence of SEQ ID NO:89, a heavy chain comprising the amino acid sequence of SEQ ID NO:90 and a light chain comprising the amino acid sequence of SEQ ID NO:83,
   (c) a fusion polypeptide comprising the amino acid sequence of SEQ ID NO:91, a heavy chain comprising the amino acid sequence of SEQ ID NO:86 and a light chain comprising the amino acid sequence of SEQ ID NO:83,
   (b) a fusion polypeptide comprising the amino acid sequence of SEQ ID NO:93, a heavy chain comprising the amino acid sequence of SEQ ID NO:92 and a light chain comprising the amino acid sequence of SEQ ID NO:83,
35. An isolated polynucleotide encoding the TNF family ligand trimer-containing antigen binding molecule of any one of paras 1 to 34.
36. A vector, particularly an expression vector, comprising the isolated polynucleotide of para 35.
37. A host cell comprising the isolated polynucleotide of para 35 or the vector of para 36.
38. A method for producing the TNF family ligand trimer-containing antigen binding molecule of any one of paras 1 to 34, comprising the steps of
   (i) culturing the host cell of para 37 under conditions suitable for expression of the antigen binding molecule, and
   (ii) recovering the antigen binding molecule.
39. A pharmaceutical composition comprising the TNF family ligand trimer-containing antigen binding molecule of any one of paras 1 to 34 and at least one pharmaceutically acceptable excipient.
40. The TNF family ligand trimer-containing antigen binding molecule of any one of points 1 to 34, or the pharmaceutical composition of para 39, for use as a medicament.
41. The TNF family ligand trimer-containing antigen binding molecule of any one of claims 1 to 34, or the pharmaceutical composition of para 39, for use in the treatment of cancer.
42. Use of the TNF family ligand trimer-containing antigen binding molecule of any one of paras 1 to 34 for the manufacture of a medicament for the treatment of cancer.
43. A method of treating a disease in an individual, comprising administering to said individual a therapeutically effective amount of a composition comprising a TNF family ligand trimer-containing antigen binding molecule of any one of paras 1 to 34 in a pharmaceutically acceptable form.
44. The method of para 43, wherein said disease is cancer.

### EXAMPLES

The following are examples of methods and compositions of the invention. It is understood that various other embodiments may be practiced, given the general description provided above.

Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

DNA Sequencing

DNA sequences were determined by double strand sequencing.

Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

Mass Spectrometry

This section describes the characterization of the multispecific antibodies with VH/VL exchange (VH/VL CrossMabs) with emphasis on their correct assembly. The expected primary structures were analyzed by electrospray ionization mass spectrometry (ESI-MS) of the deglycosylated intact CrossMabs and deglycosylated/plasmin digested or alternatively deglycosylated/limited LysC digested CrossMabs.

The VH/VL CrossMabs were deglycosylated with N-Glycosidase F in a phosphate or Tris buffer at 37° C. for up to 17 h at a protein concentration of 1 mg/ml. The plasmin or limited LysC (Roche) digestions were performed with 100 μg deglycosylated VH/VL CrossMabs in a Tris buffer pH 8 at room temperature for 120 hours and at 37° C. for 40 min, respectively. Prior to mass spectrometry the samples were desalted via HPLC on a Sephadex G25 column (GE Healthcare). The total mass was determined via ESI-MS on a maX is 4G UHR-QTOF MS system (Bruker Daltonik) equipped with a TriVersa NanoMate source (Advion).

Determination of Binding and Binding Affinity of Multispecific Antibodies to the Respective Antigens Using Surface Plasmon Resonance (SPR) (BIACORE)

Binding of the generated antibodies to the respective antigens is investigated by surface plasmon resonance using a BIACORE instrument (GE Healthcare Biosciences AB, Uppsala, Sweden). Briefly, for affinity measurements Goat-Anti-Human IgG, JIR 109-005-098 antibodies are immobilized on a CM5 chip via amine coupling for presentation of the antibodies against the respective antigen. Binding is measured in HBS buffer (HBS-P (10 mM HEPES, 150 mM NaCl, 0.005% Tween 20, ph 7.4), 25° C. (or alternatively at 37° C.). Antigen (R&D Systems or in house purified) was added in various concentrations in solution. Association was measured by an antigen injection of 80 seconds to 3 minutes; dissociation was measured by washing the chip surface with HBS buffer for 3-10 minutes and a KD value was estimated using a 1:1 Langmuir binding model. Negative control data (e.g. buffer curves) are subtracted from sample curves for correction of system intrinsic baseline drift and for noise signal reduction. The respective Biacore Evaluation Software is used for analysis of sensorgrams and for calculation of affinity data.

### Example 1

1.1 Generation of Anti-PD1 Antibodies

Immunization of Mice

NMRI mice were immunized genetically, using a plasmid expression vector coding for full-length human PD1 by intradermal application of 100 ug vector DNA (plasmid15300_hPD1-fl), followed by Electroporation (2 square pulses of 1000 V/cm, duration 0.1 ms, interval 0.125 s; followed by 4 square pulses of 287.5 V/cm, duration 10 ms, interval 0.125 s. Mice received either 6 consecutive immunizations at days 0, 14, 28, 42, 56, 70, and 84. Blood was taken at days 36, 78 and 92 and serum prepared, which was used for titer determination by ELISA (see below). Animals with highest titers were selected for boosting at day 96, by intravenous injection of 50 ug of recombinant human PD1 human Fc chimera, and monoclonal antibodies were isolated by hybridoma technology, by fusion of splenocytes to myeloma cell line 3 days after boost.

Determination of Serum Titers (ELISA)

Human recombinant PD1 human Fc chimera was immobilized on a 96-well NUNC Maxisorp plate at 0.3 ug/ml, 100 ul/well, in PBS, followed by: blocking of the plate with 2% Crotein C in PBS, 200 ul/well; application of serial dilutions of antisera, in duplicates, in 0.5% Crotein C in PBS, 100 ul/well; detection with HRP-conjugated goat anti-mouse antibody (Jackson Immunoresearch/Dianova 115-036-071; 1/16 000). For all steps, plates were incubated for 1 h at 37° C. Between all steps, plates were washed 3 times with 0.05% Tween 20 in PBS. Signal was developed by addition of BM Blue POD Substrate soluble (Roche), 100 ul/well; and stopped by addition of 1 M HCl, 100 ul/well. Absorbance was read out at 450 nm, against 690 nm as reference. Titer was defined as dilution of antisera resulting in half-maximal signal.

1.2 Characterization Anti-PD1 Antibodies/Binding of Anti-PD1 Antibodies to Human PD1

ELISA for Hu PD1

Nunc maxisorp streptavidin coated plates (MicroCoat #11974998001) were coated with 25 μl/well biotinylated PD1-ECD-AviHis and incubated at 4° C. over night. After washing (3×90 μl/well with PBST-buffer) 25 μl anti PD1 samples or reference antibodies (human anti PD1; Roche/mouse anti PD1; Biolegend; cat.:329912) were added and incubated 1 h at RT. After washing (3×90 μl/well with PBST-buffer) 25 μl/well goat-anti-human H+L-POD (JIR, JIR109-036-088)/Sheep-anti-mouse-POD (GE Healthcare; NA9310) was added in 1:2000/1:1000 dilution and incubated at RT for 1 h on shaker. After washing (3×90 μl/well with PBST-buffer) μl/well TMB substrate (Roche Catalogue No. 11835033001) was added and incubated until OD 2-3. Measurement took place at 370/492 nm.

ELISA results are listed as EC₅₀-values [ng/ml] in Summary Table 1 and 2 below.

Cell ELISA for PD1

Adherent CHO-K1 cell line stably transfected with plasmid 15311_hPD1-fl_pUC_Neo coding for full-length human PD1 and selection with G418 (Neomycin resistance marker on plasmid) were seeded at a concentration of 0.01×10E6 cells/well in 384-well flat bottom plates and grown over night.

The next day 25 μl/well PD1 sample or human anti PD1 (Roche)/mouse anti PD1(Biolegend; cat.:329912) reference antibody were added and incubated for 2h at 4° C. (to avoid internalization). After washing carefully (1×90 l/well PBST) cells were fixed by adding 30 μl/well 0.05% Glutaraldehyde (Sigma, Cat. No: G5882, 25%) diluted in 1×PBS-buffer and incubated for 10 min at RT. After washing (3×90 μl/well PBST) 25 μl/well secondary antibody was added for detection: goat-anti-human H+L-POD (JIR, JIR109-036-088)/Sheep-anti-mouse-POD (GE NA9310) followed by 1 h incubation at RT on shaker. After washing (3×90 μl/well PBST) 25 μl/well TMB substrate solution (Roche 11835033001) was added and incubated until OD 1.0-2.0. Plates were measured at 370/492 nm.

Cell ELISA results are listed as “EC₅₀ CHO-PD1”-values [ng/ml] in Summary Table 2 below.

ELISA for Cyno PD1

Nunc maxisorp streptavidin coated plates (MicroCoat #11974998001) were coated with 25 μl/well biotinylated cyno PD1-ECD-Biotin and incubated at 4° C. over night. After washing (3×90 μl/well with PBST-buffer) 25 μl anti PD1 samples or reference antibodies (human anti PD1; Roche) were added and incubated 1 h at RT on shaker. After washing (3×90 μl/well with PBST-buffer) 25 μl/well goat-anti-human H+L-POD (JIR, JIR109-036-088) was added in 1:1000 dilution and incubated at RT for 1 h on shaker. After washing (3×90 μl/well with PBST-buffer) μl/well TMB substrate (Roche, 11835033001) was added and incubated until OD 2-3. Measurement took place at 370/492 nm.

ELISA results are listed as EC₅₀-values [ng/ml] in Summary Tables 1 and 2 below.

PD Ligand 1 Replacing Assay

Nunc maxisorp streptavidin coated plates (MicroCoat #11974998001) were coated with 25 μl/well biotinylated PD1-ECD-AviHis and incubated at 4° C. over night. After washing (3×90 μl/well with PBST-buffer) 25 μl anti PD1 samples or reference antibodies (mouse anti PD1; Biolegend; cat.:329912) were added and incubated 1 h at RT on shaker. After washing (3×90 μl/well with PBST-buffer) 25 μl/well PD-L1 (Recombinant human B7-H1/PD-L1 Fc Chimera; 156-B7, R&D) was added and incubated 1 h at RT on shaker. After washing (3×90 μl/well with PBST-buffer) 25 μl/well goat-anti-human H+L-POD (JIR, 109-036-088) was added in 1:1000 dilution and incubated at RT for 1 h on shaker. After washing (3×90 μl/well with PBST-buffer) μl/well TMB substrate (Roche, 11835033001) was added and incubated until OD 2-3. Measurement took place at 370/492 nm.

ELISA results are listed as IC₅₀-values [ng/ml] in Summary Table 1 below.

PD Ligand 2 Replacing Assay

Nunc maxisorp streptavidin coated plates (MicroCoat #11974998001) were coated with 25 μl/well biotinylated PD1-ECD-AviHis and incubated at 4° C. over night. After washing (3×90 μl/well with PBST-buffer) 25 μl anti PD1 samples or reference antibodies (mouse anti huPD1; Roche) were added and incubated 1 h at RT on shaker. After washing (3×90 μl/well with PBST-buffer) 25 μl/well PD-L2 (Recombinant human B7-DC/PD-L2 Fc Chimera; 1224-PL-100, R&D) was added and incubated 1 h at RT on shaker. After washing (3×90 μl/well with PBST-buffer) 25 μl/well goat-anti-human H+L-POD (JIR, 109-036-088) was added in 1:2000 dilution and incubated at RT for 1 h on shaker. After washing (3×90 μl/well with PBST-buffer) 25 μl/well TMB substrate (Roche, 11835033001) was added and incubated until OD 2-3. Measurement took place at 370/492 nm.

ELISA results are listed as IC₅₀-values [ng/ml] in summary Table 1 below.

Epitope Mapping ELISA/Binding Competition Assay

Nunc maxisorp plates (Nunc #464718) were coated with 25 l/well capture antibody (goat anti mouse IgG; JIR; 115-006-071) and incubated for 1 h at RT on shaker. After washing (3×90 μl/well with PBST-buffer) plates were blocked for 1 h with 2% BSA containing PBS buffer at RT on shaker. After washing (3×90 μl/well with PBST-buffer) 25 μl mouse anti PD1 samples were added and incubated 1 h at RT on shaker. After washing (3×90 μl/well with PBST-buffer) capture antibody was blocked by 30 μl/well mouse IgG (JIR; 015-000-003) for 1 h at RT on shaker. At the same time biotinylated PD1-ECD-AviHis was preincubated with second sample antibody for 1 h at RT on shaker. After washing assay plate (3×90 μl/well with PBST-buffer) the PD1 antibody mix was transferred to assay plate and incubated at RT for 1 h on shaker. After washing (3×90 μl/well with PBST-buffer) 25 μl/well streptavidin POD (Roche, #11089153001) was added in 1:4000 dilution and incubated at RT for 1 h on shaker. After washing (3×90 μl/well with PBST-buffer) 25 μl/well TMB substrate (Roche, #11089153001) was added and incubated until OD 1.5-2.5. Measurement took place at 370/492 nm. Epitope groups were defined by hierarchical clustering against reference antibodies.

TABLE 1
Binding, PD-L1 inhibition and epitope region groups of
exemplary PD1 antibodies (ELISA)

| | | | | ELISA | Epitope |
|---|---|---|---|---|---|
| | | | | PD- | region |
| | ELISA | ELISA | ELISA | L2 | group |
| | huPD1 | cyPD1 | PD-L1 | inhibition | (by |
| | EC₅₀ | EC₅₀ | inhibition | IC₅₀ | competition |
| Antibody | [ng/ml] | [ng/ml] | IC₅₀ [ng/ml] | [ng/ml] | assay) |
| PD1-0050 | 17.9 | 9.8 | 128 | 34 | 1 |
| PD1-0069 | 45.7 | 22.7 | 225 | 89 | 6 |
| PD1-0073 | 15.1 | 8.3 | 124 | 65 | 5 |
| PD1-0078 | 26.3 | 22.4 | x | 86 | 2 |
| PD1-0098 | 50.8 | 54.6 | 174 | 45 | 5 |
| PD1-0102 | 34.2 | 52.7 | >35.5 μg/ml | 140 | 4 |
| PD1-0103 | 33.7 | 36.9 | 182 | 51 | 5 |

TABLE 2
Biochemial- and Cell-binding of humanized PD1 antibodies
derived from parental mouse antibody PD1-0103 (ELISA)

| Humanized | ELISA huPD1 | ELISA cyPD1 | ELISA CHO-PD1 |
|---|---|---|---|
| antibody | EC₅₀ [ng/ml] | EC₅₀ [ng/ml] | EC₅₀ [ng/ml] |
| PD1-103-0312 | 11 | 8.3 | 10.1 |
| PD1-103-0313 | 15 | 11 | 10.8 |
| PD1-103-0314 | 11 | 8.3 | 7.7 |
| PD1-103-0315 | 10 | 7.9 | 7.3 |

Biacore Characterization of the Humanized Anti-PD1 Antibodies

A surface plasmon resonance (SPR) based assay has been used to determine the kinetic parameters of the binding between several murine PD1 binders as well as commercial human PD1 binding references. Therefore, an anti-human IgG was immobilized by amine coupling to the surface of a (Biacore) CM5 sensor chip. The samples were then captured and hu PD1-ECD was bound to them. The sensor chip surface was regenerated after each analysis cycle. The equilibrium constant and kinetic rate constants were finally gained by fitting the data to a 1:1 langmuir interaction model.

About 2000 response units (RU) of 20 μg/ml anti-human IgG (GE Healthcare #BR-1008-39) were coupled onto the flow cells 1 and 2 (alternatively: 3 and 4) of a CM5 sensor chip in a Biacore T200 at pH 5.0 by using an amine coupling kit supplied by GE Healthcare.

The sample and running buffer was HBS-EP+ (0.01 M HEPES, 0.15 M NaCl, 3 mM EDTA, 0.05% v/v Surfactant P20, pH 7.4). Flow cell temperature was set to 25° C. and sample compartment temperature to 12° C. The system was primed with running buffer.

The samples were injected for 20 seconds with a concentration of 10 nM and bound to the second flow cell. Then a complete set of human PD1-ECD concentrations (144 nM, 48 nM, 16 nM, 5.33 nM, 1.78 nM, 0.59 nM, 0.20 nM and 0 nM) was injected over each sample for 120s followed by a dissociation time of 30/300s and two 20s regeneration steps with 3 M MgCl₂, of which the last one contained an “extra wash after injection” with running buffer.

Finally the double referenced data was fitted to a 1:1 langmuir interaction model with the Biacore T200 Evaluation Software. Resulting K_{D}, kₐ and k_{d} values are shown in Table 3.

TABLE 3
Kinetic rate constants and equilibrium constant for chimeric
PD1-0103 and humanized PD1-Abs determined by Biacore

| Ligand | kₐ [M⁻¹s⁻¹] | k_{d} [s⁻¹] | K_{D} [nM] |
|---|---|---|---|
| chimeric PD1-0103 | 3.86E+05 | 3.07E−04 | 0.8 |
| PD1-0103-0312 | 1.95E+05 | 3.45E−04 | 1.8 |
| PD1-0103-0313 | 1.60E+05 | 3.67E−04 | 2.3 |
| PD1-0103-0314 | 1.87E+05 | 2.79E−04 | 1.5 |
| PD1-0103-0315 | 1.89E+05 | 2.91E−04 | 1.5 |

As shown in Table 3, all the humanized versions of chimeric PD1-0103 (generation see Example 1.3) display kinetic properties similar to the parental antibody (chimeric PD1-0103).

Kinetics

A CM5 sensor series S was mounted into the Biacore 4000 System and the detection spots were hydrodynamically addressed according to the manufacturer's instructions.

The polyclonal rabbit IgG antibody <IgGFCyM>R (Jackson ImmunoResearch Laboratories Inc.) was immobilized at 10 000 Ru on the detection spots 1 and 5 in the flow cells 1, 2, 3 and 4. Coupling was done via EDC/NHS chemistry according to the manufacturer's instructions. The remaining spots in the flow cells served as a reference. The sample buffer was the system buffer supplemented with 1 mg/ml carboxymethyldextrane.

In one embodiment the assay was driven at 25° C. In another embodiment the assay was driven at 37° C. 50 nM of each murine monoclonal antibody was captured on the sensor surface by a 1 min injection at 10 μl/min. Subsequently the respective antigens were injected in a concentration series of 100 nM, 2×33 nM, 11 nM, 4 nM, 1 nM and system buffer 0 nM at 30 μl/min for 4 min association phase time. The dissociation was monitored for another 4 min. The capture system was regenerated using a 3 min injection of 10 mM glycine pH 1.5 at 30 μl/min. Relevant kinetic data was calculated using the Biacore evaluation software according to the manufacturer's instructions.

Epitope Mapping

A Biacore 4000 instrument was mounted with a Biacore CAP sensor and was prepared like recommended by the manufacturer. The instrument buffer was HBS-ET (10 mM HEPES pH 7.4, 150 mM NaCl, 3 mM EDTA, 0.005% w/v Tween 20). The instrument was running at 25° C.

All samples were diluted in system buffer. A 35 kDa biotinylated antigen PD1-ECD-AviHis was captured at 200 RU on the CAP sensor surface by a 1 min injection at 30 μl/min in the flow cells 1, 2, 3 and 4 in the spots 1 and 5. Spots 2, 3 and 4 served as a reference. In another embodiment, a 35 kDa biotinylated antigen PD1-ECD-AviHis was captured at 200 RU on the CAP sensor in the same manner.

Subsequently a primary antibody was injected at 100 nM for 3 min at 30 μl/min followed by the injection of a secondary antibody at 100 nM for 3 min at 30 μl/min. The primary antibody was injected until full saturation of the surface presented antigen. At the end of the primary and secondary antibody injection phases report points “Binding Late” (BL) were set to monitor the binding response of the respective antibodies. The Molar Ratio, a quotient between the secondary antibody binding response “BL2” and the primary antibody response “BL1” was calculated. The Molar Ratio was used as an indicator of the antigen accessibility of the secondary antibody, when the antigen was already complexed by the primary antibody.

The complexes were completely removed from the sensor surface by an injection for 2 min at 30 μl/min 2M guanidine-HCL 250 mM NaOH regeneration buffer as recommended by the manufacturer, followed by a 1 min injection at 30 μl/min of system buffer.

1.3 Effect of Different Anti-PD1 Antibodies on Cytokine Production in a Mixed Lymphocyte Reaction (MLR)

3A) The Mixed Lymphocyte Reaction (MLR) is a immune cell assay which measures the activation of lymphocytes from one individual (donor X) to lymphocytes from another individual (donor Y). A mixed lymphocyte reaction was used to demonstrate the effect of blocking the PD1 pathway to lymphocyte effector cells. T cells in the assay were tested for activation and theier IFN-gamma secretion in the presence or absence of an anti-PD1 mAbs.

To perform an allogeneic MLR, peripheral blood mononuclear cells (PBMCs) from at least four healthy donors of unknown HLA type were isolated by density gradient centrifugation using Leukosep (Greiner Bio One, 227 288). Briefly, heparinized blood samples were diluted with the three fold volume of PBS and 25 ml aliquots of the diluted blood were layered in 50 ml Leukosep tubes. After centrifugation at 800×g for 15 min at room temperature (w/o break) the lymphocyte containing fractions were harvested, washed in PBS and used directly in functional assay or resuspended in freezing medium (10% DMSO, 90% FCS) at 1.0E+07 cells/ml and stored in liquid nitrogen. Individual 2-way MLR reactions were set up by mixing PBMCs from two different donors at a 1:1 stimulator/responder cell ratio and co-cultures were done at least in duplicate in flat-bottomed 96-well plates for 6 days at 37° C., 5% CO2, in the presence or w/o of a different concentration range of purified anti-PD1 monoclonal antibodies PD1-0050, PD1-0069, PD1-0073, PD1-0078, PD1-0098, PD1-0102, PD1-0103. As reference anti-PD1 antibodies antibodies comprising the VH and VL domains of either nivolumab (also known as MDX-5C4 or MDX-1106) or pembrolizumab (also known as MK-3475 or Org 1.09A) were synthesized and cloned with backbones of human IgG1 (with mutations L234A, L235A and P329G (EU index of Kabat)). Either no antibody or an isotype control antibody was used as a negative control and rec hu IL-2 (20 EU/ml) was used as positive control. After day 6 100 μl of medium was taken from each culture for cytokine measurement. The levels of IFN-gamma were measured using OptEIA ELISA kit (BD Biosciences).

The results are shown in Table 4 (IFN-γ secretion/release). The anti-PD1 monoclonal antibodies promoted T cell activation and IFN-gamma secretion in concentration dependent manner. The value of % increase of IFNg secretion was calculated in relation to IFN-γ production of MLR w/o adding of any blocking mAbs (basal allogeneic stimulation induced IFNg value as E-c) and MLR with adding of 20 EU/ml rec hu IL-2 (positive control=100% IFNg value as E+c) and was calculated according to formula: Rel.Stimulation [%]=((Example−E−c)/(E+c −E−c)*100).

TABLE 4
Percentage of of IFN gamma secretion after allogenic
stimulation and treatment with anti-PD1
antibody in comparison to effect of recombinant human
IL-2 treatment (20 EU/ml) (=100% increase) as positive control

| | Concentration | | | | |
|---|---|---|---|---|---|
| | (μg/ml) | 1:12 | 1:120 | 1:1200 | Effect in MLR |
| PD1-0050 | 44 | 136 | 96 | 33 | +++ |
| PD1-0069 | 60 | 76 | 71 | 55 | +++ |
| PD1-0073 | 43 | 103 | 63 | 38 | ++ |
| PD1-0078 | 64 | 99 | 72 | 21 | ++ |

Several PD1 blocking antibodies PD1-0050, PD1-0069, PD1-0073, PD1-0078, PD1-0098, PD1-0102, PD1-0103 demonstrated strong immune modulating activity by enhancing secretion of interferon gamma (IFN-γ) (data not shown for all antibodies).

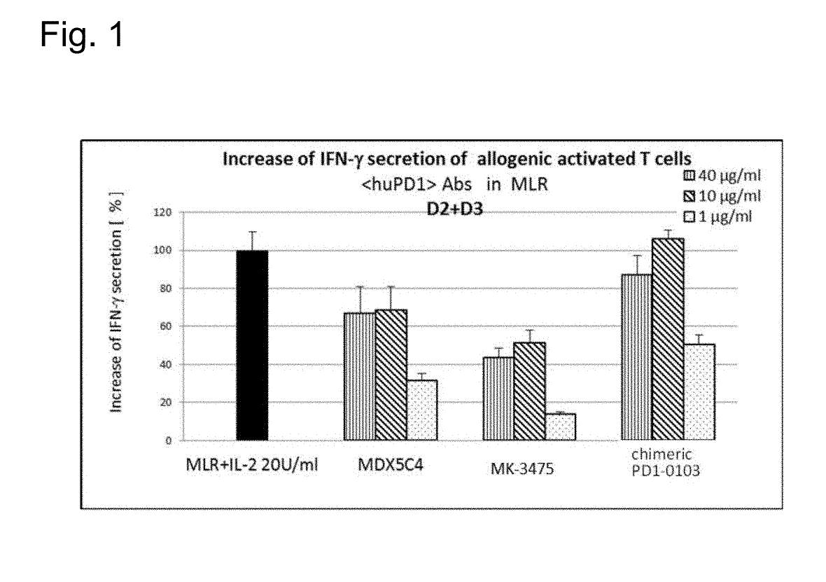

3B) In a further experiment chimeric PD1-0103 (human IgG1 isotype with mutations L234A, L235A and P329G (EU index of Kabat)) was evaluated. Blockade of PD1 with chimeric PD1-0103 strongly enhances IFN-gamma secretion by allogenic stimulated primary human T cells. Chimeric PD1-0103 is more potent than reference anti-PD1 antibodies (see FIG. 1).

For comparison the reference anti-PD1 antibodies comprising the VH and VL domains of either nivolumab (also known as MDX5C4 or MDX-1106) and pembrolizumab (also known as MK-3475 or Org 1.09A) were synthesized and cloned with backbones of human IgG1 (with mutations L234A, L235A and P329G (EU index of Kabat)) were used.

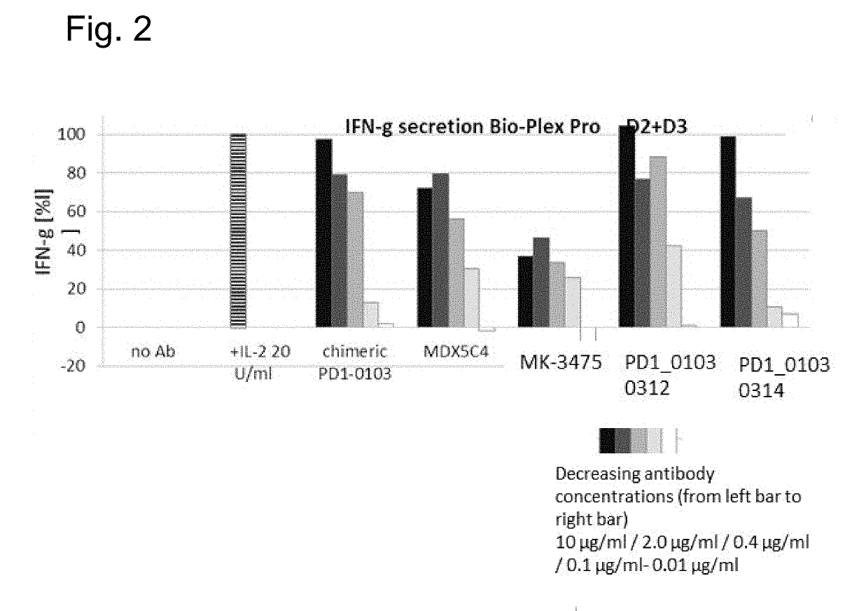

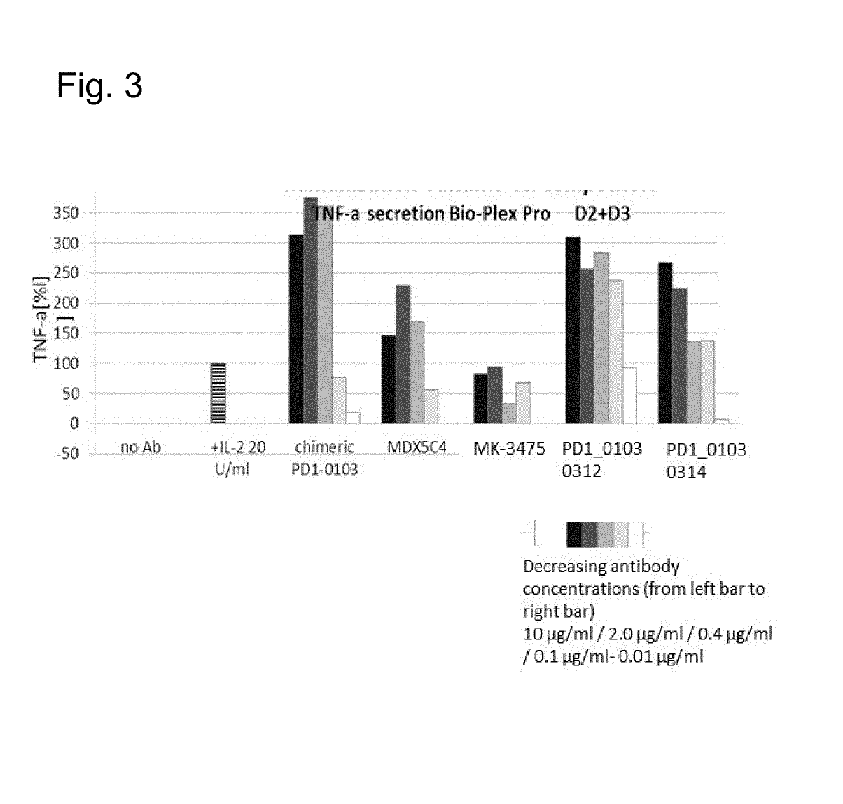

3C) In additional experiments the immune modulating activity of the humanized variants of anti-PD1 antibody PD1-0103 (humanized antibodies PD1-0103-0312, PD1-0103-0314, in FIG. 2 and FIG. 3) the a) IFNγ release (secretion) b) TNF-alpha release (secretion) was evaluated in MLR as described above. The effect of the chimeric PD1-0103 antibody and its humanized versions were compared to the reference anti-PD1 antibodies comprising the VH and VL domains of either nivolumab (also known as MDX5C4 or MDX-1106) and pembrolizumab (also known as MK-3475 or Org 1.09A) with backbones of human IgG1 (with mutations L234A, L235A and P329G (EU index of Kabat)). After 6 days of MLR culture 50 μl of supernatant was taken and multiple cytokines were measured in a single culture using Bio-Plex Pro™ Human Cytokine Th1/Th2 Assay (Bio-Rad Laboratories Inc.). (data not shown for all cytokines).

The chimeric PD1-0103 antibody and its humanized versions (PD1-0103_0312 and PD1-0103_0314) were more potent compared to the reference anti-PD1 antibodies in enhancing the T cell activation and IFN-gamma secretion (see FIG. 2).

Furthermore, the chimeric PD1-0103 antibody and its humanization variants increase tumor necrosis factor alpha (TNF alpha) (see FIG. 3) and IL-12 (data not shown) secretion by antigen presenting cells and encance capacity of monocytes/macrophages or antigen presenting cells to stimulate a T cell.

1.4 Effect of Anti-PD1 Blockade on Cytotoxic Granzyme B Release and IFN-γ Secretion by Human CD4 T Cells Cocultured with Allogeneic Mature Dendritic Cells

To further investigate the effect of anti-PD1 treatment in an allogeneic setting we developed an assay in which freshly purified CD4 T cells are cocultured for 5 days in presence of monocyte-derived allogeneic mature dendritic cells (mDCs). Monocytes were isolated from fresh PBMCs one week before through plastic adherence followed by the removal of the non-adherent cells. We then generated immature DCs from the monocytes by culturing them for 5 days in media containing GM-CSF (50 ng/ml) and IL-4 (100 ng/ml). To induce iDCs maturation, we added TNF-α, IL-1 3 and IL-6 (50 ng/ml each) to the culturing media for 2 additional days. We then assessed DCs maturation by measuring their surface expression of Major Histocompatibility Complex Class II (MHCII), CD80, CD83 and CD86 through flow cytometry (LSRFortessa, BD Biosciences).

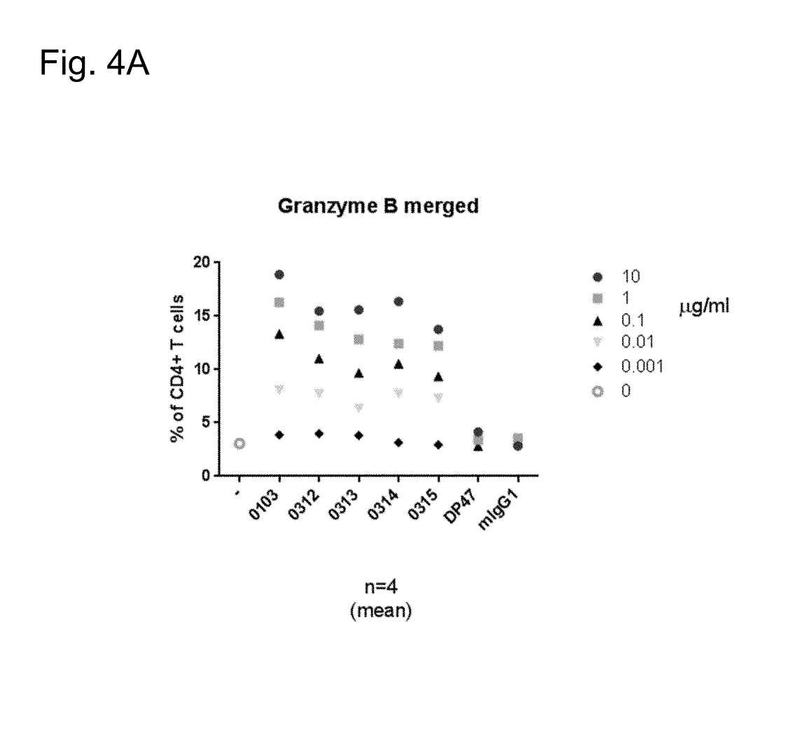

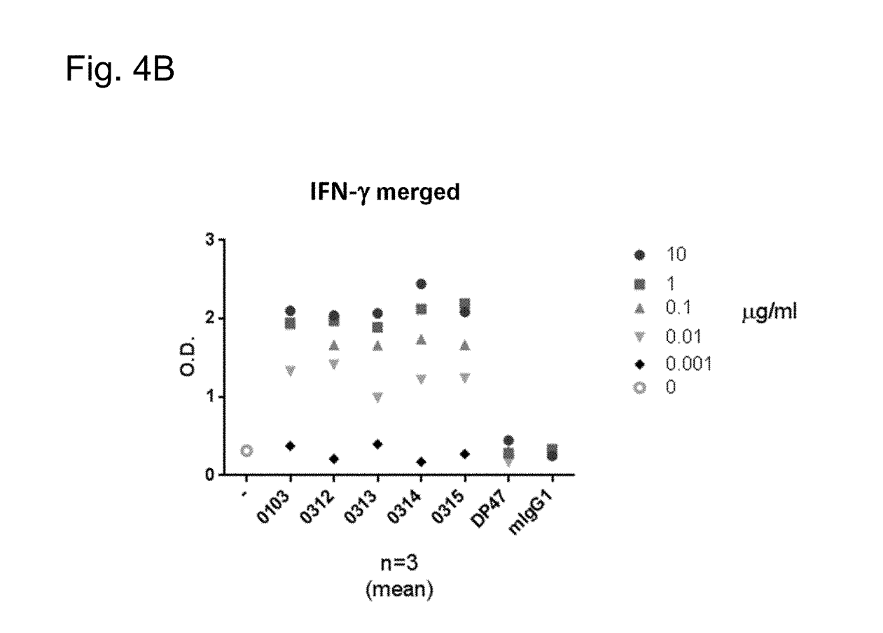

On the day of the minimal mixed lymphocyte reaction (mMLR), CD4 T cells were enriched via a microbead kit (Miltenyi Biotec) from 108 PBMCs obtained from an unrelated donor. Prior culture, CD4 T cells were labeled with 5 μM of Carboxy-Fluorescein-Succinimidyl Esther (CFSE). 10⁵ CD4 T cells were then plated in a 96 well plate together with mature allo-DCs (5:1) in presence or absence of blocking anti-PD1 antibody (either PD1-0103, chimeric PD1-0103, or humanized antibodies PD1-0103-0312, PD1-0103-0313, PD1-0103-0314, PD1-0103-0315, abbreviated as 0312, 0313, 0314, 0315 in FIG. 4A and FIG. 4B), at the concentration of 10 μg/ml if not differently indicated in the figures.

Five days later we collected the cell-culture supernatants, used later to measure the IFN-γ levels by ELISA (R&D systems), and left the cells at 37 C degrees for additional 5 hours in presence of Golgi Plug (Brefeldin A) and Golgi Stop (Monensin). The cells were then washed, stained on the surface with anti-human CD4 antibody and the Live/Dead fixable dye Aqua (Invitrogen) before being fixed/permeabilized with Fix/Perm Buffer (BD Bioscience). We performed intracellular staining for Granzyme B (BD Bioscience), IFN-γ and IL-2 (both from eBioscience).

We also tested different concentrations of the humanized variants PD1-0103 (humanized antibodies PD1-0103-0312, PD1-0103-0313, PD1-0103-0314, PD1-0103-0315, abbreviated as 0312, 0313, 0314, 0315 in the figures, see also Example 1.6 below) and found them to be equally good in enhancing granzyme B and interferon gamma. DP47 is a non binding human IgG with a LALA mutation in the Fc portion to avoid recognition by FcγR and was used as negative control. Results are shown in FIG. 4A and FIG. 4B.

1.5 Chimeric Antibodies Derivatives

Chimeric PD1 antibodies were generated by amplifying the variable heavy and light chain regions of the anti-PD1 mouse antibodies PD1-0098, PD1-0103 via PCR and cloning them into heavy chain expression vectors as fusion proteins with human IgG1 backbones/human CH1-Hinge-CH2-CH3 with mutations L234A, L235A and P329G (EU index of Kabat)) (Leucine 234 to Alanine, Leucine 235 to Alanine, Proline 329 to Glycine) abrogating effector functions and light chain expression vectors as fusion proteins to human C-kappa. LC and HC Plasmids were then cotransfected into HEK293 and purified after 7 days from supertnatants by standard methods for antibody purification. The chimeric PD1-antibodies were renamed chimeric chiPD1-0098 (chiPD1-0098) and chimeric PD1-0103 (chiPD1-0103). For comparison the reference anti-PD1 antibodies comprising the VH and VL domains of either nivolumab (also known as MDX-5C4 or MDX-1106) and pembrolizumab (also known as MK-3475 or Org 1.09A) were synthesized and cloned with backbones of human IgG1 (with mutations L234A, L235A and P329G (EU index of Kabat)) were used.

1.6 Generation, Expression and Purification of Humanized Variants of Anti-PD1 Antibody PD-0103 (huMab PD-0103) and Characterization

### Humanization of the VH and VL Domains of Murine Anti-PD1 Antibody 0103

Based upon the amino acid sequence of the murine VH and VL domains of murine anti-PD1 antibody PD1-0103 (SEQ ID NO: 43 and 44), humanized anti-anti-PD1 antibody variants were generated.

The humanized VH-variant is based on the human germline IMGT_hVH_3_23 in combination with the human J-element germline IGHJ5-01 with several mutations. (resulting in SEQ ID NO: 45).

The humanized variants of VL are based on the human germlines IMGT_hVK_4_1, IMGT_hVK_2_30, IMGT_hVK_3_11 and IMGT_hVK_1_39 in combination with the human J-element germline IGKJ1-01. Different mutations resulted in humanized variants of SEQ ID NO: 46 to SEQ ID NO: 49.

The humanized amino acid sequences for heavy and light chain variable regions of PD1-0103 were backtranslated in to DNA and the resulting cNDA were synthesized (GenArt) and then cloned into heavy chain expression vectors as fusion proteins with human IgG1 backbones/human CH1-Hinge-CH2-CH3 with LALA and PG mutations (Leucine 234 to Alanine, Leucine 235 to Alanine, Proline 329 to Glycine) abrogating effector functions or into light chain expression vectors as fusion proteins to human C-kappa. LC and HC Plasmids were then cotransfected into HEK293 and purified after 7 days from supertnatants by standard methods for antibody purification. The resulting humanized PD1-antibodies named as follows:

TABLE 5
VH and VL sequences of humanized variant antibodies of PD1-0103

| Humanized antibodies of | humanized variant of | humanized variant of |
|---|---|---|
| PD1-0103 | VH/SEQ ID NO: | VL/SEQ ID NO: |
| PD1-0103-0312 | SEQ ID NO: 21 | SEQ ID NO: 22 |
| PD1-0103-0313 | SEQ ID NO: 21 | SEQ ID NO: 23 |
| PD1-0103-0314 | SEQ ID NO: 21 | SEQ ID NO: 24 |
| PD1-0103-0315 | SEQ ID NO: 21 | SEQ ID NO: 25 |

TABLE 6
HVR sequences of humanized variant antibodies of PD1-0103

| | | HVR-L1, |
|---|---|---|
| | | HVR-L2, and HVR- |
| Humanized | HVR-H1, HVR-H2, and | L3 of humanized |
| antibodies of | HVR-H3 of humanized | variant/SEQ |
| PD1-0103 | variant/SEQ ID NO: | ID NO: |
| PD-0103-0312 | SEQ ID NOs: 13, 14 and 15 | SEQ ID NOs: 16, 17 and 18 |
| PD-0103-0313 | SEQ ID NOs: 13, 14 and 15 | SEQ ID NOs: 16, 17 and 18 |
| PD-0103-0314 | SEQ ID NOs: 13, 14 and 15 | SEQ ID NOs: 16, 17 and 18 |
| PD-0103-0315 | SEQ ID NOs: 13, 14 and 15 | SEQ ID NOs: 16, 17 and 18 |

Humanized PD1-0103 antibody variants and parental chimeric PD1-0103 were characterized as described above. Results are shown in Table 7.

TABLE 7
Summary of results for humanized PD1-0103 antibody variants
and parental chimeric PD1-0103

| | chimeric | PD-0103- | PD-0103- | PD-0103- | PD-0103- |
|---|---|---|---|---|---|
| Assay | PD1-0103 | 0312 | 0313 | 0314 | 0315 |
| Affinity K_{D} | 2.0/0.8 | 1.5/1.8 | 1.9/2.3 | 1.6/1.5 | 1.7/1.5 |
| [nM] at 37° C. | | | | | |
| ELISA EC₅₀ | 0.2 | 0.1 | 0.07 | 0.07 | 0.06 |
| [nM] | | | | | |
| CHO-PD1 EC₅₀ | + | + | + | + | + |
| IC₅₀ PD-L1, 2 | 1.35 | tbd | tbd | tbd | tbd |
| [nM] | | | | | |
| Mixed | +++ | +++ | +++ | ++++ | ++ |
| Lymphocyte | | | | | |
| Reaction assay | | | | | |
| cynomolgus | + | 0.08 | 0.06 | 0.05 | 0.04 |
| crossreactivity | | | | | |
| (EC₅₀ [nm] | | | | | |

1.7 Neutralizing Potency of PD1 Antibodies

To test the neutralizing potency of inhouse generated PD1 antibodies in mimicking a restoration of a suppressed T cell response in vitro a commercially available PD1/PD-L1 reporter assay (Promega) was used. This system consists of PD1+ NFAT Jurkat cells and a PD-L1+ CHO counterpart, which also gives the activation signal. In principle, the reporter system is based on three steps: (1) TCR-mediated NFAT activation, (2) inhibition of NFAT signal upon activation by the PD-1/PD-L1 axis and (3) recovery of the NFAT signal by PD1 blocking antibodies.

### Material and Methods:

PD-L1 Medium: PAN Biotech (#P04-03609); FBS (10%) and L-Gln (4 mM)

Assay Medium: RPMI 1640 (#31870; Invitrogen), 25 mM HEPES, 2 mM L-Gln, FBS (2%)

Cells used for this assay (both cell types purchased by Promega):
PD-L1+ CHO cells (batch no. #139147): 2-3×104 cells/96well
PD1+ NFAT Jurkat cells (batch no. #133024: 3.5×104 cells/well

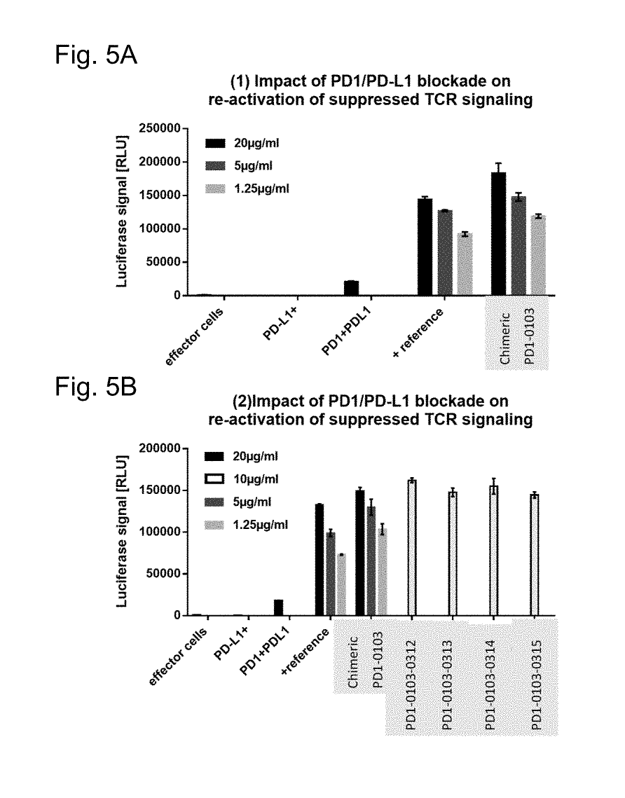

On day 1, PD-L1+ cells were thawed, seeded at the indicated cell concentration in the above mentioned medium and cultured over night at 37° C. and 5% CO₂. On the next day, medium was removed and PD-L1+ cells were incubated with the prepared antibodies at indicated concentrations (in Assay Medium). In parallel, PD1+ NFAT Jurkat cells were thawed and above mentioned cell numbers were transferred to and co-cultured with the PD-L1+ cells. After an incubation of 6 hrs at 37° C. and 5% CO₂, Bio-Glo substrate was warmed to room temperature (1-2 hrs prior addition). The cell culture plate was removed from the incubator and adjusted to room temperature (10 min) before 80 μl Bio-Glo solution was added per well, incubated for 5-10 min before the luminescence was measured at a Tecan Infinite reader according to the kit's manufacturer's recommendation. Results can be seen in the FIG. 5A and FIG. 5B where the restoration of a PD1/PD-L1 mediated suppression of the NFAT signal by different PD1 antibodies upon TCR stimulation is shown: FIG. 5 A: Chimeric PD1_0103 showed a reproducibly superior effect when compared to a reference antibody. As reference an anti-PD1 antibody comprising the VH and VL domains nivolumab (also known as MDX-5C4 or MDX-1106) was synthesized and cloned with backbones of human IgG1 (with mutations L234A, L235A and P329G (EU index of Kabat)). FIG. 5B: The four humanized variants of PD1_0103 demonstrated a similar in vitro potency to the lead antibody and were also slightly superior to the reference antibody.

### Example 2

### Preparation of PD1-Targeted 4-1BB Ligand Trimer-Containing Fc Fusion Antigen Binding Molecules

Different fragments of the DNA sequence encoding part of the ectodomain (amino acids 71-254 and 71-248) of human 4-1BB ligand were synthesized according to the P41273 sequence of Uniprot database (SEQ ID NO: 103).

2.1 Preparation of Monovalent PD1 (0314) Targeted 4-1BB Ligand (71-254) Trimer-Containing Fc (Kih) Fusion Antigen Binding Molecule with Crossed CH1-CL Domains with Charged Residues (Construct 7.1)

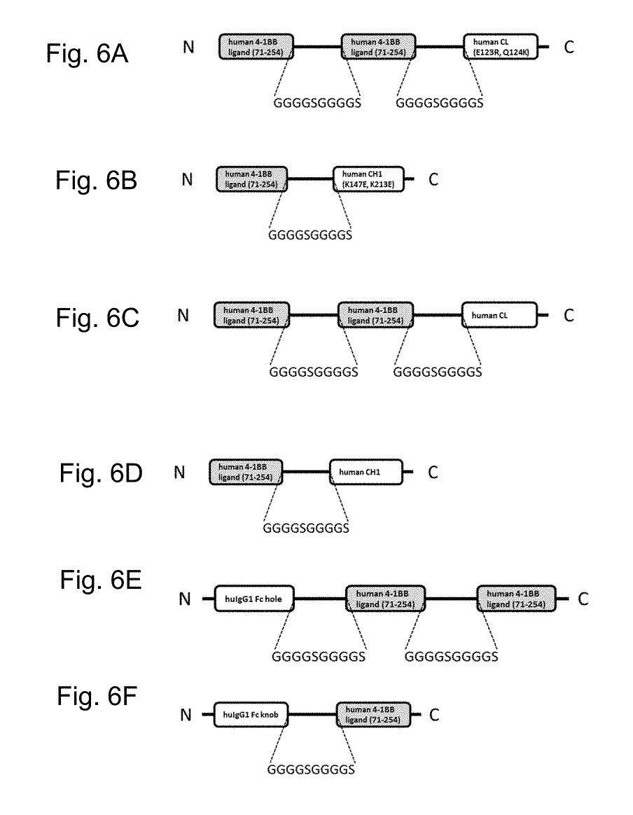

A polypeptide containing two ectodomains of 4-1BB ligand (71-254), separated by (G4S)2 linkers, and fused to the human IgG1-CL domain, was cloned as depicted in FIG. 6A: human 4-1BB ligand, (G4S)2 connector, human 4-1BB ligand, (G4S)2 connector, human CL.

A polypeptide containing one ectodomain of 4-1BB ligand (71-254) and fused to the human IgG1-CH1 domain, was cloned as described in FIG. 6B: human 4-1BB ligand, (G4S)2 connector, human CH.

To improve correct pairing the following mutations have been introduced in the crossed CH-CL. In the dimeric 4-1BB ligand fused to human CL the mutations E123R and Q124K were introduced. In the monomeric 4-1BB ligand fused to human CH1, the mutations K147E and K213E were cloned into the human CH1 domain as described in International Patent Appl. Publ. No. WO 2015/150447.

The variable region of heavy and light chain DNA sequences encoding a binder specific for PD1, clone 0314 (or 0103-0314), were subcloned in frame with either the constant heavy chain of the hole or the constant light chain of human IgG1. The generation and preparation of the PD1 binders is described in Example 1.

The Pro329Gly, Leu234Ala and Leu235Ala mutations have been introduced in the constant region of the knob and hole heavy chains to abrogate binding to Fc gamma receptors according to the method described in International Patent Appl. Publ. No. WO 2012/130831.

For all constructs the knobs into hole heterodimerization technology was used with the the S354C/T366W mutations in the knob chain and the corresponding Y349C/T366S/L368A/Y407V mutations in the hole chain.

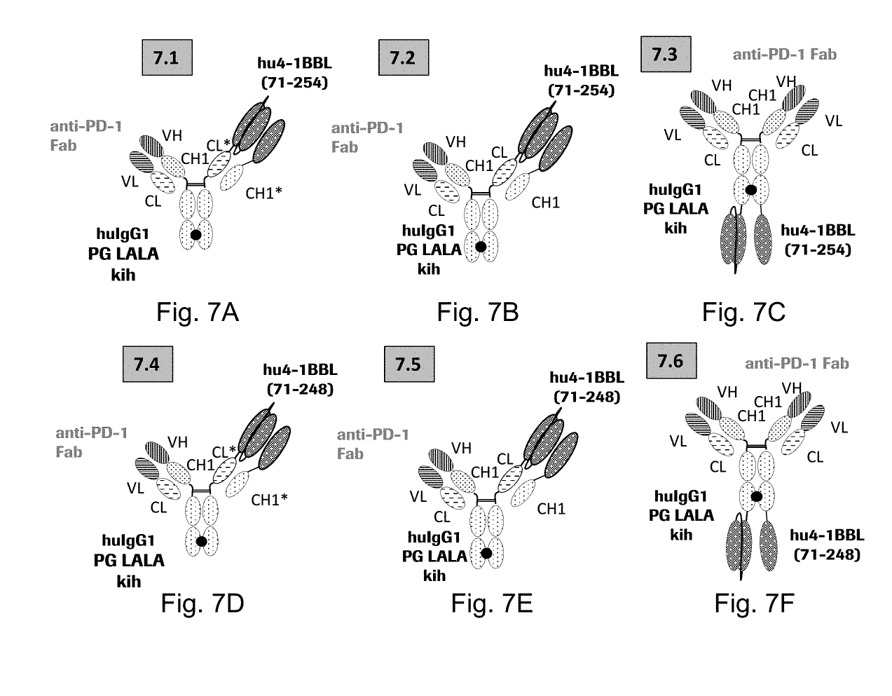

Combination of the dimeric ligand-Fc knob chain containing the S354C/T366W mutations, the monomeric CH1 fusion, the targeted anti-PD1-Fc hole chain containing the Y349C/T366S/L368A/Y407V mutations and the anti-PD1 light chain allows generation of a heterodimer, which includes an assembled trimeric 4-1BB ligand and a PD1 binding Fab (FIG. 7A, Construct 7.1).

Table 8 shows the cDNA and amino acid sequences of the monovalent PD1(0314)-human 4-1BB ligand (71-254) Fc (kih) fusion antigen binding molecule containing CH1-CL crossover and charged residues (Construct 7.1).

TABLE 8
Sequences of monovalent PD1(0314)-targeted human 4-1BB ligand
(71-254) containing Fc (kih) fusion molecule Construct 7.1

| SEQ ID NO: | Description | Sequence |
|---|---|---|
| 131 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | dimeric hu 4- | GGCCCAGAACGTGCTGCTGATCGATGGCCCCCTGTCCTG |
| | 1BBL (71-254) - | GTACAGCGATCCTGGACTGGCTGGCGTGTCACTGACAGG |
| | CL* Fc knob | CGGCCTGAGCTACAAAGAGGACACCAAAGAACTGGTGG |
| | chain | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGAAGCGCT |
| | | GCTGGCGCTGCAGCTCTGGCACTGACAGTGGATCTGCCT |
| | | CCTGCCAGCTCCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCCGGCCTGCCTTCT |
| | | CCAAGAAGCGAAGGCGGAGGCGGATCTGGCGGCGGAG |
| | | GATCTAGAGAGGGACCCGAACTGTCCCCTGACGATCCA |
| | | GCCGGGCTGCTGGATCTGAGACAGGGAATGTTCGCCCA |
| | | GCTGGTGGCTCAGAATGTGCTGCTGATTGACGGACCTCT |
| | | GAGCTGGTACTCCGACCCAGGGCTGGCAGGGGTGTCCC |
| | | TGACTGGGGGACTGTCCTACAAAGAAGATACAAAAGAA |
| | | CTGGTGGTGGCTAAAGCTGGGGTGTACTATGTGTTTTTT |
| | | CAGCTGGAACTGAGGCGGGTGGTGGCTGGGGAGGGCTC |
| | | AGGATCTGTGTCCCTGGCTCTGCATCTGCAGCCACTGCG |
| | | CTCTGCTGCTGGCGCAGCTGCACTGGCTCTGACTGTGGA |
| | | CCTGCCACCAGCCTCTAGCGAGGCCAGAAACAGCGCCT |
| | | TCGGGTTCCAAGGACGCCTGCTGCATCTGAGCGCCGGAC |
| | | AGCGCCTGGGAGTGCATCTGCATACTGAAGCCAGAGCC |
| | | CGGCATGCTTGGCAGCTGACTCAGGGGGCAACTGTGCTG |
| | | GGACTGTTTCGCGTGACACCTGAGATCCCTGCCGGACTG |
| | | CCAAGCCCTAGATCAGAAGGGGGCGGAGGTTCCGGAGG |
| | | GGGAGGATCTCGTACGGTGGCTGCACCATCTGTCTTTAT |
| | | CTTCCCACCCAGCGACCGGAAGCTGAAGTCTGGCACAG |
| | | CCAGCGTCGTGTGCCTGCTGAATAACTTCTACCCCCGCG |
| | | AGGCCAAGGTGCAGTGGAAGGTGGACAATGCCCTGCAG |
| | | AGCGGCAACAGCCAGGAAAGCGTGACCGAGCAGGACA |
| | | GCAAGGACTCCACCTACAGCCTGAGCAGCACCCTGACC |
| | | CTGAGCAAGGCCGACTACGAGAAGCACAAGGTGTACGC |
| | | CTGCGAAGTGACCCACCAGGGCCTGTCTAGCCCCGTGAC |
| | | CAAGAGCTTCAACCGGGGCGAGTGCGACAAGACCCACA |
| | | CCTGTCCTCCATGCCCTGCCCCTGAAGCTGCTGGCGGCC |
| | | CTAGCGTGTTCCTGTTCCCCCCAAAGCCCAAGGACACCC |
| | | TGATGATCAGCCGGACCCCTGAAGTGACCTGCGTGGTGG |
| | | TGGATGTGTCCCACGAGGACCCTGAAGTGAAGTTCAATT |
| | | GGTACGTGGACGGCGTGGAAGTGCACAATGCCAAGACC |
| | | AAGCCGCGGGAGGAGCAGTACAACAGCACGTACCGTGT |
| | | GGTCAGCGTCCTCACCGTCCTGCACCAGGACTGGCTGAA |
| | | TGGCAAGGAGTACAAGTGCAAGGTCTCCAACAAAGCCC |
| | | TCGGCGCCCCCATCGAGAAAACCATCTCCAAAGCCAAA |
| | | GGGCAGCCCCGAGAACCACAGGTGTACACCCTGCCCCC |
| | | ATGCCGGGATGAGCTGACCAAGAACCAGGTCAGCCTGT |
| | | GGTGCCTGGTCAAAGGCTTCTATCCCAGCGACATCGCCG |
| | | TGGAGTGGGAGAGCAATGGGCAGCCGGAGAACAACTAC |
| | | AAGACCACGCCTCCCGTGCTGGACTCCGACGGCTCCTTC |
| | | TTCCTCTACAGCAAGCTCACCGTGGACAAGAGCAGGTG |
| | | GCAGCAGGGGAACGTCTTCTCATGCTCCGTGATGCATGA |
| | | GGCTCTGCACAACCACTACACGCAGAAGAGCCTCTCCCT |
| | | GTCTCCGGGTAAA |
| 132 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | monomeric hu 4- | GGCCCAGAACGTGCTGCTGATCGATGGCCCCCTGTCCTG |
| | 1BBL (71-254) - | GTACAGCGATCCTGGACTGGCTGGCGTGTCACTGACAGG |
| | CH1* | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGAAGCGCT |
| | | GCTGGCGCTGCAGCTCTGGCTCTGACAGTGGATCTGCCT |
| | | CCTGCCAGCTCCGAGGCCCGGAATAGCGCATTTGGGTTT |
| | | CAAGGCCGGCTGCTGCACCTGTCTGCCGGCCAGAGACT |
| | | GGGAGTGCATCTGCACACAGAGGCCAGAGCCAGGCACG |
| | | CCTGGCAGCTGACACAGGGCGCTACAGTGCTGGGCCTG |
| | | TTCAGAGTGACCCCCGAGATTCCTGCCGGCCTGCCTAGC |
| | | CCTAGATCTGAAGGCGGCGGAGGTTCCGGAGGCGGAGG |
| | | ATCTGCTAGCACAAAGGGCCCCAGCGTGTTCCCTCTGGC |
| | | CCCTAGCAGCAAGAGCACATCTGGCGGAACAGCCGCCC |
| | | TGGGCTGCCTGGTGGAAGATTACTTCCCCGAGCCCGTGA |
| | | CCGTGTCCTGGAATTCTGGCGCCCTGACAAGCGGCGTGC |
| | | ACACCTTTCCAGCCGTGCTGCAGAGCAGCGGCCTGTACT |
| | | CTCTGAGCAGCGTCGTGACAGTGCCCAGCAGCTCTCTGG |
| | | GCACCCAGACCTACATCTGCAACGTGAACCACAAGCCC |
| | | AGCAACACCAAGGTGGACGAGAAGGTGGAACCCAAGTC |
| | | CTGC |
| 133 | nucleotide | GAAGTGCAGCTGCTCGAAAGCGGCGGGGGACTGGTCCA |
| | sequence of anti- | GCCCGGCGGTTCCCTGAGGCTGTCTTGCGCCGCTTCAGG |
| | PD1(0314) Fc | GTTCAGCTTTTCCTCTTACACCATGAGTTGGGTCAGACA |
| | hole chain | GGCACCTGGCAAGGGACTGGAGTGGGTCGCCACAATCA |
| | | GCGGTGGCGGGCGCGACATTTATTACCCAGATTCCGTGA |
| | | AAGGACGGTTCACCATCTCTAGGGACAACTCAAAGAAT |
| | | ACTCTGTATTTGCAGATGAACAGCCTGAGAGCTGAGGAT |
| | | ACAGCAGTTTACTACTGTGTGCTCCTGACCGGCCGCGTC |
| | | TATTTTGCCCTTGACTCCTGGGGACAAGGCACTCTGGTG |
| | | ACCGTATCTAGTGCTAGCACCAAGGGCCCCTCCGTGTTC |
| | | CCCCTGGCCCCCAGCAGCAAGAGCACCAGCGGCGGCAC |
| | | AGCCGCTCTGGGCTGCCTGGTCAAGGACTACTTCCCCGA |
| | | GCCCGTGACCGTGTCCTGGAACAGCGGAGCCCTGACCTC |
| | | CGGCGTGCACACCTTCCCCGCCGTGCTGCAGAGTTCTGG |
| | | CCTGTATAGCCTGAGCAGCGTGGTCACCGTGCCTTCTAG |
| | | CAGCCTGGGCACCCAGACCTACATCTGCAACGTGAACC |
| | | ACAAGCCCAGCAACACCAAGGTGGACAAGAAGGTGGA |
| | | GCCCAAGAGCTGCGACAAAACTCACACATGCCCACCGT |
| | | GCCCAGCACCTGAAGCTGCAGGGGGACCGTCAGTCTTC |
| | | CTCTTCCCCCCAAAACCCAAGGACACCCTCATGATCTCC |
| | | CGGACCCCTGAGGTCACATGCGTGGTGGTGGACGTGAG |
| | | CCACGAAGACCCTGAGGTCAAGTTCAACTGGTACGTGG |
| | | ACGGCGTGGAGGTGCATAATGCCAAGACAAAGCCGCGG |
| | | GAGGAGCAGTACAACAGCACGTACCGTGTGGTCAGCGT |
| | | CCTCACCGTCCTGCACCAGGACTGGCTGAATGGCAAGG |
| | | AGTACAAGTGCAAGGTCTCCAACAAAGCCCTCGGCGCC |
| | | CCCATCGAGAAAACCATCTCCAAAGCCAAAGGGCAGCC |
| | | CCGAGAACCACAGGTGTGCACCCTGCCCCCATCCCGGG |
| | | ATGAGCTGACCAAGAACCAGGTCAGCCTCTCGTGCGCA |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCGT |
| | | GAGCAAGCTCACCGTGGACAAGAGCAGGTGGCAGCAGG |
| | | GGAACGTCTTCTCATGCTCCGTGATGCATGAGGCTCTGC |
| | | ACAACCACTACACGCAGAAGAGCCTCTCCCTGTCTCCGG |
| | | GTAAA |
| 134 | nucleotide | GAGATCGTGCTGACCCAGAGCCCCGCCACACTCTCCCTG |
| | sequence of anti- | TCTCCTGGCGAAAGGGCTACCCTTTCATGCAGAGCAAGC |
| | PD1 (0314) light | GAGTCCGTGGACACTTCTGATAACAGTTTCATTCACTGG |
| | chain | TATCAGCAGAAGCCAGGGCAGAGCCCCCGCCTGCTCAT |
| | | CTATCGGTCCTCTACACTGGAGTCAGGGATTCCTGCCAG |
| | | GTTTAGCGGCTCCGGTTCTGGGACCGACTTCACTTTGAC |
| | | CATCAGTAGCCTGGAACCAGAGGATTTTGCTGTGTACTA |
| | | TTGTCAACAGAATTACGACGTCCCCTGGACATTCGGCCA |
| | | GGGAACCAAAGTGGAGATAAAGCGTACGGTGGCTGCAC |
| | | CATCTGTCTTCATCTTCCCGCCATCTGATGAGCAGTTGA |
| | | AATCTGGAACTGCCTCTGTTGTGTGCCTGCTGAATAACT |
| | | TCTATCCCAGAGAGGCCAAAGTACAGTGGAAGGTGGAT |
| | | AACGCCCTCCAATCGGGTAACTCCCAGGAGAGTGTCAC |
| | | AGAGCAGGACAGCAAGGACAGCACCTACAGCCTCAGCA |
| | | GCACCCTGACGCTGAGCAAAGCAGACTACGAGAAACAC |
| | | AAAGTCTACGCCTGCGAAGTCACCCATCAGGGCCTGAG |
| | | CTCGCCCGTCACAAAGAGCTTCAACAGGGGAGAGTGT |
| 74 | Dimeric hu 4- | REGPELSPDDPAGLLDLRQGMFAQLVAQNVLLIDGPLSWY |
| | 1BBL (71-254) - | SDPGLAGVSLTGGLSYKEDTKELVVAKAGVYYVFFQLELR |
| | CL* Fc knob | RVVAGEGSGSVSLALHLQPLRSAAGAAALALTVDLPPASS |
| | chain | EARNSAFGFQGRLLHLSAGQRLGVHLHTEARARHAWQLT |
| | | QGATVLGLFRVTPEIPAGLPSPRSEGGGGSGGGGSREGPEL |
| | | SPDDPAGLLDLRQGMFAQLVAQNVLLIDGPLSWYSDPGLA |
| | | GVSLTGGLSYKEDTKELVVAKAGVYYVFFQLELRRVVAG |
| | | EGSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGLPSPRSEGGGGSGGGGSRTVAAPSVFIFP |
| | | PSDRKLKSGTASVVCLLNNFYPREAKVQWKVDNALQSGN |
| | | SQESVTEQDSKDSTYSLSSTLTLSKADYEKHKVYACEVTH |
| | | QGLSSPVTKSFNRGECDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVYTLPPCRDELTKNQVS |
| | | LWCLVKGFYPSDIAVEWESNGQPENNYKTTPPVLDSDGSF |
| | | FLYSKLTVDKSRWQQGNVFSCSVMHEALHNHYTQKSLSL |
| | | SPGK |
| 75 | Monomeric hu 4- | REGPELSPDDPAGLLDLRQGMFAQLVAQNVLLIDGPLSWY |
| | 1BBL (71-254) - | SDPGLAGVSLTGGLSYKEDTKELVVAKAGVYYVFFQLELR |
| | CH1* | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLPSPRSEGGGGSGGGGSASTKGP |
| | | SVFPLAPSSKSTSGGTAALGCLVEDYFPEPVTVSWNSGALT |
| | | SGVHTFPAVLQSSGLYSLSSVVTVPSSSLGTQTYICNVNHK |
| | | PSNTKVDEKVEPKSC |
| 82 | anti-PD1 (0314) | EVQLLESGGGLVQPGGSLRLSCAASGFSFSSYTMSWVRQA |
| | Fc hole chain | PGKGLEWVATISGGGRDIYYPDSVKGRFTISRDNSKNTLYL |
| | | QMNSLRAEDTAVYYCVLLTGRVYFALDSWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVCTLPPSRDELTK |
| | | NQVSLSCAVKGFYPSDIAVEWESNGQPENNYKTTPPVLDS |
| | | DGSFFLVSKLTVDKSRWQQGNVFSCSVMHEALHNHYTQK |
| | | SLSLSPGK |
| 83 | anti-PD1 (0314) | EIVLTQSPATLSLSPGERATLSCRASESVDTSDNSFIHWYQQ |
| | light chain | KPGQSPRLLIYRSSTLESGIPARFSGSGSGTDFTLTISSLEPED |
| | | FAVYYCQQNYDVPWTFGQGTKVEIKRTVAAPSVFIFPPSD |
| | | EQLKSGTASVVCLLNNFYPREAKVQWKVDNALQSGNSQE |
| | | SVTEQDSKDSTYSLSSTLTLSKADYEKHKVYACEVTHQGL |
| | | SSPVTKSFNRGEC |

2. 2 Preparation of Monovalent PD1 (0314) Targeted 4-1BB Ligand (71-254) Trimer-Containing Fc (Kih) Fusion Antigen Binding Molecule with Crossed CH1-CL Domains without Charged Residues (Construct 7.2)

A polypeptide containing two ectodomains of 4-1BB ligand (71-254), separated by (G4S)2 linkers, and fused to the human IgG1-CL domain, was cloned as depicted in FIG. 6C: human 4-1BB ligand, (G4S)2 connector, human 4-1BB ligand, (G4S)2 connector, human CL.

A polypeptide containing one ectodomain of 4-1BB ligand (71-254) and fused to the human IgG1-CH1 domain, was cloned as described in FIG. 6D: human 4-1BB ligand, (G4S)2 connector, human CH1.

The variable region of heavy and light chain DNA sequences encoding a binder specific for PD1, clone 0103-0314, were subcloned in frame with either the constant heavy chain of the hole or the constant light chain of human IgG1.

The Pro329Gly, Leu234Ala and Leu235Ala mutations have been introduced in the constant region of the knob and hole heavy chains to abrogate binding to Fc gamma receptors (WO 2012/130831).

Combination of the dimeric ligand-Fc knob chain containing the S354C/T366W mutations, the monomeric CH1 fusion, the targeted anti-FAP-Fc hole chain containing the Y349C/T366S/L368A/Y407V mutations and the anti-FAP light chain allows generation of a heterodimer, which includes an assembled trimeric 4-1BB ligand and a FAP binding Fab (FIG. 7B, Construct 7.2).

Table 9 shows the cDNA and amino acid sequences of the monovalent anti-PD1(0314) human 4-1BB ligand (71-254) Fc (kih) fusion antigen binding molecule containing CH1-CL crossover without charged residues (Construct 7.2).

TABLE 9
Sequences of monovalent PD1(0314)-targeted human 4-1BB ligand (71-254)
containing Fc (kih) fusion molecule Construct 7.2

| SEQ ID NO: | Description | Sequence |
|---|---|---|
| 135 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | dimeric hu 4- | GGCCCAGAACGTGCTGCTGATCGATGGCCCCCTGTCCTG |
| | 1BBL (71-254) - | GTACAGCGATCCTGGACTGGCTGGCGTGTCACTGACAGG |
| | CL Fc knob chain | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGAAGCGCT |
| | | GCTGGCGCTGCAGCTCTGGCACTGACAGTGGATCTGCCT |
| | | CCTGCCAGCTCCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCCGGCCTGCCTTCT |
| | | CCAAGAAGCGAAGGCGGAGGCGGATCTGGCGGCGGAG |
| | | GATCTAGAGAGGGACCCGAACTGTCCCCTGACGATCCA |
| | | GCCGGGCTGCTGGATCTGAGACAGGGAATGTTCGCCCA |
| | | GCTGGTGGCTCAGAATGTGCTGCTGATTGACGGACCTCT |
| | | GAGCTGGTACTCCGACCCAGGGCTGGCAGGGGTGTCCC |
| | | TGACTGGGGGACTGTCCTACAAAGAAGATACAAAAGAA |
| | | CTGGTGGTGGCTAAAGCTGGGGTGTACTATGTGTTTTTT |
| | | CAGCTGGAACTGAGGCGGGTGGTGGCTGGGGAGGGCTC |
| | | AGGATCTGTGTCCCTGGCTCTGCATCTGCAGCCACTGCG |
| | | CTCTGCTGCTGGCGCAGCTGCACTGGCTCTGACTGTGGA |
| | | CCTGCCACCAGCCTCTAGCGAGGCCAGAAACAGCGCCT |
| | | TCGGGTTCCAAGGACGCCTGCTGCATCTGAGCGCCGGAC |
| | | AGCGCCTGGGAGTGCATCTGCATACTGAAGCCAGAGCC |
| | | CGGCATGCTTGGCAGCTGACTCAGGGGGCAACTGTGCTG |
| | | GGACTGTTTCGCGTGACACCTGAGATCCCTGCCGGACTG |
| | | CCAAGCCCTAGATCAGAAGGGGGCGGAGGTTCCGGAGG |
| | | GGGAGGATCTCGTACGGTGGCCGCTCCCTCCGTGTTTAT |
| | | CTTTCCCCCATCCGATGAACAGCTGAAAAGCGGCACCGC |
| | | CTCCGTCGTGTGTCTGCTGAACAATTTTTACCCTAGGGA |
| | | AGCTAAAGTGCAGTGGAAAGTGGATAACGCACTGCAGT |
| | | CCGGCAACTCCCAGGAATCTGTGACAGAACAGGACTCC |
| | | AAGGACAGCACCTACTCCCTGTCCTCCACCCTGACACTG |
| | | TCTAAGGCTGATTATGAGAAACACAAAGTCTACGCCTGC |
| | | GAAGTCACCCATCAGGGCCTGAGCTCGCCCGTCACAAA |
| | | GAGCTTCAACAGGGGAGAGTGTGACAAGACCCACACCT |
| | | GTCCCCCTTGTCCTGCCCCTGAAGCTGCTGGCGGCCCTT |
| | | CTGTGTTCCTGTTCCCCCCAAAGCCCAAGGACACCCTGA |
| | | TGATCAGCCGGACCCCCGAAGTGACCTGCGTGGTGGTG |
| | | GATGTGTCCCACGAGGACCCTGAAGTGAAGTTCAATTGG |
| | | TACGTGGACGGCGTGGAAGTGCACAATGCCAAGACCAA |
| | | GCCGCGGGAGGAGCAGTACAACAGCACGTACCGTGTGG |
| | | TCAGCGTCCTCACCGTCCTGCACCAGGACTGGCTGAATG |
| | | GCAAGGAGTACAAGTGCAAGGTCTCCAACAAAGCCCTC |
| | | GGCGCCCCCATCGAGAAAACCATCTCCAAAGCCAAAGG |
| | | GCAGCCCCGAGAACCACAGGTGTACACCCTGCCCCCAT |
| | | GCCGGGATGAGCTGACCAAGAACCAGGTCAGCCTGTGG |
| | | TGCCTGGTCAAAGGCTTCTATCCCAGCGACATCGCCGTG |
| | | GAGTGGGAGAGCAATGGGCAGCCGGAGAACAACTACAA |
| | | GACCACGCCTCCCGTGCTGGACTCCGACGGCTCCTTCTT |
| | | CCTCTACAGCAAGCTCACCGTGGACAAGAGCAGGTGGC |
| | | AGCAGGGGAACGTCTTCTCATGCTCCGTGATGCATGAGG |
| | | CTCTGCACAACCACTACACGCAGAAGAGCCTCTCCCTGT |
| | | CTCCGGGTAAA |
| 136 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | monomeric hu 4- | GGCCCAGAACGTGCTGCTGATCGATGGCCCCCTGTCCTG |
| | 1BBL (71-254) - | GTACAGCGATCCTGGACTGGCTGGCGTGTCACTGACAGG |
| | CH1 | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGAAGCGCT |
| | | GCTGGCGCTGCAGCTCTGGCTCTGACAGTGGATCTGCCT |
| | | CCTGCCAGCTCCGAGGCCCGGAATAGCGCATTTGGGTTT |
| | | CAAGGCCGGCTGCTGCACCTGTCTGCCGGCCAGAGACT |
| | | GGGAGTGCATCTGCACACAGAGGCCAGAGCCAGGCACG |
| | | CCTGGCAGCTGACACAGGGCGCTACAGTGCTGGGCCTG |
| | | TTCAGAGTGACCCCCGAGATTCCTGCCGGCCTGCCTAGC |
| | | CCTAGATCTGAAGGCGGCGGAGGTTCCGGAGGCGGAGG |
| | | ATCTGCTAGCACCAAAGGCCCTTCCGTGTTTCCTCTGGC |
| | | TCCTAGCTCCAAGTCCACCTCTGGAGGCACCGCTGCTCT |
| | | CGGATGCCTCGTGAAGGATTATTTTCCTGAGCCTGTGAC |
| | | AGTGTCCTGGAATAGCGGAGCACTGACCTCTGGAGTGC |
| | | ATACTTTCCCCGCTGTGCTGCAGTCCTCTGGACTGTACA |
| | | GCCTGAGCAGCGTGGTGACAGTGCCCAGCAGCAGCCTG |
| | | GGCACCCAGACCTACATCTGCAACGTGAACCACAAGCC |
| | | CAGCAACACCAAGGTGGACAAGAAGGTGGAACCCAAGT |
| | | CTTGT |
| 133 | nucleotide | see Table 8 |
| | sequence of anti- | |
| | PD1(0314) Fc | |
| | hole chain | |
| 134 | nucleotide | see Table 8 |
| | sequence of anti- | |
| | PD1(0314) light | |
| | chain | |
| 76 | Dimeric hu 4- | REGPELSPDDPAGLLDLRQGMFAQLVAQNVLLIDGPLSWY |
| | 1BBL (71-254) - | SDPGLAGVSLTGGLSYKEDTKELVVAKAGVYYVFFQLELR |
| | CL Fc knob chain | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLPSPRSEGGGGSGGGGSREGPEL |
| | | SPDDPAGLLDLRQGMFAQLVAQNVLLIDGPLSWYSDPGLA |
| | | GVSLTGGLSYKEDTKELVVAKAGVYYVFFQLELRRVVAG |
| | | EGSGSVSLALHLQPLRSAAGAAALALTVDLPPASSEARNS |
| | | AFGFQGRLLHLSAGQRLGVHLHTEARARHAWQLTQGATV |
| | | LGLFRVTPEIPAGLPSPRSEGGGGSGGGGSRTVAAPSVFIFP |
| | | PSDEQLKSGTASVVCLLNNFYPREAKVQWKVDNALQSGN |
| | | SQESVTEQDSKDSTYSLSSTLTLSKADYEKHKVYACEVTH |
| | | QGLSSPVTKSFNRGECDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVYTLPPCRDELTKNQVS |
| | | LWCLVKGFYPSDIAVEWESNGQPENNYKTTPPVLDSDGSF |
| | | FLYSKLTVDKSRWQQGNVFSCSVMHEALHNHYTQKSLSL |
| | | SPGK |
| 77 | Monomeric hu 4- | REGPELSPDDPAGLLDLRQGMFAQLVAQNVLLIDGPLSWY |
| | 1BBL (71-254) - | SDPGLAGVSLTGGLSYKEDTKELVVAKAGVYYVFFQLELR |
| | CH1 | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLPSPRSEGGGGSGGGGSASTKGP |
| | | SVFPLAPSSKSTSGGTAALGCLVKDYFPEPVTVSWNSGALT |
| | | SGVHTFPAVLQSSGLYSLSSVVTVPSSSLGTQTYICNVNHK |
| | | PSNTKVDKKVEPKSC |
| 82 | anti-PD1(0314) | see Table 8 |
| | Fc hole chain | |
| 83 | anti-PD1(0314) | see Table 8 |
| | light chain | |

2.3 Preparation of Bivalent PD1(0314) Targeted 4-1BB Ligand (71-254) Trimer-Containing Fc (Kih) Fusion Antigen Binding Molecule with the Dimeric and Monomeric 4-1BB Ligands Fused at the C-Terminus of Each Heavy Chain (Construct 7.3)

A polypeptide containing two ectodomains of 4-1BB ligand (71-254), separated by (G4S)2 linkers was fused to the C-terminus of human IgG1 Fc hole chain, as depicted in FIG. 6E: human IgG1 Fc hole, (G4S)2 connector, human 4-1BB ligand, (G4S)2 connector, human 4-1BB ligand.

A polypeptide containing one ectodomain of 4-1BB ligand (71-254) was fused to the C-terminus of human IgG1 Fc knob chain as described in FIG. 6F: human IgG1 Fc knob, (G4S)2 connector, human 4-1BB ligand.

The variable region of heavy and light chain DNA sequences encoding a binder specific for PD1, clone 0103-0314, were subcloned in frame with either the constant heavy chain of the hole, the knob or the constant light chain of human IgG1.

The Pro329Gly, Leu234Ala and Leu235Ala mutations have been introduced in the constant region of the knob and hole heavy chains to abrogate binding to Fc gamma receptors according to the method described in WO 2012/130831.

Combination of the anti-FAP huIgG1 hole dimeric ligand chain containing the Y349C/T366S/L368A/Y407V mutations, the anti-FAP huIgG1 knob monomeric ligand chain containing the S354C/T366W mutations and the anti-FAP light chain allows generation of a heterodimer, which includes an assembled trimeric 4-1BB ligand and two PD1 binding Fabs (FIG. 7C, Construct 7.3)

Table 10 shows the cDNA and amino acid sequences of the bivalent PD1(0314)-targeted 4-1BB ligand trimer-containing Fc (kih) fusion molecule Construct 7.3 (PD1 split trimer with 2 anti-PD1 Fabs, dimeric and monomeric 4-1BB ligand fused at the C-terminus of each heavy chain, respectively).

TABLE 10
Sequences of bivalent PD1(0314)-targeted human 4-1BB ligand (71-254)
containing Fc (kih) fusion molecule Construct 7.3

| SEQ ID NO: | Description | Sequence |
|---|---|---|
| 137 | nucleotide | GAAGTGCAGCTGCTCGAAAGCGGCGGGGGACTGGTCCA |
| | sequence of anti- | GCCCGGCGGTTCCCTGAGGCTGTCTTGCGCCGCTTCAGG |
| | PD1(0314) Fc | GTTCAGCTTTTCCTCTTACACCATGAGTTGGGTCAGACA |
| | hole chain fused | GGCACCTGGCAAGGGACTGGAGTGGGTCGCCACAATCA |
| | to dimeric hu 4- | GCGGTGGCGGGCGCGACATTTATTACCCAGATTCCGTGA |
| | 1BBL (71-254) | AAGGACGGTTCACCATCTCTAGGGACAACTCAAAGAAT |
| | | ACTCTGTATTTGCAGATGAACAGCCTGAGAGCTGAGGAT |
| | | ACAGCAGTTTACTACTGTGTGCTCCTGACCGGCCGCGTC |
| | | TATTTTGCCCTTGACTCCTGGGGACAAGGCACTCTGGTG |
| | | ACCGTATCTAGTGCTAGCACCAAGGGCCCCTCCGTGTTC |
| | | CCCCTGGCCCCCAGCAGCAAGAGCACCAGCGGCGGCAC |
| | | AGCCGCTCTGGGCTGCCTGGTCAAGGACTACTTCCCCGA |
| | | GCCCGTGACCGTGTCCTGGAACAGCGGAGCCCTGACCTC |
| | | CGGCGTGCACACCTTCCCCGCCGTGCTGCAGAGTTCTGG |
| | | CCTGTATAGCCTGAGCAGCGTGGTCACCGTGCCTTCTAG |
| | | CAGCCTGGGCACCCAGACCTACATCTGCAACGTGAACC |
| | | ACAAGCCCAGCAACACCAAGGTGGACAAGAAGGTGGA |
| | | GCCCAAGAGCTGCGACAAAACTCACACATGCCCACCGT |
| | | GCCCAGCACCTGAAGCTGCAGGGGGACCGTCAGTCTTC |
| | | CTCTTCCCCCCAAAACCCAAGGACACCCTCATGATCTCC |
| | | CGGACCCCTGAGGTCACATGCGTGGTGGTGGACGTGAG |
| | | CCACGAAGACCCTGAGGTCAAGTTCAACTGGTACGTGG |
| | | ACGGCGTGGAGGTGCATAATGCCAAGACAAAGCCGCGG |
| | | GAGGAGCAGTACAACAGCACGTACCGTGTGGTCAGCGT |
| | | CCTCACCGTCCTGCACCAGGACTGGCTGAATGGCAAGG |
| | | AGTACAAGTGCAAGGTCTCCAACAAAGCCCTCGGCGCC |
| | | CCCATCGAGAAAACCATCTCCAAAGCCAAAGGGCAGCC |
| | | CCGAGAACCACAGGTGTGCACCCTGCCCCCATCCCGGG |
| | | ATGAGCTGACCAAGAACCAGGTCAGCCTCTCGTGCGCA |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCGT |
| | | GAGCAAGCTCACCGTGGACAAGAGCAGGTGGCAGCAGG |
| | | GGAACGTCTTCTCATGCTCCGTGATGCATGAGGCTCTGC |
| | | ACAACCACTACACGCAGAAGAGCCTCTCCCTGTCTCCGG |
| | | GTGGAGGCGGCGGAAGCGGAGGAGGAGGATCCAGAGA |
| | | GGGCCCTGAGCTGAGCCCCGATGATCCTGCTGGACTGCT |
| | | GGACCTGCGGCAGGGCATGTTTGCTCAGCTGGTGGCCCA |
| | | GAACGTGCTGCTGATCGATGGCCCCCTGTCCTGGTACAG |
| | | CGATCCTGGACTGGCTGGCGTGTCACTGACAGGCGGCCT |
| | | GAGCTACAAAGAGGACACCAAAGAACTGGTGGTGGCCA |
| | | AGGCCGGCGTGTACTACGTGTTCTTTCAGCTGGAACTGC |
| | | GGAGAGTGGTGGCCGGCGAAGGATCTGGCTCTGTGTCTC |
| | | TGGCCCTGCATCTGCAGCCTCTGAGAAGCGCTGCTGGCG |
| | | CTGCAGCTCTGGCACTGACAGTGGATCTGCCTCCTGCCA |
| | | GCTCCGAGGCCCGGAATAGCGCATTTGGGTTTCAAGGCA |
| | | GGCTGCTGCACCTGTCTGCCGGCCAGAGGCTGGGAGTG |
| | | CATCTGCACACAGAGGCCAGGGCTAGACACGCCTGGCA |
| | | GCTGACACAGGGCGCTACAGTGCTGGGCCTGTTCAGAGT |
| | | GACCCCCGAGATTCCAGCCGGCCTGCCTTCTCCAAGAAG |
| | | CGAAGGCGGAGGCGGATCTGGCGGCGGAGGATCTAGAG |
| | | AGGGACCCGAACTGTCCCCTGACGATCCAGCCGGGCTG |
| | | CTGGATCTGAGACAGGGAATGTTCGCCCAGCTGGTGGCT |
| | | CAGAATGTGCTGCTGATTGACGGACCTCTGAGCTGGTAC |
| | | TCCGACCCAGGGCTGGCAGGGGTGTCCCTGACTGGGGG |
| | | ACTGTCCTACAAAGAAGATACAAAAGAACTGGTGGTGG |
| | | CTAAAGCTGGGGTGTACTATGTGTTTTTTCAGCTGGAAC |
| | | TGAGGCGGGTGGTGGCTGGGGAGGGCTCAGGATCTGTG |
| | | TCCCTGGCTCTGCATCTGCAGCCACTGCGCTCTGCTGCT |
| | | GGCGCAGCTGCACTGGCTCTGACTGTGGACCTGCCACCA |
| | | GCCTCTAGCGAGGCCAGAAACAGCGCCTTCGGGTTCCA |
| | | AGGACGCCTGCTGCATCTGAGCGCCGGACAGCGCCTGG |
| | | GAGTGCATCTGCATACTGAAGCCAGAGCCCGGCATGCTT |
| | | GGCAGCTGACTCAGGGGGCAACTGTGCTGGGACTGTTTC |
| | | GCGTGACACCTGAGATCCCTGCCGGACTGCCAAGCCCTA |
| | | GATCAGAA |
| 138 | nucleotide | GAAGTGCAGCTGCTCGAAAGCGGCGGGGGACTGGTCCA |
| | sequence of anti- | GCCCGGCGGTTCCCTGAGGCTGTCTTGCGCCGCTTCAGG |
| | PD1(0314) Fc | GTTCAGCTTTTCCTCTTACACCATGAGTTGGGTCAGACA |
| | knob chain fused | GGCACCTGGCAAGGGACTGGAGTGGGTCGCCACAATCA |
| | to monomeric hu | GCGGTGGCGGGCGCGACATTTATTACCCAGATTCCGTGA |
| | 4-1BBL (71-254) | AAGGACGGTTCACCATCTCTAGGGACAACTCAAAGAAT |
| | | ACTCTGTATTTGCAGATGAACAGCCTGAGAGCTGAGGAT |
| | | ACAGCAGTTTACTACTGTGTGCTCCTGACCGGCCGCGTC |
| | | TATTTTGCCCTTGACTCCTGGGGACAAGGCACTCTGGTG |
| | | ACCGTATCTAGTGCTAGCACCAAGGGCCCATCGGTCTTC |
| | | CCCCTGGCACCCTCCTCCAAGAGCACCTCTGGGGGCACA |
| | | GCGGCCCTGGGCTGCCTGGTCAAGGACTACTTCCCCGAA |
| | | CCGGTGACGGTGTCGTGGAACTCAGGCGCCCTGACCAG |
| | | CGGCGTGCACACCTTCCCGGCTGTCCTACAGTCCTCAGG |
| | | ACTCTACTCCCTCAGCAGCGTGGTGACCGTGCCCTCCAG |
| | | CAGCTTGGGCACCCAGACCTACATCTGCAACGTGAATCA |
| | | CAAGCCCAGCAACACCAAGGTGGACAAGAAAGTTGAGC |
| | | CCAAATCTTGTGACAAAACTCACACATGCCCACCGTGCC |
| | | CAGCACCTGAAGCTGCAGGGGGACCGTCAGTCTTCCTCT |
| | | TCCCCCCAAAACCCAAGGACACCCTCATGATCTCCCGGA |
| | | CCCCTGAGGTCACATGCGTGGTGGTGGACGTGAGCCAC |
| | | GAAGACCCTGAGGTCAAGTTCAACTGGTACGTGGACGG |
| | | CGTGGAGGTGCATAATGCCAAGACAAAGCCGCGGGAGG |
| | | AGCAGTACAACAGCACGTACCGTGTGGTCAGCGTCCTC |
| | | ACCGTCCTGCACCAGGACTGGCTGAATGGCAAGGAGTA |
| | | CAAGTGCAAGGTCTCCAACAAAGCCCTCGGCGCCCCCA |
| | | TCGAGAAAACCATCTCCAAAGCCAAAGGGCAGCCCCGA |
| | | GAACCACAGGTGTACACCCTGCCCCCCTGCAGAGATGA |
| | | GCTGACCAAGAACCAGGTGTCCCTGTGGTGTCTGGTCAA |
| | | GGGCTTCTACCCCAGCGATATCGCCGTGGAGTGGGAGA |
| | | GCAACGGCCAGCCTGAGAACAACTACAAGACCACCCCC |
| | | CCTGTGCTGGACAGCGACGGCAGCTTCTTCCTGTACTCC |
| | | AAACTGACCGTGGACAAGAGCCGGTGGCAGCAGGGCAA |
| | | CGTGTTCAGCTGCAGCGTGATGCACGAGGCCCTGCACA |
| | | ACCACTACACCCAGAAGTCCCTGAGCCTGAGCCCCGGC |
| | | GGAGGCGGCGGAAGCGGAGGAGGAGGATCCAGAGAGG |
| | | GCCCTGAGCTGAGCCCCGATGATCCTGCTGGACTGCTGG |
| | | ACCTGCGGCAGGGCATGTTTGCTCAGCTGGTGGCCCAGA |
| | | ACGTGCTGCTGATCGATGGCCCCCTGTCCTGGTACAGCG |
| | | ATCCTGGACTGGCTGGCGTGTCACTGACAGGCGGCCTGA |
| | | GCTACAAAGAGGACACCAAAGAACTGGTGGTGGCCAAG |
| | | GCCGGCGTGTACTACGTGTTCTTTCAGCTGGAACTGCGG |
| | | AGAGTGGTGGCCGGCGAAGGATCTGGCTCTGTGTCTCTG |
| | | GCCCTGCATCTGCAGCCTCTGAGAAGCGCTGCTGGCGCT |
| | | GCAGCTCTGGCACTGACAGTGGATCTGCCTCCTGCCAGC |
| | | TCCGAGGCCCGGAATAGCGCATTTGGGTTTCAAGGCAG |
| | | GCTGCTGCACCTGTCTGCCGGCCAGAGGCTGGGAGTGC |
| | | ATCTGCACACAGAGGCCAGGGCTAGACACGCCTGGCAG |
| | | CTGACACAGGGCGCTACAGTGCTGGGCCTGTTCAGAGTG |
| | | ACCCCCGAGATTCCAGCCGGCCTGCCTTCTCCAAGAAGC |
| | | GAA |
| 134 | nucleotide | see Table 8 |
| | sequence of anti- | |
| | PD1(0314) light | |
| | chain | |
| 84 | anti-PD1(0314) | EVQLLESGGGLVQPGGSLRLSCAASGFSFSSYTMSWVRQA |
| | Fc hole chain | PGKGLEWVATISGGGRDIYYPDSVKGRFTISRDNSKNTLYL |
| | fused to dimeric | QMNSLRAEDTAVYYCVLLTGRVYFALDSWGQGTLVTVSS |
| | hu 4-1BBL (71- | ASTKGPSVFPLAPSSKSTSGGTAALGCLVKDYFPEPVTVSW |
| | 254) | NSGALTSGVHTFPAVLQSSGLYSLSSVVTVPSSSLGTQTYIC |
| | | NVNHKPSNTKVDKKVEPKSCDKTHTCPPCPAPEAAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVCTLPPSRDELTK |
| | | NQVSLSCAVKGFYPSDIAVEWESNGQPENNYKTTPPVLDS |
| | | DGSFFLVSKLTVDKSRWQQGNVFSCSVMHEALHNHYTQK |
| | | SLSLSPGGGGGSGGGGSREGPELSPDDPAGLLDLRQGMFA |
| | | QLVAQNVLLIDGPLSWYSDPGLAGVSLTGGLSYKEDTKEL |
| | | VVAKAGVYYVFFQLELRRVVAGEGSGSVSLALHLQPLRSA |
| | | AGAAALALTVDLPPASSEARNSAFGFQGRLLHLSAGQRLG |
| | | VHLHTEARARHAWQLTQGATVLGLFRVTPEIPAGLPSPRSE |
| | | GGGGSGGGGSREGPELSPDDPAGLLDLRQGMFAQLVAQN |
| | | VLLIDGPLSWYSDPGLAGVSLTGGLSYKEDTKELVVAKAG |
| | | VYYVFFQLELRRVVAGEGSGSVSLALHLQPLRSAAGAAAL |
| | | ALTVDLPPASSEARNSAFGFQGRLLHLSAGQRLGVHLHTE |
| | | ARARHAWQLTQGATVLGLFRVTPEIPAGLPSPRSE |
| 85 | anti-PD1(0314) | EVQLLESGGGLVQPGGSLRLSCAASGFSFSSYTMSWVRQA |
| | Fc knob chain | PGKGLEWVATISGGGRDIYYPDSVKGRFTISRDNSKNTLYL |
| | fused to | QMNSLRAEDTAVYYCVLLTGRVYFALDSWGQGTLVTVSS |
| | monomeric hu 4- | ASTKGPSVFPLAPSSKSTSGGTAALGCLVKDYFPEPVTVSW |
| | 1BBL (71-254) | NSGALTSGVHTFPAVLQSSGLYSLSSVVTVPSSSLGTQTYIC |
| | | NVNHKPSNTKVDKKVEPKSCDKTHTCPPCPAPEAAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVYTLPPCRDELTK |
| | | NQVSLWCLVKGFYPSDIAVEWESNGQPENNYKTTPPVLDS |
| | | DGSFFLYSKLTVDKSRWQQGNVFSCSVMHEALHNHYTQK |
| | | SLSLSPGGGGGSGGGGSREGPELSPDDPAGLLDLRQGMFA |
| | | QLVAQNVLLIDGPLSWYSDPGLAGVSLTGGLSYKEDTKEL |
| | | VVAKAGVYYVFFQLELRRVVAGEGSGSVSLALHLQPLRSA |
| | | AGAAALALTVDLPPASSEARNSAFGFQGRLLHLSAGQRLG |
| | | VHLHTEARARHAWQLTQGATVLGLFRVTPEIPAGLPSPRSE |
| 83 | anti-PD1(0314) | see Table 21 |
| | light chain | |

2.4 Preparation of Monovalent PD1(0314) Targeted 4-1BB Ligand (71-248) Trimer-Containing Fc (Kih) Fusion Antigen Binding Molecule with Crossed CH1-CL Domains with Charged Residues (Construct 7.4)

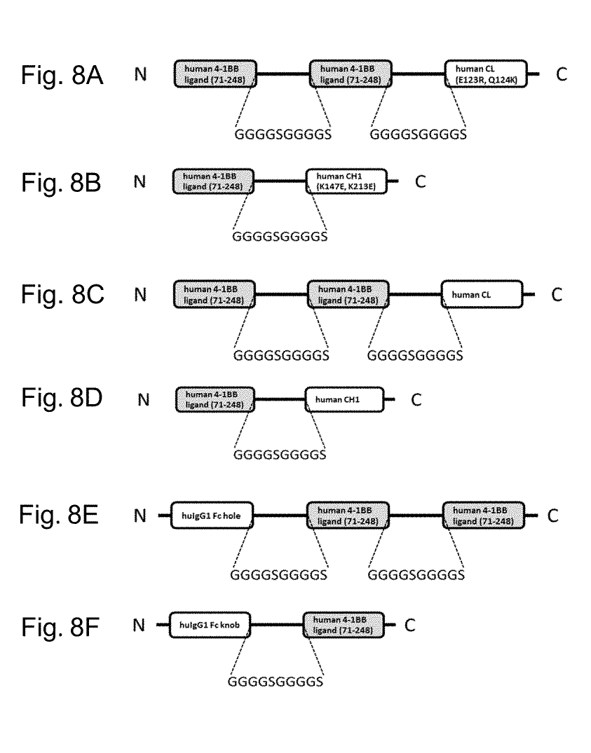

A polypeptide containing two ectodomains of 4-1BB ligand (71-248), separated by (G4S)2 linkers, and fused to the human IgG1-CL domain, was cloned as depicted in FIG. 8A: human 4-1BB ligand, (G4S)2 connector, human 4-1BB ligand, (G4S)2 connector, human CL. A polypeptide containing one ectodomain of 4-1BB ligand (71-248) and fused to the human IgG1-CH domain, was cloned as described in FIG. 8B: human 4-1BB ligand, (G4S)2 connector, human CH1.

To improve correct pairing the following mutations have been introduced in the crossed CH-CL. In the dimeric 4-1BB ligand fused to human CL the mutations E123R and Q124K were introduced. In the monomeric 4-1BB ligand fused to human CH1, the mutations K147E and K213E were cloned into the human CH1 domain as described in International Patent Appl. Publ. No. WO 2015/150447.

The variable region of heavy and light chain DNA sequences encoding a binder specific for PD1, clone 0103-0314, were subcloned in frame with either the constant heavy chain of the hole or the constant light chain of human IgG1.

The Pro329Gly, Leu234Ala and Leu235Ala mutations have been introduced in the constant region of the knob and hole heavy chains to abrogate binding to Fc gamma receptors according to the method described in WO 2012/130831.

Combination of the dimeric ligand-Fc knob chain containing the S354C/T366W mutations, the monomeric CH1 fusion, the targeted anti-PD1(0314)-Fc hole chain containing the Y349C/T366S/L368A/Y407V mutations and the anti-PD1(0314) light chain allows generation of a heterodimer, which includes an assembled trimeric 4-1BB ligand and a FAP binding Fab (FIG. 7D, Construct 7.4).

Table 11 shows the cDNA and amino acid sequences of the monovalent PD1(0314)-human 4-1BB ligand (71-248) Fc (kih) fusion antigen binding molecule containing CH1-CL crossover with charged residues (Construct 7.4).

TABLE 11
Sequences of monovalent PD1(0314)-targeted human 4-1BB ligand
(71-248) containing Fc (kih) fusion molecule Construct 7.4

| SEQ ID NO: | Description | Sequence |
|---|---|---|
| 139 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | dimeric hu 4- | GGCCCAGAACGTGCTGCTGATCGATGGCCCCCTGTCCTG |
| | 1BBL (71-248) - | GTACAGCGATCCTGGACTGGCTGGCGTGTCACTGACAGG |
| | CL* Fc knob | CGGCCTGAGCTACAAAGAGGACACCAAAGAACTGGTGG |
| | chain | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGATCTGCT |
| | | GCTGGCGCCGCTGCTCTGGCACTGACAGTGGATCTGCCT |
| | | CCTGCCAGCAGCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCCGGACTGGGAGG |
| | | CGGCGGATCTGGCGGCGGAGGATCTAGAGAAGGACCCG |
| | | AGCTGTCCCCTGACGATCCAGCCGGGCTGCTGGATCTGA |
| | | GACAGGGAATGTTCGCCCAGCTGGTGGCTCAGAATGTG |
| | | CTGCTGATTGACGGACCTCTGAGCTGGTACTCCGACCCA |
| | | GGGCTGGCAGGGGTGTCCCTGACTGGGGGACTGTCCTAC |
| | | AAAGAAGATACAAAAGAACTGGTGGTGGCTAAAGCTGG |
| | | GGTGTACTATGTGTTTTTTCAGCTGGAACTGAGGCGGGT |
| | | GGTGGCTGGGGAGGGCTCAGGATCTGTGTCCCTGGCTCT |
| | | GCATCTGCAGCCACTGCGCTCTGCAGCAGGGGCTGCAG |
| | | CACTGGCCCTGACTGTGGACCTGCCCCCAGCTTCTTCCG |
| | | AGGCCAGAAACAGCGCCTTCGGGTTCCAAGGACGCCTG |
| | | CTGCATCTGAGCGCCGGACAGCGCCTGGGAGTGCATCT |
| | | GCATACTGAAGCCAGAGCCCGGCATGCTTGGCAGCTGA |
| | | CTCAGGGGGCAACTGTGCTGGGACTGTTTCGCGTGACAC |
| | | CTGAGATCCCCGCTGGACTGGGCGGAGGCGGTTCCGGA |
| | | GGGGGAGGATCTCGTACGGTGGCTGCACCATCTGTCTTT |
| | | ATCTTCCCACCCAGCGACCGGAAGCTGAAGTCTGGCAC |
| | | AGCCAGCGTCGTGTGCCTGCTGAATAACTTCTACCCCCG |
| | | CGAGGCCAAGGTGCAGTGGAAGGTGGACAATGCCCTGC |
| | | AGAGCGGCAACAGCCAGGAAAGCGTGACCGAGCAGGA |
| | | CAGCAAGGACTCCACCTACAGCCTGAGCAGCACCCTGA |
| | | CCCTGAGCAAGGCCGACTACGAGAAGCACAAGGTGTAC |
| | | GCCTGCGAAGTGACCCACCAGGGCCTGTCTAGCCCCGTG |
| | | ACCAAGAGCTTCAACCGGGGCGAGTGCGACAAGACCCA |
| | | CACCTGTCCTCCATGCCCTGCCCCTGAAGCTGCTGGCGG |
| | | CCCTAGCGTGTTCCTGTTCCCCCCAAAGCCCAAGGACAC |
| | | CCTGATGATCAGCCGGACCCCTGAAGTGACCTGCGTGGT |
| | | GGTGGATGTGTCCCACGAGGACCCTGAAGTGAAGTTCA |
| | | ATTGGTACGTGGACGGCGTGGAAGTGCACAATGCCAAG |
| | | ACCAAGCCGCGGGAGGAGCAGTACAACAGCACGTACCG |
| | | TGTGGTCAGCGTCCTCACCGTCCTGCACCAGGACTGGCT |
| | | GAATGGCAAGGAGTACAAGTGCAAGGTCTCCAACAAAG |
| | | CCCTCGGCGCCCCCATCGAGAAAACCATCTCCAAAGCC |
| | | AAAGGGCAGCCCCGAGAACCACAGGTGTACACCCTGCC |
| | | CCCATGCCGGGATGAGCTGACCAAGAACCAGGTCAGCC |
| | | TGTGGTGCCTGGTCAAAGGCTTCTATCCCAGCGACATCG |
| | | CCGTGGAGTGGGAGAGCAATGGGCAGCCGGAGAACAAC |
| | | TACAAGACCACGCCTCCCGTGCTGGACTCCGACGGCTCC |
| | | TTCTTCCTCTACAGCAAGCTCACCGTGGACAAGAGCAGG |
| | | TGGCAGCAGGGGAACGTCTTCTCATGCTCCGTGATGCAT |
| | | GAGGCTCTGCACAACCACTACACGCAGAAGAGCCTCTC |
| | | CCTGTCTCCGGGTAAA |
| 140 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | monomeric hu 4- | GGCCCAGAACGTGCTGCTGATCGATGGCCCCCTGTCCTG |
| | 1BBL (71-248) - | GTACAGCGATCCTGGACTGGCTGGCGTGTCACTGACAGG |
| | CH1* | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGATCTGCT |
| | | GCTGGCGCCGCTGCTCTGGCACTGACAGTGGATCTGCCT |
| | | CCTGCCAGCAGCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCCGGACTGGGAGG |
| | | CGGAGGTTCCGGAGGCGGAGGATCTGCTAGCACAAAGG |
| | | GCCCCAGCGTGTTCCCTCTGGCCCCTAGCAGCAAGAGCA |
| | | CATCTGGCGGAACAGCCGCCCTGGGCTGCCTGGTGGAA |
| | | GATTACTTCCCCGAGCCCGTGACCGTGTCCTGGAATTCT |
| | | GGCGCCCTGACAAGCGGCGTGCACACCTTTCCAGCCGTG |
| | | CTGCAGAGCAGCGGCCTGTACTCTCTGAGCAGCGTCGTG |
| | | ACAGTGCCCAGCAGCTCTCTGGGCACCCAGACCTACATC |
| | | TGCAACGTGAACCACAAGCCCAGCAACACCAAGGTGGA |
| | | CGAGAAGGTGGAACCCAAGTCCTGC |
| 133 | nucleotide | see Table 8 |
| | sequence of anti- | |
| | PD1(0314) Fc | |
| | hole chain | |
| 134 | nucleotide | see Table 8 |
| | sequence of anti- | |
| | PD1(0314) light | |
| | chain | |
| 78 | Dimeric hu 4- | REGPELSPDDPAGLLDLRQGMFAQLVAQNVLLIDGPLSWY |
| | 1BBL (71-248) - | SDPGLAGVSLTGGLSYKEDTKELVVAKAGVYYVFFQLELR |
| | CL* Fc knob | RVVAGEGSGSVSLALHLQPLRSAAGAAALALTVDLPPASS |
| | chain | EARNSAFGFQGRLLHLSAGQRLGVHLHTEARARHAWQLT |
| | | QGATVLGLFRVTPEIPAGLGGGGSGGGGSREGPELSPDDPA |
| | | GLLDLRQGMFAQLVAQNVLLIDGPLSWYSDPGLAGVSLTG |
| | | GLSYKEDTKELVVAKAGVYYVFFQLELRRVVAGEGSGSV |
| | | SLALHLQPLRSAAGAAALALTVDLPPASSEARNSAFGFQG |
| | | RLLHLSAGQRLGVHLHTEARARHAWQLTQGATVLGLFRV |
| | | TPEIPAGLGGGGSGGGGSRTVAAPSVFIFPPSDRKLKSGTAS |
| | | VVCLLNNFYPREAKVQWKVDNALQSGNSQESVTEQDSKD |
| | | STYSLSSTLTLSKADYEKHKVYACEVTHQGLSSPVTKSFNR |
| | | GECDKTHTCPPCPAPEAAGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALGAPIEKTI |
| | | SKAKGQPREPQVYTLPPCRDELTKNQVSLWCLVKGFYPSD |
| | | IAVEWESNGQPENNYKTTPPVLDSDGSFFLYSKLTVDKSR |
| | | WQQGNVFSCSVMHEALHNHYTQKSLSLSPGK |
| 79 | Monomeric hu 4- | REGPELSPDDPAGLLDLRQGMFAQLVAQNVLLIDGPLSWY |
| | 1BBL (71-248) - | SDPGLAGVSLTGGLSYKEDTKELVVAKAGVYYVFFQLELR |
| | CH1* | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSASTKGPSVFPLA |
| | | PSSKSTSGGTAALGCLVEDYFPEPVTVSWNSGALTSGVHTF |
| | | PAVLQSSGLYSLSSVVTVPSSSLGTQTYICNVNHKPSNTKV |
| | | DEKVEPKSC |
| 82 | anti-PD1(0314) | see Table 8 |
| | Fc hole chain | |
| 83 | anti-PD1(0314) | see Table 8 |
| | light chain | |

2.5 Preparation of Monovalent PD1(0314) Targeted 4-1BB Ligand (71-248) Trimer-Containing Fc (Kih) Fusion Antigen Binding Molecule with Crossed CH1-CL Domains without Charged Residues (Construct 7.5)

A polypeptide containing two ectodomains of 4-1BB ligand (71-248), separated by (G4S)2 linkers, and fused to the human IgG1-CL domain, was cloned as depicted in FIG. 8C: human 4-1BB ligand, (G4S)2 connector, human 4-1BB ligand, (G4S)2 connector, human CL. A polypeptide containing one ectodomain of 4-1BB ligand (71-248) and fused to the human IgG1-CH domain, was cloned as described in FIG. 8D: human 4-1BB ligand, (G4S)2 connector, human CH.

The variable region of heavy and light chain DNA sequences encoding a binder specific for PD1, clone 0103-0314, were subcloned in frame with either the constant heavy chain of the hole or the constant light chain of human IgG1.

The Pro329Gly, Leu234Ala and Leu235Ala mutations have been introduced in the constant region of the knob and hole heavy chains to abrogate binding to Fc gamma receptors according to the method described in WO 2012/130831.

Combination of the dimeric ligand-Fc knob chain containing the S354C/T366W mutations, the monomeric CH1 fusion, the targeted anti-PD1 Fc hole chain containing the Y349C/T366S/L368A/Y407V mutations and the anti-PD1 light chain allows generation of a heterodimer, which includes an assembled trimeric 4-1BB ligand and a FAP binding Fab (FIG. 7E, Construct 7.5)

Table 12 shows the cDNA and amino acid sequences of the monovalent PD1(0314)-human 4-1BB ligand (71-248) Fc (kih) fusion antigen binding molecule containing CH1-CL crossover without charged residues (Construct 7.5).

TABLE 12
Sequences of monovalent PD1(0314)-targeted human 4-1BB ligand
(71-248) containing Fc (kih) fusion molecule Construct 7.5

| SEQ ID NO: | Description | Sequence |
|---|---|---|
| 141 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | dimeric hu 4- | GGCCCAGAACGTGCTGCTGATCGATGGCCCCCTGTCCTG |
| | 1BBL (71-248) - | GTACAGCGATCCTGGACTGGCTGGCGTGTCACTGACAGG |
| | CL Fc knob chain | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGATCTGCT |
| | | GCTGGCGCCGCTGCTCTGGCACTGACAGTGGATCTGCCT |
| | | CCTGCCAGCAGCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCCGGACTGGGAGG |
| | | CGGCGGATCTGGCGGCGGAGGATCTAGAGAAGGACCCG |
| | | AGCTGTCCCCTGACGATCCAGCCGGGCTGCTGGATCTGA |
| | | GACAGGGAATGTTCGCCCAGCTGGTGGCTCAGAATGTG |
| | | CTGCTGATTGACGGACCTCTGAGCTGGTACTCCGACCCA |
| | | GGGCTGGCAGGGGTGTCCCTGACTGGGGGACTGTCCTAC |
| | | AAAGAAGATACAAAAGAACTGGTGGTGGCTAAAGCTGG |
| | | GGTGTACTATGTGTTTTTTCAGCTGGAACTGAGGCGGGT |
| | | GGTGGCTGGGGAGGGCTCAGGATCTGTGTCCCTGGCTCT |
| | | GCATCTGCAGCCACTGCGCTCTGCAGCAGGGGCTGCAG |
| | | CACTGGCCCTGACTGTGGACCTGCCCCCAGCTTCTTCCG |
| | | AGGCCAGAAACAGCGCCTTCGGGTTCCAAGGACGCCTG |
| | | CTGCATCTGAGCGCCGGACAGCGCCTGGGAGTGCATCT |
| | | GCATACTGAAGCCAGAGCCCGGCATGCTTGGCAGCTGA |
| | | CTCAGGGGGCAACTGTGCTGGGACTGTTTCGCGTGACAC |
| | | CTGAGATCCCCGCTGGACTGGGCGGAGGCGGTTCCGGA |
| | | GGGGGAGGATCTCGTACGGTGGCCGCTCCCTCCGTGTTT |
| | | ATCTTTCCCCCATCCGATGAACAGCTGAAAAGCGGCACC |
| | | GCCTCCGTCGTGTGTCTGCTGAACAATTTTTACCCTAGG |
| | | GAAGCTAAAGTGCAGTGGAAAGTGGATAACGCACTGCA |
| | | GTCCGGCAACTCCCAGGAATCTGTGACAGAACAGGACT |
| | | CCAAGGACAGCACCTACTCCCTGTCCTCCACCCTGACAC |
| | | TGTCTAAGGCTGATTATGAGAAACACAAAGTCTACGCCT |
| | | GCGAAGTCACCCATCAGGGCCTGAGCTCGCCCGTCACA |
| | | AAGAGCTTCAACAGGGGAGAGTGTGACAAGACCCACAC |
| | | CTGTCCCCCTTGTCCTGCCCCTGAAGCTGCTGGCGGCCC |
| | | TTCTGTGTTCCTGTTCCCCCCAAAGCCCAAGGACACCCT |
| | | GATGATCAGCCGGACCCCCGAAGTGACCTGCGTGGTGG |
| | | TGGATGTGTCCCACGAGGACCCTGAAGTGAAGTTCAATT |
| | | GGTACGTGGACGGCGTGGAAGTGCACAATGCCAAGACC |
| | | AAGCCGCGGGAGGAGCAGTACAACAGCACGTACCGTGT |
| | | GGTCAGCGTCCTCACCGTCCTGCACCAGGACTGGCTGAA |
| | | TGGCAAGGAGTACAAGTGCAAGGTCTCCAACAAAGCCC |
| | | TCGGCGCCCCCATCGAGAAAACCATCTCCAAAGCCAAA |
| | | GGGCAGCCCCGAGAACCACAGGTGTACACCCTGCCCCC |
| | | ATGCCGGGATGAGCTGACCAAGAACCAGGTCAGCCTGT |
| | | GGTGCCTGGTCAAAGGCTTCTATCCCAGCGACATCGCCG |
| | | TGGAGTGGGAGAGCAATGGGCAGCCGGAGAACAACTAC |
| | | AAGACCACGCCTCCCGTGCTGGACTCCGACGGCTCCTTC |
| | | TTCCTCTACAGCAAGCTCACCGTGGACAAGAGCAGGTG |
| | | GCAGCAGGGGAACGTCTTCTCATGCTCCGTGATGCATGA |
| | | GGCTCTGCACAACCACTACACGCAGAAGAGCCTCTCCCT |
| | | GTCTCCGGGTAAA |
| 142 | nucleotide | AGAGAGGGCCCTGAGCTGAGCCCCGATGATCCTGCTGG |
| | sequence of | ACTGCTGGACCTGCGGCAGGGCATGTTTGCTCAGCTGGT |
| | monomeric hu 4- | GGCCCAGAACGTGCTGCTGATCGATGGCCCCCTGTCCTG |
| | 1BBL (71-248) - | GTACAGCGATCCTGGACTGGCTGGCGTGTCACTGACAGG |
| | CH1 | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGATCTGCT |
| | | GCTGGCGCCGCTGCTCTGGCACTGACAGTGGATCTGCCT |
| | | CCTGCCAGCAGCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCCGGACTGGGAGG |
| | | CGGAGGTTCCGGAGGCGGAGGATCTGCTAGCACCAAAG |
| | | GCCCTTCCGTGTTTCCTCTGGCTCCTAGCTCCAAGTCCAC |
| | | CTCTGGAGGCACCGCTGCTCTCGGATGCCTCGTGAAGGA |
| | | TTATTTTCCTGAGCCTGTGACAGTGTCCTGGAATAGCGG |
| | | AGCACTGACCTCTGGAGTGCATACTTTCCCCGCTGTGCT |
| | | GCAGTCCTCTGGACTGTACAGCCTGAGCAGCGTGGTGAC |
| | | AGTGCCCAGCAGCAGCCTGGGCACCCAGACCTACATCT |
| | | GCAACGTGAACCACAAGCCCAGCAACACCAAGGTGGAC |
| | | AAGAAGGTGGAACCCAAGTCTTGT |
| 133 | nucleotide | see Table 8 |
| | sequence of anti- | |
| | PD1(0314) Fc | |
| | hole chain | |
| 134 | nucleotide | see Table 8 |
| | sequence of anti- | |
| | PD1(0314) light | |
| | chain | |
| 80 | Dimeric hu 4- | REGPELSPDDPAGLLDLRQGMFAQLVAQNVLLIDGPLSWY |
| | 1BBL (71-248) - | SDPGLAGVSLTGGLSYKEDTKELVVAKAGVYYVFFQLELR |
| | CL Fc knob chain | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSREGPELSPDDPA |
| | | GLLDLRQGMFAQLVAQNVLLIDGPLSWYSDPGLAGVSLTG |
| | | GLSYKEDTKELVVAKAGVYYVFFQLELRRVVAGEGSGSV |
| | | SLALHLQPLRSAAGAAALALTVDLPPASSEARNSAFGFQG |
| | | RLLHLSAGQRLGVHLHTEARARHAWQLTQGATVLGLFRV |
| | | TPEIPAGLGGGGSGGGGSRTVAAPSVFIFPPSDEQLKSGTAS |
| | | VVCLLNNFYPREAKVQWKVDNALQSGNSQESVTEQDSKD |
| | | STYSLSSTLTLSKADYEKHKVYACEVTHQGLSSPVTKSFNR |
| | | GECDKTHTCPPCPAPEAAGGPSVFLFPPKPKDTLMISRTPE |
| | | VTCVVVDVSHEDPEVKFNWYVDGVEVHNAKTKPREEQY |
| | | NSTYRVVSVLTVLHQDWLNGKEYKCKVSNKALGAPIEKTI |
| | | SKAKGQPREPQVYTLPPCRDELTKNQVSLWCLVKGFYPSD |
| | | IAVEWESNGQPENNYKTTPPVLDSDGSFFLYSKLTVDKSR |
| | | WQQGNVFSCSVMHEALHNHYTQKSLSLSPGK |
| 81 | Monomeric hu 4- | REGPELSPDDPAGLLDLRQGMFAQLVAQNVLLIDGPLSWY |
| | 1BBL (71-248) - | SDPGLAGVSLTGGLSYKEDTKELVVAKAGVYYVFFQLELR |
| | CH1 | RVVAGEGSGSVSLALHLQPLRSAAGAAALALTVDLPPASS |
| | | EARNSAFGFQGRLLHLSAGQRLGVHLHTEARARHAWQLT |
| | | QGATVLGLFRVTPEIPAGLGGGGSGGGGSASTKGPSVFPLA |
| | | PSSKSTSGGTAALGCLVKDYFPEPVTVSWNSGALTSGVHT |
| | | FPAVLQSSGLYSLSSVVTVPSSSLGTQTYICNVNHKPSNTK |
| | | VDKKVEPKSC |
| 82 | anti-PD1(0314) | see Table 8 |
| | Fc hole chain | |
| 83 | anti-PD1(0314) | see Table 8 |
| | light chain | |

2.6 Preparation of Bivalent PD1(0314) Targeted 4-1BB Ligand (71-248) Trimer-Containing Fc (Kih) Fusion Antigen Binding Molecule with the Dimeric and Monomeric 4-1BB Ligands Fused at the C-Terminus of Each Heavy Chain (Construct 7.6)

A polypeptide containing two ectodomains of 4-1BB ligand (71-248), separated by (G4S)2 linkers was fused to the C-terminus of human IgG1 Fc hole chain, as depicted in FIG. 8E: human IgG1 Fc hole, (G4S)2 connector, human 4-1BB ligand, (G4S)2 connector, human 4-1BB ligand. A polypeptide containing one ectodomain of 4-1BB ligand (71-248) and fused to the C-terminus of human IgG1 Fc knob chain as described in FIG. 8F: human IgG1 Fc knob, (G4S)2 connector, human 4-1BB ligand.

The variable region of heavy and light chain DNA sequences encoding a binder specific for PD1, clone 0103-0314, were subcloned in frame with either the constant heavy chain of the hole, the knob or the constant light chain of human IgG1.

The Pro329Gly, Leu234Ala and Leu235Ala mutations have been introduced in the constant region of the knob and hole heavy chains to abrogate binding to Fc gamma receptors according to the method described in WO 2012/130831.

Combination of the anti-PD1 huIgG1 hole dimeric ligand chain containing the Y349C/T366S/L368A/Y407V mutations, the anti-PD1 huIgG1 knob monomeric ligand chain containing the S354C/T366W mutations and the anti-PD1 light chain allows generation of a heterodimer, which includes an assembled trimeric 4-1BB ligand and two PD1 binding Fabs (FIG. 7F, Construct 7.6).

Table 13 shows the cDNA and amino acid sequences of the bivalent PD1(0314)-targeted 4-1BB ligand trimer-containing Fc (kih) fusion molecule Construct 7.6 (PD1 split trimer with 2 anti-PD1 Fabs, dimeric and monomeric 4-1BB ligand fused at the C-terminus of each heavy chain, respectively).

TABLE 13
Sequences of bivalent PD1(0314)-targeted human 4-1BB ligand (71-248)
containing Fc (kih) fusion molecule (Construct 7.6)

| SEQ ID NO: | Description | Sequence |
|---|---|---|
| 143 | nucleotide | GAAGTGCAGCTGCTCGAAAGCGGCGGGGGACTGGTCCA |
| | sequence of anti- | GCCCGGCGGTTCCCTGAGGCTGTCTTGCGCCGCTTCAGG |
| | PD1(0314) Fc | GTTCAGCTTTTCCTCTTACACCATGAGTTGGGTCAGACA |
| | hole chain fused | GGCACCTGGCAAGGGACTGGAGTGGGTCGCCACAATCA |
| | to dimeric hu 4- | GCGGTGGCGGGCGCGACATTTATTACCCAGATTCCGTGA |
| | 1BBL (71-248) | AAGGACGGTTCACCATCTCTAGGGACAACTCAAAGAAT |
| | | ACTCTGTATTTGCAGATGAACAGCCTGAGAGCTGAGGAT |
| | | ACAGCAGTTTACTACTGTGTGCTCCTGACCGGCCGCGTC |
| | | TATTTTGCCCTTGACTCCTGGGGACAAGGCACTCTGGTG |
| | | ACCGTATCTAGTGCTAGCACCAAGGGCCCCTCCGTGTTC |
| | | CCCCTGGCCCCCAGCAGCAAGAGCACCAGCGGCGGCAC |
| | | AGCCGCTCTGGGCTGCCTGGTCAAGGACTACTTCCCCGA |
| | | GCCCGTGACCGTGTCCTGGAACAGCGGAGCCCTGACCTC |
| | | CGGCGTGCACACCTTCCCCGCCGTGCTGCAGAGTTCTGG |
| | | CCTGTATAGCCTGAGCAGCGTGGTCACCGTGCCTTCTAG |
| | | CAGCCTGGGCACCCAGACCTACATCTGCAACGTGAACC |
| | | ACAAGCCCAGCAACACCAAGGTGGACAAGAAGGTGGA |
| | | GCCCAAGAGCTGCGACAAAACTCACACATGCCCACCGT |
| | | GCCCAGCACCTGAAGCTGCAGGGGGACCGTCAGTCTTC |
| | | CTCTTCCCCCCAAAACCCAAGGACACCCTCATGATCTCC |
| | | CGGACCCCTGAGGTCACATGCGTGGTGGTGGACGTGAG |
| | | CCACGAAGACCCTGAGGTCAAGTTCAACTGGTACGTGG |
| | | ACGGCGTGGAGGTGCATAATGCCAAGACAAAGCCGCGG |
| | | GAGGAGCAGTACAACAGCACGTACCGTGTGGTCAGCGT |
| | | CCTCACCGTCCTGCACCAGGACTGGCTGAATGGCAAGG |
| | | AGTACAAGTGCAAGGTCTCCAACAAAGCCCTCGGCGCC |
| | | CCCATCGAGAAAACCATCTCCAAAGCCAAAGGGCAGCC |
| | | CCGAGAACCACAGGTGTGCACCCTGCCCCCATCCCGGG |
| | | ATGAGCTGACCAAGAACCAGGTCAGCCTCTCGTGCGCA |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCGT |
| | | GAGCAAGCTCACCGTGGACAAGAGCAGGTGGCAGCAGG |
| | | GGAACGTCTTCTCATGCTCCGTGATGCATGAGGCTCTGC |
| | | ACAACCACTACACGCAGAAGAGCCTCTCCCTGTCTCCGG |
| | | GTGGAGGCGGCGGAAGCGGAGGAGGAGGATCCAGAGA |
| | | GGGCCCTGAGCTGAGCCCTGATGATCCTGCCGGACTGCT |
| | | GGACCTGCGGCAGGGAATGTTTGCCCAGCTGGTGGCCC |
| | | AGAACGTGCTGCTGATCGATGGCCCCCTGTCCTGGTACA |
| | | GCGATCCTGGACTGGCTGGCGTGTCACTGACAGGCGGC |
| | | CTGAGCTACAAAGAGGACACCAAAGAACTGGTGGTGGC |
| | | CAAGGCCGGCGTGTACTACGTGTTCTTTCAGCTGGAACT |
| | | GCGGAGAGTGGTGGCCGGCGAAGGATCTGGCTCTGTGT |
| | | CTCTGGCCCTGCATCTGCAGCCTCTGAGATCTGCTGCTG |
| | | GCGCCGCTGCTCTGGCACTGACAGTGGATCTGCCTCCTG |
| | | CCAGCAGCGAGGCCCGGAATAGCGCATTTGGGTTTCAA |
| | | GGCAGGCTGCTGCACCTGTCTGCCGGCCAGAGGCTGGG |
| | | AGTGCATCTGCACACAGAGGCCAGGGCTAGACACGCCT |
| | | GGCAGCTGACACAGGGCGCTACAGTGCTGGGCCTGTTC |
| | | AGAGTGACCCCCGAGATTCCAGCAGGCCTGGGAGGCGG |
| | | CGGATCTGGCGGCGGAGGATCTAGAGAAGGACCCGAGC |
| | | TGTCCCCCGACGATCCCGCTGGGCTGCTGGATCTGAGAC |
| | | AGGGCATGTTCGCTCAGCTGGTGGCTCAGAATGTGCTGC |
| | | TGATTGACGGACCTCTGAGCTGGTACTCCGACCCAGGGC |
| | | TGGCAGGGGTGTCCCTGACTGGGGGACTGTCCTACAAA |
| | | GAAGATACAAAAGAACTGGTGGTGGCTAAAGCTGGGGT |
| | | GTACTATGTGTTTTTTCAGCTGGAACTGAGGCGGGTGGT |
| | | GGCTGGGGAGGGCTCAGGATCTGTGTCCCTGGCTCTGCA |
| | | TCTGCAGCCACTGCGCTCTGCAGCAGGGGCTGCAGCACT |
| | | GGCCCTGACTGTGGACCTGCCCCCAGCTTCTTCCGAGGC |
| | | CAGAAACAGCGCCTTCGGGTTCCAAGGACGCCTGCTGC |
| | | ATCTGAGCGCCGGACAGCGCCTGGGAGTGCATCTGCAT |
| | | ACTGAAGCCAGAGCCCGGCATGCTTGGCAGCTGACTCA |
| | | GGGGGCAACTGTGCTGGGACTGTTTCGCGTGACACCTGA |
| | | GATCCCAGCCGGGCTC |
| 144 | nucleotide | GAAGTGCAGCTGCTCGAAAGCGGCGGGGGACTGGTCCA |
| | sequence of anti- | GCCCGGCGGTTCCCTGAGGCTGTCTTGCGCCGCTTCAGG |
| | PD1(0314) Fc | GTTCAGCTTTTCCTCTTACACCATGAGTTGGGTCAGACA |
| | knob chain fused | GGCACCTGGCAAGGGACTGGAGTGGGTCGCCACAATCA |
| | to monomeric hu | GCGGTGGCGGGCGCGACATTTATTACCCAGATTCCGTGA |
| | 4-1BBL (71-248) | AAGGACGGTTCACCATCTCTAGGGACAACTCAAAGAAT |
| | | ACTCTGTATTTGCAGATGAACAGCCTGAGAGCTGAGGAT |
| | | ACAGCAGTTTACTACTGTGTGCTCCTGACCGGCCGCGTC |
| | | TATTTTGCCCTTGACTCCTGGGGACAAGGCACTCTGGTG |
| | | ACCGTATCTAGTGCTAGCACCAAGGGCCCATCGGTCTTC |
| | | CCCCTGGCACCCTCCTCCAAGAGCACCTCTGGGGGCACA |
| | | GCGGCCCTGGGCTGCCTGGTCAAGGACTACTTCCCCGAA |
| | | CCGGTGACGGTGTCGTGGAACTCAGGCGCCCTGACCAG |
| | | CGGCGTGCACACCTTCCCGGCTGTCCTACAGTCCTCAGG |
| | | ACTCTACTCCCTCAGCAGCGTGGTGACCGTGCCCTCCAG |
| | | CAGCTTGGGCACCCAGACCTACATCTGCAACGTGAATCA |
| | | CAAGCCCAGCAACACCAAGGTGGACAAGAAAGTTGAGC |
| | | CCAAATCTTGTGACAAAACTCACACATGCCCACCGTGCC |
| | | CAGCACCTGAAGCTGCAGGGGGACCGTCAGTCTTCCTCT |
| | | TCCCCCCAAAACCCAAGGACACCCTCATGATCTCCCGGA |
| | | CCCCTGAGGTCACATGCGTGGTGGTGGACGTGAGCCAC |
| | | GAAGACCCTGAGGTCAAGTTCAACTGGTACGTGGACGG |
| | | CGTGGAGGTGCATAATGCCAAGACAAAGCCGCGGGAGG |
| | | AGCAGTACAACAGCACGTACCGTGTGGTCAGCGTCCTC |
| | | ACCGTCCTGCACCAGGACTGGCTGAATGGCAAGGAGTA |
| | | CAAGTGCAAGGTCTCCAACAAAGCCCTCGGCGCCCCCA |
| | | TCGAGAAAACCATCTCCAAAGCCAAAGGGCAGCCCCGA |
| | | GAACCACAGGTGTACACCCTGCCCCCCTGCAGAGATGA |
| | | GCTGACCAAGAACCAGGTGTCCCTGTGGTGTCTGGTCAA |
| | | GGGCTTCTACCCCAGCGATATCGCCGTGGAGTGGGAGA |
| | | GCAACGGCCAGCCTGAGAACAACTACAAGACCACCCCC |
| | | CCTGTGCTGGACAGCGACGGCAGCTTCTTCCTGTACTCC |
| | | AAACTGACCGTGGACAAGAGCCGGTGGCAGCAGGGCAA |
| | | CGTGTTCAGCTGCAGCGTGATGCACGAGGCCCTGCACA |
| | | ACCACTACACCCAGAAGTCCCTGAGCCTGAGCCCCGGC |
| | | GGAGGCGGCGGAAGCGGAGGAGGAGGATCCAGAGAGG |
| | | GCCCTGAGCTGAGCCCTGATGATCCTGCCGGACTGCTGG |
| | | ACCTGCGGCAGGGAATGTTTGCCCAGCTGGTGGCCCAG |
| | | AACGTGCTGCTGATCGATGGCCCCCTGTCCTGGTACAGC |
| | | GATCCTGGACTGGCTGGCGTGTCACTGACAGGCGGCCTG |
| | | AGCTACAAAGAGGACACCAAAGAACTGGTGGTGGCCAA |
| | | GGCCGGCGTGTACTACGTGTTCTTTCAGCTGGAACTGCG |
| | | GAGAGTGGTGGCCGGCGAAGGATCTGGCTCTGTGTCTCT |
| | | GGCCCTGCATCTGCAGCCTCTGAGATCTGCTGCTGGCGC |
| | | CGCTGCTCTGGCACTGACAGTGGATCTGCCTCCTGCCAG |
| | | CAGCGAGGCCCGGAATAGCGCATTTGGGTTTCAAGGCA |
| | | GGCTGCTGCACCTGTCTGCCGGCCAGAGGCTGGGAGTG |
| | | CATCTGCACACAGAGGCCAGGGCTAGACACGCCTGGCA |
| | | GCTGACACAGGGCGCTACAGTGCTGGGCCTGTTCAGAGT |
| | | GACCCCCGAGATTCCTGCCGGGCTC |
| 134 | nucleotide | see Table 8 |
| | sequence of anti- | |
| | PD1(0314) light | |
| | chain | |
| 86 | anti-PD1(0314) | EVQLLESGGGLVQPGGSLRLSCAASGFSFSSYTMSWVRQA |
| | Fc hole chain | PGKGLEWVATISGGGRDIYYPDSVKGRFTISRDNSKNTLYL |
| | fused to dimeric | QMNSLRAEDTAVYYCVLLTGRVYFALDSWGQGTLVTVSS |
| | hu 4-1BBL (71- | ASTKGPSVFPLAPSSKSTSGGTAALGCLVKDYFPEPVTVSW |
| | 248) | NSGALTSGVHTFPAVLQSSGLYSLSSVVTVPSSSLGTQTYIC |
| | | NVNHKPSNTKVDKKVEPKSCDKTHTCPPCPAPEAAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVCTLPPSRDELTK |
| | | NQVSLSCAVKGFYPSDIAVEWESNGQPENNYKTTPPVLDS |
| | | DGSFFLVSKLTVDKSRWQQGNVFSCSVMHEALHNHYTQK |
| | | SLSLSPGGGGGSGGGGSREGPELSPDDPAGLLDLRQGMFA |
| | | QLVAQNVLLIDGPLSWYSDPGLAGVSLTGGLSYKEDTKEL |
| | | VVAKAGVYYVFFQLELRRVVAGEGSGSVSLALHLQPLRSA |
| | | AGAAALALTVDLPPASSEARNSAFGFQGRLLHLSAGQRLG |
| | | VHLHTEARARHAWQLTQGATVLGLFRVTPEIPAGLGGGGS |
| | | GGGGSREGPELSPDDPAGLLDLRQGMFAQLVAQNVLLIDG |
| | | PLSWYSDPGLAGVSLTGGLSYKEDTKELVVAKAGVYYVF |
| | | FQLELRRVVAGEGSGSVSLALHLQPLRSAAGAAALALTVD |
| | | LPPASSEARNSAFGFQGRLLHLSAGQRLGVHLHTEARARH |
| | | AWQLTQGATVLGLFRVTPEIPAGL |
| 87 | anti-PD1(0314) | EVQLLESGGGLVQPGGSLRLSCAASGFSFSSYTMSWVRQA |
| | Fc knob chain | PGKGLEWVATISGGGRDIYYPDSVKGRFTISRDNSKNTLYL |
| | fused to | QMNSLRAEDTAVYYCVLLTGRVYFALDSWGQGTLVTVSS |
| | monomeric hu 4- | ASTKGPSVFPLAPSSKSTSGGTAALGCLVKDYFPEPVTVSW |
| | 1BBL (71-248) | NSGALTSGVHTFPAVLQSSGLYSLSSVVTVPSSSLGTQTYIC |
| | | NVNHKPSNTKVDKKVEPKSCDKTHTCPPCPAPEAAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVYTLPPCRDELTK |
| | | NQVSLWCLVKGFYPSDIAVEWESNGQPENNYKTTPPVLDS |
| | | DGSFFLYSKLTVDKSRWQQGNVFSCSVMHEALHNHYTQK |
| | | SLSLSPGGGGGSGGGGSREGPELSPDDPAGLLDLRQGMFA |
| | | QLVAQNVLLIDGPLSWYSDPGLAGVSLTGGLSYKEDTKEL |
| | | VVAKAGVYYVFFQLELRRVVAGEGSGSVSLALHLQPLRSA |
| | | AGAAALALTVDLPPASSEARNSAFGFQGRLLHLSAGQRLG |
| | | VHLHTEARARHAWQLTQGATVLGLFRVTPEIPAGL |
| 83 | anti-PD1(0314) | see Table 8 |
| | light chain | |

2.7 Preparation of Monovalent PD1(0314) Targeted 4-1BB Ligand (71-248) Trimer-Containing Fc (Kih) Fusion Antigen Binding Molecules with the Dimeric and Monomeric 4-1BB Ligands Fused at the C-Terminus of Each Heavy Chain (Anti-PD1 on the Knob Chain, Constructs 7.11 and 7.12)

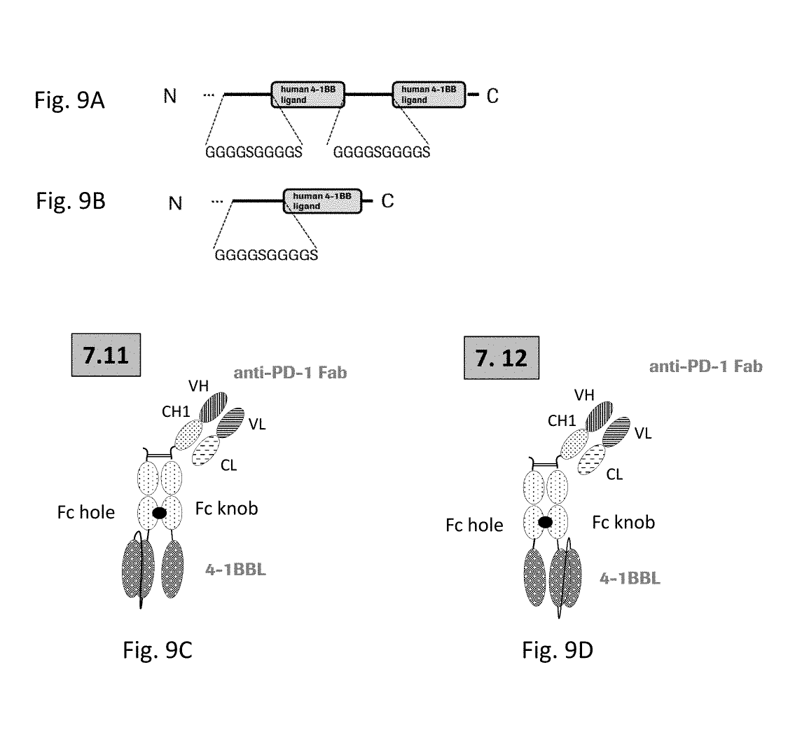

A polypeptide containing two ectodomains of 4-1BB ligand, separated by (G4S)2 linkers was subcloned in frame to the C-terminus of human IgG1 Fc hole or knob chain, as depicted in FIG. 9A: human IgG1 Fc, (G4S)2 connector, human 4-1BB ligand, (G4S)2 connector, human 4-1BB ligand. A polypeptide containing one ectodomain of 4-1BB ligand was subcloned in frame to the C-terminus of human IgG1 Fc knob or hole chain as described in FIG. 9B: human IgG1 Fc, (G4S)2 connector, human 4-1BB ligand.

The variable region of heavy and light chain DNA sequences encoding a binder specific for PD1, clone 0314, were subcloned in frame with either the constant heavy chain of the knob or the constant light chain of human IgG1.

The Pro329Gly, Leu234Ala and Leu235Ala mutations have been introduced in the constant region of the knob and hole heavy chains to abrogate binding to Fc gamma receptors according to the method described in WO 2012/130831.

Combination of the huIgG1 hole chain containing the Y349C/T366S/L368A/Y407V mutations, the anti-PD1 huIgG1 knob chain containing the S354C/T366W mutations and the anti-PD1 light chain allows generation of a heterodimer, which includes an assembled trimeric 4-1BB ligand and one PD1 binding Fab (FIG. 9C and FIG. 9D, Constructs 7.11 and 7.12).

Table 14 shows the cDNA and amino acid sequences of the monovalent PD1(0314)-targeted 4-1BB ligand trimer-containing Fc (kih) fusion molecule Construct 7.11 (PD1 split trimer with one anti-PD1 Fab, dimeric 4-1BB ligand fused at the C-terminus of the Fc hole chain and monomeric 4-1BB ligand fused at the C-terminus of the Fc knob chain).

TABLE 14
Sequences of monovalent PD1(0314)-targeted human 4-1BB ligand
(71-248) containing Fc (kih) fusion molecule (Construct 7.11)

| SEQ ID NO: | Description | Sequence |
|---|---|---|
| 145 | nucleotide | GACAAAACTCACACATGCCCACCGTGCCCAGCACCTGA |
| | sequence of Fc | AGCTGCAGGGGGACCGTCAGTCTTCCTCTTCCCCCCAAA |
| | hole dimeric | ACCCAAGGACACCCTCATGATCTCCCGGACCCCTGAGGT |
| | ligand (71-248) | CACATGCGTGGTGGTGGACGTGAGCCACGAAGACCCTG |
| | chain | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTGCACCCTGCCCCCATCCCGGGATGAGCTGACCAAGA |
| | | ACCAGGTCAGCCTCTCGTGCGCAGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCGTGAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTGGAGGCGGCGGA |
| | | AGCGGAGGAGGAGGATCCAGAGAGGGCCCTGAGCTGAG |
| | | CCCTGATGATCCTGCCGGACTGCTGGACCTGCGGCAGGG |
| | | AATGTTTGCCCAGCTGGTGGCCCAGAACGTGCTGCTGAT |
| | | CGATGGCCCCCTGTCCTGGTACAGCGATCCTGGACTGGC |
| | | TGGCGTGTCACTGACAGGCGGCCTGAGCTACAAAGAGG |
| | | ACACCAAAGAACTGGTGGTGGCCAAGGCCGGCGTGTAC |
| | | TACGTGTTCTTTCAGCTGGAACTGCGGAGAGTGGTGGCC |
| | | GGCGAAGGATCTGGCTCTGTGTCTCTGGCCCTGCATCTG |
| | | CAGCCTCTGAGATCTGCTGCTGGCGCCGCTGCTCTGGCA |
| | | CTGACAGTGGATCTGCCTCCTGCCAGCAGCGAGGCCCG |
| | | GAATAGCGCATTTGGGTTTCAAGGCAGGCTGCTGCACCT |
| | | GTCTGCCGGCCAGAGGCTGGGAGTGCATCTGCACACAG |
| | | AGGCCAGGGCTAGACACGCCTGGCAGCTGACACAGGGC |
| | | GCTACAGTGCTGGGCCTGTTCAGAGTGACCCCCGAGATT |
| | | CCAGCAGGCCTGGGAGGCGGCGGATCTGGCGGCGGAGG |
| | | ATCTAGAGAAGGACCCGAGCTGTCCCCCGACGATCCCG |
| | | CTGGGCTGCTGGATCTGAGACAGGGCATGTTCGCTCAGC |
| | | TGGTGGCTCAGAATGTGCTGCTGATTGACGGACCTCTGA |
| | | GCTGGTACTCCGACCCAGGGCTGGCAGGGGTGTCCCTG |
| | | ACTGGGGGACTGTCCTACAAAGAAGATACAAAAGAACT |
| | | GGTGGTGGCTAAAGCTGGGGTGTACTATGTGTTTTTTCA |
| | | GCTGGAACTGAGGCGGGTGGTGGCTGGGGAGGGCTCAG |
| | | GATCTGTGTCCCTGGCTCTGCATCTGCAGCCACTGCGCT |
| | | CTGCAGCAGGGGCTGCAGCACTGGCCCTGACTGTGGAC |
| | | CTGCCCCCAGCTTCTTCCGAGGCCAGAAACAGCGCCTTC |
| | | GGGTTCCAAGGACGCCTGCTGCATCTGAGCGCCGGACA |
| | | GCGCCTGGGAGTGCATCTGCATACTGAAGCCAGAGCCC |
| | | GGCATGCTTGGCAGCTGACTCAGGGGGCAACTGTGCTG |
| | | GGACTGTTTCGCGTGACACCTGAGATCCCAGCCGGGCTC |
| 144 | nucleotide | see Table 13 |
| | sequence of anti- | |
| | PD1(0314) Fc | |
| | knob chain fused | |
| | to monomeric hu | |
| | 4-1BBL (71-248) | |
| 134 | nucleotide | see Table 8 |
| | sequence of anti- | |
| | PD1(0314) light | |
| | chain | |
| 88 | Fc hole dimeric | DKTHTCPPCPAPEAAGGPSVFLFPPKPKDTLMISRTPEVTC |
| | ligand (71-248) | VVVDVSHEDPEVKFNWYVDGVEVHNAKTKPREEQYNSTY |
| | chain | RVVSVLTVLHQDWLNGKEYKCKVSNKALGAPIEKTISKAK |
| | | GQPREPQVCTLPPSRDELTKNQVSLSCAVKGFYPSDIAVEW |
| | | ESNGQPENNYKTTPPVLDSDGSFFLVSKLTVDKSRWQQGN |
| | | VFSCSVMHEALHNHYTQKSLSLSPGGGGGSGGGGSREGPE |
| | | LSPDDPAGLLDLRQGMFAQLVAQNVLLIDGPLSWYSDPGL |
| | | AGVSLTGGLSYKEDTKELVVAKAGVYYVFFQLELRRVVA |
| | | GEGSGSVSLALHLQPLRSAAGAAALALTVDLPPASSEARN |
| | | SAFGFQGRLLHLSAGQRLGVHLHTEARARHAWQLTQGAT |
| | | VLGLFRVTPEIPAGLGGGGSGGGGSREGPELSPDDPAGLLD |
| | | LRQGMFAQLVAQNVLLIDGPLSWYSDPGLAGVSLTGGLSY |
| | | KEDTKELVVAKAGVYYVFFQLELRRVVAGEGSGSVSLAL |
| | | HLQPLRSAAGAAALALTVDLPPASSEARNSAFGFQGRLLH |
| | | LSAGQRLGVHLHTEARARHAWQLTQGATVLGLFRVTPEIP |
| | | AGL |
| 87 | anti-PD1(0314) | see Table 13 |
| | Fc knob chain | |
| | fused to | |
| | monomeric hu 4- | |
| | 1BBL (71-248) | |
| 83 | anti-PD1(0314) | see Table 8 |
| | light chain | |

Table 15 shows the cDNA and amino acid sequences of the monovalent PD1(0314)-targeted 4-1BB ligand trimer-containing Fc (kih) fusion molecule Construct 7.12 (PD1 split trimer with one anti-PD1 Fab, dimeric 4-1BB ligand fused at the C-terminus of the Fc knob chain and monomeric 4-1BB ligand fused at the C-terminus of the Fc hole chain).

TABLE 15
Sequences of monovalent PD1(0314)-targeted
human 4-1BB ligand (71-248) containing Fc (kih)
fusion molecule (Construct 7.12)

| SEQ ID | | |
|---|---|---|
| NO: | Description | Sequence |
| 146 | nucleotide | GACAAAACTCACACATGCCCACCGTGCCCAGCACCTGA |
| | sequence of Fc | AGCTGCAGGGGGACCGTCAGTCTTCCTCTTCCCCCCAAA |
| | hole monomeric | ACCCAAGGACACCCTCATGATCTCCCGGACCCCTGAGGT |
| | ligand (71-248) | CACATGCGTGGTGGTGGACGTGAGCCACGAAGACCCTG |
| | chain | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTGCACCCTGCCCCCATCCCGGGATGAGCTGACCAAGA |
| | | ACCAGGTCAGCCTCTCGTGCGCAGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCGTGAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAGAGAGGGCCCT |
| | | GAGCTGAGCCCTGATGATCCTGCCGGACTGCTGGACCTG |
| | | CGGCAGGGAATGTTTGCCCAGCTGGTGGCCCAGAACGT |
| | | GCTGCTGATCGATGGCCCCCTGTCCTGGTACAGCGATCC |
| | | TGGACTGGCTGGCGTGTCACTGACAGGCGGCCTGAGCTA |
| | | CAAAGAGGACACCAAAGAACTGGTGGTGGCCAAGGCCG |
| | | GCGTGTACTACGTGTTCTTTCAGCTGGAACTGCGGAGAG |
| | | TGGTGGCCGGCGAAGGATCTGGCTCTGTGTCTCTGGCCC |
| | | TGCATCTGCAGCCTCTGAGATCTGCTGCTGGCGCCGCTG |
| | | CTCTGGCACTGACAGTGGATCTGCCTCCTGCCAGCAGCG |
| | | AGGCCCGGAATAGCGCATTTGGGTTTCAAGGCAGGCTG |
| | | CTGCACCTGTCTGCCGGCCAGAGGCTGGGAGTGCATCTG |
| | | CACACAGAGGCCAGGGCTAGACACGCCTGGCAGCTGAC |
| | | ACAGGGCGCTACAGTGCTGGGCCTGTTCAGAGTGACCC |
| | | CCGAGATTCCTGCCGGGCTC |
| 147 | nucleotide | GAAGTGCAGCTGCTCGAAAGCGGCGGGGGACTGGTCCA |
| | sequence of anti- | GCCGGCGGTTCCCTGAGGCTGTCTTGCGCCGCTTCAGG |
| | PD1(0314) Fc | GTTCAGCTTTTCCTCTTACACCATGAGTTGGGTCAGACA |
| | knob chain fused | GGCACCTGGCAAGGGACTGGAGTGGGTCGCCACAATCA |
| | to dimeric hu 4- | GCGGTGGCGGGCGCGACATTTATTACCCAGATTCCGTGA |
| | 1BBL (71-248) | AAGGACGGTTCACCATCTCTAGGGACAACTCAAAGAAT |
| | | ACTCTGTATTTGCAGATGAACAGCCTGAGAGCTGAGGAT |
| | | ACAGCAGTTTACTACTGTGTGCTCCTGACCGGCCGCGTC |
| | | TATTTTGCCCTTGACTCCTGGGGACAAGGCACTCTGGTG |
| | | ACCGTATCTAGTGCTAGCACCAAGGGCCCATCGGTCTTC |
| | | CCCCTGGCACCCTCCTCCAAGAGCACCTCTGGGGGCACA |
| | | GCGGCCCTGGGCTGCCTGGTCAAGGACTACTTCCCCGAA |
| | | CCGGTGACGGTGTCGTGGAACTCAGGCGCCCTGACCAG |
| | | CGGCGTGCACACCTTCCCGGCTGTCCTACAGTCCTCAGG |
| | | ACTCTACTCCCTCAGCAGCGTGGTGACCGTGCCCTCCAG |
| | | CAGCTTGGGCACCCAGACCTACATCTGCAACGTGAATCA |
| | | CAAGCCCAGCAACACCAAGGTGGACAAGAAAGTTGAGC |
| | | CCAAATCTTGTGACAAAACTCACACATGCCCACCGTGCC |
| | | CAGCACCTGAAGCTGCAGGGGGACCGTCAGTCTTCCTCT |
| | | TCCCCCCAAAACCCAAGGACACCCTCATGATCTCCCGGA |
| | | CCCCTGAGGTCACATGCGTGGTGGTGGACGTGAGCCAC |
| | | GAAGACCCTGAGGTCAAGTTCAACTGGTACGTGGACGG |
| | | CGTGGAGGTGCATAATGCCAAGACAAAGCCGCGGGAGG |
| | | AGCAGTACAACAGCACGTACCGTGTGGTCAGCGTCCTC |
| | | ACCGTCCTGCACCAGGACTGGCTGAATGGCAAGGAGTA |
| | | CAAGTGCAAGGTCTCCAACAAAGCCCTCGGCGCCCCCA |
| | | TCGAGAAAACCATCTCCAAAGCCAAAGGGCAGCCCCGA |
| | | GAACCACAGGTGTACACCCTGCCCCCCTGCAGAGATGA |
| | | GCTGACCAAGAACCAGGTGTCCCTGTGGTGTCTGGTCAA |
| | | GGGCTTCTACCCCAGCGATATCGCCGTGGAGTGGGAGA |
| | | GCAACGGCCAGCCTGAGAACAACTACAAGACCACCCCC |
| | | CCTGTGCTGGACAGCGACGGCAGCTTCTTCCTGTACTCC |
| | | AAACTGACCGTGGACAAGAGCCGGTGGCAGCAGGGCAA |
| | | CGTGTTCAGCTGCAGCGTGATGCACGAGGCCCTGCACA |
| | | ACCACTACACCCAGAAGTCCCTGAGCCTGAGCCCCGGC |
| | | AGAGAGGGCCCTGAGCTGAGCCCTGATGATCCTGCCGG |
| | | ACTGCTGGACCTGCGGCAGGGAATGTTTGCCCAGCTGGT |
| | | GGCCCAGAACGTGCTGCTGATCGATGGCCCCCTGTCCTG |
| | | GTACAGCGATCCTGGACTGGCTGGCGTGTCACTGACAGG |
| | | CGGCCTGAGCTACAAAGAGGACACCAAAGAACTGGTGG |
| | | TGGCCAAGGCCGGCGTGTACTACGTGTTCTTTCAGCTGG |
| | | AACTGCGGAGAGTGGTGGCCGGCGAAGGATCTGGCTCT |
| | | GTGTCTCTGGCCCTGCATCTGCAGCCTCTGAGATCTGCT |
| | | GCTGGCGCCGCTGCTCTGGCACTGACAGTGGATCTGCCT |
| | | CCTGCCAGCAGCGAGGCCCGGAATAGCGCATTTGGGTTT |
| | | CAAGGCAGGCTGCTGCACCTGTCTGCCGGCCAGAGGCT |
| | | GGGAGTGCATCTGCACACAGAGGCCAGGGCTAGACACG |
| | | CCTGGCAGCTGACACAGGGCGCTACAGTGCTGGGCCTG |
| | | TTCAGAGTGACCCCCGAGATTCCAGCAGGCCTGGGAGG |
| | | CGGCGGATCTGGCGGCGGAGGATCTAGAGAAGGACCCG |
| | | AGCTGTCCCCCGACGATCCCGCTGGGCTGCTGGATCTGA |
| | | GACAGGGCATGTTCGCTCAGCTGGTGGCTCAGAATGTGC |
| | | TGCTGATTGACGGACCTCTGAGCTGGTACTCCGACCCAG |
| | | GGCTGGCAGGGGTGTCCCTGACTGGGGGACTGTCCTACA |
| | | AAGAAGATACAAAAGAACTGGTGGTGGCTAAAGCTGGG |
| | | GTGTACTATGTGTTTTTTCAGCTGGAACTGAGGCGGGTG |
| | | GTGGCTGGGGAGGGCTCAGGATCTGTGTCCCTGGCTCTG |
| | | CATCTGCAGCCACTGCGCTCTGCAGCAGGGGCTGCAGC |
| | | ACTGGCCCTGACTGTGGACCTGCCCCCAGCTTCTTCCGA |
| | | GGCCAGAAACAGCGCCTTCGGGTTCCAAGGACGCCTGC |
| | | TGCATCTGAGCGCCGGACAGCGCCTGGGAGTGCATCTG |
| | | CATACTGAAGCCAGAGCCCGGCATGCTTGGCAGCTGAC |
| | | TCAGGGGGCAACTGTGCTGGGACTGTTTCGCGTGACACC |
| | | TGAGATCCCAGCCGGGCTC |
| 134 | nucleotide | see Table 8 |
| | sequence of anti- | |
| | PD1(0314) light | |
| | chain | |
| 89 | Fc hole | DKTHTCPPCPAPEAAGGPSVFLFPPKPKDTLMISRTPEVTC |
| | monomeric ligand | VVVDVSHEDPEVKFNWYVDGVEVHNAKTKPREEQYNSTY |
| | (71-248) chain | RVVSVLTVLHQDWLNGKEYKCKVSNKALGAPIEKTISKAK |
| | | GQPREPQVCTLPPSRDELTKNQVSLSCAVKGFYPSDIAVEW |
| | | ESNGQPENNYKTTPPVLDSDGSFFLVSKLTVDKSRWQQGN |
| | | VFSCSVMHEALHNHYTQKSLSLSPGREGPELSPDDPAGLL |
| | | DLRQGMFAQLVAQNVLLIDGPLSWYSDPGLAGVSLTGGLS |
| | | YKEDTKELVVAKAGVYYVFFQLELRRVVAGEGSGSVSLA |
| | | LHLQPLRSAAGAAALALTVDLPPASSEARNSAFGFQGRLL |
| | | HLSAGQRLGVHLHTEARARHAWQLTQGATVLGLFRVTPEI |
| | | PAGL |
| 90 | anti-PD1(0314) | EVQLLESGGGLVQPGGSLRLSCAASGFSFSSYTMSWVRQA |
| | Fc knob chain | PGKGLEWVATISGGGRDIYYPDSVKGRFTISRDNSKNTLYL |
| | fused to dimeric | QMNSLRAEDTAVYYCVLLTGRVYFALDSWGQGTLVTVSS |
| | hu 4-1BBL (71- | ASTKGPSVFPLAPSSKSTSGGTAALGCLVKDYFPEPVTVSW |
| | 248) | NSGALTSGVHTFPAVLQSSGLYSLSSVVTVPSSSLGTQTYIC |
| | | NVNHKPSNTKVDKKVEPKSCDKTHTCPPCPAPEAAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVYTLPPCRDELTK |
| | | NQVSLWCLVKGFYPSDIAVEWESNGQPENNYKTTPPVLDS |
| | | DGSFFLYSKLTVDKSRWQQGNVFSCSVMHEALHNHYTQK |
| | | SLSLSPGREGPELSPDDPAGLLDLRQGMFAQLVAQNVLLID |
| | | GPLSWYSDPGLAGVSLTGGLSYKEDTKELVVAKAGVYYV |
| | | FFQLELRRVVAGEGSGSVSLALHLQPLRSAAGAAALALTV |
| | | DLPPASSEARNSAFGFQGRLLHLSAGQRLGVHLHTEARAR |
| | | HAWQLTQGATVLGLFRVTPEIPAGLGGGGSGGGGSREGPE |
| | | LSPDDPAGLLDLRQGMFAQLVAQNVLLIDGPLSWYSDPGL |
| | | AGVSLTGGLSYKEDTKELVVAKAGVYYVFFQLELRRVVA |
| | | GEGSGSVSLALHLQPLRSAAGAAALALTVDLPPASSEARN |
| | | SAFGFQGRLLHLSAGQRLGVHLHTEARARHAWQLTQGAT |
| | | VLGLFRVTPEIPAGL |
| 83 | anti-PD1(0314) | see Table 8 |
| | light chain | |

2.8 Preparation of Monovalent PD1(0314) Targeted 4-1BB Ligand (71-248) Trimer-Containing Fc (Kih) Fusion Antigen Binding Molecules with the Dimeric and Monomeric 4-1BB Ligands Fused at the C-Terminus of Each Heavy Chain (Anti-PD1 on the Hole Chain, Constructs 7.13 and 7.14)

A polypeptide containing two ectodomains of 4-1BB ligand, separated by (G4S)2 linkers was subcloned in frame to the C-terminus of human IgG1 Fc hole or knob chain, as depicted in FIG. 9A: human IgG1 Fc, (G4S)2 connector, human 4-1BB ligand, (G4S)2 connector, human 4-1BB ligand. A polypeptide containing one ectodomain of 4-1BB ligand was subcloned in frame to the C-terminus of human IgG1 Fc knob or hole chain as described in FIG. 9B: human IgG1 Fc, (G4S)2 connector, human 4-1BB ligand.

The variable region of heavy and light chain DNA sequences encoding a binder specific for PD1, clone 0314, were subcloned in frame with either the constant heavy chain of the hole or the constant light chain of human IgG1.

The Pro329Gly, Leu234Ala and Leu235Ala mutations have been introduced in the constant region of the knob and hole heavy chains to abrogate binding to Fc gamma receptors according to the method described in WO 2012/130831.

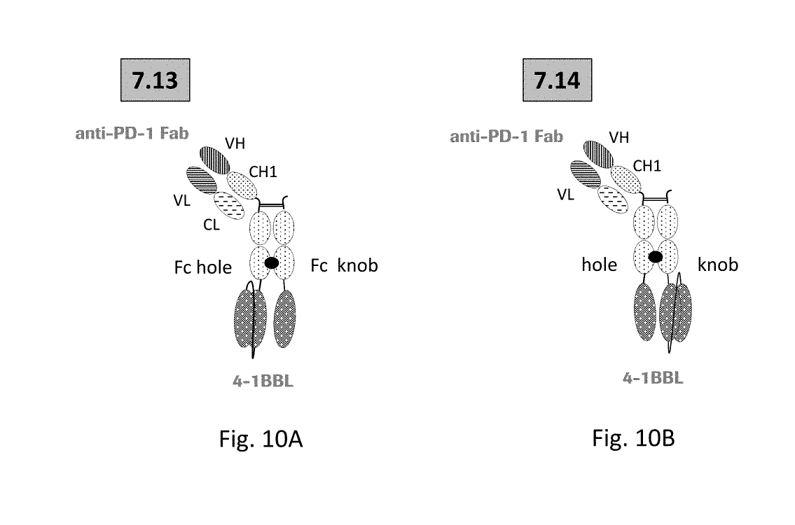

Combination of the anti-PD1 huIgG1 hole chain containing the Y349C/T366S/L368A/Y407V mutations, the huIgG1 knob chain containing the S354C/T366W mutations and the anti-PD1 light chain allows generation of a heterodimer, which includes an assembled trimeric 4-1BB ligand and one PD1 binding Fab (FIG. 10A and FIG. 10B, Constructs 7.13 and 7.14).

Table 16 shows the cDNA and amino acid sequences of the monovalent PD1(0314)-targeted 4-1BB ligand trimer-containing Fc (kih) fusion molecule Construct 7.13 (PD1 split trimer with one anti-PD1 Fab, dimeric 4-1BB ligand fused at the C-terminus of the Fc hole chain and monomeric 4-1BB ligand fused at the C-terminus of the Fc knob chain).

TABLE 16
Sequences of monovalent PD1(0314)-targeted
human 4-1BB ligand (71-248) containing Fc (kih)
fusion molecule (Construct 7.13)

| SEQ ID | | |
|---|---|---|
| NO: | Description | Sequence |
| 143 | nucleotide | see Table 13 |
| | sequence of anti- | |
| | PD1(0314) Fc | |
| | hole dimeric | |
| | ligand (71-248) | |
| | chain | |
| 148 | nucleotide | GACAAAACTCACACATGCCCACCGTGCCCAGCACCTGA |
| | sequence of Fc | AGCTGCAGGGGGACCGTCAGTCTTCCTCTTCCCCCCAAA |
| | knob chain fused | ACCCAAGGACACCCTCATGATCTCCCGGACCCCTGAGGT |
| | to monomeric hu | CACATGCGTGGTGGTGGACGTGAGCCACGAAGACCCTG |
| | 4-1BBL (71-248) | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTACACCCTGCCCCCCTGCAGAGATGAGCTGACCAAGA |
| | | ACCAGGTGTCCCTGTGGTGTCTGGTCAAGGGCTTCTACC |
| | | CCAGCGATATCGCCGTGGAGTGGGAGAGCAACGGCCAG |
| | | CCTGAGAACAACTACAAGACCACCCCCCCTGTGCTGGA |
| | | CAGCGACGGCAGCTTCTTCCTGTACTCCAAACTGACCGT |
| | | GGACAAGAGCCGGTGGCAGCAGGGCAACGTGTTCAGCT |
| | | GCAGCGTGATGCACGAGGCCCTGCACAACCACTACACC |
| | | CAGAAGTCCCTGAGCCTGAGCCCCGGCGGAGGCGGCGG |
| | | AAGCGGAGGAGGAGGATCCAGAGAGGGCCCTGAGCTGA |
| | | GCCCTGATGATCCTGCCGGACTGCTGGACCTGCGGCAGG |
| | | GAATGTTTGCCCAGCTGGTGGCCCAGAACGTGCTGCTGA |
| | | TCGATGGCCCCCTGTCCTGGTACAGCGATCCTGGACTGG |
| | | CTGGCGTGTCACTGACAGGCGGCCTGAGCTACAAAGAG |
| | | GACACCAAAGAACTGGTGGTGGCCAAGGCCGGCGTGTA |
| | | CTACGTGTTCTTTCAGCTGGAACTGCGGAGAGTGGTGGC |
| | | CGGCGAAGGATCTGGCTCTGTGTCTCTGGCCCTGCATCT |
| | | GCAGCCTCTGAGATCTGCTGCTGGCGCCGCTGCTCTGGC |
| | | ACTGACAGTGGATCTGCCTCCTGCCAGCAGCGAGGCCC |
| | | GGAATAGCGCATTTGGGTTTCAAGGCAGGCTGCTGCACC |
| | | TGTCTGCCGGCCAGAGGCTGGGAGTGCATCTGCACACA |
| | | GAGGCCAGGGCTAGACACGCCTGGCAGCTGACACAGGG |
| | | CGCTACAGTGCTGGGCCTGTTCAGAGTGACCCCCGAGAT |
| | | TCCTGCCGGGCTC |
| 134 | nucleotide | see Table 8 |
| | sequence of anti- | |
| | PD1(0314) light | |
| | chain | |
| 86 | anti-PD1(0314) | see Table 13 |
| | Fc hole dimeric | |
| | ligand (71-248) | |
| | chain | |
| 91 | Fc knob chain | DKTHTCPPCPAPEAAGGPSVFLFPPKPKDTLMISRTPEVTC |
| | fused to | VVVDVSHEDPEVKFNWYVDGVEVHNAKTKPREEQYNSTY |
| | monomeric hu 4- | RVVSVLTVLHQDWLNGKEYKCKVSNKALGAPIEKTISKAK |
| | 1BBL (71-248) | GQPREPQVYTLPPCRDELTKNQVSLWCLVKGFYPSDIAVE |
| | | WESNGQPENNYKTTPPVLDSDGSFFLYSKLTVDKSRWQQ |
| | | GNVFSCSVMHEALHNHYTQKSLSLSPGGGGGSGGGGSRE |
| | | GPELSPDDPAGLLDLRQGMFAQLVAQNVLLIDGPLSWYSD |
| | | PGLAGVSLTGGLSYKEDTKELVVAKAGVYYVFFQLELRRV |
| | | VAGEGSGSVSLALHLQPLRSAAGAAALALTVDLPPASSEA |
| | | RNSAFGFQGRLLHLSAGQRLGVHLHTEARARHAWQLTQG |
| | | ATVLGLFRVTPEIPAGL |
| 83 | anti-PD1(0314) | see Table 8 |
| | light chain | |

Table 17 shows the cDNA and amino acid sequences of the monovalent PD1(0314)-targeted 4-1BB ligand trimer-containing Fc (kih) fusion molecule Construct 7.14 (PD1 split trimer with one anti-PD1 Fab, dimeric 4-1BB ligand fused at the C-terminus of the Fc knob chain and monomeric 4-1BB ligand fused at the C-terminus of the Fc hole chain).

TABLE 17
Sequences of monovalent PD1(0314)-targeted
human 4-1BB ligand (71-248) containing Fc (kih)
fusion molecule (Construct 7.14)

| SEQ ID | | |
|---|---|---|
| NO: | Description | Sequence |
| 149 | nucleotide | GAAGTGCAGCTGCTCGAAAGCGGCGGGGGACTGGTCCA |
| | sequence of anti- | GCCCGGCGGTTCCCTGAGGCTGTCTTGCGCCGCTTCAGG |
| | PD1(0314) Fc | GTTCAGCTTTTCCTCTTACACCATGAGTTGGGTCAGACA |
| | hole monomeric | GGCACCTGGCAAGGGACTGGAGTGGGTCGCCACAATCA |
| | ligand (71-248) | GCGGTGGCGGGCGCGACATTTATTACCCAGATTCCGTGA |
| | chain | AAGGACGGTTCACCATCTCTAGGGACAACTCAAAGAAT |
| | | ACTCTGTATTTGCAGATGAACAGCCTGAGAGCTGAGGAT |
| | | ACAGCAGTTTACTACTGTGTGCTCCTGACCGGCCGCGTC |
| | | TATTTTGCCCTTGACTCCTGGGGACAAGGCACTCTGGTG |
| | | ACCGTATCTAGTGCTAGCACCAAGGGCCCCTCCGTGTTC |
| | | CCCCTGGCCCCCAGCAGCAAGAGCACCAGCGGCGGCAC |
| | | AGCCGCTCTGGGCTGCCTGGTCAAGGACTACTTCCCCGA |
| | | GCCCGTGACCGTGTCCTGGAACAGCGGAGCCCTGACCTC |
| | | CGGCGTGCACACCTTCCCCGCCGTGCTGCAGAGTTCTGG |
| | | CCTGTATAGCCTGAGCAGCGTGGTCACCGTGCCTTCTAG |
| | | CAGCCTGGGCACCCAGACCTACATCTGCAACGTGAACC |
| | | ACAAGCCCAGCAACACCAAGGTGGACAAGAAGGTGGA |
| | | GCCCAAGAGCTGCGACAAAACTCACACATGCCCACCGT |
| | | GCCCAGCACCTGAAGCTGCAGGGGGACCGTCAGTCTTC |
| | | CTCTTCCCCCCAAAACCCAAGGACACCCTCATGATCTCC |
| | | CGGACCCCTGAGGTCACATGCGTGGTGGTGGACGTGAG |
| | | CCACGAAGACCCTGAGGTCAAGTTCAACTGGTACGTGG |
| | | ACGGCGTGGAGGTGCATAATGCCAAGACAAAGCCGCGG |
| | | GAGGAGCAGTACAACAGCACGTACCGTGTGGTCAGCGT |
| | | CCTCACCGTCCTGCACCAGGACTGGCTGAATGGCAAGG |
| | | AGTACAAGTGCAAGGTCTCCAACAAAGCCCTCGGCGCC |
| | | CCCATCGAGAAAACCATCTCCAAAGCCAAAGGGCAGCC |
| | | CCGAGAACCACAGGTGTGCACCCTGCCCCCATCCCGGG |
| | | ATGAGCTGACCAAGAACCAGGTCAGCCTCTCGTGCGCA |
| | | GTCAAAGGCTTCTATCCCAGCGACATCGCCGTGGAGTGG |
| | | GAGAGCAATGGGCAGCCGGAGAACAACTACAAGACCAC |
| | | GCCTCCCGTGCTGGACTCCGACGGCTCCTTCTTCCTCGT |
| | | GAGCAAGCTCACCGTGGACAAGAGCAGGTGGCAGCAGG |
| | | GGAACGTCTTCTCATGCTCCGTGATGCATGAGGCTCTGC |
| | | ACAACCACTACACGCAGAAGAGCCTCTCCCTGTCTCCGG |
| | | GTGGAGGCGGCGGAAGCGGAGGAGGAGGATCCAGAGA |
| | | GGGCCCTGAGCTGAGCCCTGATGATCCTGCCGGACTGCT |
| | | GGACCTGCGGCAGGGAATGTTTGCCCAGCTGGTGGCCC |
| | | AGAACGTGCTGCTGATCGATGGCCCCCTGTCCTGGTACA |
| | | GCGATCCTGGACTGGCTGGCGTGTCACTGACAGGCGGC |
| | | CTGAGCTACAAAGAGGACACCAAAGAACTGGTGGTGGC |
| | | CAAGGCCGGCGTGTACTACGTGTTCTTTCAGCTGGAACT |
| | | GCGGAGAGTGGTGGCCGGCGAAGGATCTGGCTCTGTGT |
| | | CTCTGGCCCTGCATCTGCAGCCTCTGAGATCTGCTGCTG |
| | | GCGCCGCTGCTCTGGCACTGACAGTGGATCTGCCTCCTG |
| | | CCAGCAGCGAGGCCCGGAATAGCGCATTTGGGTTTCAA |
| | | GGCAGGCTGCTGCACCTGTCTGCCGGCCAGAGGCTGGG |
| | | AGTGCATCTGCACACAGAGGCCAGGGCTAGACACGCCT |
| | | GGCAGCTGACACAGGGCGCTACAGTGCTGGGCCTGTTC |
| | | AGAGTGACCCCCGAGATTCCTGCCGGGCTC |
| 150 | nucleotide | GACAAAACTCACACATGCCCACCGTGCCCAGCACCTGA |
| | sequence of Fc | AGCTGCAGGGGGACCGTCAGTCTTCCTCTTCCCCCCAAA |
| | knob chain fused | ACCCAAGGACACCCTCATGATCTCCCGGACCCCTGAGGT |
| | to dimeric hu 4- | CACATGCGTGGTGGTGGACGTGAGCCACGAAGACCCTG |
| | 1BBL (71-248) | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTACACCCTGCCCCCCTGCAGAGATGAGCTGACCAAGA |
| | | ACCAGGTGTCCCTGTGGTGTCTGGTCAAGGGCTTCTACC |
| | | CCAGCGATATCGCCGTGGAGTGGGAGAGCAACGGCCAG |
| | | CCTGAGAACAACTACAAGACCACCCCCCCTGTGCTGGA |
| | | CAGCGACGGCAGCTTCTTCCTGTACTCCAAACTGACCGT |
| | | GGACAAGAGCCGGTGGCAGCAGGGCAACGTGTTCAGCT |
| | | GCAGCGTGATGCACGAGGCCCTGCACAACCACTACACC |
| | | CAGAAGTCCCTGAGCCTGAGCCCCGGCGGAGGCGGCGG |
| | | AAGCGGAGGAGGAGGATCCAGAGAGGGCCCTGAGCTGA |
| | | GCCCTGATGATCCTGCCGGACTGCTGGACCTGCGGCAGG |
| | | GAATGTTTGCCCAGCTGGTGGCCCAGAACGTGCTGCTGA |
| | | TCGATGGCCCCCTGTCCTGGTACAGCGATCCTGGACTGG |
| | | CTGGCGTGTCACTGACAGGCGGCCTGAGCTACAAAGAG |
| | | GACACCAAAGAACTGGTGGTGGCCAAGGCCGGCGTGTA |
| | | CTACGTGTTCTTTCAGCTGGAACTGCGGAGAGTGGTGGC |
| | | CGGCGAAGGATCTGGCTCTGTGTCTCTGGCCCTGCATCT |
| | | GCAGCCTCTGAGATCTGCTGCTGGCGCCGCTGCTCTGGC |
| | | ACTGACAGTGGATCTGCCTCCTGCCAGCAGCGAGGCCC |
| | | GGAATAGCGCATTTGGGTTTCAAGGCAGGCTGCTGCACC |
| | | TGTCTGCCGGCCAGAGGCTGGGAGTGCATCTGCACACA |
| | | GAGGCCAGGGCTAGACACGCCTGGCAGCTGACACAGGG |
| | | CGCTACAGTGCTGGGCCTGTTCAGAGTGACCCCCGAGAT |
| | | TCCAGCAGGCCTGGGAGGCGGCGGATCTGGCGGCGGAG |
| | | GATCTAGAGAAGGACCCGAGCTGTCCCCCGACGATCCC |
| | | GCTGGGCTGCTGGATCTGAGACAGGGCATGTTCGCTCAG |
| | | CTGGTGGCTCAGAATGTGCTGCTGATTGACGGACCTCTG |
| | | AGCTGGTACTCCGACCCAGGGCTGGCAGGGGTGTCCCT |
| | | GACTGGGGGACTGTCCTACAAAGAAGATACAAAAGAAC |
| | | TGGTGGTGGCTAAAGCTGGGGTGTACTATGTGTTTTTTC |
| | | AGCTGGAACTGAGGCGGGTGGTGGCTGGGGAGGGCTCA |
| | | GGATCTGTGTCCCTGGCTCTGCATCTGCAGCCACTGCGC |
| | | TCTGCAGCAGGGGCTGCAGCACTGGCCCTGACTGTGGA |
| | | CCTGCCCCCAGCTTCTTCCGAGGCCAGAAACAGCGCCTT |
| | | CGGGTTCCAAGGACGCCTGCTGCATCTGAGCGCCGGAC |
| | | AGCGCCTGGGAGTGCATCTGCATACTGAAGCCAGAGCC |
| | | CGGCATGCTTGGCAGCTGACTCAGGGGGCAACTGTGCTG |
| | | GGACTGTTTCGCGTGACACCTGAGATCCCAGCCGGGCTC |
| 134 | nucleotide | see Table 8 |
| | sequence of anti- | |
| | PD1(0314) light | |
| | chain | |
| 92 | anti-PD1(0314) | EVQLLESGGGLVQPGGSLRLSCAASGFSFSSYTMSWVRQA |
| | Fc hole | PGKGLEWVATISGGGRDIYYPDSVKGRFTISRDNSKNTLYL |
| | monomeric ligand | QMNSLRAEDTAVYYCVLLTGRVYFALDSWGQGTLVTVSS |
| | (71-248) chain | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPEAAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALGAPIEKTISKAKGQPREPQVCTLPPSRDELTK |
| | | NQVSLSCAVKGFYPSDIAVEWESNGQPENNYKTTPPVLDS |
| | | DGSFFLVSKLTVDKSRWQQGNVFSCSVMHEALHNHYTQK |
| | | SLSLSPGGGGGSGGGGSREGPELSPDDPAGLLDLRQGMFA |
| | | QLVAQNVLLIDGPLSWYSDPGLAGVSLTGGLSYKEDTKEL |
| | | VVAKAGVYYVFFQLELRRVVAGEGSGSVSLALHLQPLRSA |
| | | AGAAALALTVDLPPASSEARNSAFGFQGRLLHLSAGQRLG |
| | | VHLHTEARARHAWQLTQGATVLGLFRVTPEIPAGL |
| 93 | Fc knob chain | DKTHTCPPCPAPEAAGGPSVFLFPPKPKDTLMISRTPEVTC |
| | fused to dimeric | VVVDVSHEDPEVKFNWYVDGVEVHNAKTKPREEQYNSTY |
| | hu 4-1BBL (71- | RVVSVLTVLHQDWLNGKEYKCKVSNKALGAPIEKTISKAK |
| | 248) | GQPREPQVYTLPPCRDELTKNQVSLWCLVKGFYPSDIAVE |
| | | WESNGQPENNYKTTPPVLDSDGSFFLYSKLTVDKSRWQQ |
| | | GNVFSCSVMHEALHNHYTQKSLSLSPGGGGGSGGGGSRE |
| | | GPELSPDDPAGLLDLRQGMFAQLVAQNVLLIDGPLSWYSD |
| | | PGLAGVSLTGGLSYKEDTKELVVAKAGVYYVFFQLELRRV |
| | | VAGEGSGSVSLALHLQPLRSAAGAAALALTVDLPPASSEA |
| | | RNSAFGFQGRLLHLSAGQRLGVHLHTEARARHAWQLTQG |
| | | ATVLGLFRVTPEIPAGLGGGGSGGGGSREGPELSPDDPAGL |
| | | LDLRQGMFAQLVAQNVLLIDGPLSWYSDPGLAGVSLTGGL |
| | | SYKEDTKELVVAKAGVYYVFFQLELRRVVAGEGSGSVSL |
| | | ALHLQPLRSAAGAAALALTVDLPPASSEARNSAFGFQGRL |
| | | LHLSAGQRLGVHLHTEARARHAWQLTQGATVLGLFRVTP |
| | | EIPAGL |
| 83 | anti-PD1(0314) | see Table 8 |
| | light chain | |

2.9 Preparation of Untargeted Human 4-1BB Ligand Trimer-Containing Fc Fusion Antigen Binding Molecules (Control Molecules)

The control molecules were prepared as described above for the PD1-targeted antigen binding molecules, with the only difference that the anti-PD1 binder (VH and VL) was replaced by a germline control, termed DP47, not binding to the antigen.

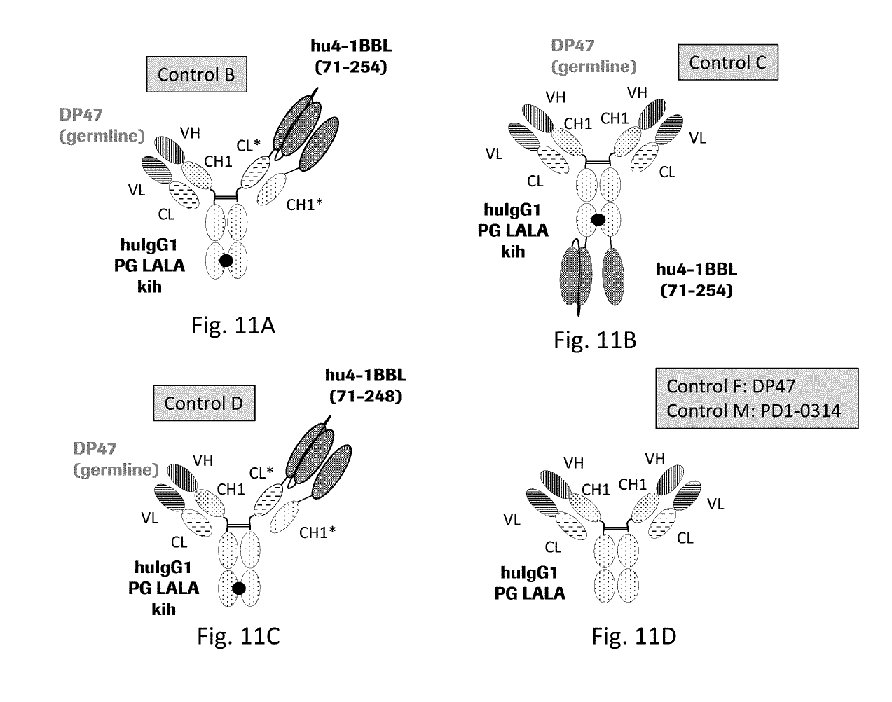

Control B is an untargeted monovalent split trimeric human 4-1BB ligand (71-254) Fc (kih) antigen binding molecule (FIG. 11A) containing a CH-CL crossover with charged residues. It corresponds to Construct 7.1, however the variable region of heavy and light chain DNA sequences of the PD1 binder were replaced with those of the germline control (DP47) and subcloned in frame with either the constant heavy chain of the hole or the constant light chain of human IgG1.

A bivalent variant Control C (FIG. 11B) was prepared in analogy to the bivalent Constructs 7.3 and 7.6 and a monovalent variant Control D (FIG. 11C) was prepared in analogy to Construct 7.3 (containing a 4-1BB ligand (71-248) trimer), with the only difference that the anti-PD1 binder (VH and VL) was replaced by a germline control, termed DP47, not binding to the antigen.

Table 18 shows the cDNA and amino acid sequences of the DP47-untargeted 4-1BB ligand (71-254) trimer-containing Fc (kih) fusion antigen binding molecule with CH1-CL crossover and charged residues in the 4-1BB ligand containing arms (Control B).

TABLE 18
Sequences of DP47 untargeted 4-1BB ligand (71-254)
trimer-containing Fc (kih) fusion antigen binding molecule
(DP47 split 4-1BBL trimer) Control B

| SEQ ID | | |
|---|---|---|
| NO: | Description | Sequence |
| 131 | nucleotide | see Table 8 |
| | sequence of | |
| | dimeric hu 4- | |
| | 1BBL (71-254) - | |
| | CL* Fc knob | |
| | chain | |
| 132 | nucleotide | see Table 8 |
| | sequence of | |
| | monomeric hu | |
| | 4-1BBL (71-254) - | |
| | CH1* | |
| 151 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | Fc hole chain | ATTCACCTTTAGCAGTTATGCCATGAGCTGGGTCCGCCA |
| | | GGCTCCAGGGAAGGGGCTGGAGTGGGTCTCAGCTATTA |
| | | GTGGTAGTGGTGGTAGCACATACTACGCAGACTCCGTGA |
| | | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGTGC |
| | | TAGCACCAAGGGCCCATCGGTCTTCCCCCTGGCACCCTC |
| | | CTCCAAGAGCACCTCTGGGGGCACAGCGGCCCTGGGCT |
| | | GCCTGGTCAAGGACTACTTCCCCGAACCGGTGACGGTGT |
| | | CGTGGAACTCAGGCGCCCTGACCAGCGGCGTGCACACC |
| | | TTCCCGGCTGTCCTACAGTCCTCAGGACTCTACTCCCTC |
| | | AGCAGCGTGGTGACCGTGCCCTCCAGCAGCTTGGGCAC |
| | | CCAGACCTACATCTGCAACGTGAATCACAAGCCCAGCA |
| | | ACACCAAGGTGGACAAGAAAGTTGAGCCCAAATCTTGT |
| | | GACAAAACTCACACATGCCCACCGTGCCCAGCACCTGA |
| | | AGCTGCAGGGGGACCGTCAGTCTTCCTCTTCCCCCCAAA |
| | | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTGCACCCTGCCCCCATCCCGGGATGAGCTGACCAAGA |
| | | ACCAGGTCAGCCTCTCGTGCGCAGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCGTGAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAAA |
| 152 | nucleotide | GAAATCGTGTTAACGCAGTCTCCAGGCACCCTGTCTTTG |
| | sequence of DP47 | TCTCCAGGGGAAAGAGCCACCCTCTCTTGCAGGGCCAGT |
| | light chain | CAGAGTGTTAGCAGCAGCTACTTAGCCTGGTACCAGCAG |
| | | AAACCTGGCCAGGCTCCCAGGCTCCTCATCTATGGAGCA |
| | | TCCAGCAGGGCCACTGGCATCCCAGACAGGTTCAGTGG |
| | | CAGTGGATCCGGGACAGACTTCACTCTCACCATCAGCAG |
| | | ACTGGAGCCTGAAGATTTTGCAGTGTATTACTGTCAGCA |
| | | GTATGGTAGCTCACCGCTGACGTTCGGCCAGGGGACCA |
| | | AAGTGGAAATCAAACGTACGGTGGCTGCACCATCTGTCT |
| | | TCATCTTCCCGCCATCTGATGAGCAGTTGAAATCTGGAA |
| | | CTGCCTCTGTTGTGTGCCTGCTGAATAACTTCTATCCCAG |
| | | AGAGGCCAAAGTACAGTGGAAGGTGGATAACGCCCTCC |
| | | AATCGGGTAACTCCCAGGAGAGTGTCACAGAGCAGGAC |
| | | AGCAAGGACAGCACCTACAGCCTCAGCAGCACCCTGAC |
| | | GCTGAGCAAAGCAGACTACGAGAAACACAAAGTCTACG |
| | | CCTGCGAAGTCACCCATCAGGGCCTGAGCTCGCCCGTCA |
| | | CAAAGAGCTTCAACAGGGGAGAGTGT |
| 74 | Dimeric hu 4- | see Table 8 |
| | 1BBL (71-254) - | |
| | CL* Fc knob | |
| | chain | |
| 75 | Monomeric hu 4- | see Table 8 |
| | 1BBL (71-254) - | |
| | CH1* | |
| 153 | DP47 Fc hole | EVQLLESGGGLVQPGGSLRLSCAASGFTFSSYAMSWVRQA |
| | chain | PGKGLEWVSAISGSGGSTYYADSVKGRFTISRDNSKNTLYL |
| | | QMNSLRAEDTAVYYCAKGSGFDYWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVCTLPPSRDELTKNQVS |
| | | LSCAVKGFYPSDIAVEWESNGQPENNYKTTPPVLDSDGSFF |
| | | LVSKLTVDKSRWQQGNVFSCSVMHEALHNHYTQKSLSLS |
| | | PGK |
| 154 | DP47 light chain | EIVLTQSPGTLSLSPGERATLSCRASQSVSSSYLAWYQQKP |
| | | GQAPRLLIYGASSRATGIPDRFSGSGSGTDFTLTISRLEPEDF |
| | | AVYYCQQYGSSPLTFGQGTKVEIKRTVAAPSVFIFPPSDEQ |
| | | LKSGTASVVCLLNNFYPREAKVQWKVDNALQSGNSQESV |
| | | TEQDSKDSTYSLSSTLTLSKADYEKHKVYACEVTHQGLSS |
| | | PVTKSFNRGEC |

Table 19 shows the cDNA and amino acid sequences of the bivalent DP47-untargeted split trimeric 4-1BB ligand (71-254) Fc (kih) fusion molecule Control C.

TABLE 19
Sequences of bivalent DP47-untargeted human 4-1BB
ligand (71-254) containing Fc (kih) fusion molecule Control C

| SEQ ID | | |
|---|---|---|
| NO: | Description | Sequence |
| 155 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | Fc hole chain | ATTCACCTTTAGCAGTTATGCCATGAGCTGGGTCCGCCA |
| | fused to dimeric | GGCTCCAGGGAAGGGGCTGGAGTGGGTCTCAGCTATTA |
| | hu 4-1BBL (71- | GTGGTAGTGGTGGTAGCACATACTACGCAGACTCCGTGA |
| | 254) | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGTGC |
| | | TAGCACCAAGGGCCCCTCCGTGTTCCCCCTGGCCCCCAG |
| | | CAGCAAGAGCACCAGCGGCGGCACAGCCGCTCTGGGCT |
| | | GCCTGGTCAAGGACTACTTCCCCGAGCCCGTGACCGTGT |
| | | CCTGGAACAGCGGAGCCCTGACCTCCGGCGTGCACACC |
| | | TTCCCCGCCGTGCTGCAGAGTTCTGGCCTGTATAGCCTG |
| | | AGCAGCGTGGTCACCGTGCCTTCTAGCAGCCTGGGCACC |
| | | CAGACCTACATCTGCAACGTGAACCACAAGCCCAGCAA |
| | | CACCAAGGTGGACAAGAAGGTGGAGCCCAAGAGCTGCG |
| | | ACAAAACTCACACATGCCCACCGTGCCCAGCACCTGAA |
| | | GCTGCAGGGGGACCGTCAGTCTTCCTCTTCCCCCCAAAA |
| | | CCCAAGGACACCCTCATGATCTCCCGGACCCCTGAGGTC |
| | | ACATGCGTGGTGGTGGACGTGAGCCACGAAGACCCTGA |
| | | GGTCAAGTTCAACTGGTACGTGGACGGCGTGGAGGTGC |
| | | ATAATGCCAAGACAAAGCCGCGGGAGGAGCAGTACAAC |
| | | AGCACGTACCGTGTGGTCAGCGTCCTCACCGTCCTGCAC |
| | | CAGGACTGGCTGAATGGCAAGGAGTACAAGTGCAAGGT |
| | | CTCCAACAAAGCCCTCGGCGCCCCCATCGAGAAAACCA |
| | | TCTCCAAAGCCAAAGGGCAGCCCCGAGAACCACAGGTG |
| | | TGCACCCTGCCCCCATCCCGGGATGAGCTGACCAAGAA |
| | | CCAGGTCAGCCTCTCGTGCGCAGTCAAAGGCTTCTATCC |
| | | CAGCGACATCGCCGTGGAGTGGGAGAGCAATGGGCAGC |
| | | CGGAGAACAACTACAAGACCACGCCTCCCGTGCTGGAC |
| | | TCCGACGGCTCCTTCTTCCTCGTGAGCAAGCTCACCGTG |
| | | GACAAGAGCAGGTGGCAGCAGGGGAACGTCTTCTCATG |
| | | CTCCGTGATGCATGAGGCTCTGCACAACCACTACACGCA |
| | | GAAGAGCCTCTCCCTGTCTCCGGGTGGAGGCGGCGGAA |
| | | GCGGAGGAGGAGGATCCAGAGAGGGCCCTGAGCTGAGC |
| | | CCCGATGATCCTGCTGGACTGCTGGACCTGCGGCAGGGC |
| | | ATGTTTGCTCAGCTGGTGGCCCAGAACGTGCTGCTGATC |
| | | GATGGCCCCCTGTCCTGGTACAGCGATCCTGGACTGGCT |
| | | GGCGTGTCACTGACAGGCGGCCTGAGCTACAAAGAGGA |
| | | CACCAAAGAACTGGTGGTGGCCAAGGCCGGCGTGTACT |
| | | ACGTGTTCTTTCAGCTGGAACTGCGGAGAGTGGTGGCCG |
| | | GCGAAGGATCTGGCTCTGTGTCTCTGGCCCTGCATCTGC |
| | | AGCCTCTGAGAAGCGCTGCTGGCGCTGCAGCTCTGGCAC |
| | | TGACAGTGGATCTGCCTCCTGCCAGCTCCGAGGCCCGGA |
| | | ATAGCGCATTTGGGTTTCAAGGCAGGCTGCTGCACCTGT |
| | | CTGCCGGCCAGAGGCTGGGAGTGCATCTGCACACAGAG |
| | | GCCAGGGCTAGACACGCCTGGCAGCTGACACAGGGCGC |
| | | TACAGTGCTGGGCCTGTTCAGAGTGACCCCCGAGATTCC |
| | | AGCCGGCCTGCCTTCTCCAAGAAGCGAAGGCGGAGGCG |
| | | GATCTGGCGGCGGAGGATCTAGAGAGGGACCCGAACTG |
| | | TCCCCTGACGATCCAGCCGGGCTGCTGGATCTGAGACAG |
| | | GGAATGTTCGCCCAGCTGGTGGCTCAGAATGTGCTGCTG |
| | | ATTGACGGACCTCTGAGCTGGTACTCCGACCCAGGGCTG |
| | | GCAGGGGTGTCCCTGACTGGGGGACTGTCCTACAAAGA |
| | | AGATACAAAAGAACTGGTGGTGGCTAAAGCTGGGGTGT |
| | | ACTATGTGTTTTTTCAGCTGGAACTGAGGCGGGTGGTGG |
| | | CTGGGGAGGGCTCAGGATCTGTGTCCCTGGCTCTGCATC |
| | | TGCAGCCACTGCGCTCTGCTGCTGGCGCAGCTGCACTGG |
| | | CTCTGACTGTGGACCTGCCACCAGCCTCTAGCGAGGCCA |
| | | GAAACAGCGCCTTCGGGTTCCAAGGACGCCTGCTGCATC |
| | | TGAGCGCCGGACAGCGCCTGGGAGTGCATCTGCATACT |
| | | GAAGCCAGAGCCCGGCATGCTTGGCAGCTGACTCAGGG |
| | | GGCAACTGTGCTGGGACTGTTTCGCGTGACACCTGAGAT |
| | | CCCTGCCGGACTGCCAAGCCCTAGATCAGAA |
| 156 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | Fc knob chain | ATTCACCTTTAGCAGTTATGCCATGAGCTGGGTCCGCCA |
| | fused to | GGCTCCAGGGAAGGGGCTGGAGTGGGTCTCAGCTATTA |
| | monomeric hu 4- | GTGGTAGTGGTGGTAGCACATACTACGCAGACTCCGTGA |
| | 1BBL (71-254) | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGTGC |
| | | TAGCACCAAGGGCCCATCGGTCTTCCCCCTGGCACCCTC |
| | | CTCCAAGAGCACCTCTGGGGGCACAGCGGCCCTGGGCT |
| | | GCCTGGTCAAGGACTACTTCCCCGAACCGGTGACGGTGT |
| | | CGTGGAACTCAGGCGCCCTGACCAGCGGCGTGCACACC |
| | | TTCCCGGCTGTCCTACAGTCCTCAGGACTCTACTCCCTC |
| | | AGCAGCGTGGTGACCGTGCCCTCCAGCAGCTTGGGCAC |
| | | CCAGACCTACATCTGCAACGTGAATCACAAGCCCAGCA |
| | | ACACCAAGGTGGACAAGAAAGTTGAGCCCAAATCTTGT |
| | | GACAAAACTCACACATGCCCACCGTGCCCAGCACCTGA |
| | | AGCTGCAGGGGGACCGTCAGTCTTCCTCTTCCCCCCAAA |
| | | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTACACCCTGCCCCCCTGCAGAGATGAGCTGACCAAGA |
| | | ACCAGGTGTCCCTGTGGTGTCTGGTCAAGGGCTTCTACC |
| | | CCAGCGATATCGCCGTGGAGTGGGAGAGCAACGGCCAG |
| | | CCTGAGAACAACTACAAGACCACCCCCCCTGTGCTGGA |
| | | CAGCGACGGCAGCTTCTTCCTGTACTCCAAACTGACCGT |
| | | GGACAAGAGCCGGTGGCAGCAGGGCAACGTGTTCAGCT |
| | | GCAGCGTGATGCACGAGGCCCTGCACAACCACTACACC |
| | | CAGAAGTCCCTGAGCCTGAGCCCCGGCGGAGGCGGCGG |
| | | AAGCGGAGGAGGAGGATCCAGAGAGGGCCCTGAGCTGA |
| | | GCCCCGATGATCCTGCTGGACTGCTGGACCTGCGGCAGG |
| | | GCATGTTTGCTCAGCTGGTGGCCCAGAACGTGCTGCTGA |
| | | TCGATGGCCCCCTGTCCTGGTACAGCGATCCTGGACTGG |
| | | CTGGCGTGTCACTGACAGGCGGCCTGAGCTACAAAGAG |
| | | GACACCAAAGAACTGGTGGTGGCCAAGGCCGGCGTGTA |
| | | CTACGTGTTCTTTCAGCTGGAACTGCGGAGAGTGGTGGC |
| | | CGGCGAAGGATCTGGCTCTGTGTCTCTGGCCCTGCATCT |
| | | GCAGCCTCTGAGAAGCGCTGCTGGCGCTGCAGCTCTGGC |
| | | ACTGACAGTGGATCTGCCTCCTGCCAGCTCCGAGGCCCG |
| | | GAATAGCGCATTTGGGTTTCAAGGCAGGCTGCTGCACCT |
| | | GTCTGCCGGCCAGAGGCTGGGAGTGCATCTGCACACAG |
| | | AGGCCAGGGCTAGACACGCCTGGCAGCTGACACAGGGC |
| | | GCTACAGTGCTGGGCCTGTTCAGAGTGACCCCCGAGATT |
| | | CCAGCCGGCCTGCCTTCTCCAAGAAGCGAA |
| 152 | nucleotide | see Table 18 |
| | sequence of DP47 | |
| | light chain | |
| 157 | DP47 Fc hole | EVQLLESGGGLVQPGGSLRLSCAASGFTFSSYAMSWVRQA |
| | chain fused to | PGKGLEWVSAISGSGGSTYYADSVKGRFTISRDNSKNTLYL |
| | dimeric hu 4- | QMNSLRAEDTAVYYCAKGSGFDYWGQGTLVTVSSASTKG |
| | 1BBL (71-254) | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVCTLPPSRDELTKNQVS |
| | | LSCAVKGFYPSDIAVEWESNGQPENNYKTTPPVLDSDGSFF |
| | | LVSKLTVDKSRWQQGNVFSCSVMHEALHNHYTQKSLSLS |
| | | PGGGGGSGGGGSREGPELSPDDPAGLLDLRQGMFAQLVA |
| | | QNVLLIDGPLSWYSDPGLAGVSLTGGLSYKEDTKELVVAK |
| | | AGVYYVFFQLELRRVVAGEGSGSVSLALHLQPLRSAAGAA |
| | | ALALTVDLPPASSEARNSAFGFQGRLLHLSAGQRLGVHLH |
| | | TEARARHAWQLTQGATVLGLFRVTPEIPAGLPSPRSEGGG |
| | | GSGGGGSREGPELSPDDPAGLLDLRQGMFAQLVAQNVLLI |
| | | DGPLSWYSDPGLAGVSLTGGLSYKEDTKELVVAKAGVYY |
| | | VFFQLELRRVVAGEGSGSVSLALHLQPLRSAAGAAALALT |
| | | VDLPPASSEARNSAFGFQGRLLHLSAGQRLGVHLHTEARA |
| | | RHAWQLTQGATVLGLFRVTPEIPAGLPSPRSE |
| 158 | DP47 Fc knob | EVQLLESGGGLVQPGGSLRLSCAASGFTFSSYAMSWVRQA |
| | chain fused to | PGKGLEWVSAISGSGGSTYYADSVKGRFTISRDNSKNTLYL |
| | monomeric hu 4- | QMNSLRAEDTAVYYCAKGSGFDYWGQGTLVTVSSASTKG |
| | 1BBL (71-254) | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVYTLPPCRDELTKNQVS |
| | | LWCLVKGFYPSDIAVEWESNGQPENNYKTTPPVLDSDGSF |
| | | FLYSKLTVDKSRWQQGNVFSCSVMHEALHNHYTQKSLSL |
| | | SPGGGGGSGGGGSREGPELSPDDPAGLLDLRQGMFAQLVA |
| | | QNVLLIDGPLSWYSDPGLAGVSLTGGLSYKEDTKELVVAK |
| | | AGVYYVFFQLELRRVVAGEGSGSVSLALHLQPLRSAAGAA |
| | | ALALTVDLPPASSEARNSAFGFQGRLLHLSAGQRLGVHLH |
| | | TEARARHAWQLTQGATVLGLFRVTPEIPAGLPSPRSE |
| 154 | DP47 light chain | see Table 18 |

Table 20 shows, respectively, the cDNA and amino acid sequences of the monovalent DP47-untargeted split trimeric 4-1BB ligand (71-248) Fc (kih) fusion containing CH-CL cross with charged residues, control D.

TABLE 20
cDNA and amino acid sequences of monovalent DP47 untargeted split
trimeric human 4-1BB ligand (71-248) Fc (kih) fusion with CH-CL
cross and with charged residues (control D). * charged residues

| SEQ ID | | |
|---|---|---|
| NO: | Description | Sequence |
| 139 | nucleotide sequence of dimeric hu 4-1BBL | see Table 11 |
| | (71-248)-CL* Fc knob chain | |
| 140 | nucleotide sequence of monomeric hu 4- | see Table 11 |
| | 1BBL (71-248)-CH1* | |
| 151 | nucleotide sequence DP47 Fc hole chain | see Table 18 |
| 152 | nucleotide sequence DP47 light chain | see Table 18 |
| 78 | Dimeric hu 4-1BBL (71-254)-CL* Fc | see Table 11 |
| | knob chain | |
| 79 | Monomeric hu 4-1BBL (71-254)-CH1* | see Table 11 |
| 153 | DP47 Fc hole chain | see Table 18 |
| 154 | DP47 light chain | see Table 18 |

2.10 Preparation of Human IgG1 Antigen Binding Molecules as Controls

An additional control molecule (Control F) used in the assays was an untargeted DP47, germline control, human IgG1, containing the Pro329Gly, Leu234Ala and Leu235Ala mutations, to abrogate binding to Fc gamma receptors according to the method described in International Patent Appl. Publ. No. WO 2012/130831).

Table 21 shows the cDNA and amino acid sequences of the cDNA and amino acid sequences of the untargeted DP47 huIgG1 PGLALA (Control F).

TABLE 21
Sequences of untargeted DP47 huIgG1 (Control F)

| SEQ ID | | |
|---|---|---|
| NO: | Description | Sequence |
| 159 | nucleotide | GAGGTGCAATTGTTGGAGTCTGGGGGAGGCTTGGTACA |
| | sequence of DP47 | GCCTGGGGGGTCCCTGAGACTCTCCTGTGCAGCCTCCGG |
| | heavy chain (hu | ATTCACCTTTAGCAGTTATGCCATGAGCTGGGTCCGCCA |
| | IgG1 PGLALA) | GGCTCCAGGGAAGGGGCTGGAGTGGGTCTCAGCTATTA |
| | | GTGGTAGTGGTGGTAGCACATACTACGCAGACTCCGTGA |
| | | AGGGCCGGTTCACCATCTCCAGAGACAATTCCAAGAAC |
| | | ACGCTGTATCTGCAGATGAACAGCCTGAGAGCCGAGGA |
| | | CACGGCCGTATATTACTGTGCGAAAGGCAGCGGATTTGA |
| | | CTACTGGGGCCAAGGAACCCTGGTCACCGTCTCGAGTGC |
| | | TAGCACCAAGGGCCCATCGGTCTTCCCCCTGGCACCCTC |
| | | CTCCAAGAGCACCTCTGGGGGCACAGCGGCCCTGGGCT |
| | | GCCTGGTCAAGGACTACTTCCCCGAACCGGTGACGGTGT |
| | | CGTGGAACTCAGGCGCCCTGACCAGCGGCGTGCACACC |
| | | TTCCCGGCTGTCCTACAGTCCTCAGGACTCTACTCCCTC |
| | | AGCAGCGTGGTGACCGTGCCCTCCAGCAGCTTGGGCAC |
| | | CCAGACCTACATCTGCAACGTGAATCACAAGCCCAGCA |
| | | ACACCAAGGTGGACAAGAAAGTTGAGCCCAAATCTTGT |
| | | GACAAAACTCACACATGCCCACCGTGCCCAGCACCTGA |
| | | AGCTGCAGGGGGACCGTCAGTCTTCCTCTTCCCCCCAAA |
| | | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCGGCGCCCCCATCGAGAAAACC |
| | | ATCTCCAAAGCCAAAGGGCAGCCCCGAGAACCACAGGT |
| | | GTACACCCTGCCCCCATCCCGGGATGAGCTGACCAAGA |
| | | ACCAGGTCAGCCTGACCTGCCTGGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCTACAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAAA |
| 152 | nucleotide | see Table 18 |
| | sequence of DP47 | |
| | light chain | |
| 160 | DP47 heavy chain | EVQLLESGGGLVQPGGSLRLSCAASGFTFSSYAMSWVRQA |
| | (hu IgG1 | PGKGLEWVSAISGSGGSTYYADSVKGRFTISRDNSKNTLYL |
| | PGLALA) | QMNSLRAEDTAVYYCAKGSGFDYWGQGTLVTVSSASTKG |
| | | PSVFPLAPSSKSTSGGTAALGCLVKDYFPEPVTVSWNSGAL |
| | | TSGVHTFPAVLQSSGLYSLSSVVTVPSSSLGTQTYICNVNH |
| | | KPSNTKVDKKVEPKSCDKTHTCPPCPAPEAAGGPSVFLFPP |
| | | KPKDTLMISRTPEVTCVVVDVSHEDPEVKFNWYVDGVEV |
| | | HNAKTKPREEQYNSTYRVVSVLTVLHQDWLNGKEYKCKV |
| | | SNKALGAPIEKTISKAKGQPREPQVYTLPPSRDELTKNQVS |
| | | LTCLVKGFYPSDIAVEWESNGQPENNYKTTPPVLDSDGSFF |
| | | LYSKLTVDKSRWQQGNVFSCSVMHEALHNHYTQKSLSLS |
| | | PGK |
| 154 | DP47 light chain | see Table 18 |

Table 22 shows the cDNA and amino acid sequences of the anti-PD1 huIgG1 PGLALA (clone 0314), i.e. control M.

TABLE 22
cDNA and amino acid sequences of anti-PD1(0314) huIgG1
PGLALA (control M)

| SEQ ID | | |
|---|---|---|
| NO: | Description | Sequence |
| 161 | nucleotide sequence of | GAAGTGCAGCTGCTCGAAAGCGGCGGGGGAC |
| | PD1(0314) heavy chain | TGGTCCAGCCCGGCGGTTCCCTGAGGCTGTCT |
| | (huIgG1 PGLALA) | TGCGCCGCTTCAGGGTTCAGCTTTTCCTCTTAC |
| | | ACCATGAGTTGGGTCAGACAGGCACCTGGCAA |
| | | GGGACTGGAGTGGGTCGCCACAATCAGCGGTG |
| | | GCGGGCGCGACATTTATTACCCAGATTCCGTG |
| | | AAAGGACGGTTCACCATCTCTAGGGACAACTC |
| | | AAAGAATACTCTGTATTTGCAGATGAACAGCC |
| | | TGAGAGCTGAGGATACAGCAGTTTACTACTGT |
| | | GTGCTCCTGACCGGCCGCGTCTATTTTGCCCTT |
| | | GACTCCTGGGGACAAGGCACTCTGGTGACCGT |
| | | ATCTAGTGCTAGCACCAAGGGCCCATCGGTCT |
| | | TCCCCCTGGCACCCTCCTCCAAGAGCACCTCT |
| | | GGGGGCACAGCGGCCCTGGGCTGCCTGGTCAA |
| | | GGACTACTTCCCCGAACCGGTGACGGTGTCGT |
| | | GGAACTCAGGCGCCCTGACCAGCGGCGTGCAC |
| | | ACCTTCCCGGCTGTCCTACAGTCCTCAGGACTC |
| | | TACTCCCTCAGCAGCGTGGTGACCGTGCCCTC |
| | | CAGCAGCTTGGGCACCCAGACCTACATCTGCA |
| | | ACGTGAATCACAAGCCCAGCAACACCAAGGT |
| | | GGACAAGAAAGTTGAGCCCAAATCTTGTGACA |
| | | AAACTCACACATGCCCACCGTGCCCAGCACCT |
| | | GAAGCTGCAGGGGGACCGTCAGTCTTCCTCTT |
| | | CCCCCCAAAACCCAAGGACACCCTCATGATCT |
| | | CCCGGACCCCTGAGGTCACATGCGTGGTGGTG |
| | | GACGTGAGCCACGAAGACCCTGAGGTCAAGTT |
| | | CAACTGGTACGTGGACGGCGTGGAGGTGCATA |
| | | ATGCCAAGACAAAGCCGCGGGAGGAGCAGTA |
| | | CAACAGCACGTACCGTGTGGTCAGCGTCCTCA |
| | | CCGTCCTGCACCAGGACTGGCTGAATGGCAAG |
| | | GAGTACAAGTGCAAGGTCTCCAACAAAGCCCT |
| | | CGGCGCCCCCATCGAGAAAACCATCTCCAAAG |
| | | CCAAAGGGCAGCCCCGAGAACCACAGGTGTA |
| | | CACCCTGCCCCCATCCCGGGATGAGCTGACCA |
| | | AGAACCAGGTCAGCCTGACCTGCCTGGTCAAA |
| | | GGCTTCTATCCCAGCGACATCGCCGTGGAGTG |
| | | GGAGAGCAATGGGCAGCCGGAGAACAACTAC |
| | | AAGACCACGCCTCCCGTGCTGGACTCCGACGG |
| | | CTCCTTCTTCCTCTACAGCAAGCTCACCGTGGA |
| | | CAAGAGCAGGTGGCAGCAGGGGAACGTCTTCT |
| | | CATGCTCCGTGATGCATGAGGCTCTGCACAAC |
| | | CACTACACGCAGAAGAGCCTCTCCCTGTCTCC |
| | | GGGTAAA |
| 134 | nucleotide sequence of | see Table 8 |
| | anti-PD1(0314) light chain | |
| 162 | PD1(0314) heavy chain | EVQLLESGGGLVQPGGSLRLSCAASGFSFSSYTM |
| | (huIgG1 PGLALA) | SWVRQAPGKGLEWVATISGGGRDIYYPDSVKGR |
| | | FTISRDNSKNTLYLQMNSLRAEDTAVYYCVLLT |
| | | GRVYFALDSWGQGTLVTVSSASTKGPSVFPLAP |
| | | SSKSTSGGTAALGCLVKDYFPEPVTVSWNSGAL |
| | | TSGVHTFPAVLQSSGLYSLSSVVTVPSSSLGTQT |
| | | YICNVNHKPSNTKVDKKVEPKSCDKTHTCPPCP |
| | | APEAAGGPSVFLFPPKPKDTLMISRTPEVTCVVV |
| | | DVSHEDPEVKFNWYVDGVEVHNAKTKPREEQY |
| | | NSTYRVVSVLTVLHQDWLNGKEYKCKVSNKAL |
| | | GAPIEKTISKAKGQPREPQVYTLPPSRDELTKNQ |
| | | VSLTCLVKGFYPSDIAVEWESNGQPENNYKTTPP |
| | | VLDSDGSFFLYSKLTVDKSRWQQGNVFSCSVMH |
| | | EALHNHYTQKSLSLSPGK |
| 83 | anti-PD1(0314) light chain | see Table 8 |

### Example 3

### Production and Purification of Monovalent and Bivalent Anti-PD1 Targeted Split Trimeric 4-1BB Ligand Fc Fusion Antigen Binding Molecules and Control Molecules

The targeted and untargeted split trimeric 4-1BB ligand Fc (kih) fusion molecule encoding sequences were cloned into a plasmid vector, which drives expression of the insert from an MPSV promoter and contains a synthetic polyA sequence located at the 3′ end of the CDS. In addition, the vector contains an EBV OriP sequence for episomal maintenance of the plasmid.

The split trimeric 4-1BB ligand Fc (kih) fusion molecules were produced by co-transfecting HEK293-EBNA cells with the mammalian expression vectors using polyethylenimine. The cells were transfected with the corresponding expression vectors. For constructs 7.1, 7.2, 7.4, 7.6 and the corresponding control molecules control B and D, at a 1:1:1:1 ratio (“vector Fc hole chain”: “vector PD1 light chain”: “vector Fc knob chain”: “vector 4-1BBL light chain”). For constructs 7.3, 7.6, 7.11, 7.12, 7.13, 7.14 and the control C, at a 1:1:1 ratio (“vector Fc hole chain”: “vector Fc knob chain”: “vector anti-PD1 light chain”). Human IgGs, used as control in the assay, were produced as for the bispecific constructs (for transfection only a vector for light and a vector for heavy chain were used at a 1:1 ratio).

For production in 500 mL shake flasks, 300 million HEK293 EBNA cells were seeded 24 hours before transfection. For transfection cells were centrifuged for 10 minutes at 210×g, and the supernatant was replaced by 20 mL pre-warmed CD CHO medium. Expression vectors (200 g of total DNA) were mixed in 20 mL CD CHO medium. After addition of 540 μL PEI, the solution was vortexed for 15 seconds and incubated for 10 minutes at room temperature. Afterwards, cells were mixed with the DNA/PEI solution, transferred to a 500 mL shake flask and incubated for 3 hours at 37° C. in an incubator with a 5% CO₂ atmosphere. After the incubation, 160 mL of Excell medium supplemented with 6 mM L-Glutamine, 5 g/L PEPSOY and 1.2 mM valproic acid was added and cells were cultured for 24 hours. One day after transfection 12% Feed 7 and Glucose (final conc. 3 g/L) were added. After culturing for 7 days, the supernatant was collected by centrifugation for 30-40 minutes at least 400×g. The solution was sterile filtered (0.22 m filter), supplemented with sodium azide to a final concentration of 0.01% (w/v), and kept at 4° C.

Secreted proteins were purified from cell culture supernatants by affinity chromatography using Protein A, followed by size exclusion chromatography. For affinity chromatography, the supernatant was loaded on a MabSelect Sure column (CV=5-15 mL, resin from GE Healthcare) equilibrated with Sodium Phosphate (20 mM), Sodium Citrate (20 mM) buffer (pH 7.5). Unbound protein was removed by washing with at least 6 column volumes of the same buffer. The bound protein was eluted using either a linear gradient (20 CV) or a step elution (8 CV) with 20 mM sodium citrate, 100 mM Sodium chloride, 100 mM Glycine buffer (pH 2.5). For the linear gradient an additional 4 column volumes step elution was applied.

The pH of collected fractions was adjusted by adding 1/10 (v/v) of 0.5M sodium phosphate, pH8.0. The protein was concentrated prior to loading on a HiLoad Superdex 200 column (GE Healthcare) equilibrated with 20 mM Histidine, 140 mM sodium chloride, 0.01% (v/v) Tween20 solution of pH 6.0.

The protein concentration was determined by measuring the optical density (OD) at 280 nm, using a molar extinction coefficient calculated on the basis of the amino acid sequence. Purity and molecular weight of the targeted trimeric 4-1BB ligand Fc (kih) fusion was analyzed by SDS-PAGE in the presence and absence of a reducing agent (5 mM 1,4-dithiotreitol) and staining with Coomassie SimpleBlue™ SafeStain (Invitrogen USA) or CE-SDS using Caliper LabChip GXII (Perkin Elmer). The aggregate content of samples was analyzed using a TSKgel G3000 SW XL analytical size-exclusion column (Tosoh) equilibrated in 25 mM K₂HPO₄, 125 mM NaCl, 200 mM L-Arginine Monohydrocloride, 0.02% (w/v) NaN3, pH 6.7 running buffer at 25° C.

Table 23 summarizes the yield and final monomer content of the FAP (4B9) targeted and untargeted 4-1BB ligand trimer-containing Fc (kih) fusion antigen binding molecules and control molecules.

TABLE 23
Biochemical analysis of PD1 targeted and untargeted
4-1BB ligand trimer-containing Fc (kih) fusion antigen
binding molecules and control molecules

| | Monomer | |
|---|---|---|
| | [%] | Yield |
| Construct | (SEC) | [mg/l] |
| Construct 7.1 | 97.2 | 110.2 |
| Construct 7.3 | 96.6 | 8.9 |
| Construct 7.5 | 85 | 5.1 |
| Construct 7.6 | 100 | 16.5 |
| Control B | 99 | 15.4 |
| Control C | 98 | 12.6 |
| Control D | 99.5 | 25.9 |
| Control M | 99 | 34.4 |
| Control F | 100 | 50 |

### Example 4

### Simultaneous Binding of PD1-Targeted 4-1BB Split Trimeric Ligand Fc Fusion Antigen Binding Molecules by Surface Plasmon Resonance

4.1 Preparation, Purification and Characterization of Antigens for Surface Plasmon Resonance

Preparation of 4-1BB Fc (Kih) Fusion Molecule

A DNA sequence encoding the ectodomain of human 4-1BB (amino acids 24 to 186 of human 4-1BB according to Q07011, SEQ ID NO: 163) were subcloned in frame with the human IgG1 heavy chain CH2 and CH3 domains on the knob. An AcTEV protease cleavage site was introduced between an antigen ectodomain and the Fc of human IgG1. An Avi tag for directed biotinylation was introduced at the C-terminus of the antigen-Fc knob. Combination of the antigen-Fc knob chain containing the S354C/T366W mutations, with a Fc hole chain containing the Y349C/T366S/L368A/Y407V mutations allows generation of a heterodimer which includes a single copy of 4-1BB ectodomain containing chain, thus creating a monomeric form of Fc-linked antigen. Table 24 shows the cDNA and amino acid sequences of the antigen Fc-fusion construct.

TABLE 24
cDNA and Amino acid sequences of monomeric antigen Fc(kih)
fusion molecules

| SEQ ID | | |
|---|---|---|
| NO: | Antigen | Sequence |
| 164 | Nucleotide | GACAAAACTCACACATGCCCACCGTGCCCAGCACCTGA |
| | sequence of Fc | ACTCCTGGGGGGACCGTCAGTCTTCCTCTTCCCCCCAAA |
| | hole chain | ACCCAAGGACACCCTCATGATCTCCCGGACCCCTGAGGT |
| | | CACATGCGTGGTGGTGGACGTGAGCCACGAAGACCCTG |
| | | AGGTCAAGTTCAACTGGTACGTGGACGGCGTGGAGGTG |
| | | CATAATGCCAAGACAAAGCCGCGGGAGGAGCAGTACAA |
| | | CAGCACGTACCGTGTGGTCAGCGTCCTCACCGTCCTGCA |
| | | CCAGGACTGGCTGAATGGCAAGGAGTACAAGTGCAAGG |
| | | TCTCCAACAAAGCCCTCCCAGCCCCCATCGAGAAAACC |
| | | ATCTCCAAAGCCAAAGGGCAGCCCCGAGAACCACAGGT |
| | | GTGCACCCTGCCCCCATCCCGGGATGAGCTGACCAAGA |
| | | ACCAGGTCAGCCTCTCGTGCGCAGTCAAAGGCTTCTATC |
| | | CCAGCGACATCGCCGTGGAGTGGGAGAGCAATGGGCAG |
| | | CCGGAGAACAACTACAAGACCACGCCTCCCGTGCTGGA |
| | | CTCCGACGGCTCCTTCTTCCTCGTGAGCAAGCTCACCGT |
| | | GGACAAGAGCAGGTGGCAGCAGGGGAACGTCTTCTCAT |
| | | GCTCCGTGATGCATGAGGCTCTGCACAACCACTACACGC |
| | | AGAAGAGCCTCTCCCTGTCTCCGGGTAAA |
| 165 | Nucleotide | CTGCAGGACCCCTGCAGCAACTGCCCTGCCGGCACCTTC |
| | sequence of | TGCGACAACAACCGGAACCAGATCTGCAGCCCCTGCCC |
| | human 4-1BB | CCCCAACAGCTTCAGCTCTGCCGGCGGACAGCGGACCT |
| | antigen Fc knob | GCGACATCTGCAGACAGTGCAAGGGCGTGTTCAGAACC |
| | chain | CGGAAAGAGTGCAGCAGCACCAGCAACGCCGAGTGCGA |
| | | CTGCACCCCCGGCTTCCATTGTCTGGGAGCCGGCTGCAG |
| | | CATGTGCGAGCAGGACTGCAAGCAGGGCCAGGAACTGA |
| | | CCAAGAAGGGCTGCAAGGACTGCTGCTTCGGCACCTTC |
| | | AACGACCAGAAGCGGGGCATCTGCCGGCCCTGGACCAA |
| | | CTGTAGCCTGGACGGCAAGAGCGTGCTGGTCAACGGCA |
| | | CCAAAGAACGGGACGTCGTGTGCGGCCCCAGCCCTGCT |
| | | GATCTGTCTCCTGGGGCCAGCAGCGTGACCCCTCCTGCC |
| | | CCTGCCAGAGAGCCTGGCCACTCTCCTCAGGTCGACGAA |
| | | CAGTTATATTTTCAGGGCGGCTCACCCAAATCTGCAGAC |
| | | AAAACTCACACATGCCCACCGTGCCCAGCACCTGAACTC |
| | | CTGGGGGGACCGTCAGTCTTCCTCTTCCCCCCAAAACCC |
| | | AAGGACACCCTCATGATCTCCCGGACCCCTGAGGTCACA |
| | | TGCGTGGTGGTGGACGTGAGCCACGAAGACCCTGAGGT |
| | | CAAGTTCAACTGGTACGTGGACGGCGTGGAGGTGCATA |
| | | ATGCCAAGACAAAGCCGCGGGAGGAGCAGTACAACAGC |
| | | ACGTACCGTGTGGTCAGCGTCCTCACCGTCCTGCACCAG |
| | | GACTGGCTGAATGGCAAGGAGTACAAGTGCAAGGTCTC |
| | | CAACAAAGCCCTCCCAGCCCCCATCGAGAAAACCATCT |
| | | CCAAAGCCAAAGGGCAGCCCCGAGAACCACAGGTGTAC |
| | | ACCCTGCCCCCATGCCGGGATGAGCTGACCAAGAACCA |
| | | GGTCAGCCTGTGGTGCCTGGTCAAAGGCTTCTATCCCAG |
| | | CGACATCGCCGTGGAGTGGGAGAGCAATGGGCAGCCGG |
| | | AGAACAACTACAAGACCACGCCTCCCGTGCTGGACTCC |
| | | GACGGCTCCTTCTTCCTCTACAGCAAGCTCACCGTGGAC |
| | | AAGAGCAGGTGGCAGCAGGGGAACGTCTTCTCATGCTC |
| | | CGTGATGCATGAGGCTCTGCACAACCACTACACGCAGA |
| | | AGAGCCTCTCCCTGTCTCCGGGTAAATCCGGAGGCCTGA |
| | | ACGACATCTTCGAGGCCCAGAAGATTGAATGGCACGAG |
| 166 | Fc hole chain | DKTHTCPPCPAPELLGGPSVFLFPPKPKDTLMISRTPEVTCV |
| | | VVDVSHEDPEVKFNWYVDGVEVHNAKTKPREEQYNSTYR |
| | | VVSVLTVLHQDWLNGKEYKCKVSNKALPAPIEKTISKAKG |
| | | QPREPQVCTLPPSRDELTKNQVSLSCAVKGFYPSDIAVEWE |
| | | SNGQPENNYKTTPPVLDSDGSFFLVSKLTVDKSRWQQGNV |
| | | FSCSVMHEALHNHYTQKSLSLSPGK |
| 167 | human 4-1BB | LQDPCSNCPAGTFCDNNRNQICSPCPPNSFSSAGGQRTCDI |
| | antigen Fc knob | CRQCKGVFRTRKECSSTSNAECDCTPGFHCLGAGCSMCEQ |
| | chain | DCKQGQELTKKGCKDCCFGTFNDQKRGICRPWTNCSLDG |
| | | KSVLVNGTKERDVVCGPSPADLSPGASSVTPPAPAREPGHS |
| | | PQVDEQLYFQGGSPKSADKTHTCPPCPAPELLGGPSVFLFP |
| | | PKPKDTLMISRTPEVTCVVVDVSHEDPEVKFNWYVDGVE |
| | | VHNAKTKPREEQYNSTYRVVSVLTVLHQDWLNGKEYKCK |
| | | VSNKALPAPIEKTISKAKGQPREPQVYTLPPCRDELTKNQV |
| | | SLWCLVKGFYPSDIAVEWESNGQPENNYKTTPPVLDSDGS |
| | | FFLYSKLTVDKSRWQQGNVFSCSVMHEALHNHYTQKSLS |
| | | LSPGKSGGLNDIFEAQKIEWHE |

All 4-1BB-Fc-fusion molecule encoding sequences were cloned into a plasmid vector, which drives expression of the insert from an MPSV promoter and contains a synthetic polyA signal sequence located at the 3′ end of the CDS. In addition, the vector contains an EBV OriP sequence for episomal maintenance of the plasmid.

For preparation of the biotinylated monomeric antigen/Fc fusion molecule, exponentially growing suspension HEK293 EBNA cells were co-transfected with three vectors encoding the two components of fusion protein (knob and hole chains) as well as BirA, an enzyme necessary for the biotinylation reaction. The corresponding vectors were used at a 2:1:0.05 ratio (“antigen ECD-AcTEV-Fc knob”:“Fc hole”:“BirA”).

For protein production in 500 ml shake flasks, 400 million HEK293 EBNA cells were seeded 24 hours before transfection. For transfection cells were centrifuged for 5 minutes at 210 g, and the supernatant was replaced by pre-warmed CD CHO medium. Expression vectors were resuspended in 20 mL of CD CHO medium containing 200 μg of vector DNA. After addition of 540 μL of polyethylenimine (PEI), the solution was vortexed for 15 seconds and incubated for 10 minutes at room temperature. Afterwards, cells were mixed with the DNA/PEI solution, transferred to a 500 mL shake flask and incubated for 3 hours at 37° C. in an incubator with a 5% CO2 atmosphere. After the incubation, 160 mL of F17 medium was added and cells were cultured for 24 hours. One day after transfection, 1 mM valproic acid and 7% Feed 1 with supplements were added to the culture. After 7 days of culturing, the cell supernatant was collected by spinning down cells for 15 min at 210 g. The solution was sterile filtered (0.22 μm filter), supplemented with sodium azide to a final concentration of 0.01% (w/v), and kept at 4° C.

Secreted proteins were purified from cell culture supernatants by affinity chromatography using Protein A, followed by size exclusion chromatography. For affinity chromatography, the supernatant was loaded on a HiTrap ProteinA HP column (CV=5 mL, GE Healthcare) equilibrated with 40 mL 20 mM sodium phosphate, 20 mM sodium citrate pH 7.5. Unbound protein was removed by washing with at least 10 column volumes of 20 mM sodium phosphate, 20 mM sodium citrate, 0.5 M sodium chloride containing buffer (pH 7.5). The bound protein was eluted using a linear pH-gradient of sodium chloride (from 0 to 500 mM) created over 20 column volumes of 20 mM sodium citrate, 0.01% (v/v) Tween-20, pH 3.0. The column was then washed with 10 column volumes of 20 mM sodium citrate, 500 mM sodium chloride, 0.01% (v/v) Tween-20, pH 3.0.

The pH of collected fractions was adjusted by adding 1/40 (v/v) of 2M Tris, pH8.0. The protein was concentrated and filtered prior to loading on a HiLoad Superdex 200 column (GE Healthcare) equilibrated with 2 mM MOPS, 150 mM sodium chloride, 0.02% (w/v) sodium azide solution of pH 7.4.

For affinity determination to the human receptor, the ectodomain of human 4-1BB was also subcloned in frame with an avi (GLNDIFEAQKIEWHE, SEQ ID NO: 168) and a hexahistidine tag.

Protein production was performed as described above for the Fc-fusion protein. Secreted proteins were purified from cell culture supernatants by chelating chromatography, followed by size exclusion chromatography. The first chromatographic step was performed on a NiNTA Superflow Cartridge (5 ml, Qiagen) equilibrated in 20 mM sodium phosphate, 500 nM sodium chloride, pH7.4. Elution was performed by applying a gradient over 12 column volume from 5% to 45% of elution buffer (20 mM sodium phosphate, 500 nM sodium chloride, 500 mM Imidazole, pH7.4). The protein was concentrated and filtered prior to loading on a HiLoad Superdex 75 column (GE Healthcare) equilibrated with 2 mM MOPS, 150 mM sodium chloride, 0.02% (w/v) sodium azide solution of pH 7.4 (Table 25).

TABLE 25
Sequences of monomeric human 4-1BB His molecule

| SEQ ID | | |
|---|---|---|
| NO: | antigen | Sequence |
| 169 | nucleotide | CTGCAGGACCCCTGCAGCAACTGCCCTGCCGGCACCTTCTG |
| | sequence | CGACAACAACCGGAACCAGATCTGCAGCCCCTGCCCCCCC |
| | of human | AACAGCTTCAGCTCTGCCGGCGGACAGCGGACCTGCGACA |
| | 4-1BB His | TCTGCAGACAGTGCAAGGGCGTGTTCAGAACCCGGAAAGA |
| | | GTGCAGCAGCACCAGCAACGCCGAGTGCGACTGCACCCCC |
| | | GGCTTCCATTGTCTGGGAGCCGGCTGCAGCATGTGCGAGC |
| | | AGGACTGCAAGCAGGGCCAGGAACTGACCAAGAAGGGCT |
| | | GCAAGGACTGCTGCTTCGGCACCTTCAACGACCAGAAGCG |
| | | GGGCATCTGCCGGCCCTGGACCAACTGTAGCCTGGACGGC |
| | | AAGAGCGTGCTGGTCAACGGCACCAAAGAACGGGACGTCG |
| | | TGTGCGGCCCCAGCCCTGCTGATCTGTCTCCTGGGGCCAGC |
| | | AGCGTGACCCCTCCTGCCCCTGCCAGAGAGCCTGGCCACTC |
| | | TCCTCAGGTCGACGAACAGTTATATTTTCAGGGCGGCTCAG |
| | | GCCTGAACGACATCTTCGAGGCCCAGAAGATCGAGTGGCA |
| | | CGAGGCTCGAGCTCACCACCATCACCATCAC |
| 170 | human 4- | LQDPCSNCPAGTFCDNNRNQICSPCPPNSFSSAGGQRTCDICR |
| | 1BB His | QCKGVFRTRKECSSTSNAECDCTPGFHCLGAGCSMCEQDCK |
| | | QGQELTKKGCKDCCFGTFNDQKRGICRPWTNCSLDGKSVLV |
| | | NGTKERDVVCGPSPADLSPGASSVTPPAPAREPGHSPQVDEQL |
| | | YFQGGSGLNDIFEAQKIEWHEARAHHHHHH |

Human PD1 Fc fusion antigen was bought from R&D systems (Cat No 1086-PD-050).

4.2 Determination of Simultaneous Binding of Targeted C-Terminal 4-1BB Split Trimeric Ligand Fc Fusion Antigen Binding Molecules by Surface Plasmon Resonance

The capacity of binding simultaneously to human 4-1BB Fc(kih) and human PD1, was assessed by surface plasmon resonance (SPR). All SPR experiments were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany).

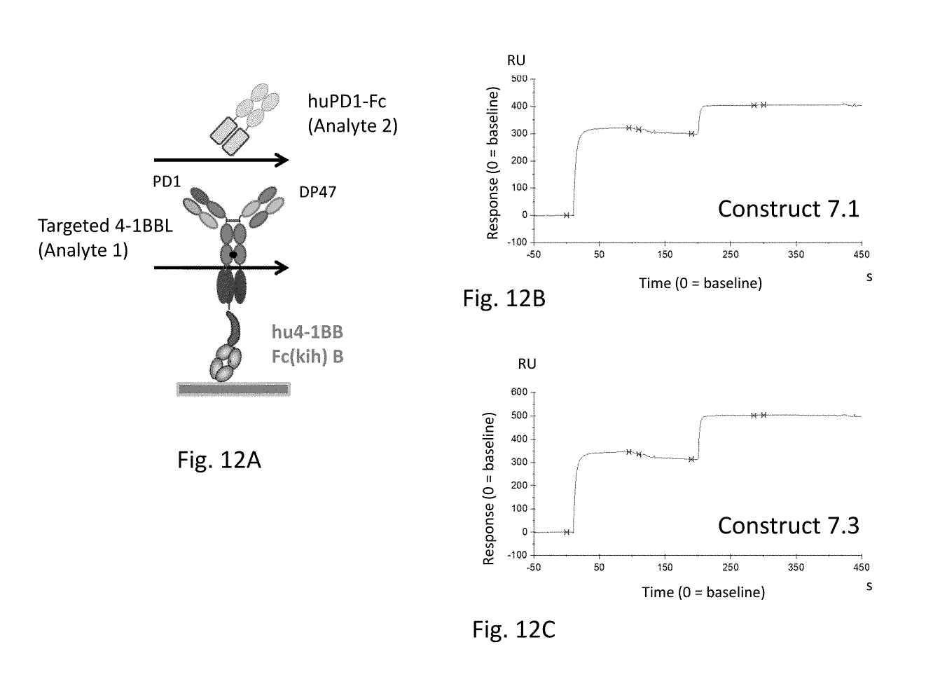

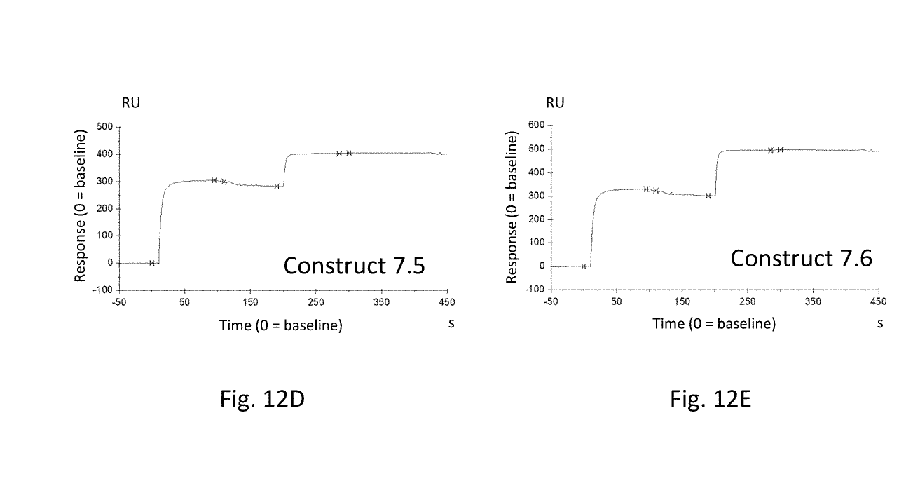

Biotinylated human 4-1BB Fc (kih) was directly coupled to a flow cell of a streptavidin (SA) sensor chip. Immobilization levels up to 200 resonance units (RU) were used. The targeted trimeric C-terminal 4-1BB ligand Fc (kih) fusion molecules were passed at a concentration range of 200 nM with a flow of 30 μL/minute through the flow cells over 90 seconds and dissociation was set to zero sec. Human PD1-Fc (R&D systems) was injected as second analyte with a flow of 30 μL/minute through the flow cells over 90 seconds at a concentration of 500 nM (FIG. 12A). The dissociation was monitored for 120 sec. Bulk refractive index differences were corrected for by subtracting the response obtained in a reference flow cell, where no protein was immobilized. All bispecific constructs could bind simultaneously human 4-1BB and human PD1 (FIG. 12B, FIG. 12C, FIG. 12D, and FIG. 12E).

### Example 5

### Functional Characterization of the PD1 Targeted 4-1BB Ligand Trimer-Containing Fc Fusion Antigen Binding Molecules

5.1. Binding on Fresh and Activated Human PMBCs of the PD1 Targeted 4-1BB Ligand Trimer-Containing Fc (Kih) Fusion Antigen Binding Molecules

Buffy coats were obtained from the Zurich blood donation center. To isolate fresh peripheral blood mononuclear cells (PBMCs) the buffy coat was diluted with the same volume of DPBS (Gibco by Life Technologies, Cat. No. 14190 326). 50 mL polypropylene centrifuge tubes (TPP, Cat.-No. 91050) were supplied with 15 mL Histopaque 1077 (SIGMA Life Science, Cat.-No. 10771, polysucrose and sodium diatrizoate, adjusted to a density of 1.077 g/mL) and the diluted buffy coat contents were layered above the Histopaque 1077. The tubes were centrifuged for 30 min at 450×g. PBMCs were then collected from the interface, washed three times with DPBS and resuspended in T cell medium consisting of RPMI 1640 medium (Gibco by Life Technology, Cat. No. 42401-042) supplied with 10% (v/v) Fetal Bovine Serum (FBS, US-origin, PAN biotech, P30-2009, Lot P150307GI, gamma irradiated mycoplasma free, heat inactivated 35 min 56° C.), 1% (v/v) GlutaMAX I (GIBCO by Life Technologies, Cat. No. 35050 038), 1 mM Sodium-Pyruvat (SIGMA, Cat. No. S8636), 1% (v/v) MEM non-essential amino acids (SIGMA, Cat.-No. M7145) and 50 tM 3-Mercaptoethanol (SIGMA, M3148).

PBMCs were used directly after isolation (binding on fresh human PBMCs) or they were stimulated to induce 4-1BB expression on the cell surface of T cells (binding on activated human PBMCs). To induce 4-1BB upregulation on human T cells, PBMCs were cultured for 3 days in RPMI 1640 (GIBCO by life technologies, Cat.-No. 42401-042) supplied with 10% Fetal Bovine Serum (FBS, US-origin, PAN biotech, P30-2009, Lot P150307GI, gamma irradiated mycoplasma free, heat inactivated 35 min 56° C.), 1% GlutaMAX-I (GIBCO by life technologies, Cat.-No. 35050-038), 200 U/mL Proleukin (Novartis Pharma Schweiz AG, CHCLB-P-476-700-10340, Lot AA4493BAL) and 2 ug/mL PHA-L (SIGMA, Cat.-No. L2769) at a concentration of 1×10⁶ cells/mL. After three days pre-activated human PBMCs were harvested, resupended in RPMI 1640 supplied with 10% FBS, 1 mM Sodium-Pyruvat (SIGMA, Cat.-No. S8636), 1% Gluta-MAX-I, 1% MEM-non essential amino acid Solution (SIGMA, Cat.-No. M7145) and 50 μM beta-Mercaptoethanol (Sigma M3148) to a final concentration of 1×10⁶ cells/mL. Afterwards cells were seeded in 6-well tissue culture plates (TTP, Cat.-No. 92006) which had been coated overnight at 4° C. with 10 ug/mL anti-human CD3 (clone OKT3, Mouse IgG2a, BioLegend, Cat.-No. 317315) and 2 ug/mL anti-human CD28 (clone CD28.2, Mouse IgG1, BioLegend, Cat.-No. 302923). Cells were further incubated for 2 days at 37° C. and 5% CO₂ For the binding assay 0.1×10⁶ fresh or activated PBMCs were added to each well of a round-bottom suspension cell 96-well plates (greiner bio-one, cellstar, Cat. No. 650185). Plates were centrifuged 5 minutes with 350×g and at 4° C. and supernatant were discarded. Afterwards cells were stained in 100 μL/well DPBS containing 1:5000 diluted fixable viability dye eF660 (eBioscience, Cat.-No. 65-0864-18) for 30 minutes at 4° C. in the dark. Cells were washed once with 200 μL ice cold cold DPBS buffer. Next, 50 μL/well of 4° C. cold FACS buffer (DPBS supplied with 2% FBS, 5 mM EDTA pH8 (Amresco, Cat. No. E177) and 7.5 mM Sodium azide (Sigma-Aldrich, Cat.-No. S2002)) containing titrated concentrations of construct 7.1, construct 7.3, construct 7.5 and construct 7.6 and as controls control B, control C, control D, control M and control F were added and cells were incubated for 60 minutes at 4° C., washed three times with 200 μL/well 4° C. FACS buffer. Cells were further resuspended in 50 pμL/well of 4° C. cold FACS buffer containing 0.67 μg/mL anti-human CD45-AF488 (clone HI30, mouse igG1k, BioLegend, Cat.-No. 304019), 0.33 μg/mL anti-human CD8a-BV510 (clone SK1, mouse IgG1k, BioLegend, Cat.-No. 344732), 0.23 μg/mL anti-human CD4-BV421 (clone OKT4, mouse IgG2bK, BioLegend, Cat.-No. 317434), 0.67 μg/mL anti-human CD3-PerCP-Cy5.5 (clone UCHT1, mouse IgG1k, BioLegend, Cat.-No. 300430), 0.67 μg/mL anti-human CD19-PE/Cy7 (clone HIB19, mouse IgG1k, BioLegend, Cat.-No. 302216), 5 μg/mL PE-conjugated AffiniPure anti-human IgG F(ab′)2-fragment-specific goat F(ab′)2 fragment (Jackson ImmunoResearch, Cat. No. 109 116 098) and incubated for 30 min at 4° C. Cells were washed twice with 200 μL/well 4° C. FACS buffer and then fixed with DPBS supplied with 1% Formaldehyde (Sigma, HT501320-9.5L). Cells were resuspended in 100 μL/well FACS-buffer and acquired using the MACS Quant Analyzer 10 (Miltenyi Biotech). Data was analyzed using FlowJo V10 (FlowJo, LLC) and GraphPad Prism 6.04 (GraphPad Software, Inc)

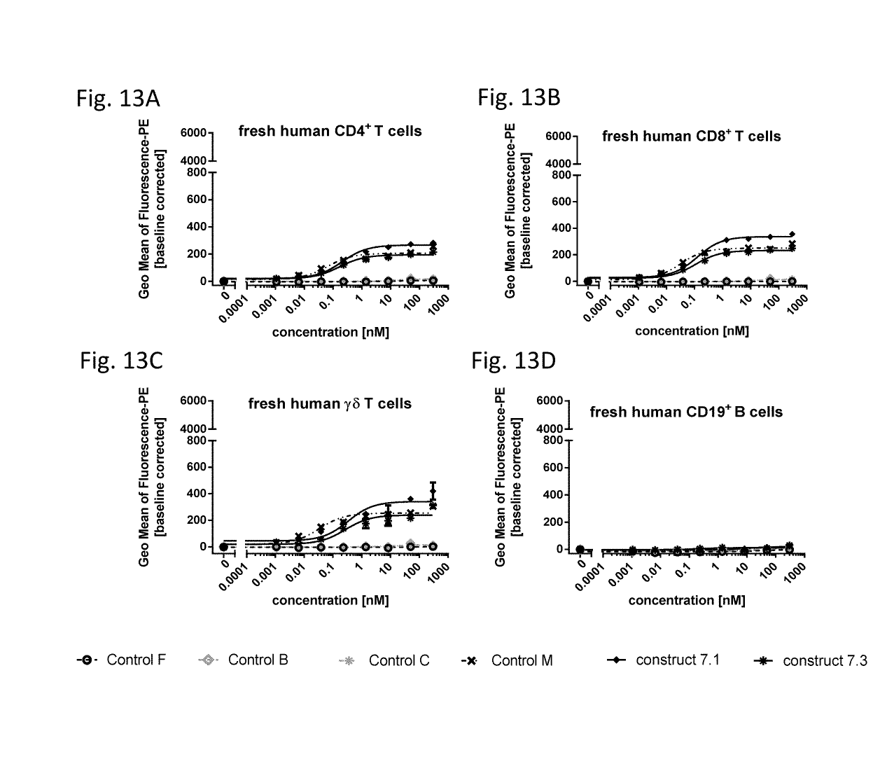

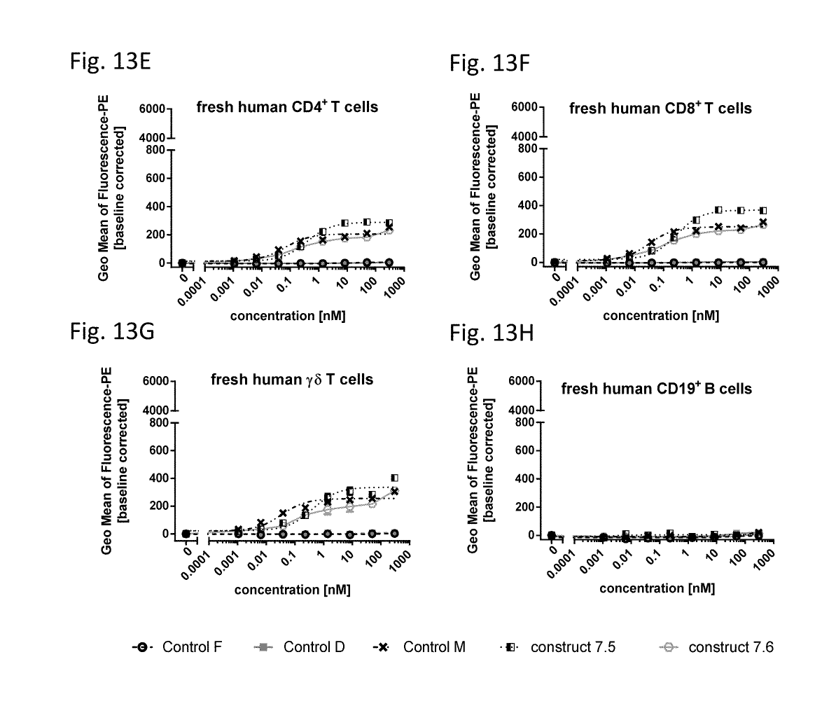

Gates were set on living CD45⁺ CD3⁺ CD19^{neg} CD8^{neg} CD4⁺ T cells, CD45⁺ CD3⁺ CD19 neg CD4^{neg} CD8⁺ T cells, CD45⁺ CD3⁺ CD4^{neg} CD8^{neg} γδ T cells and CD45⁺ CD3^{neg} CD19⁺ B cells and geo means of fluorescence intensity of PE-conjugated AffiniPure anti-human IgG IgG Fcγ-fragment-specific goat F(ab′)2 fragment were blotted against the titrated concentration of PD1-targeted or DP47-untargeted 4-1BB split trimeric ligand Fc fusion antigen binding molecules. As shown in FIG. 13A, FIG. 13B, FIG. 13C, FIG. 13D, FIG. 13E, FIG. 13F, FIG. 13G, and FIG. 13H, on resting fresh isolated human PBMCs from healthy donors only the PD1 targeted 4-1BB split trimeric ligand Fc fusion antigen binding molecules (construct 7.1., construct 7.3, construct 7.5 and construct 7.6) and the PD1 targeted human IgG control M bound to CD3⁺ CD4⁺, CD3⁺ CD8⁺ and CD3⁺ CD4^{neg} CD8^{neg} γδ T cells. Whereas the DP47-untargeted 4-1BB split trimeric ligand Fc fusion antigen binding molecules (control B, C and D) did not bind to any freshly isolated PBMCs. Therefore on freshly isolated human PBMCs the majority of cells do not express 4-1BB whereas a population of T cells expresses a detectable amount of PD1. In Table 26 the EC₅₀ values are summarized for the binding curves to freshly isolated human PBMCs.

TABLE 26
EC₅₀ values of binding curves to freshly
isolated resting human PBMCs

| | Control | Construct | Construct | Construct | Construct |
|---|---|---|---|---|---|
| | M | 7.1 | 7.3 | 7.5 | 7.6 |
| EC₅₀ [nM] on | 0.03 | 0.13 | 0.11 | 0.28 | 0.11 |
| activated | | | | | |
| CD4 T cells | | | | | |
| EC₅₀ [nM] on | 0.07 | 0.21 | 0.17 | 0.38 | 0.19 |
| activated | | | | | |
| CD8 T cells | | | | | |
| EC₅₀ [nM] on | 0.03 | 0.34 | 0.13 | 0.42 | 0.27 |
| activated | | | | | |
| γδ T cells | | | | | |

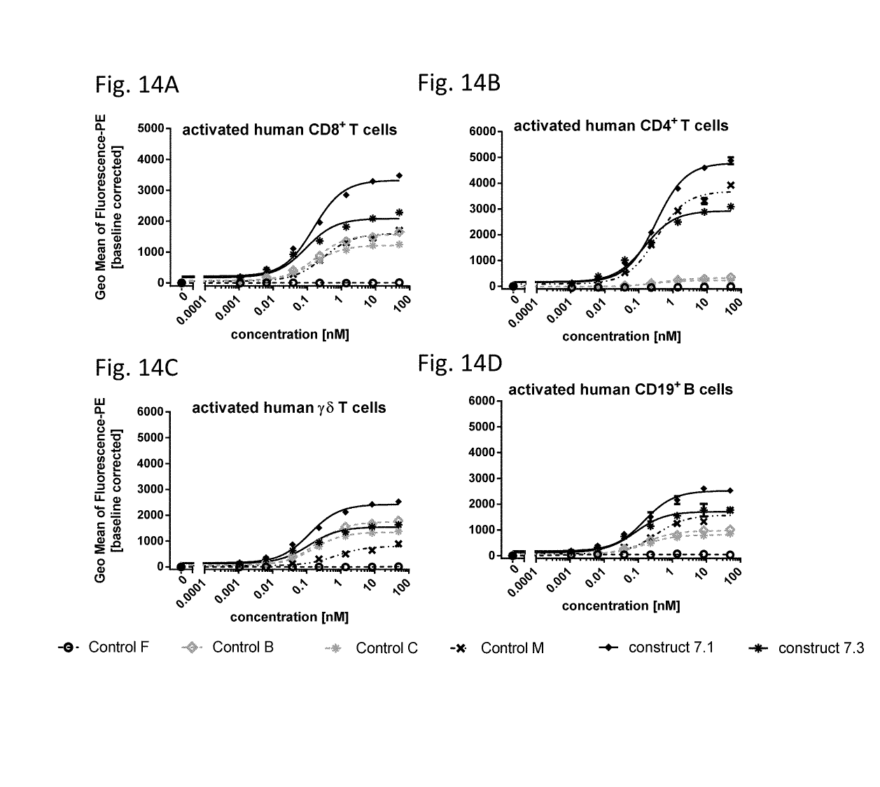

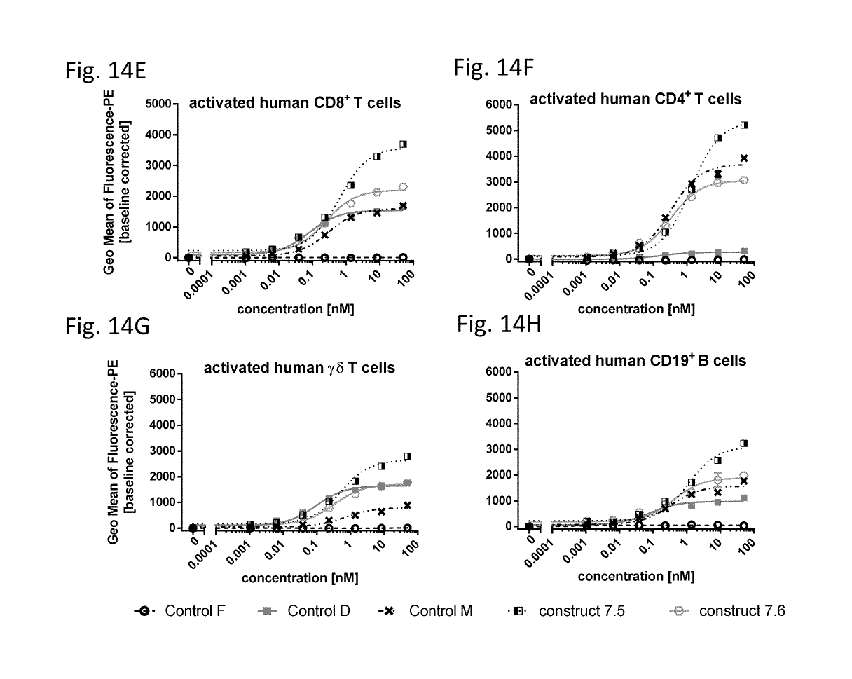

As shown in FIG. 14A, FIG. 14B, FIG. 14C, FIG. 14D, FIG. 14E, FIG. 14F, FIG. 14G, and FIG. 14H, after activation living CD45⁺ CD3⁺ CD19^{neg} CD8^{neg} CD4⁺ T cells, CD45⁺ CD3⁺ CD19^{neg} CD4^{neg} CD8⁺ T cells, CD45⁺ CD3⁺ CD4neg CD8^{neg} γδ T cells and CD45⁺ CD3^{neg} CD19+B cells express human 4-1BB and human PD1. The only PD1 binding molecules (control M) or only human 4-1BB binding molecules (controls B, control C and control D) proof thereby, that PD1 is highly upregulated on activated CD4⁺ T cells, CD8⁺ T cells, γδ T cells and B cells, whereas 4-1BB is mainly upregulated on CD8⁺ T cells, γδ T cells and B cells and to a lesser extent on CD4⁺ T cells. PD1-targeted 4-1BB split trimeric ligand Fc fusion antigen binding molecules (construct 7.1., construct 7.3, construct 7.5 and construct 7.6) bind to PD1 and human 4-1BB and this is reflected by higher geo means of fluorescence intensity compared to the fitting controls. In Table 27 the EC₅₀ values are summarized for the binding curves to activated human PBMCs.

TABLE 27
EC₅₀ values of binding curves to activated human PBMCs

| | Control | Control | Control | Control | Construct | Construct | Construct | Construct |
|---|---|---|---|---|---|---|---|---|
| | B | C | D | M | 7.1 | 7.3 | 7.5 | 7.6 |
| EC₅₀ [nM] on | 0.19 | 0.15 | 0.08 | 0.30 | 0.15 | 0.09 | 0.62 | 0.25 |
| activated CD8 T cells | | | | | | | | |
| EC₅₀ [nM] on | 0.43 | 0.25 | 0.15 | 0.33 | 0.32 | 0.15 | 1.32 | 0.33 |
| activated CD4 T cells | | | | | | | | |
| EC₅₀ [nM] on | 0.20 | 0.16 | 0.09 | 0.71 | 0.12 | 0.12 | 0.52 | 0.32 |
| activated γδ T cells | | | | | | | | |
| EC₅₀ [nM] on | 0.15 | 0.15 | 0.08 | 0.33 | 0.15 | 0.08 | 1.13 | 0.40 |
| activated CD19 cells | | | | | | | | |

5.2 Binding of Human PD1 and Human 4-1BB Transfected Tumor Cells

For binding assays on PD1 and 4-1BB expressing tumor cells two genetically modified tumor cells were used. The generation of human expressing HeLa-hu4-1BB-NFκB-luc clone 26 cells will be described in Example 5.3.1. For human PD1 expressing tumor cell lines, CHO-k1 cells were transfected with human PD1 as following: parental adherent CHO-K1 cell line (ATCC CCL-61) was stably transfected with plasmid 15311_hPD1-fl_pUC_Neo coding for full-length human PD1 and selection with G418 (Neomycin resistance marker on plasmid).

0.1×10⁶ tumor cells resuspended in DPBS (Gibco by Life Technologies, Cat. No. 14190 326) were added to each well of a round-bottom suspension cell 96-well plates (greiner bio-one, cellstar, Cat. No. 650185). Cells were washed once with 200 μL DPBS. Cells were resuspended in 100 μL/well of 4° C. cold DPBS buffer containing 1:5000 diluted Fixable Viability Dye eFluor 660 (eBioscience, Cat. No. 65-0864-18) and plates were incubated for 30 minutes at 4° C. Cells were washed once with 200 μL/well 4° C. cold DPBS buffer and resuspended in 50 μL/well of 4° C. cold FACS buffer (DPBS supplied with 2% FBS, 5 mM EDTA pH8 (Amresco, Cat. No. E177) and 7.5 mM Sodium azide (Sigma-Aldrich S2002)) containing PD1-targeted 4-1BB split trimeric ligand Fc fusion antigen binding molecules at a series of concentrations, followed by incubation for 1 hour at 4° C. After extensive washing, cells were further stained with 50 μL/well of 4° C. cold FACS buffer containing 5 μg/mL PE-conjugated AffiniPure anti-human IgG F(ab′)2-fragment-specific goat F(ab′)2 fragment (Jackson ImmunoResearch, Cat. No. 109 116 098) for 30 minutes at 4° C. Cells were washed twice with 200 μL/well 4° C. FACS buffer and cells were fixed in 50 μL/well DPBS containing 1% Formaldehyde (Sigma, HT501320-9.5L). Cells were resuspended in 100 μL/well FACS-buffer and acquired using the MACS Quant Analyzer 10 (Miltenyi Biotech). Data was analyzed using FlowJo V10 (FlowJo, LLC) and GraphPad Prism 6.04 (GraphPad Software, Inc).

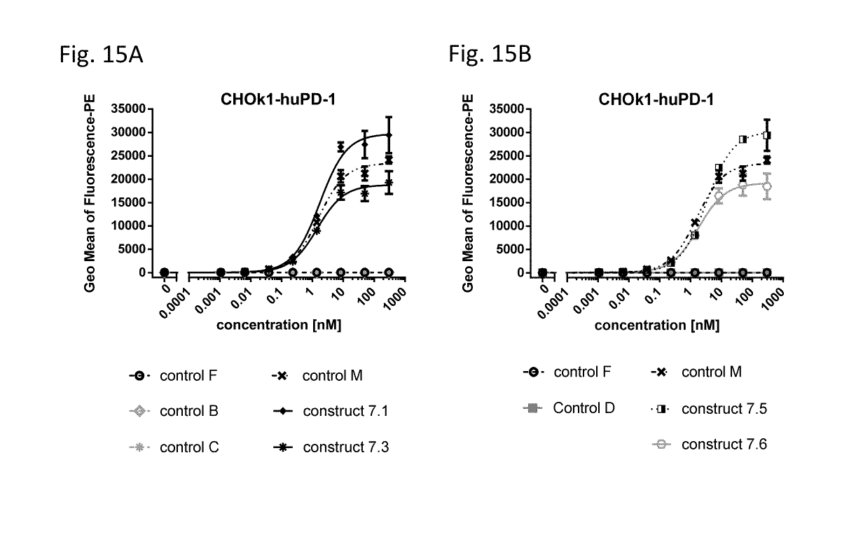

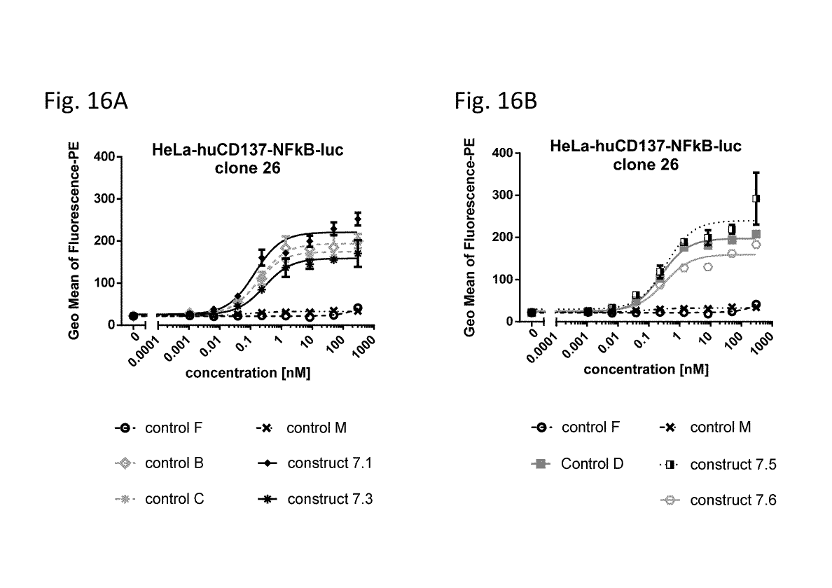

Gates were set on living tumor cells and geo means of fluorescence intensity of PE-conjugated AffiniPure anti-human IgG IgG Fcγ-fragment-specific goat F(ab′)2 fragment were blotted against the titrated concentration of PD1-targeted 4-1BB split trimeric ligand Fc fusion antigen binding molecules or their controls. As shown in FIG. 15A and FIG. 15B, only the PD1-targeted split trimeric 4-1BB ligand fusion molecules construct 7.1., construct 7.3, construct 7.5 and construct 7.6 and control M bound to PD1-expressing CHO-k1-huPD1 clone 5 whereas the not PD1 targeted control molecules control B, control C, control D and control F did not bind to the tumor cells. In FIG. 16A and FIG. 16B, only the split trimeric 4-1BB ligand containing fusion molecules construct 7.1., construct 7.3, construct 7.5 and construct 7.6 and control B, control C and control D bound to human 4-1BB-expressing HeLa-hu4-1BB-NFkB-luc clone 26 whereas the controls containing no fused split trimeric 4-1BB ligand (control F and control M) did not bind to the human 4-1BB expressing tumor cells. In Table 28 the EC₅₀ values of the curves are listed.

TABLE 28
EC₅₀ values of binding curves to human PD1 or human 4-1BB expressing tumor cells

| | Control | Control | Control | Control | Construct | Construct | Construct | Construct |
|---|---|---|---|---|---|---|---|---|
| | C | B | D | M | 7.1 | 7.3 | 7.5 | 7.6 |
| EC₅₀ [nM] on | 0.16 | 0.19 | 0.25 | n.d. | 0.14 | 0.28 | 0.36 | 0.32 |
| HeLa-huCD137- | | | | | | | | |
| NFkB-luc clone | | | | | | | | |
| 26 cells | | | | | | | | |
| EC₅₀ [nM] on | n.d. | n.d. | n.d. | 1.59 | 1.73 | 1.43 | 3.33 | 1.82 |
| CHO-K1-huPD1 | | | | | | | | |
| clone 5 cells | | | | | | | | |

### Example 6

### Biological Activity of the PD1 Targeted 4-1BB Ligand Trimer-Containing Fc Fusion Antigen Binding Molecules

NF-κB Activation of Human 4-1BB Expressing HeLa Reporter Cell Line

6.1 Generation of HeLa Cells Expressing Human 4-1BB and NF-κB-Luciferase

The human-papilloma-virus-18-induced cervix carcinoma cell line HeLa (ATCC CCL-2) was transduced with a plasmid based on the expression vector pETR10829, which contains the sequence of human 4-1BB (Uniprot accession Q07011) under control of a CMV-promoter and a puromycin resistance gene. Cells were cultured in DMEM-medium (Gibco by Life Technologies Cat. No. 42430-025) supplied with 10% Fetal Bovine Serum (FBS, GIBCO by Life Technologies, Cat. No. 16000-044, Lot. No. 941273, gamma-irradiated mycoplasma free, heat inactivated at 56° C. for 35 minutes), 1% GlutaMAX-I (GIBCO by Life Technologies, Cat.-No. 35050-038) and 3 μg/mL Puromycin (InvivoGen, Cat No. ant-pr-1).

4-1BB-transduced HeLa cells were tested for 4-1BB expression by flow cytometry: 0.2×10⁶ living cells were resuspended in 100 μL FACS buffer (DPBS supplied with 2% FBS, 5 mM EDTA pH 8 (Amresco, Cat. No. E177) and 7.5 mM Sodium Azide (Sigma-Aldrich, Cat. No. S2002)) containing 0.1 μg PerCP/Cy5.5 conjugated anti-human 4-1BB mouse IgG1K clone 4B4-1 (BioLegend Cat. No. 309814) or its isotype control (PerCP/Cy5.5 conjugated mouse IgG1K isotype control antibody clone MOPC 21, BioLegend Cat. No. 400150) and incubated for 30 minutes at 4° C. Cells were washed twice with FACS buffer, resuspended in 300 μL FACS buffer containing 0.06 μg DAPI (Santa Cruz Biotec, Cat. No. Sc-3598) and acquired using a 5-laser LSR-Fortessa (BD Bioscience, DIVA software). Limited dilutions were performed to generate single clones as following: Human-4-1BB transduced HeLa cells were resuspended in medium to a concentration of 10, 5 and 2.5 cells/mL and 200 μL of cell suspensions were transferred to round bottom tissue-culture treated 96-well plates (6 plates/cell concentration, TPP Cat. No. 92697). Single clones were harvested, expanded and tested for 4-1BB expression as described above. The clone with the highest expression of 4-1BB (clone 5) was chosen for subsequent transfection with the NFKB-luciferase expression-vector 5495p Tranlucent HygB. The vector confers transfected cells both with resistance to hygromycin B and capacity to express luciferase under control of NFkB-response element (Panomics, Cat. No. LR0051). Human-4-1BB HeLa clone 5 cells were cultured to 70% confluence. 50 μg (40 μL) linearized (restriction enzymes AseI and SalI) 5495p Tranlucent HygB expression vector were added to a sterile 0.4 cm Gene Pulser/MicroPulser Cuvette (Biorad, Cat.-No, 165-2081). 2.5×10⁶ human-4-1BB HeLa clone 5 cells in 400 μl supplement-free DMEM were added and mixed carefully with the plasmid solution. Transfection of cells was performed using a Gene Pulser Xcell total system (Biorad, Cat No. 165 2660) under the following settings: exponential pulse, capacitance 500 μF, voltage 160 V, resistance ∞. Immediately after the pulse transfected cells were transferred to a tissue culture flask 75 cm² (TPP, Cat. No. 90075) with 15 mL 37° C. warm DMEM-Medium (Gibco by Life Technologies Cat. No. 42430-025) supplied with 10% FBS and 1% GlutaMAX I (GIBCO by Life Technologies, Cat. No. 35050 038). Next day, culture medium containing 3 μg/mL Puromycin (InvivoGen, Cat No. ant-pr-1) and 200 μg/mL Hygromycin B (Roche, Cat. No. 10843555001) was added. Surviving cells were expanded and limited dilution was performed as described above to generate single clones. Clones were tested for 4-1BB expression as described above and for NFKB-Luciferase activity as following: Clones were harvested in selection medium and counted using a Cell Counter Vi-cell xr 2.03 (Beckman Coulter, Cat. No. 731050). Cells were set to a cell density of 0.33×10⁶ cells/mL and 150 μL of this cell suspension were transferred to each well of a sterile white 96-well flat bottom tissue culture plate with lid (greiner bio one, Cat. No. 655083) and as a control to normal 96 well flat bottom tissue culture plate (TPP Cat. No. 92096) to test survival and cell density the next day. Cells were incubated at 37° C. and 5% CO₂ overnight. The next day 50 μL of medium containing different concentrations of recombinant human tumor necrosis factor alpha (rhTNF α, PeproTech, Cat.-No. 300 01A) were added to each well of a 96-well plate resulting in final concentration of rhTNF α of 100, 50, 25, 12.5, 6.25 and 0 ng/well. Cells were incubated for 6 hours at 37° C. and 5% CO₂ and then washed three times with 200 μL/well DPBS. Reporter Lysis Buffer (Promega, Cat-No: E3971) was added to each well (30 μl) and the plates were stored over night at −20° C. The next day frozen cell plates and Detection Buffer (Luciferase 1000 Assay System, Promega, Cat. No. E4550) were thawed to room temperature. 100 uL of detection buffer were added to each well and the plate was measured as fast as possible using a SpectraMax M5/M5e microplate reader and the SoftMax Pro Software (Molecular Devices). Measured URLs above control (no rhTNF α added) were taken as luciferase activity. The NFKB-luc-4-1BB-HeLa clone 26 was chosen for further use exhibiting the highest luciferase activity and a considerable level of 4-1BB-expression.

6.2 NF-κB Activation of HeLa Reporter Cells Expressing Human 4-1BB Co-Cultured with PD1-Expressing CHO-k1-huPD1 Clone 5 Cells

NFκB-luc-4-1BB-HeLa clone 26 cells were washed with DPBS (Gibco by Life Technologies, Cat. No. 14190 326) and treated with 0.05% Trypsin EDTA (Gibco by Life Technologies Cat. No. 25300 054) for 5 min at 37° C. Cells were harvested and resuspended in DMEM medium (Gibco by Life Technologies Cat. No. 42430 025) supplied with 10% Fetal Calf Serum (FBS, US-origin, PAN biotech, P30-2009, Lot P150307GI, gamma irradiated mycoplasma free, heat inactivated 35 min 56° C.) and 1% GlutaMAX-I (GIBCO by Life Technologies, Cat. No. 35050 038). Cells were counted using Cell Counter Vi-cell xr 2.03 (Beckman Coulter, Cat. No. 731050) and set to a cell density of 0.2×10⁶ cells/mL. 100 μL (2×10⁴ cells) of this cell suspension were transferred to each well of a sterile white 96-well flat bottom tissue culture plate with lid (greiner bio one, Cat. No. 655083) and the plates were incubated at 37° C. and 5% CO2 overnight. The next day 50 μL of medium containing different titrated split trimeric 4-1BB ligand containing fusion molecules construct 7.1., construct 7.3, construct 7.5 and construct 7.6 or the fitting controls control B, control C, control D, control F and control M. For PD1-binding-mediated crosslinking the PD1-expressing CHO-k1-huPD1 clone 5 cells were used.

Adherent CHO-k1-huPD1 clone 5 cells were washed with DPBS (Gibco by Life Technologies, Cat. No. 14190 326) and treated with enzyme-free, PBS-based Cell Dissociation Buffer (Gibco by Life Technologies, Cat.-No. 13151-014) for 10 minutes at 37° C. Afterwards cell were harvested and counted using Cell Counter Vi-cell xr 2.03. Cells were resuspended in DEM supplied with 10% FBS and 1% L-GlutaMAX-I to 2×10⁶ cells/mL and 50 μL were added/well. After adding crosslinking PD1-expressing CHO-k1-huPD1 clone 5 cells, plates were incubated for 6 hours at 37° C. and 5% CO₂. White flat bottom 96-well plates were washed three times with 200 μL/well DPBS. 40 μl fresh prepared Reporter Lysis Buffer (Promega, Cat-No: E3971) were added to each well and the plate were stored over night at −20° C. The next day frozen plates and detection buffer (Luciferase 1000 Assay System, Promega, Cat. No. E4550) were thawed to room temperature. 100 uL of detection buffer were added to each well and plates were measured as fast as possible using the SpectraMax M5/M5e microplate reader and SoftMax Pro Software (Molecular Devices) with following settings: for luciferase (RLUs), 500 ms integration time, no filter, collecting all wave length and top reading.

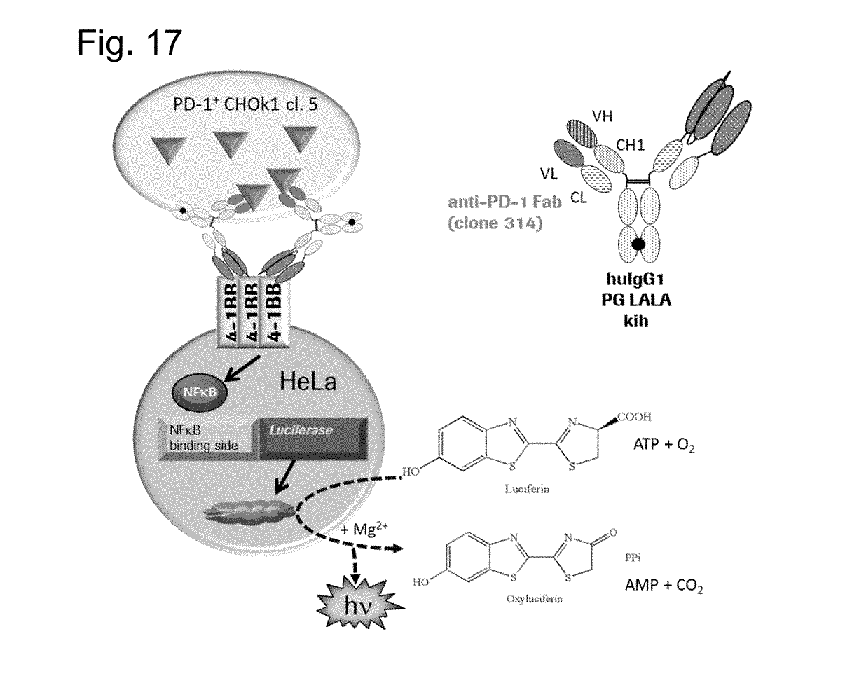

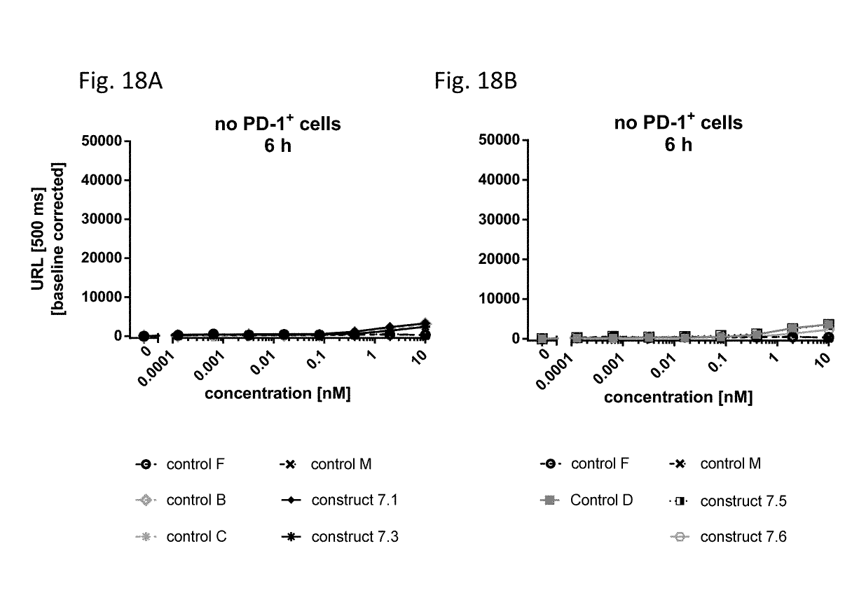

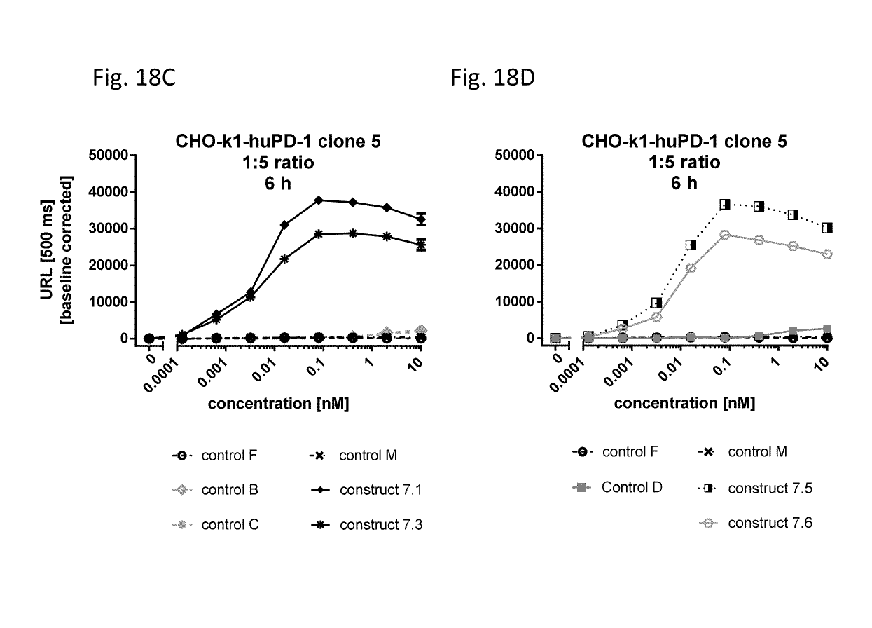

PD1-targeted 4-1BB ligand trimer-containing Fc fusion antigen binding molecule (PD1 split 4-1BBL trimer) triggered activation of the NFKB signaling pathway in the reporter cell line in the presence of PD1-expressing tumor cells. In contrast, the untargeted variant of the same molecule failed to trigger such an effect at any of the tested concentrations (FIG. 17C and FIG. 17D). This activity of targeted 4-1BBL was strictly dependent on the expression of PD1 at the cell surface of tumor cells as no NF-kB activation could be detected upon culturing of the NF-kB reporter cell line with PD1-negative tumor cells even in the presence of PD1-targeted 4-1BB ligand trimer-containing Fc fusion antigen binding molecule (FIG. 17A and FIG. 17B). The activities as measured for Constructs 7.1, 7.3, 7.5 and 7.6 are shown. The EC₅₀ values as measured in the presence of PD1 expressing tumor cells are given in Table 29 below.

TABLE 29
EC₅₀ values of NFκB-activation-induced Luciferase activity-curves

| | Constuct | Construct | Construct | Construct |
|---|---|---|---|---|
| | 7.1 | 7.3 | 7.5 | 7.6 |
| EC₅₀ [nM] with CHO- | 0.004 | 0.004 | 0.006 | 0.007 |
| k1-huPD1 clone 5 | | | | |

### CITATIONS

Ascierto, P. A., E. Simeone, M. Sznol, Y. X. Fu, and I. Melero (2010), Clinical experiences with anti-CD137 and anti-PD1 therapeutic antibodies. Semin Oncol 37:508-516.
Aggarwal B. B. (2003), Signalling pathways of the TNF superfamily: a double-edged sword. Nat. Rev. Immunol. 3(9),745-56.
Banner D. et al (1993), Crystal structure of the soluble human 55 kd TNF receptor-human TNF beta complex: implications for TNF receptor activation. Cell 73, 431-445.
Bodmer J., Schneider P. and Tschopp, J. (2002), The molecular architecture of the TNF superfamily. Trends in Biochemical Sciences 27(1), 19-26.
Broll, K., Richter, G., Pauly, S., Hofstaedter, F., and Schwarz, H. (2001). CD137 expression in tumor vessel walls. High correlation with malignant tumors. Am J Clin Pathol 115, 543-549.
Buechele, C., Baessler, T., Schmiedel, B. J., Schumacher, C. E., Grosse-Hovest, L., Rittig, K., and Salih, H. R. (2012). 4-1BB ligand modulates direct and Rituximab-induced NK-cell reactivity in chronic lymphocytic leukemia. Eur J Immunol 42, 737-748.
Chen S., Lee L., Fisher T., Jessen B., Elliott M., Evering W., Logronio K., Tu K. H., Tsaparikos K., Li X., Wang H., Ying C., Xiong M., Van Arsdale T., and. Lin J. C. (2015), Combination of 4-1BB Agonist and PD-1 Antagonist Promotes Antitumor Effector/Memory CD8 T Cells in a Poorly Immunogenic Tumor Model. Cancer Immunology Research 3(2), 149-160. Published online 11 Nov. 2014.
Choi, B. K., Kim, Y. H., Kwon, P. M., Lee, S. C., Kang, S. W., Kim, M. S., Lee, M. J., and Kwon, B. S. (2009). 4-1BB functions as a survival factor in dendritic cells. J Immunol 182, 4107-4115.
Cuadros, C., Dominguez, A. L., Lollini, P. L., Croft, M., Mittler, R. S., Borgstrom, P., and Lustgarten, J. (2005). Vaccination with dendritic cells pulsed with apoptotic tumors in combination with anti-OX40 and anti-4-1BB monoclonal antibodies induces T cell-mediated protective immunity in Her-2/neu transgenic mice. Int J Cancer 116, 934-943.
Curran, M. A., Kim, M., Montalvo, W., Al-Shamkhani, A., and Allison, J. P. (2011). Combination CTLA-4 blockade and 4-1BB activation enhances tumor rejection by increasing T-cell infiltration, proliferation, and cytokine production. PLoS One 6, e19499.
Diehl, L., van Mierlo, G. J., den Boer, A. T., van der Voort, E., Fransen, M., van Bostelen, L., Krimpenfort, P., Melief, C. J., Mittler, R., Toes, R. E., and Offringa, R. (2002). In vivo triggering through 4-1BB enables Th-independent priming of CTL in the presence of an intact CD28 costimulatory pathway. J Immunol 168, 3755-3762.
Dubrot, J., Milheiro, F., Alfaro, C., Palazon, A., Martinez-Forero, I., Perez-Gracia, J. L., Morales-Kastresana, A., Romero-Trevejo, J. L., Ochoa, M. C., Hervas-Stubbs, S., et al. (2010). Treatment with anti-CD137 mAbs causes intense accumulations of liver T cells without selective antitumor immunotherapeutic effects in this organ. Cancer Immunol Immunother 59, 1223-1233.
Futagawa, T., Akiba, H., Kodama, T., Takeda, K., Hosoda, Y., Yagita, H., and Okumura, K. (2002). Expression and function of 4-1BB and 4-1BB ligand on murine dendritic cells. Int Immunol 14, 275-286.
Guo, Z., Cheng, D., Xia, Z., Luan, M., Wu, L., Wang, G., and Zhang, S. (2013). Combined TIM-3 blockade and CD137 activation affords the long-term protection in a murine model of ovarian cancer. J Transl Med 11, 215.
Heinisch, I. V., Daigle, I., Knopfli, B., and Simon, H. U. (2000). CD137 activation abrogates granulocyte-macrophage colony-stimulating factor-mediated anti-apoptosis in neutrophils. Eur J Immunol 30, 3441-3446.
Hornig, N., Kermer, V., Frey, K., Diebolder, P., Kontermann, R. E., Mueller, D. (2012), Combination of a bispecific antibody and costimulatory antibody-ligand fusion proteins for targeted cancer immunotherapy. J. Immunother. 35, 418-429.
Ju, S. A., Cheon, S. H., Park, S. M., Tam, N. Q., Kim, Y. M., An, W. G., and Kim, B. S. (2008). Eradication of established renal cell carcinoma by a combination of 5-fluorouracil and anti-4-1BB monoclonal antibody in mice. Int J Cancer 122, 2784-2790.
Kienzle, G., and von Kempis, J. (2000). CD137 (ILA/4-1BB), expressed by primary human monocytes, induces monocyte activation and apoptosis of B lymphocytes. Int Immunol 12, 73-82.
Kim, D. H., Chang, W. S., Lee, Y. S., Lee, K. A., Kim, Y. K., Kwon, B. S., and Kang, C. Y. (2008). 4-1BB engagement costimulates NKT cell activation and exacerbates NKT cell ligand-induced airway hyperresponsiveness and inflammation. J Immunol 180, 2062-2068.
Kim, Y. H., Choi, B. K., Oh, H. S., Kang, W. J., Mittler, R. S., and Kwon, B. S. (2009). Mechanisms involved in synergistic anticancer effects of anti-4-1BB and cyclophosphamide therapy. Mol Cancer Ther 8, 469-478.
Kwon, B. S., and Weissman, S. M. (1989). cDNA sequences of two inducible T-cell genes. Proc Natl Acad Sci USA 86, 1963-1967.
Lee, H., Park, H. J., Sohn, H. J., Kim, J. M., and Kim, S. J. (2011). Combinatorial therapy for liver metastatic colon cancer: dendritic cell vaccine and low-dose agonistic anti-4-1BB antibody co-stimulatory signal. J Surg Res 169, e43-50.
Levitsky, V., de Campos-Lima, P. O., Frisan, T., and Masucci, M. G. (1998). The clonal composition of a peptide-specific oligoclonal CTL repertoire selected in response to persistent EBV infection is stable over time. J Immunol 161, 594-601.
Li, F., and Ravetch, J. V. (2011). Inhibitory Fcgamma receptor engagement drives adjuvant and anti-tumor activities of agonistic CD40 antibodies. Science 333, 1030-1034.
Lin, W., Voskens, C. J., Zhang, X., Schindler, D. G., Wood, A., Burch, E., Wei, Y., Chen, L., Tian, G., Tamada, K., et al. (2008). Fc-dependent expression of CD137 on human NK cells: insights into “agonistic” effects of anti-CD137 monoclonal antibodies. Blood 112, 699-707.
Melero, I., Johnston, J. V., Shufford, W. W., Mittler, R. S., and Chen, L. (1998). NK1.1 cells express 4-1BB (CDw137) costimulatory molecule and are required for tumor immunity elicited by anti-4-1BB monoclonal antibodies. Cell Immunol 190, 167-172.
Melero, I., Shuford, W. W., Newby, S. A., Aruffo, A., Ledbetter, J. A., Hellstrom, K. E., Mittler, R. S., and Chen, L. (1997). Monoclonal antibodies against the 4-1BB T-cell activation molecule eradicate established tumors. Nat Med 3, 682-685.
Merchant, A. M., Zhu, Z., Yuan, J. Q., Goddard, A., Adams, C. W., Presta, L. G., and Carter, P. (1998). An efficient route to human bispecific IgG. Nat Biotechnol 16, 677-681.
Morales-Kastresana, A., Sanmamed, M. F., Rodriguez, I., Palazon, A., Martinez-Forero, I., Labiano, S., Hervas-Stubbs, S., Sangro, B., Ochoa, C., Rouzaut, A., et al. (2013). Combined immunostimulatory monoclonal antibodies extend survival in an aggressive transgenic hepatocellular carcinoma mouse model. Clin Cancer Res 19, 6151-6162.
Mueller, D., Frey, K., Kontermann, R. E. (2008), A novel antibody-4-1BB1 fusion protein for targeted costimulation in cancer immunotherapy, J. Immunother. 31, 714-722.
Murillo, O., Dubrot, J., Palazon, A., Arina, A., Azpilikueta, A., Alfaro, C., Solano, S., Ochoa, M. C., Berasain, C., Gabari, I., et al. (2009). In vivo depletion of DC impairs the anti-tumor effect of agonistic anti-CD137 mAb. Eur J Immunol 39, 2424-2436.
Narazaki, H., Zhu, Y., Luo, L., Zhu, G., and Chen, L. (2010). CD137 agonist antibody prevents cancer recurrence: contribution of CD137 on both hematopoietic and nonhematopoietic cells. Blood 115, 1941-1948.
Nishimoto, H., Lee, S. W., Hong, H., Potter, K. G., Maeda-Yamamoto, M., Kinoshita, T., Kawakami, Y., Mittler, R. S., Kwon, B. S., Ware, C. F., et al. (2005). Costimulation of mast cells by 4-1BB, a member of the tumor necrosis factor receptor superfamily, with the high-affinity IgE receptor. Blood 106, 4241-4248.
Olofsson, P. S., Soderstrom, L. A., Wagsater, D., Sheikine, Y., Ocaya, P., Lang, F., Rabu, C., Chen, L., Rudling, M., Aukrust, P., et al. (2008). CD137 is expressed in human atherosclerosis and promotes development of plaque inflammation in hypercholesterolemic mice. Circulation 117, 1292-1301.
Palazon, A., Teijeira, A., Martinez-Forero, I., Hervas-Stubbs, S., Roncal, C., Penuelas, I., Dubrot, J., Morales-Kastresana, A., Perez-Gracia, J. L., Ochoa, M. C., et al. (2011). Agonist anti-CD137 mAb act on tumor endothelial cells to enhance recruitment of activated T lymphocytes. Cancer Res 71, 801-811.
Schwarz, H., Valbracht, J., Tuckwell, J., von Kempis, J., and Lotz, M. (1995). ILA, the human 4-1BB homologue, is inducible in lymphoid and other cell lineages. Blood 85, 1043-1052.
Shao, Z., and Schwarz, H. (2011). CD137 ligand, a member of the tumor necrosis factor family, regulates immune responses via reverse signal transduction. J Leukoc Biol 89, 21-29.
Shi, W., and Siemann, D. W. (2006). Augmented antitumor effects of radiation therapy by 4-1BB antibody (BMS-469492) treatment. Anticancer Res 26, 3445-3453.
Shindo Y., Yoshimura K., Kuramasu A., Watanabe Y., Ito H.; Kondo T., et al. (2015). Combination Immunotherapy with 4-1BB activation and PD-1 blockade enhances antitumor efficacy in a mouse model of subcutaneous tumor. Anticancer Res. 35(1), 129-136.
Simeone, E., and Ascierto, P. A. (2012). Immunomodulating antibodies in the treatment of metastatic melanoma: the experience with anti-CTLA-4, anti-CD137, and anti-PD1. J Immunotoxicol 9, 241-247.
Snell, L. M., Lin, G. H., McPherson, A. J., Moraes, T. J., and Watts, T. H. (2011). T-cell intrinsic effects of GITR and 4-1BB during viral infection and cancer immunotherapy. Immunol Rev 244, 197-217.
Stagg, J., Loi, S., Divisekera, U., Ngiow, S. F., Duret, H., Yagita, H., Teng, M. W., and Smyth, M. J. (2011). Anti-ErbB-2 mAb therapy requires type I and II interferons and synergizes with anti-PD-1 or anti-CD137 mAb therapy. Proc Natl Acad Sci USA 108, 7142-7147.
Teng, M. W., Sharkey, J., McLaughlin, N. M., Exley, M. A., and Smyth, M. J. (2009). CD1d-based combination therapy eradicates established tumors in mice. J Immunol 183, 1911-1920.
von Kempis, J., Schwarz, H., and Lotz, M. (1997). Differentiation-dependent and stimulus-specific expression of ILA, the human 4-1BB-homologue, in cells of mesenchymal origin. Osteoarthritis Cartilage 5, 394-406.
Wei, H., Zhao, L., Li, W., Fan, K., Qian, W., Hou, S., Wang, H., Dai, M., Hellstrom, I., Hellstrom, K. E., and Guo, Y. (2013). Combinatorial PD-1 blockade and CD137 activation has therapeutic efficacy in murine cancer models and synergizes with cisplatin. PLoS One 8, e84927.
Wei H., Zhao L., Hellstrom I., Hellstrom K E. and Guo Y. (2014). Dual targeting of CD137 co-stimulatory and PD-1 co-inhibitory molecules for ovarian cancer immunotherapy, Oncolmmunology, 3:4, e28248, DOI: 10.4161/onci.28248.
Wilcox, R. A., Chapoval, A. I., Gorski, K. S., Otsuji, M., Shin, T., Flies, D. B., Tamada, K., Mittler, R. S., Tsuchiya, H., Pardoll, D. M., and Chen, L. (2002). Cutting edge: Expression of functional CD137 receptor by dendritic cells. J Immunol 168, 4262-4267.
Wilcox, R. A., Tamada, K., Flies, D. B., Zhu, G., Chapoval, A. I., Blazar, B. R., Kast, W. M., and Chen, L. (2004). Ligation of CD137 receptor prevents and reverses established anergy of CD8+ cytolytic T lymphocytes in vivo. Blood 103, 177-184.
Zhang, N., Sadun, R. E., Arias, R. S., Flanagan, M. L., Sachsman, S. M., Nien, Y, Khawli, L. A., Hu, P., Epstein, A. L. (2007). Targeted and untargeted CD137L fusion proteins for the immunotherapy of experimental solid tumors. Clin. Cancer Res. 13, 2758-2767.
Zhang, X., Voskens, C. J., Sallin, M., Maniar, A., Montes, C. L., Zhang, Y., Lin, W., Li, G., Burch, E., Tan, M., et al. (2010). CD137 promotes proliferation and survival of human B cells. J Immunol 184, 787-795.

1. A TNF family ligand trimer-containing antigen binding molecule comprising
(a) at least one moiety capable of specific binding to PD1 and
(b) a first and a second polypeptide that are linked to each other by a disulfide bond,
wherein the antigen binding molecule is characterized in that the first polypeptide comprises two ectodomains of a TNF ligand family member or a fragment thereof that are connected to each other by a peptide linker and in that the second polypeptide comprises only one ectodomain of said TNF ligand family member or a fragment thereof.

2. The TNF family ligand trimer-containing antigen binding molecule of claim 1, further comprising
(c) an Fc domain composed of a first and a second subunit capable of stable association.

3. The TNF family ligand trimer-containing antigen binding molecule of claim 1, comprising
(a) at least one moiety capable of specific binding to PD1 and
(b) a first and a second polypeptide that are linked to each other by a disulfide bond,
wherein the antigen binding molecule is characterized in that
(i) the first polypeptide contains a CH1 or CL domain and the second polypeptide contains a CL or CH1 domain, respectively, wherein the second polypeptide is linked to the first polypeptide by a disulfide bond between the CH1 and CL domain, and wherein the first polypeptide comprises two ectodomains of a TNF ligand family member or fragments thereof that are connected to each other and to the CH1 or CL domain by a peptide linker and wherein the second polypeptide comprises one ectodomain of said TNF ligand family member or a fragment thereof connected via a peptide linker to the CL or CH1 domain of said polypeptide, or
(ii) the first polypeptide contains a CH3 domain and the second polypeptide contains a CH3 domain, respectively, and wherein the first polypeptide comprises two ectodomains of a TNF ligand family member or fragments thereof that are connected to each other and to the C-terminus of the CH3 domain by a peptide linker and wherein the second polypeptide comprises only one ectodomain of said TNF ligand family member or a fragment thereof connected via a peptide linker to the C-terminus of the CH3 domain of said polypeptide, or
(iii) the first polypeptide contains a VH-CL or a VL-CH1 domain and the second polypeptide contains a VL-CH1 domain or a VH-CL domain, respectively, wherein the second polypeptide is linked to the first polypeptide by a disulfide bond between the CH1 and CL domain, and wherein the first polypeptide comprises two ectodomains of a TNF ligand family member or fragments thereof that are connected to each other and to to VH or VL by a peptide linker and wherein the second polypeptide comprises one ectodomain of said TNF ligand family member or a fragment thereof connected via a peptide linker to VL or VH of said polypeptide.

4. The TNF family ligand trimer-containing antigen binding molecule of claim 1, wherein the TNF ligand family member is selected from 4-1BBL and OX40L.

5. The TNF family ligand trimer-containing antigen binding molecule of claim 1, wherein the TNF ligand family member is 4-1BBL.

6. The TNF family ligand trimer-containing antigen binding molecule of claim 1, wherein the ectodomain of a TNF ligand family member comprises the amino acid sequence selected from the group consisting of SEQ ID NO: 1, SEQ ID NO: 2, SEQ ID NO:3, SEQ ID NO:4, SEQ ID NO:5, SEQ ID NO: 6, SEQ ID NO:7 and SEQ ID NO:8, particularly the amino acid sequence of SEQ ID NO: 1 or SEQ ID NO:5.

7. The TNF family ligand trimer-containing antigen binding molecule of claim 1, comprising
(a) at least one moiety capable of specific binding to PD1 and
(b) a first and a second polypeptide that are linked to each other by a disulfide bond,
wherein the antigen binding molecule is characterized in that the first polypeptide comprises the amino acid sequence selected from the group consisting of SEQ ID NO:9, SEQ ID NO: 11, SEQ ID NO: 12 and SEQ ID NO: 10 and in that the second polypeptide comprises the amino acid sequence selected from the group consisting of SEQ ID NO: 1, SEQ ID NO:3, SEQ ID NO:4 and SEQ ID NO:5.

8. The TNF family ligand trimer-containing antigen binding molecule of claim 1, comprising
(a) at least one moiety capable of specific binding to PD1, and
(b) a first polypeptide containing a CH1 or CL domain and a second polypeptide containing a CL or CH1 domain, respectively, wherein the second polypeptide is linked to the first polypeptide by a disulfide bond between the CH1 and CL domain,
and wherein the antigen binding molecule is characterized in that the first polypeptide comprises two ectodomains of a TNF ligand family member or fragments thereof that are connected to each other and to the CH1 or CL domain by a peptide linker and in that the second polypeptide comprises one ectodomain of said TNF ligand family member or a fragment thereof connected via a peptide linker to the CL or CH1 domain of said polypeptide.

9. The TNF family ligand trimer-containing antigen binding molecule of claim 1, wherein the moiety capable of specific binding to PD1 is a Fab molecule capable of specific binding to PD1.

10. The TNF family ligand trimer-containing antigen binding molecule of claim 1, wherein the moiety capable of specific binding to PD1 comprises
(a) a VH domain comprising (i) HVR-H1 comprising the amino acid sequence of SEQ ID NO: 13,
(ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16 (v) HVR-L2 comprising the amino acid sequence of SEQ ID NO: 17, and (vi) HVR-L3 comprising the amino acid sequence of SEQ ID NO: 18,
(b) a VH domain comprising (i) HVR-H1 comprising the amino acid sequence of SEQ ID NO:26,
(ii) HVR-H2 comprising the amino acid sequence of SEQ ID NO:27, and (iii) HVR-H3 comprising the amino acid sequence of SEQ ID NO:28, and a VL domain comprising (iv) HVR-L1 comprising the amino acid sequence of SEQ ID NO:29 (v) HVR-L2 comprising the amino acid sequence of SEQ ID NO:30, and (vi) HVR-L3 comprising the amino acid sequence of SEQ ID NO:31,
(c) a VH domain comprising (i) HVR-H1 comprising the amino acid sequence of SEQ ID NO:34,
(ii) HVR-H2 comprising the amino acid sequence of SEQ ID NO:35, and (iii) HVR-H3 comprising the amino acid sequence of SEQ ID NO:36, and a VL domain comprising (iv) HVR-L1 comprising the amino acid sequence of SEQ ID NO:37 (v) HVR-L2 comprising the amino acid sequence of SEQ ID NO:38, and (vi) HVR-L3 comprising the amino acid sequence of SEQ ID NO:39,
(d) a VH domain comprising (i) HVR-H1 comprising the amino acid sequence of SEQ ID NO:42,
(ii) HVR-H2 comprising the amino acid sequence of SEQ ID NO:43, and (iii) HVR-H3 comprising the amino acid sequence of SEQ ID NO:44, and a VL domain comprising (iv) HVR-L1 comprising the amino acid sequence of SEQ ID NO:45 (v) HVR-L2 comprising the amino acid sequence of SEQ ID NO:46, and (vi) HVR-L3 comprising the amino acid sequence of SEQ ID NO:47,
(e) a VH domain comprising (i) HVR-H1 comprising the amino acid sequence of SEQ ID NO:50,
(ii) HVR-H2 comprising the amino acid sequence of SEQ ID NO:51, and (iii) HVR-H3 comprising the amino acid sequence of SEQ ID NO:52, and a VL domain comprising (iv) HVR-L1 comprising the amino acid sequence of SEQ ID NO:53 (v) HVR-L2 comprising the amino acid sequence of SEQ ID NO:54, and (vi) HVR-L3 comprising the amino acid sequence of SEQ ID NO:55,
(f) a VH domain comprising (i) HVR-H1 comprising the amino acid sequence of SEQ ID NO:58,
(ii) HVR-H2 comprising the amino acid sequence of SEQ ID NO:59, and (iii) HVR-H3 comprising the amino acid sequence of SEQ ID NO:60, and a VL domain comprising (iv) HVR-L1 comprising the amino acid sequence of SEQ ID NO:61 (v) HVR-L2 comprising the amino acid sequence of SEQ ID NO:62, and (vi) HVR-L3 comprising the amino acid sequence of SEQ ID NO:63, or
(g) a VH domain comprising (i) HVR-H1 comprising the amino acid sequence of SEQ ID NO:66,
(ii) HVR-H2 comprising the amino acid sequence of SEQ ID NO:67, and (iii) HVR-H3 comprising the amino acid sequence of SEQ ID NO:68, and a VL domain comprising (iv) HVR-L1 comprising the amino acid sequence of SEQ ID NO:69 (v) HVR-L2 comprising the amino acid sequence of SEQ ID NO:70, and (vi) HVR-L3 comprising the amino acid sequence of SEQ ID NO:71.

11. The TNF family ligand trimer-containing antigen binding molecule of claim 1, wherein the moiety capable of specific binding to PD1 comprises
(a) a VH domain comprising an amino acid sequence of SEQ ID NO: 19 and a VL domain comprising an amino acid sequence of SEQ ID NO:20,
(b) a VH domain comprising an amino acid sequence of SEQ ID NO:21 and a VL domain comprising an amino acid sequence of SEQ ID NO:22,
(c) a VH domain comprising an amino acid sequence of SEQ ID NO:21 and a VL domain comprising an amino acid sequence of SEQ ID NO:23,
(d) a VH domain comprising an amino acid sequence of SEQ ID NO:21 and a VL domain comprising an amino acid sequence of SEQ ID NO:24,
(e) a VH domain comprising an amino acid sequence of SEQ ID NO:21 and a VL domain comprising an amino acid sequence of SEQ ID NO:25,
(f) a VH domain comprising an amino acid sequence of SEQ ID NO:32 and a VL domain comprising an amino acid sequence of SEQ ID NO:33,
(g) a VH domain comprising an amino acid sequence of SEQ ID NO:40 and a VL domain comprising an amino acid sequence of SEQ ID NO:41,
(h) a VH domain comprising an amino acid sequence of SEQ ID NO:48 and a VL domain comprising an amino acid sequence of SEQ ID NO:49,
(i) a VH domain comprising an amino acid sequence of SEQ ID NO:56 and a VL domain comprising an amino acid sequence of SEQ ID NO:57,
(k) a VH domain comprising an amino acid sequence of SEQ ID NO:64 and a VL domain comprising an amino acid sequence of SEQ ID NO:65, or
(l) a VH domain comprising an amino acid sequence of SEQ ID NO:72 and a VL domain comprising an amino acid sequence of SEQ ID NO:73.12. The TNF family ligand trimer-containing antigen binding molecule of any one of claims 1 to 11, wherein the moiety capable of specific binding to PD1 comprises a VH domain comprising (i) HVR-H1 comprising the amino acid sequence of SEQ ID NO: 13, (ii) HVR-H2 comprising the amino acid sequence of SEQ ID NO: 14, and (iii) HVR-H3 comprising the amino acid sequence of SEQ ID NO: 15, and a VL domain comprising (iv) HVR-L1 comprising the amino acid sequence of SEQ ID NO: 16, (v) HVR-L2 comprising the amino acid sequence of SEQ ID NO: 17, and (vi) HVR-L3 comprising the amino acid sequence of SEQ ID NO: 18.

12. The TNF family ligand trimer-containing antigen binding molecule of claim 1, wherein the moiety capable of specific binding to PD1 comprises
(a) a VH domain comprising an amino acid sequence of SEQ ID NO: 19 and a VL domain comprising an amino acid sequence of SEQ ID NO:20,
(b) a VH domain comprising an amino acid sequence of SEQ ID NO:21 and a VL domain comprising an amino acid sequence of SEQ ID NO:22,
(c) a VH domain comprising an amino acid sequence of SEQ ID NO:21 and a VL domain comprising an amino acid sequence of SEQ ID NO:23,
(d) a VH domain comprising an amino acid sequence of SEQ ID NO:21 and a VL domain comprising an amino acid sequence of SEQ ID NO:24, or
(e) a VH domain comprising an amino acid sequence of SEQ ID NO:21 and a VL domain comprising an amino acid sequence of SEQ ID NO:25.

13. The TNF family ligand trimer-containing antigen binding molecule of claim 1, wherein the antigen binding molecule comprises
a first heavy chain and a first light chain, both comprising a Fab molecule capable of specific binding to PD1,
a first peptide comprising two ectodomains of a TNF ligand family member or fragments thereof connected to each other by a first peptide linker fused at its C-terminus by a second peptide linker to a second heavy or light chain,
and a second peptide comprising one ectodomain of said TNF ligand family member fused at its C-terminus by a third peptide linker to a second light or heavy chain, respectively.

14. The TNF family ligand trimer-containing antigen binding molecule of claim 1, wherein the first peptide comprising two ectodomains of a TNF ligand family member or fragments thereof connected to each other by a first peptide linker is fused at its C-terminus by a second peptide linker to a CL domain that is part of a heavy chain, and the second peptide comprising one ectodomain of said TNF ligand family member or a fragment thereof is fused at its C-terminus by a third peptide linker to a CH1 domain that is part of a light chain.

15. The TNF family ligand trimer-containing antigen binding molecule of claim 1, comprising
(a) at least one Fab domain capable of specific binding to PD1, and
(b) a first and a second polypeptide that are linked to each other by a disulfide bond,
wherein the antigen binding molecule is characterized in that the first polypeptide contains a CH3 domain and the second polypeptide contains a CH3 domain, respectively, and wherein the first polypeptide comprises two ectodomains of a TNF ligand family member or fragments thereof that are connected to each other and to the C-terminus of the CH3 domain by a peptide linker and wherein the second polypeptide comprises one ectodomain of said TNF ligand family member or a fragment thereof connected via a peptide linker to C-terminus of the CH3 domain of said polypeptide.

16. The TNF family ligand trimer-containing antigen binding molecule of claim 1, wherein the antigen binding molecule comprises
(i) a fusion polypeptide comprising a first subunit of a Fc domain and two ectodomains of a TNF ligand family member or fragments thereof that are connected to each other and to the C-terminus of the CH3 domain by a peptide linker, (ii) a heavy chain comprising the VH domain of the Fab domain capable of specific binding to PD1, a second subunit of the Fc domain and one ectodomain of said TNF ligand family member or a fragment thereof connected via a peptide linker to C-terminus of the CH3 domain, and (iii) a light chain comprising the VL domain of the Fab domain capable of specific binding to PD1, or
(i) a fusion polypeptide comprising a first subunit of a Fc domain and one ectodomain of a TNF ligand family member or a fragment thereof that is connected to the C-terminus of the CH3 domain by a peptide linker, (ii) a heavy chain comprising the VH domain of the Fab domain capable of specific binding to PD1, a second subunit of the Fc domain and two ectodomains of said TNF ligand family member or fragments thereof that are connected to each other and to the C-terminus of the CH3 domain via a peptide linker, and (iii) a light chain comprising the VL domain of the Fab domain capable of specific binding to PD1.

17. The TNF family ligand trimer-containing antigen binding molecule of claim 2, wherein the Fc domain comprises one or more amino acid substitution that reduces binding to an Fc receptor, in particular towards Fcγ receptor.

18. The TNF family ligand trimer-containing antigen binding molecule of claim 2, wherein the Fc domain is an IgG1 Fc domain comprising the amino acid substitutions the amino acid substitutions L234A, L235A and P329G (numbering according to Kabat EU index).

19. The TNF family ligand trimer-containing antigen binding molecule of claim 2, wherein the Fc domain comprises knob-into-hole modifications promoting association of the first and the second subunit of the Fc domain.

20. The TNF family ligand trimer-containing antigen binding molecule of claim 1, wherein the antigen binding molecule comprises
(i) a first heavy chain comprising the VH domain comprising the amino acid sequence of SEQ ID NO: 19 and a first light chain comprising the VL domain comprising the amino acid sequence of SEQ ID NO:20 or
a first heavy chain comprising the VH domain comprising the amino acid sequence of SEQ ID NO:21 and a first light chain comprising the VL domain comprising the amino acid sequence selected from the group consisting of SEQ ID NO:22, SEQ ID NO:23, SEQ ID NO:24 and SEQ ID NO:25,
(ii) a second heavy chain comprising the amino acid sequence selected from the group consisting of SEQ ID NO:74, SEQ ID NO:76, SEQ ID NO:78 and SEQ ID NO:80, and
(iii) a second light chain comprising the amino acid sequence selected from the group consisting of SEQ ID NO:75, SEQ ID NO:77, SEQ ID NO:79 and SEQ ID NO:81.

21. The TNF family ligand trimer-containing antigen binding molecule of claim 1, wherein the antigen binding molecule comprises
(a) a first heavy chain comprising the amino acid sequence of SEQ ID NO:82, a first light chain comprising the amino acid sequence of SEQ ID NO:83 or SEQ ID NO: 171, a second heavy chain comprising the amino acid sequence of SEQ ID NO:74 and a second light chain comprising the amino acid sequence of SEQ ID NO:75,
(b) a first heavy chain comprising the amino acid sequence of SEQ ID NO:82, a first light chain comprising the amino acid sequence of SEQ ID NO:83 or SEQ ID NO: 171, a second heavy chain comprising the amino acid sequence of SEQ ID NO:76 and a second light chain comprising the amino acid sequence of SEQ ID NO:77,
(c) a first heavy chain comprising the amino acid sequence of SEQ ID NO:82, a first light chain comprising the amino acid sequence of SEQ ID NO:83 or SEQ ID NO: 171, a second heavy chain comprising the amino acid sequence of SEQ ID NO:78 and a second light chain comprising the amino acid sequence of SEQ ID NO:79, or
(d) a first heavy chain comprising the amino acid sequence of SEQ ID NO:82, a first light chain comprising the amino acid sequence of SEQ ID NO:83 or SEQ ID NO: 171, a second heavy chain comprising the amino acid sequence of SEQ ID NO:80 and a second light chain comprising the amino acid sequence of SEQ ID NO:81.

22. The TNF family ligand trimer-containing antigen binding molecule of claim 1 comprising two Fab domains capable of specific binding to PD1.

23. The TNF family ligand trimer-containing antigen binding molecule of claim 1, wherein the antigen binding molecule comprises
(a) a first heavy chain comprising the amino acid sequence of SEQ ID NO:84, a second heavy chain comprising the amino acid sequence of SEQ ID NO:85, and two light chains comprising the amino acid sequence of SEQ ID NO:83 or SEQ ID NO: 171, or
(b) a first heavy chain comprising the amino acid sequence of SEQ ID NO:86, a second heavy chain comprising the amino acid sequence of SEQ ID NO:87, and two light chains comprising the amino acid sequence of SEQ ID NO:83 or SEQ ID NO:171.

24. The TNF family ligand trimer-containing antigen binding molecule of claim 1, wherein the antigen binding molecule comprises
(a) a fusion polypeptide comprising the amino acid sequence of SEQ ID NO:88, a heavy chain comprising the amino acid sequence of SEQ ID NO:87 and a light chain comprising the amino acid sequence of SEQ ID NO:83 or SEQ ID NO:171,
(b) a fusion polypeptide comprising the amino acid sequence of SEQ ID NO:89, a heavy chain comprising the amino acid sequence of SEQ ID NO:90 and a light chain comprising the amino acid sequence of SEQ ID NO:83 or SEQ ID NO:171,
(c) a fusion polypeptide comprising the amino acid sequence of SEQ ID NO:91, a heavy chain comprising the amino acid sequence of SEQ ID NO:86 and a light chain comprising the amino acid sequence of SEQ ID NO:83 or SEQ ID NO:171,
(b) a fusion polypeptide comprising the amino acid sequence of SEQ ID NO:93, a heavy chain comprising the amino acid sequence of SEQ ID NO:92 and a light chain comprising the amino acid sequence of SEQ ID NO:83 or SEQ ID NO:171.

25. An isolated polynucleotide encoding the TNF family ligand trimer-containing antigen binding molecule of claim 1.

26. A vector comprising the isolated polynucleotide of claim 25.

27. A host cell comprising the isolated polynucleotide of claim 25.

28. A method of producing an TNF family ligand trimer-containing antigen binding molecule of, comprising culturing the host cell of claim 27 under conditions suitable for expression of the TNF family ligand trimer-containing antigen binding molecule, and isolating the TNF family ligand trimer-containing antigen binding molecule.

29. A pharmaceutical composition comprising the TNF family ligand trimer-containing antigen binding molecule of claim 1 and at least one pharmaceutically acceptable excipient.

30. (canceled)

31. (canceled)

32. (canceled)

33. (canceled)

34. A method of treating an individual having cancer comprising administering to the individual an effective amount of the TNF family ligand trimer-containing antigen binding molecule of claim 1.

35. A method of up-regulating or prolonging cytotoxic T cell activity in an individual having cancer, comprising administering to the individual an effective amount of the TNF family ligand trimer-containing antigen binding molecule of claim 1.
